(19) United States
(12) Patent Application Publication
(10) Pub. No.: US 2026/0055162 A1
(43) Pub. Date: Feb. 26, 2026

(54) CELLULAR THERAPEUTICS ENGINEERED WITH SIGNAL MODULATORS AND METHODS OF USE THEREOF

(71) Applicant: Catamaran Bio, Inc., Cambridge, MA (US)

(72) Inventors: Celeste Richardson, Brookline, MA (US); Luke Barron, Cambridge, MA (US); James Alexander Storer, Medford, MA (US)

(73) Assignee: Catamaran Bio, Inc., Cambridge, MA (US)

(21) Appl. No.: 19/095,896

(22) Filed: Mar. 31, 2025

Related U.S. Application Data
(63) Continuation of application No. 17/535,071, filed on Nov. 24, 2021, now abandoned.
(60) Provisional application No. 63/118,008, filed on Nov. 25, 2020.

(51) Int. Cl. C07K 14/71; A61K 40/15; A61K 40/42; C07K 14/47; C12N 5/0783

(52) U.S. Cl. CPC C07K 14/71; A61K 40/15; A61K 40/42; C07K 14/4705; C12N 5/0646; A61K 2239/31; A61K 2239/38; C07K 2319/03

(57) ABSTRACT

The present disclosure is directed to an engineered protein (e.g., a chimeric protein) comprising one or more of an extracellular domain, a transmembrane domain and/or an intracellular domain, which are capable of binding a negative signal and functioning as a sink, dominant negative, or signal inverter for the negative signal. The disclosure is further directed to methods of generating a modified cell expressing one or more of the engineered proteins (e.g., chimeric proteins), and methods of using the modified cells in treating a disease or a condition in a subject in need thereof.

### CROSS-REFERENCE TO RELATED APPLICATIONS

This application is a continuation of U.S. patent application Ser. No. 17/535,071, filed on Nov. 24, 2021, which claims priority to U.S. Provisional Patent Application Ser. No. 63/118,008, filed Nov. 25, 2020, the entire contents of which are herein incorporated by reference in its entirety.

### SEQUENCE LISTING

This application contains a Sequence Listing that has been submitted electronically as an XML file named 52526-0018002_SL_ST26.xml. The XML file, created on Mar. 31, 2025, is 700,832 bytes in size. The material in the XML file is hereby incorporated by reference in its entirety.

### FIELD

The present invention relates generally to the fields of molecular biology, immunology, oncology, cell therapy, and medicine. More particularly, it concerns a cell expressing an engineered protein (e.g., a chimeric protein) comprising one or more of an extracellular domain, a transmembrane domain and/or an intracellular domain.

### BACKGROUND

Despite expanded appreciation for the diversity of cellular mechanisms fostering solid tumor development, anti-cancer therapy remains heavily reliant on cytotoxic modalities, including chemotherapy and radiation therapy, that kill rapidly proliferating (neoplastic) cells within tumors. Conventional chemotherapy and radiotherapy often produce insufficient benefit, underscoring the need for novel therapeutics. Effective tumor immunotherapy is also hindered by immunological obstacles, such as the ability of tumors to foster a tolerant microenvironment and the activation of a plethora of immunosuppressive mechanisms, which may act in concert to counteract effective immune responses. Genetically engineered immune cells have more recently been used to treat cancer and induce immune responses. The tumor microenvironment is a hostile environment surrounding tumors, which is highly immunosuppressive and a major barrier for cancer therapies to eliminate solid tumors effectively. Immunosuppressive factors, like PD-L1 and TGFβ, produced by the tumor or stromal cells and resident in the tumor microenvironment, suppress the activity of immune cells, thereby limiting the ability of the immune system to act against the invading cancer. Therefore, autologous immune cell therapies are not sufficient for efficiently treating cancers, especially solid cancers.

There is a need for more effective and safer classes of cell therapies that could treat cancer and induce immune responses in vivo, that also overcome the immunological obstacles of the tumor microenvironment. The present disclosure addresses this unmet need.

### SUMMARY

The present disclosure relates to engineered proteins (e.g., chimeric proteins) that are capable, when present on a cell, of inhibiting immunosuppressive signals that exist in the tumor microenvironment. The present disclosure further relates to engineered cells, e.g., immune cells such as natural killer (NK) cells, comprising one or more of said engineered proteins (e.g., chimeric proteins), as well as to methods of using the engineered cells for treating a disease or disorder, such as cancer. The engineered proteins (e.g., chimeric proteins) comprise an extracellular domain, a transmembrane domain, and optionally an intracellular domain, and in some embodiments, are chimeric proteins. These proteins, when present on a cell, such as an NK cell, are capable of inhibiting immunosuppressive signals by binding to the negative signaling molecule and acting as a sink or as a dominant negative receptor, thereby neutralizing the negative signal, or acting as a signal inverter to convert the negative signal that would have otherwise been inhibitory into an activating signal, thereby enhancing the anti-tumor activity of the cell.

The activation of NK cells relies more heavily on, and on a broader repertoire of, signaling-dependent receptors, such as DAP10 and DAP12, in comparison to other types of leukocytes, such as T cells, B cells, monocytes, and macrophages. Therefore, signal inverters (e.g., TGF-βR/DAP10 or TGF-βR/DAP12) that convert negative signals associated with the cell's immunosuppressive activity (e.g., by TGF-βR) to a positive signal, offer a selective advantage to NK cells over other immune cell types. Furthermore, while T cell activation and behavior is highly dependent on TCR engagement, NK cell activation and subsequent target cell killing is, in contrast, determined by a balance of activating and inhibitory signals, and is not as dependent on a single signal. Therefore, while not wishing to be bound by theory, it is believed that incorporating an additional activating signal into NK cells could facilitate a meaningful change in that balance and substantially alter NK cell behavior. It is further postulated that the activating signals provided by, for example, a TGF-βR signal inverter could facilitate intrinsic gains in function of the cell (e.g., NK cell), by preventing antigen escape, by enhancing the expression of endogenous NK activating receptors and/or by promoting a favorable phenotype for anti-tumor activity (e.g., differentiation state) by improving metabolic fitness within the tumor microenvironment. The activating signals provided by, for example, a TGF-βR signal inverter may also facilitate extrinsic gains in function of the cell (e.g., NK cell) by disrupting immunosuppression in the tumor microenvironment, for example, by improving inflammation mediated by chemokines or cytokines, and/or by driving immune activation and epitope spreading with costimulatory ligands or cytokines.

Also provided herein are chimeric proteins that include an extracellular domain, a transmembrane domain, and an intracellular domain, wherein the extracellular domain is capable of binding a negative signal, and wherein the intracellular domain comprises an intracellular domain, or a portion thereof, of a stimulatory polypeptide.

In some embodiments, the chimeric protein is capable of activating an immune cell selected from an NK cell, an NKT cell, a T-cell, and a macrophage.

In some embodiments, the chimeric protein is capable of activating an NK cell.

In some embodiments, the extracellular domain comprises an antigen-binding domain that specifically binds to the negative signal. In some embodiments, the antigen-binding domain comprises a fragment of an antibody. In some embodiments, the antigen-binding domain comprises an scFv, a Fab, or a VHH. In some embodiments, the scFv is an scFv from a monoclonal antibody. In some embodiments, the scFv is connected to the transmembrane domain by a linker.

In some embodiments, the antigen-binding domain specifically binds to a negative signal selected from the group consisting of TGF-β, IL-10, IL-1, IL-6, PD-L1, PD-L2, B7-1, B7-2, MHCII, HVEM, CD155, CD112, CD111, CD200, B7-H6, HS-GAG, HLA, N-cadherin, E-cadherin, FasL, and MHCII.

In some embodiments, the extracellular domain comprises the extracellular domain, or a portion thereof, of an inhibitory polypeptide that binds the negative signal. In some embodiments, the inhibitory polypeptide is an inflammatory mediator receptor, an inhibitory cytokine receptor, an immune checkpoint receptor, or a dual activator-checkpoint receptor.

In some embodiments, the inhibitory polypeptide is selected from the inhibitory polypeptides presented in Table 1 or Table 1.1.

In some embodiments, the transmembrane domain comprises the transmembrane domain, or a portion thereof, of an inhibitory polypeptide presented in Table 1 or Table 1.2.

In some embodiments, the transmembrane domain comprises the transmembrane domain, or a portion thereof, of a stimulatory polypeptide presented in Table 2 or Table 2.2.

In some embodiments, the intracellular domain comprises the intracellular domain, or a portion thereof, of a stimulatory polypeptide selected from the stimulatory polypeptides presented in Table 2 or Table 2.1. In some embodiments, the stimulatory polypeptide is selected from one or more isoforms of the stimulatory polypeptide.

In some embodiments, the chimeric protein comprises the intracellular domain, or a portion thereof, of two or more different stimulatory polypeptides.

In some embodiments, each of the extracellular domain, the transmembrane domain, and the intracellular domain have the same N-terminal to C-terminal orientation.

In some embodiments, the inhibitory polypeptide is a type I receptor, and the stimulatory polypeptide is a type I receptor; the inhibitory polypeptide is a type I receptor, and the stimulatory polypeptide is a type III receptor; the inhibitory polypeptide is a type II receptor, and the stimulatory polypeptide is a type II receptor; the inhibitory polypeptide is a type I receptor, and the stimulatory polypeptide is not associated with the plasma membrane; or the inhibitory polypeptide is a type I receptor, the stimulatory polypeptide is a type II receptor, and the transmembrane domain, or portion thereof, is from a type I receptor.

In some embodiments, the inhibitory polypeptide is capable of forming a dimer and the stimulatory polypeptide is capable of forming a dimer; or wherein the inhibitory polypeptide is capable of forming a trimer and the stimulatory polypeptide is capable of forming a trimer.

In some embodiments, a combination of the extracellular domain, or a portion thereof, of an inhibitory polypeptide and the intracellular domain, or a portion thereof, of a stimulatory polypeptide is selected from the combinations presented in any one of Tables 6-14.

In some embodiments, the extracellular domain and the transmembrane domain are connected by a linker. In some embodiments, the linker is selected from the linkers presented in Table 3.

In some embodiments, the transmembrane domain and the intracellular domain are connected by a linker. In some embodiments, the linker is selected from the linkers presented in Table 3.

Also provided herein are modified immune cells engineered to express a chimeric protein comprising an extracellular domain, a transmembrane domain, and an intracellular domain, wherein the extracellular domain is capable of binding a negative signal, and wherein the intracellular domain comprises an intracellular domain, or a portion thereof, of a stimulatory polypeptide.

In some embodiments, the immune cell is selected from the group consisting of an NK cell, an NKT cell, a T-cell, and a macrophage. In some embodiments, the immune cell is an NK cell.

In some embodiments, the extracellular domain comprises an antigen-binding domain that specifically binds to the negative signal. In some embodiments, the antigen-binding domain comprises a fragment of an antibody. In some embodiments, the antigen-binding domain comprises an scFv, a Fab, or a VHH. In some embodiments, the scFv is an scFv from a monoclonal antibody. In some embodiments, the scFv is connected to the transmembrane domain by a linker.

In some embodiments, the antigen-binding domain specifically binds to a negative signal selected from the group consisting of TGF-β, IL-10, IL-1, IL-6, PD-L1, PD-L2, B7-1, B7-2, MHCII, HVEM, CD155, CD112, CD111, CD200, B7-H6, HS-GAG, HLA, N-cadherin, E-cadherin, FasL, and MHCII.

In some embodiments, the extracellular domain comprises the extracellular domain, or a portion thereof, of an inhibitory polypeptide. In some embodiments, the inhibitory polypeptide is an inflammatory mediator receptor, an inhibitory cytokine receptor, an immune checkpoint receptor, or a dual activator-checkpoint receptor. In some embodiments, the inhibitory polypeptide is selected from the inhibitory polypeptides presented in Table 1 or Table 1.1.

In some embodiments, the transmembrane domain comprises the transmembrane domain, or a portion thereof, of an inhibitory polypeptide presented in Table 1 or Table 1.2.

In some embodiments, the transmembrane domain comprises the transmembrane domain, or a portion thereof, of a stimulatory polypeptide presented in Table 2 or Table 2.2.

In some embodiments, the intracellular domain comprises the intracellular domain, or a portion thereof, of a stimulatory polypeptide selected from the stimulatory polypeptides presented in Table 2 or Table 2.1.

In some embodiments, the chimeric protein comprises the intracellular domain, or a portion thereof, of two or more different stimulatory polypeptides.

In some embodiments, the extracellular domain and the transmembrane domain are connected with a linker. In some embodiments, the linker is selected from the linkers presented in Table 3.

In some embodiments, the transmembrane domain and the intracellular domain are connected with a linker. In some embodiments, the linker is selected from the linkers presented in Table 3.

In some embodiments, the immune cell is engineered to further comprise a chimeric antigen receptor (CAR). In some embodiments, the CAR targets a tumor antigen.

In some embodiments, the immune cell is engineered to further comprise a cytokine. In some embodiments, the cytokine can be selected from the group consisting of a chemokine, an interferon, an interleukin, a lymphokine, a tumor necrosis factor, or a variant or combination thereof. In some embodiments, the cytokine is an IL-15 or a fragment or variant thereof.

Also provided herein are chimeric proteins that include an extracellular domain, a transmembrane domain, and an intracellular domain, wherein the extracellular domain binds to TGF-β, and wherein the intracellular domain comprises an intracellular domain, or a portion thereof, of a stimulatory polypeptide.

In some embodiments, the chimeric protein is capable of activating an immune cell selected from the group consisting of an NK cell, an NKT cell, a T-cell, and a macrophage.

In some embodiments, the chimeric protein is capable of activating an NK cell.

In some embodiments, the extracellular domain comprises an antigen-binding domain that specifically binds TGF-β. In some embodiments, the antigen-binding domain comprises a fragment of an antibody. In some embodiments, the antigen-binding domain comprises an scFv, a Fab, or a VHH. In some embodiments, the scFv is an scFv from a monoclonal antibody. In some embodiments, the scFv is connected to the transmembrane domain by a linker.

In some embodiments, the extracellular domain comprises the extracellular domain, or a portion thereof, of a TGF-β receptor polypeptide.

In some embodiments, the extracellular domain comprises the extracellular domain, or a portion thereof, of a TGF-βR1 polypeptide or a TGF-βR2 polypeptide presented in Table 1.1.

In some embodiments, the intracellular domain comprises the intracellular domain, or a portion thereof, of a stimulatory polypeptide selected from the stimulatory polypeptides presented in Table 2 or Table 2.1.

In some embodiments, the intracellular domain comprises the intracellular domain, or a portion thereof, of two or more different stimulatory polypeptides.

In some embodiments, the stimulatory polypeptide is selected from the group consisting of DAP10, DAP12, 2B4, CD2, LFA1, IL-21, and PILRB.

In some embodiments, the stimulatory polypeptide is not one or more of BMP, IL-1, IL-2, IL-7, IL-15, IL-21, IL-12, IL-18, IL-19, IFN-gamma, TLR1, TLR2, TLR3, TLR4, TLR5, TLR6, TLR7, TLR8, TLR9, CD28, 4-1BB, OX40, CD3 (CD3zeta), CD40, CD27, IL-12R, IL-7R, CD137, and ICOS.

In some embodiments, the stimulatory polypeptide is not DAP12.

In some embodiments, the extracellular domain further comprises at least a portion of an extracellular domain of the stimulatory polypeptide.

In some embodiments, the transmembrane domain comprises the transmembrane domain, or a portion thereof, of a stimulatory polypeptide presented in Table 2 or Table 2.2.

In some embodiments, the transmembrane domain comprises the transmembrane domain, or a portion thereof, of the TGF-β receptor.

In some embodiments, the transmembrane domain comprises the transmembrane domain, or a portion thereof, of the stimulatory polypeptide.

In some embodiments, the transmembrane domain and the stimulatory polypeptide are respectively selected from the group consisting of: DAP12 and DAP12; TGF-β R2 and DAP12; 2B4 and 2B4; TGF-β R2 and 2B4; LFA1 and LFA1; TGF-β R2 and LFA1; CD2 and CD2; TGF-β R2 and CD2; CD28 and CD28+CD3zeta; and CD28H and CD28H+CD3zeta.

In some embodiments, the extracellular domain and transmembrane domain are connected by a linker. In some embodiments, the linker is selected from the linkers presented in Table 3.

In some embodiments, the transmembrane domain and intracellular domain are connected by a linker. In some embodiments, the linker is selected from the linkers presented in Table 3.

Also provided herein are modified immune cells engineered to express a chimeric protein comprising an extracellular domain, a transmembrane domain, and an intracellular domain, wherein the extracellular domain binds to TGF-β, and wherein the intracellular domain comprises an intracellular domain, or a portion thereof, of a stimulatory polypeptide.

In some embodiments, the immune cell is selected from the group consisting of an NK cell, an NKT cell, a T-cell, and a macrophage. In some embodiments, the immune cell is an NK cell.

In some embodiments, the intracellular domain comprises the intracellular domain, or a portion thereof, of a stimulatory polypeptide that promotes activation of an NK cell.

In some embodiments, the binding of the extracellular domain to TGF-β activates the immune cell.

In some embodiments, the extracellular domain comprises an antigen-binding domain that specifically binds TGF-β. In some embodiments, the antigen-binding domain comprises a fragment of an antibody. In some embodiments, the antigen-binding domain comprises an scFv, a Fab, or a VHH. In some embodiments, the scFv is an scFv from a monoclonal antibody.

In some embodiments, the extracellular domain comprises the extracellular domain, or a portion thereof, of a TGF-β receptor (TGF-βR or TGF-βR) polypeptide.

In some embodiments, the extracellular domain comprises the extracellular domain, or a portion thereof, of a TGF-βR1 polypeptide or a TGF-βR2 polypeptide presented in Table 1.1.

In some embodiments, the intracellular domain comprises the intracellular domain, or a portion thereof, of a stimulatory polypeptide selected from the stimulatory polypeptides presented in Table 2 or Table 2.1.

In some embodiments, the intracellular domain comprises the intracellular domain, or a portion thereof, of two or more different stimulatory polypeptides.

In some embodiments, the stimulatory polypeptide is DAP10, DAP12, 2B4, CD2, LFA1, IL-21, or PILRB.

In some embodiments, the stimulatory polypeptide is not one or more of BMP, IL-1, IL-2, IL-7, IL-15, IL-21, IL-12, IL-18, IL-19, IFN-gamma, TLR1, TLR2, TLR3, TLR4, TLR5, TLR6, TLR7, TLR8, TLR9, CD28, 4-1BB, OX40, CD3 (CD3zeta), CD40, CD27, IL-12R, IL-7R, CD137, and ICOS.

In some embodiments, the stimulatory polypeptide is not DAP12.

In some embodiments, the transmembrane domain comprises the transmembrane domain, or a portion thereof, of a stimulatory polypeptide presented in Table 2 or Table 2.2.

In some embodiments, the transmembrane domain comprises the transmembrane domain, or a portion thereof, of the TGF-β receptor (TGF-βR).

In some embodiments, the transmembrane domain comprises the transmembrane domain, or a portion thereof, of the stimulatory polypeptide.

In some embodiments, the transmembrane domain and the stimulatory polypeptide are respectively selected from the group consisting of: DAP12 and DAP12; TGF-β R2 and DAP12; 2B4 and 2B4; TGF-β R2 and 2B4; LFA1 and LFA1; TGF-β R2 and LFA1; CD2 and CD2; TGF-β R2 and CD2; CD28 and CD28+CD3zeta; and CD28H and CD28H+CD3zeta.

In some embodiments, the immune cell is engineered to further comprise a chimeric antigen receptor (CAR). In some embodiments, the CAR targets a tumor antigen.

In some embodiments, the immune cell is engineered to further comprise a cytokine. In some embodiments, the cytokine can be selected from the group consisting of a chemokine, an interferon, an interleukin, a lymphokine, a tumor necrosis factor, or a variant or combination thereof. In some embodiments, the cytokine is an IL-15 or a fragment or variant thereof.

Also provided herein are proteins that include an extracellular domain and a transmembrane domain, wherein the extracellular domain is capable of binding a negative signal, and wherein the protein lacks a fully functional intracellular domain.

In some embodiments, the protein lacks an intracellular domain.

In some embodiments, the extracellular domain comprises an antigen-binding domain that specifically binds to the negative signal. In some embodiments, the antigen-binding domain comprises a fragment of an antibody. In some embodiments, the antigen-binding domain comprises an scFv, a Fab, or a VHH. In some embodiments, the scFv is an scFv from a monoclonal antibody. In some embodiments, the scFv is connected to the transmembrane domain by a linker.

In some embodiments, the extracellular domain comprises the extracellular domain, or a portion thereof, of an inhibitory polypeptide that binds to the negative signal. In some embodiments, the inhibitory polypeptide is an inflammatory mediator receptor, an inhibitory cytokine receptor, an immune checkpoint receptor, or a dual activator-checkpoint receptor.

In some embodiments, the inhibitory polypeptide is selected from the inhibitory polypeptides presented in Table 1 or Table 1.1.

In some embodiments, the transmembrane domain comprises the transmembrane domain, or a portion thereof, of an inhibitory polypeptide presented in Table 1 or Table 1.2.

In some embodiments, the transmembrane domain comprises the transmembrane domain, or a portion thereof, of a stimulatory polypeptide presented in Table 2 or Table 2.2.

In some embodiments, the extracellular domain and the transmembrane domain are connected by a linker. In some embodiments, the linker is selected from the linkers presented in Table 3.

In some embodiments, the transmembrane domain comprises the transmembrane domain, or a portion thereof, of a different polypeptide than the extracellular domain. In some embodiments, the different polypeptide is selected from the inhibitory polypeptides presented in Table 1 or Table 1.2; or the stimulatory polypeptides presented in Table 2 or Table 2.2.

Also provided herein are modified cells engineered to express a protein comprising an extracellular domain and a transmembrane domain, wherein the extracellular domain is capable of binding a negative signal, and wherein the chimeric protein lacks a fully functional intracellular domain.

In some embodiments, the cell is selected from the group consisting of an artificial cell, an immune cell, a fibrocyte, a mesenchymal stem cell, an induced neural stem cell, or an induced pluripotent stem cell (iPSC)-derived cell, and an erythrocyte. In some embodiments, the immune cell is selected from the group consisting of a T cell, an NK cell, an NKT cell (e.g., an invariant NKT (iNKT) cell), a type 1 innate lymphoid cell (ILC1), an intraepithelial type 1 innate lymphoid cell (ieILC1), a type 2 innate lymphoid cell (ILC2), a type 3 innate lymphoid cell (ILC3), a lymphoid tissue inducer cell (LTi), a monocyte, a macrophage, a dendritic cell (DC), a platelet, a marrow-infiltrating lymphocyte (MIL), and a B cell. In some embodiments, the immune cell is an NK cell.

In some embodiments, the chimeric protein lacks an intracellular domain.

In some embodiments, the extracellular domain comprises an antigen-binding domain that specifically binds to the negative signal. In some embodiments, the antigen-binding domain comprises a fragment of an antibody. In some embodiments, the antigen-binding domain comprises an scFv, a Fab, or a VHH. In some embodiments, the scFv is an scFv from a monoclonal antibody. In some embodiments, the extracellular domain does not comprise an antigen-binding domain (e.g., an antibody or a fragment thereof, scFv, Fab, and a VHH).

In some embodiments, the extracellular domain comprises the extracellular domain, or a portion thereof, of an inhibitory polypeptide that binds to the negative signal. In some embodiments, the inhibitory polypeptide is an inflammatory mediator receptor, an inhibitory cytokine receptor, an immune checkpoint receptor, or a dual activator-checkpoint receptor.

In some embodiments, the inhibitory polypeptide is selected from the inhibitory polypeptides presented in Table 1 or Table 1.1.

In some embodiments, the transmembrane domain comprises the transmembrane domain, or a portion thereof, of an inhibitory polypeptide presented in Table 1 or Table 1.2.

In some embodiments, the transmembrane domain comprises the transmembrane domain, or a portion thereof, of a stimulatory polypeptide presented in Table 2 or Table 2.2.

In some embodiments, the extracellular domain and the transmembrane domain are connected by a linker. In some embodiments, the linker is selected from the linkers presented in Table 3.

In some embodiments, the immune cell is engineered to further comprise a chimeric antigen receptor (CAR). In some embodiments, the CAR targets a tumor antigen.

In some embodiments, the immune cell is engineered to further comprise a cytokine. In some embodiments, the cytokine can be selected from the group consisting of a chemokine, an interferon, an interleukin, a lymphokine, a tumor necrosis factor, or a variant or combination thereof. In some embodiments, the cytokine is an IL-15 or a fragment or variant thereof.

Also provided herein are modified cells engineered to express a protein comprising a dominant negative isoform of a protein, wherein the dominant negative isoform of the protein competes with a wild-type isoform of the protein for binding a negative signal.

In some embodiments, the cell is selected from the group consisting of an artificial cell, an immune cell, a fibrocyte, a mesenchymal stem cell, an induced neural stem cell, and an induced pluripotent stem cell (iPSC)-derived cell, and an erythrocyte. In some embodiments, the immune cell is a tumor infiltrating lymphocyte (TIL). In some embodiments, the immune cell is selected from the group consisting of a T cell, an NK cell, an NKT cell, a type 1 innate lymphoid cell (ILC1), an intraepithelial type 1 innate lymphoid cell (ieILC1), a type 2 innate lymphoid cell (ILC2), a type 3 innate lymphoid cell (ILC3), a lymphoid tissue inducer cell (LTi), a monocyte, a macrophage, a dendritic cell (DC), a platelet, a marrow-infiltrating lymphocyte (MIL), and a B cell. In some embodiments, the immune cell is an NK cell.

In some embodiments, the dominant negative isoform of the protein is a dominant negative isoform of an inhibitory polypeptide selected from Table 1 or Table 1.1.

In some embodiments, the dominant negative isoform of the protein is a dominant negative isoform of TGF-BR1. In some embodiments, the dominant negative isoform of TGF-BR1 is selected from the dominant negative isoforms of TGF-BR1 presented in Table 4.

In some embodiments, the immune cells comprising a CAR described herein are T cells (e.g., alpha beta T cells and gamma delta T cells). In some embodiments, the T cells are one or more of CD3⁺, CD28⁺, CD4⁺, CD8⁺, CD45RA⁺, CD25⁺ and CD45RO⁺. In some embodiments, the T cells are isolated tumor infiltrating lymphocytes (TIL). In some embodiments, the T cells are CD4⁺ T cells. In some embodiments, the T cells are CD8⁺ T cells. In some embodiments, the T cells are regulatory T cell (e.g., a CD4⁺, CD25⁺, CD62L^{hi}, GITR⁺ and FoxP3⁺ T cells). In some embodiments, the T cells are memory T cells (T_{CM}) (e.g., CD62L⁺, CCR7⁺, CD45RO⁻ and CD45RA⁻). In some embodiments, the T cells are stem cell memory T cells. In some embodiments, the T cells are naïve T cells. In some embodiments, the T cells are a mixed population of CD4⁺ T cells, CD8⁺ T cells, stem cell memory T cells and naïve T cells. In some embodiments, the immune cells comprising a protein described herein (e.g., a CAR) are natural killer T (NKT) cells. NKT cells recognize glycolipid antigen presented by a molecule called CD1d.

In some embodiments, the dominant negative isoform of the protein is a dominant negative isoform of TGF-BR2. In some embodiments, the dominant negative isoform of TGF-BR2 is selected from the dominant negative isoforms of TGF-BR2 presented in Table 5.

In some embodiments, the immune cell is engineered to further comprise a chimeric antigen receptor (CAR). In some embodiments, the CAR targets a tumor antigen. In some embodiments, the immune cell is engineered to further comprise a cytokine. In some embodiments, the cytokine can be selected from the group consisting of a chemokine, an interferon, an interleukin, a lymphokine, a tumor necrosis factor, or a variant or combination thereof. In some embodiments, the cytokine is an IL-15 or a fragment or variant thereof.

Also provided herein are modified cells engineered to express at least two proteins selected from the group consisting of: (a) a chimeric protein comprising an extracellular domain, a transmembrane domain, and an intracellular domain, wherein the extracellular domain is capable of binding a negative signal, and wherein the intracellular domain comprises an intracellular domain, or a portion thereof, of a stimulatory polypeptide; (b) a protein comprising a dominant negative isoform of a protein, wherein the dominant negative isoform of the protein competes with a wild-type isoform of the protein for binding a negative signal that prevents or decreases the activation of an immune response; and (c) a protein comprising an extracellular domain and a transmembrane domain, wherein the extracellular domain is capable of binding a negative signal, and wherein the protein lacks a fully functional intracellular domain.

In some embodiments, the cell is selected from the group consisting of an artificial cell, an immune cell, a fibrocyte, a mesenchymal stem cell, an induced neural stem cell, and an induced pluripotent stem cell (iPSC)-derived cell. In some embodiments, the immune cell is selected from the group consisting of a T cell, an NK cell, an NKT cell, a type 1 innate lymphoid cell (ILC1), an intraepithelial type 1 innate lymphoid cell (ieILC1), a type 2 innate lymphoid cell (ILC2), a type 3 innate lymphoid cell (ILC3), a lymphoid tissue inducer cell (LTi), a monocyte, a macrophage, a dendritic cell (DC), a platelet, a marrow-infiltrating lymphocyte (MIL), and a B cell. In some embodiments, the immune cell is a tumor infiltrating lymphocyte (TIL). In some embodiments, the immune cell is an NK cell.

Also provided herein are polynucleotides that include a nucleic acid sequence encoding any of the chimeric proteins, or engineered proteins (e.g., chimeric proteins) described herein.

Also provided herein are pharmaceutical compositions that include any of the modified cells described herein, and a pharmaceutically acceptable excipient.

Also provided herein are methods of treating a subject in need of an altered immune response that include administering to the subject an effective amount of a composition comprising any of the modified cells described herein, thereby treating the subject in need of the altered immune response.

Also provided herein are methods of treating a disease or pathological condition in a subject that include administering to the subject an effective amount of a composition comprising any of the modified cell described herein, thereby treating the disease or pathological condition in the subject.

Also provided herein are methods of treating a cancer in a subject that include administering to the subject a therapeutically effective amount of a composition comprising any of the modified cells described herein, thereby treating the cancer in the subject.

Also provided herein are methods of generating any of the modified cells described herein that include: (a) introducing a nucleic acid encoding any of the chimeric proteins or engineered proteins (e.g., chimeric proteins) described herein, into a cell; (b) culturing the cell under conditions allowing the expression of the protein in or on the cell; and (c) recovering the cell from the culture, thereby generating the modified cell.

Also provided herein are cells obtained by the methods described herein.

Also provided herein are kits that include any of the chimeric proteins or any of the engineered proteins (e.g., chimeric proteins) described herein, any of the modified cells described herein, and/or any of the nucleic acids encoding any of the chimeric proteins or the engineered proteins described herein.

### BRIEF DESCRIPTION OF THE DRAWINGS

FIG. 1 is a general schematic of the sink, dominant negative, and signal inverter modalities of the disclosure.

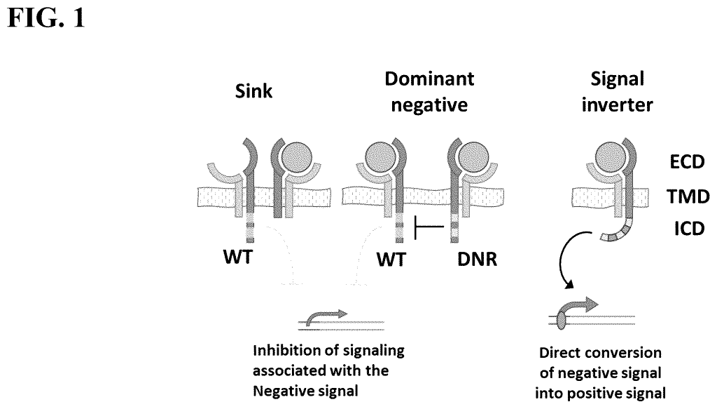

FIG. 2 is a schematic of an exemplary protein of the sink modality of the disclosure.

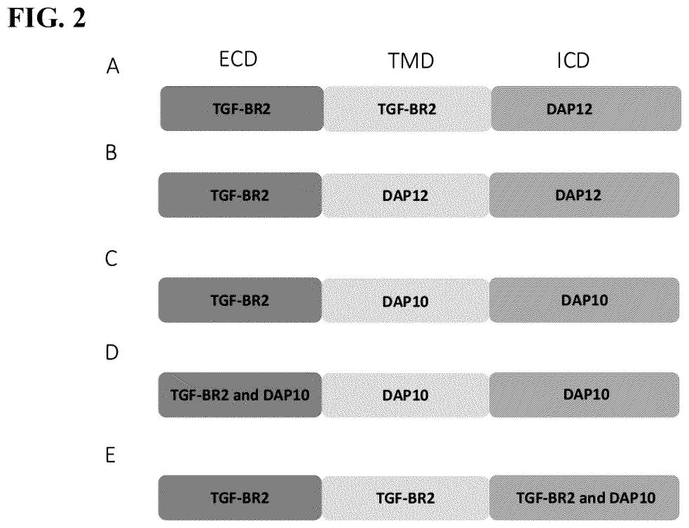

FIG. 3 is a schematic of an exemplary protein of the dominant negative receptor modality of the disclosure.

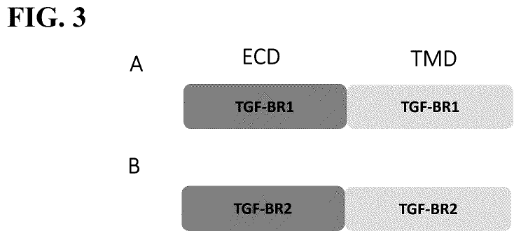

FIG. 4 is a schematic of an exemplary chimeric protein of the signal inverter modality of the disclosure.

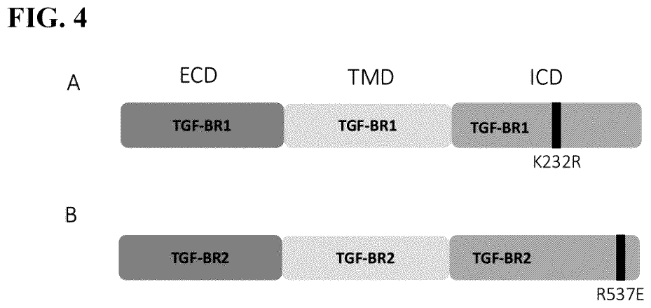

FIG. 5 is a graph showing that stimulation of reporter cells expressing different chimeric proteins including the extracellular domains of inhibitory receptors induces NF-κB activation.

FIG. 6 is a graph showing that stimulation of reporter cells expressing different chimeric proteins including the extracellular domains of inhibitory receptors induces CD69 expression.

FIG. 7A is a graph showing that TGF-B1 stimulation of reporter cells expressing different chimeric proteins including the extracellular domain of TGF-BR2 induces NF-κB activation.

FIG. 7B is a graph showing that TGF-B1 stimulation of reporter cells expressing different chimeric proteins including the extracellular domain of TGF-BR2 induces CD69 expression.

FIG. 8 is a graph showing the fold expansion of NK cells expressing chimeric proteins including the extracellular domain of TGF-BR2.

FIG. 9 is a graph showing interferon gamma cytokine production in NK cells expressing different chimeric proteins including the extracellular domain of TGF-BR2.

FIG. 10 is a graph showing IP-10 production in NK cells expressing different chimeric proteins including the extracellular domain of TGF-BR2.

FIG. 11 is a graph showing the cytotoxicity against SKOV-3 target cells by NK cells expressing different chimeric proteins including the extracellular domain of TGF-BR2.

### DETAILED DESCRIPTION

The present disclosure overcomes problems associated with current technologies by providing engineered cells (e.g., immune cells, such as NK cells) for cell-based therapies, such as adoptive immunotherapy, for the treatment of diseases including cancer.

In some embodiments, provided herein are modified immune cells engineered to express a chimeric protein, where contacting the modified immune cells with a negative signal that binds to the extracellular domain of the chimeric protein results in the increased activation of nuclear factor kappa B (NF-κB) activity, activator protein 1 (AP-1) activity, nuclear factor of activated T-cells (NFAT) activity, and a signal transducer and activator of transcription protein (STAT; e.g., STAT1, STAT3, STAT4, STAT5, and/or STAT6) activity in the cell, e.g., as compared to a wildtype immune cell or a modified immune cell not contacted with the negative signal. In some embodiments, the modified immune cells engineered to express a chimeric protein, where expression of the chimeric protein results in the increased activation of NF-κB activity, AP-1 activity, NFAT activity, and STAT (e.g., STAT1, STAT3, STAT4, STAT5, and/or STAT6) activity in the cell, e.g., as compared to a wildtype immune cell.

In some embodiments, provided herein are modified immune cells engineered to express a chimeric protein, where contacting the modified immune cells with a negative signal that binds to the extracellular domain of the chimeric protein results in increased production levels and/or secretion levels of (e.g., at least a 0.1-fold, at least a 1-fold, at least a 5-fold, at least a 10-fold, at least a 20-fold, at least a 30-fold, at least a 40-fold, at least a 50-fold, at least a 60-fold, at least a 80-fold, or at least a 100-fold increase, or about a 0.1-fold to about a 100-fold, about a 0.1-fold to about a 80-fold, about a 0.1-fold to about a 60-fold, about a 0.1-fold to about a 50-fold, about a 0.1-fold to about a 40-fold, about a 0.1-fold to about a 30-fold, about a 0.1-fold to about a 20-fold, about a 0.1-fold to about a 10-fold, about a 0.1-fold to about a 5-fold, about a 1-fold to about a 100-fold, about a 1-fold to about a 80-fold, about a 1-fold to about a 60-fold, about a 1-fold to about a 50-fold, about a 1-fold to about a 40-fold, about a 1-fold to about a 30-fold, about a 1-fold to about a 20-fold, about a 1-fold to about a 10-fold, about a 1-fold to about a 5-fold, about a 5-fold to about a 100-fold, about a 5-fold to about a 80-fold, about a 5-fold to about a 60-fold, about a 5-fold to about a 50-fold, about a 5-fold to about a 40-fold, about a 5-fold to about a 30-fold, about a 5-fold to about a 20-fold, about a 5-fold to about a 10-fold, about a 10-fold to about a 100-fold, about a 10-fold to about a 80-fold, about a 10-fold to about a 60-fold, about a 10-fold to about a 50-fold, about a 10-fold to about a 40-fold, about a 10-fold to about a 30-fold, about a 10-fold to about a 20-fold, about a 20-fold to about a 100-fold, about a 20-fold to about a 80-fold, about a 20-fold to about a 60-fold, about a 20-fold to about a 50-fold, about a 20-fold to about a 40-fold, about a 20-fold to about a 30-fold, about a 30-fold to about a 100-fold, about a 30-fold to about a 80-fold, about a 30-fold to about a 60-fold, about a 30-fold to about a 50-fold, about a 30-fold to about a 40-fold, about a 40-fold to about a 100-fold, about a 40-fold to about a 80-fold, about a 40-fold to about a 60-fold, about a 40-fold to about a 50-fold, about a 50-fold to about a 100-fold, about a 50-fold to about a 80-fold, about a 50-fold to about a 60-fold, about a 60-fold to about a 100-fold, about a 60-fold to about a 80-fold, or about a 80-fold to about a 100-fold)) of one or more (e.g., two, three, four, five, six, or seven) cytokines selected from the group of interferon-gamma, IL-10, TNF-alpha, IL-8, IP-10, MCP-1, MIP-1a, and MIP-1b and/or CD69 expression by the cells, e.g., as compared to a wildtype immune cell or a modified immune cell not contacted with the negative signal. In some embodiments, the modified immune cells engineered to express a chimeric protein, where expression of the chimeric protein results in the increased production levels and/or secretion levels (e.g., at least a 0.1-fold, at least a 1-fold, at least a 5-fold, at least a 10-fold, at least a 20-fold, at least a 30-fold, at least a 40-fold, at least a 50-fold, at least a 60-fold, at least a 80-fold, or at least a 100-fold increase, or about a 0.1-fold to about a 100-fold (or any of the subranges of this range described herein)) of one or more (e.g., two, three, four, five, six, or seven) cytokines selected from the group of interferon-gamma, IL-10, TNF-alpha, IL-8, IP-10, MCP-1, MIP-1a, and MIP-1b and/or CD69 expression by the cells (e.g., in the absence of a negative signal that binds to the extracellular domain of the chimeric protein), e.g., as compared to a wildtype immune cell.

In some embodiments, provided herein are modified immune cells engineered to express a chimeric protein, where contacting the modified immune cells with a negative signal that binds to the extracellular domain of the chimeric protein results in an increased level (e.g., at least a 0.1-fold, at least a 1-fold, at least a 5-fold, at least a 10-fold, at least a 20-fold, at least a 30-fold, at least a 40-fold, at least a 50-fold, at least a 60-fold, at least a 80-fold, or at least a 100-fold increase, or about a 0.1-fold to about a 100-fold (or any of the subranges of this range described herein)) of cytotoxicity (e.g., percent killing) against target cells (e.g., target cancer cells) by the modified immune cell, e.g., as compared to a wildtype immune cell or a modified immune cell not contacted with the negative signal. In some embodiments, the modified immune cells engineered to express a chimeric protein, where expression of the chimeric protein results in an increased level (e.g., at least a 0.1-fold, at least a 1-fold, at least a 5-fold, at least a 10-fold, at least a 20-fold, at least a 30-fold, at least a 40-fold, at least a 50-fold, at least a 60-fold, at least a 80-fold, or at least a 100-fold increase, or about a 0.1-fold to about a 100-fold (or any of the subranges of this range described herein)) of cytotoxicity (e.g., percent killing) against target cells (e.g., target cancer cells) by the modified immune cells (e.g., in the absence of a negative signal that binds to the extracellular domain of the chimeric protein), e.g., as compared to a wildtype immune cell.

In some embodiments, provided herein are modified immune cells engineered to express a chimeric protein, where contacting the modified immune cells with a negative signal that binds to the extracellular domain of the chimeric protein results in increased proliferation (e.g., expansion) and/or survival of the cells (e.g., in vivo or in vitro) e.g., as compared to a wildtype immune cell or a modified immune cell not contacted with the negative signal. In some embodiments, the modified immune cells engineered to express a chimeric protein, where expression of the chimeric protein results in increase proliferation (e.g., expansion) and/or survival of the cells (e.g., in vivo or in vitro) (e.g., in the absence of a negative signal that binds to the extracellular domain of the chimeric protein), e.g., as compared to a wildtype immune cell or a modified immune cell not contacted with the negative signal.

In some embodiments, provided herein are modified immune cells engineered to express a chimeric protein, where expression of the chimeric protein results in an increase (e.g., at least a 0.1-fold, at least a 1-fold, at least a 5-fold, at least a 10-fold, at least a 20-fold, at least a 30-fold, at least a 40-fold, at least a 50-fold, at least a 60-fold, at least a 80-fold, or at least a 100-fold increase, or about a 0.1-fold to about a 100-fold (or any of the subranges of this range described herein)) in the expansion (e.g., in vivo or in vitro) of the immune cell in the presence of a negative signal, e.g., as compared to a wildtype immune cell or the modified immune cell in the absence of the negative signal. In some embodiments, provided herein are modified immune cells engineered to express a chimeric protein, where expression of the chimeric protein results in an increase (e.g., at least a 0.1-fold, at least a 1-fold, at least a 5-fold, at least a 10-fold, at least a 20-fold, at least a 30-fold, at least a 40-fold, at least a 50-fold, at least a 60-fold, at least a 80-fold, or at least a 100-fold increase, or about a 0.1-fold to about a 100-fold (or any of the subranges of this range described herein)) in the expansion (e.g., in vivo or in vitro) of the immune cell (e.g., in the absence of a negative signal that binds to the extracellular domain of the chimeric protein), e.g., as compared to a wildtype immune cell.

In some embodiments, provided herein are modified immune cells engineered to express a chimeric protein, where expression of the chimeric protein results in an increase (e.g., at least a 0.1-fold, at least a 1-fold, at least a 5-fold, at least a 10-fold, at least a 20-fold, at least a 30-fold, at least a 40-fold, at least a 50-fold, at least a 60-fold, at least a 80-fold, or at least a 100-fold increase, or about a 0.1-fold to about a 100-fold (or any of the subranges of this range described herein)) in the proliferation (e.g., in vivo or in vitro) of the immune cell in the presence of a negative signal, e.g., as compared to a wildtype immune cell or the modified immune cell in the absence of the negative signal. In some embodiments, provided herein are modified immune cells engineered to express a chimeric protein, where expression of the chimeric protein results in an increase (e.g., at least a 0.1-fold, at least a 1-fold, at least a 5-fold, at least a 10-fold, at least a 20-fold, at least a 30-fold, at least a 40-fold, at least a 50-fold, at least a 60-fold, at least a 80-fold, or at least a 100-fold increase, or about a 0.1-fold to about a 100-fold (or any of the subranges of this range described herein)) in the proliferation (e.g., in vivo or in vitro) of the immune cell (e.g., in the absence of a negative signal that binds to the extracellular domain of the chimeric protein), e.g., as compared to a wildtype immune cell.

### I. Definitions

As used in this specification and the appended claims, the singular forms “a”, “an” and “the” include plural references unless the content clearly dictates otherwise.

The use of the alternative (e.g., “or”) should be understood to mean either one, both, or any combination thereof of the alternatives.

As used herein, the term “about,” when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed methods.

As used herein, any concentration range, percentage range, ratio range, or integer range is to be understood to include the value of any integer within the recited range and, when appropriate, fractions thereof (such as one tenth and one hundredth of an integer), unless otherwise indicated.

As used herein, “comprise,” “comprising,” “comprises,” and “comprised of” are meant to be synonymous with “include,” “including.” “includes,” “contain,” “containing,” or “contains” and are inclusive or open-ended terms that specify the presence of what follows, e.g., component, and do not exclude or preclude the presence of additional, non-recited components, features, element, members, steps, known in the art or disclosed therein.

As used herein, the terms “such as,” “for example,” and the like are intended to refer to exemplary embodiments and not to limit the scope of the present disclosure.

Unless defined otherwise, all technical and scientific terms used herein have the same meaning as commonly understood by one of ordinary skill in the art to which the disclosure pertains. Although any methods and materials similar or equivalent to those described herein can be used in the practice for testing of embodiments of the present disclosure, preferred materials and methods are described herein.

As used herein, the term “chimeric protein” refers to any single polypeptide unit that comprises at least two distinct polypeptide domains, wherein the two domains are not naturally occurring within the same polypeptide unit. Typically, such chimeric proteins are made by expression of a cDNA construct, but could be made by protein synthesis methods known in the art. A domain, for example, can be a contiguous primary amino acid sequence in a protein.

The terms “polypeptide” and “protein” are used interchangeably herein.

As used herein, the term “chimeric antigen receptor” or “CAR” refers to engineered receptors (e.g., chimeric receptors), which graft a specificity (e.g., a selected specificity) onto a cell. CARs typically comprise an extracellular domain (which comprises an antigen-binding domain), a transmembrane domain, and an intracellular domain.

The term “exogenous,” when used in relation to a protein, gene, nucleic acid, or polynucleotide in a cell or organism, refers to a protein, gene, nucleic acid, or polynucleotide that has been introduced into the cell or organism by artificial or natural means; or in relation to a cell, the term refers to a cell that was isolated and subsequently introduced to other cells or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from where it would be in natural cells (e.g., a wild-type cell) or is otherwise flanked by a different nucleic acid sequence than that found in nature.

As used herein, the term “expression construct” or “expression cassette” is used to mean a nucleic acid molecule that is capable of directing transcription. An expression construct includes, at a minimum, one or more transcriptional control elements (such as promoters, enhancers, or a structure functionally equivalent thereof) that direct gene expression in one or more desired cell types, tissues, or organs. Additional elements, such as a transcription termination signal, may also be included.

As used herein, the term “extracellular domain” refers to the fragment or portion of a receptor or protein that is generally present on the outside of a cell (e.g., following cellular processing). In some embodiments, the extracellular domain of a receptor or polypeptide includes a ligand binding or recognition domain. The extracellular domain of a receptor may be identified, for example, using databases known in the art, e.g., UNIPROT.

As used herein, the term “intracellular domain” refers to the fragment or portion of a receptor or protein that is generally present on the inside (e.g., the cytoplasm) of a cell and mediates activation of at least one effector function. The term “effector function” refers to a specialized function of a cell. Effector function of an NK cell, for example, may be its cytolytic activity including the secretion of cytokines. Effector function of a T cell, for example, may be cytolytic activity or helper activity including the secretion of cytokines. Thus, the term intracellular domain refers to the portion of a protein which transduces the effector function signal and directs the cell to perform a specialized function. The intracellular domain of a signaling receptor may include a signaling domain, a protein interaction domain, an enzymatic domain, or a combination thereof. While the entire intracellular domain of a source protein can be employed, in some embodiments, it is not necessary to use the entire chain of the intracellular domain of a source protein. To the extent that a truncated portion of a source protein intracellular domain is used, such truncated portion may be used in place of the intact chain as long as it transduces the effector function signal. The term intracellular domain is thus meant to include any truncated portion of the source protein intracellular domain sufficient to transduce the effector function signal. The intracellular domain of a source protein (e.g, a receptor) may be identified, for example, by databases known in the art, e.g., UNIPROT.

As used herein, the term “transmembrane domain” refers to a domain that anchors a polypeptide to the plasma membrane of a cell. The transmembrane domain may be derived either from a natural, synthetic, semi-synthetic, or recombinant sources. In some embodiments, the transmembrane domain of a chimeric protein is a transmembrane domain of an inhibitory polypeptide, or a portion thereof (e.g., any of the inhibitory polypeptides described herein). In some embodiments, the transmembrane domain is a transmembrane domain of a stimulatory polypeptide, or a portion thereof (e.g., any of the stimulatory polypeptides described herein). The transmembrane domain of a polypeptide may be identified, for example, by databases known in the art, e.g., UNIPROT. In some embodiments, the transmembrane domain comprises up to 5, up to 10, or up to 15 amino acids of the intracellular domain. In some embodiments, the transmembrane domain comprises a charged amino acid residue at the terminus oriented towards the cytoplasm.

As used herein, the term “vector” or “construct” (sometimes referred to as a gene delivery system or gene transfer “vehicle”) refers to a macromolecule or complex of molecules comprising a polynucleotide to be delivered to a host cell, either in vitro or in vivo. In some embodiments, a construct refers to a polypeptide construct (e.g., a chimeric protein) that is is not a gene delivery system or gene transfer vehicle.

By “operably linked” or “co-expressed” with reference to nucleic acid molecules is meant that two or more nucleic acid molecules (e.g., a nucleic acid molecule to be transcribed, a promoter, and a CAR) are connected in such a way as to permit transcription of the nucleic acid molecule. “Operably linked” or “co-expressed” with reference to peptide and/or polypeptide molecules means that two or more peptide and/or polypeptide molecules are connected in such a way as to yield a single polypeptide chain, i.e., a fusion polypeptide, having at least one property of each peptide and/or polypeptide component of the fusion. The fusion polypeptide is preferably chimeric, i.e., composed of heterologous molecules.

The term “homology” refers to the percent of identity between two polynucleotides or two polypeptides. The correspondence between one sequence and another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions that promote the formation of stable duplexes between homologous regions, followed by digestion with single strand-specific nuclease(s), and size determination of the digested fragments. Two DNA, or two polypeptide, sequences are “substantially homologous” to each other when at least about 80%, preferably at least about 90%, and most preferably at least about 95% of the nucleotides, or amino acids, respectively match over a defined length of the molecules, as determined using the methods above.

The term “stem cell” refers herein to a cell that under suitable conditions is capable of differentiating into a diverse range of specialized cell types, while under other suitable conditions is capable of self-renewing and remaining in an essentially undifferentiated pluripotent state. The term “stem cell” also encompasses a pluripotent cell, multipotent cell, precursor cell, and progenitor cell. Exemplary human stem cells can be obtained from hematopoietic or mesenchymal stem cells obtained from bone marrow tissue, embryonic stem cells obtained from embryonic tissue, or embryonic germ cells obtained from genital tissue of a fetus. Exemplary pluripotent stem cells can also be produced from somatic cells by reprogramming them to a pluripotent state by the expression of certain transcription factors associated with pluripotency; these cells are called “induced pluripotent stem cells” or “iPScs,” “iPSCs,” or “iPS cells.”

An “embryonic stem (ES) cell” is an undifferentiated pluripotent cell which is obtained from an embryo in an early stage, such as the inner cell mass at the blastocyst stage, or produced by artificial means (e.g., nuclear transfer) and can give rise to any differentiated cell type in an embryo or an adult.

As used herein, the term “immune response” refers to a process that results in the activation and/or invocation of an effector function in either T cells, B cells, natural killer (NK) cells, and/or antigen-presenting cells. Thus, an immune response, as would be understood by the skilled artisan, includes, but is not limited to, any detectable activation of an NK cell, helper T cell, or cytotoxic T cell response, production of antibodies, T cell-mediated activation of allergic reactions, and the like.

Immune response may also refer to any particular measurable aspect of an immune response, including, but not limited to, cytokine secretion (IL-6, IL-10, IFN-γ, etc.), chemokine secretion, altered migration or cell accumulation, immunoglobulin production, dendritic cell maturation, regulatory activity, number of immune cells and proliferation of any cell of the immune system. Another parameter of an immune response is structural damage or functional deterioration of any organ resulting from immunological attack. One of skill in the art can readily determine an increase in any one of these parameters, using known laboratory assays. In one specific non-limiting example, to assess cell proliferation, incorporation of 3H-thymidine can be assessed. A “substantial” increase in a parameter of the immune response is a significant increase in this parameter as compared to a control. Specific, non-limiting examples of a substantial increase are at least about a 10% increase, at least about a 20% increase, at least about a 30% increase, at least about a 40% increase, at least about a 50% increase, at least about a 75% increase, at least about a 90% increase, at least about a 100% increase, at least about a 200% increase, at least about a 300% increase, or at least about a 500% increase. Similarly, an inhibition or decrease in a parameter of the immune response is a significant decrease in this parameter as compared to a control. Specific, non-limiting examples of a substantial decrease are at least about a 10% decrease, at least about a 20% decrease, at least about a 30% decrease, at least about a 40% decrease, at least about a 50% decrease, at least about a 75% decrease, at least about a 90% decrease, or at least about a 99% decrease. A statistical test, such as a non-parametric ANOVA, or a T-test, can be used to compare differences in the magnitude of the response induced by one agent as compared to the percent of samples that respond using a second agent. In some examples, p≤0.05 is significant, and indicates that the chance that an increase or decrease in any observed parameter is due to random variation is less than 5%. One of skill in the art can readily identify other statistical assays of use.

As used herein, the term “immune cell” refers to any cell involved in the mounting of an immune response. Such cells include, but are not limited to, T cells, B cells, NK cells, NKT cells, antigen-presenting cells, macrophages, and the like.

“Induced pluripotent stem cells” (“iPScs,” “iPSCs,” or “iPS cells”) are cells generated by reprogramming a somatic cell by expressing or inducing expression of a combination of factors (herein referred to as reprogramming factors). iPS cells can be generated using fetal, postnatal, newborn, juvenile, or adult somatic cells. In certain embodiments, factors that can be used to reprogram somatic cells to pluripotent stem cells include, for example, Oct4 (sometimes referred to as October 3/4), Sox2, c-Myc, Klf4, Nanog, and Lin28. In some embodiments, somatic cells are reprogrammed by expressing at least two reprogramming factors, at least three reprogramming factors, at least four reprogramming factors, at least five reprogramming factors, at least six reprogramming factors, or at least seven reprogramming factors to reprogram a somatic cell to a pluripotent stem cell.

“Hematopoietic progenitor cells” or “hematopoietic precursor cells” refers to cells which are committed to a hematopoietic lineage but are capable of further hematopoietic differentiation and include hematopoietic stem cells, multipotential hematopoietic stem cells, common myeloid progenitors, megakaryocyte progenitors, erythrocyte progenitors, and lymphoid progenitors. Hematopoietic stem cells (HSCs) are multipotent stem cells that give rise to all the blood cell types including myeloid (monocytes and macrophages, granulocytes (neutrophils, basophils, eosinophils, and mast cells), erythrocytes, megakaryocytes/platelets, dendritic cells), and lymphoid lineages (T cells, B cells, NK cells).

As used herein, the term “membrane receptor” refers to any receptor found on the surface of a cell, e.g., an immune cell. The membrane receptor may include receptors for hormones, cytokines, growth factors, cell recognition molecules, or other signaling receptors. Examples of membrane receptors include but are not limited to those listed in Table 1.

As used herein, the term “modulating an immune response” refers to mediating a detectable increase or decrease in the level of an immune response in a mammal compared with the level of an immune response in the mammal in the absence of a treatment or compound, and/or compared with the level of an immune response in an otherwise identical but untreated mammal. The term encompasses perturbing and/or affecting a native signal or response thereby mediating a beneficial therapeutic response in a mammal, preferably, a human.

As used herein, the term “negative signal” or “inhibitory signal” refers to a signal, i.e., signaling molecule, that induces the typical cascade of intracellular events associated with among other things, decreased proliferation, decreased activation, decreased cellular processing, and the like, of an immune cell, e.g., as compared to a like cell not contacted with the signal. In embodiments, the negative signal or inhibitory signal decreases activation of an immune response.

As used herein, the term “inhibitory polypeptide” refers to a polypeptide, or a portion thereof, that is capable of associating with or binding to a negative signal. The inhibitory polypeptide may associate with a negative signal to induce the typical cascade of intracellular events associated with among other things, decreased proliferation, decreased activation, decreased cellular processing, and the like, of an immune cell. In some embodiments, the inhibitory polypeptide is an inhibitory polypeptide listed in Table 1 or Table 1.1.

As used herein, the term “positive signal” or “activating signal” refers to a signal, i.e., signaling molecule, that induces the typical cascade of intracellular events associated with, among other things, increased proliferation, increased activation, increased cellular processing, and the like, of an immune cell, e.g., as compared to a like immune cell not contacted with the signal. In embodiments, the positive signal or activating signal increases activation of an immune response.

As used herein, the term “stimulatory polypeptide” refers to a polypeptide, or a portion thereof, that is capable of associating with or binding to a positive signal. The stimulatory polypeptide may induce the typical cascade of intracellular events associated with, among other things, increased proliferation, increased activation, and/or increased cellular processing, and the like, of an immune cell. In some embodiments, the stimulatory polypeptide is selected from the polypeptides listed in Table 2 or Table 2.1.

As used herein, the term “pluripotent stem cell” refers to a stem cell that has the potential to differentiate into all cells constituting one or more tissues or organs, or preferably, any of the three germ layers: endoderm (e.g., interior stomach lining, gastrointestinal tract, the lungs), mesoderm (e.g., muscle, bone, blood, urogenital), or ectoderm (e.g., epidermal tissues and nervous system).

“Programming” is a process that alters the type of progeny a cell can produce. For example, a cell has been programmed when it has been altered so that it can form progeny of at least one new cell type, either in culture or in vivo, as compared to what it would have been able to form under the same conditions without programming. This means that after sufficient proliferation, a measurable proportion of progeny having phenotypic characteristics of the new cell type are observed, if essentially no such progeny could form before programming; alternatively, the proportion having characteristics of the new cell type is measurably more than before programming. This process includes differentiation, dedifferentiation, and transdifferentiation.

“Differentiation” is the process by which a less specialized cell becomes a more specialized cell type. “Dedifferentiation” is a cellular process in which a partially or terminally differentiated cell reverts to an earlier developmental stage, such as pluripotency or multipotency. “Transdifferentiation” is a process of transforming one differentiated cell type into another differentiated cell type. Typically, transdifferentiation by programming occurs without the cells passing through an intermediate pluripotency stage—i.e., the cells are programmed directly from one differentiated cell type to another differentiated cell type. Under certain conditions, the proportion of progeny with characteristics of the new cell type may be at least about 1%, 5%, 25% or more.

As used herein, the term “subject” or “subject in need thereof” refers to a mammal, preferably a human being, male or female, at any age that is in need of a therapeutic intervention, a cell transplantation, or a tissue transplantation. Typically, the subject is in need of therapeutic intervention, cell, or tissue transplantation (also referred to herein as recipient) due to a disorder or a pathological or undesired condition, state, or syndrome, or a physical, morphological or physiological abnormality which is amenable to treatment via therapeutic intervention, cell, or tissue transplantation.

As used herein, a “disruption” or “alteration” of a gene refers to the elimination or reduction of expression of one or more gene products encoded by the subject gene in a cell, compared to the level of expression of the gene product in the absence of the alteration. Exemplary gene products include mRNA and protein products encoded by the gene. Alteration in some cases is transient or reversible and in other cases is permanent. Alteration in some cases is of a functional or full-length protein or mRNA, despite the fact that a truncated or nonfunctional product may be produced. In some embodiments herein, gene activity or function, as opposed to expression, is disrupted. Gene alteration is generally induced by artificial methods, i.e., by addition or introduction of a compound, molecule, complex, or composition, and/or by alteration of nucleic acid of or associated with the gene, such as at the DNA level. Exemplary methods for gene alteration include gene silencing, knockdown, knockout, and/or gene alteration techniques, such as gene editing. Examples of gene editing methods include the use of CRISPR/Cas systems, meganuclease systems, Zinc Finger Protein (ZFP), and Zinc Finger Nuclease (ZFN) systems and/or transcription activator-like protein (TAL), transcription activator-like effector protein (TALE), or TALE nuclease protein (TALEN) systems. Examples of gene alteration also include the use of antisense technology, such as RNAi, siRNA, shRNA, and/or ribozymes, which generally result in transient reduction of expression, as well as gene editing techniques which result in targeted gene inactivation or alteration, e.g., by induction of breaks and/or homologous recombination. Examples include insertions, mutations, and deletions. The alterations typically result in the repression and/or complete absence of expression of a normal or “wild type” product encoded by the gene. Examples of such gene alterations are insertions, frameshift, and missense mutations, deletions, knock-in, and knock-out of the gene or part of the gene, including deletions of the entire gene. Such alterations can occur in the coding region, e.g., in one or more exons, resulting in the inability to produce a full-length product, functional product, or any product, such as by insertion of a stop codon. Such alterations may also occur by alterations in the promoter or enhancer or other region affecting activation of transcription, so as to prevent transcription of the gene. Gene alterations include gene targeting, including targeted gene inactivation by homologous recombination.

The terms “tumor-associated antigen,” “tumor antigen” and “cancer cell antigen” are used interchangeably herein. The terms refer to any antigenic substance produced, expressed, or overexpressed in tumor cells which may, for example, trigger an immune response in the host. The terms also refer to proteins, glycoproteins or carbohydrates that are specifically or preferentially expressed by cancer cells.

“Treating” or “treatment of a disease or condition” refers to executing a protocol or treatment plan, which may include administering one or more drugs to a subject (e.g., a patient), in an effort to alleviate signs or symptoms of the disease or the recurrence of the disease. Desirable effects of treatment include decreasing the rate of disease progression, ameliorating, or palliating the disease state, and remission, increased survival, improved quality of life or improved prognosis. Alleviation or prevention can occur prior to signs or symptoms of the disease or condition appearing, as well as after their appearance. In addition, “treating” or “treatment” does not require complete alleviation of signs or symptoms, and does not require a cure.

The term “therapeutic benefit” or “therapeutically effective” as used throughout this application refers to anything that promotes or enhances the well-being of the subject with respect to the medical treatment of this condition. This includes, but is not limited to, a reduction in the frequency, severity, or rate of progression of the signs or symptoms of a disease. For example, treatment of cancer may involve, for example, a reduction in the size of a tumor, a reduction in the invasiveness of a tumor, reduction in the growth rate of the cancer, or a reduction in the rate of metastasis or recurrence. Treatment of cancer may also refer to prolonging survival of a subject with cancer.

“Antigen recognition moiety or domain” or “antigen-binding domain,” refers to a molecule or portion of a molecule that specifically binds to an antigen. In some embodiments, the antigen recognition moiety is an antibody, antibody like molecule or fragment thereof and the antigen is a negative signaling molecule or a tumor antigen.

“Antibody” as used herein refers to monoclonal or polyclonal antibodies. An antibody can be an IgG1, IgG2, IgG3, IgG4, IgM, IgE, or IgA antibody. In some embodiments, an antibody can be a human or humanized antibody.

“Antibody like molecules” may be for example proteins that are members of the Ig-superfamily which are able to selectively bind a partner.

The terms “fragment of an antibody.” “antibody fragment,” “functional fragment of an antibody,” and “antigen-binding portion” are used interchangeably herein to mean one or more fragments or portions of an antibody that retain the ability to specifically bind to an antigen (see, generally, Holliger et al., Nat. Biotech. 23(9):1126-1129, 2005). The antibody fragment desirably comprises, for example, one or more CDRs, the variable region (or portions thereof), the constant region (or portions thereof), or combinations thereof. Examples of antibody fragments include, but are not limited to, (i) a Fab fragment; (ii) a F (ab) 2 fragment: (iii) a Fv fragment; (iv) a single chain Fv (scFv); and (v) a diabody.

The term “antibody mimetic” is intended to describe an organic compound that specifically binds a target sequence and has a structure distinct from a naturally occurring antibody. Antibody mimetics may comprise a protein, a nucleic acid, or a small molecule. The target sequence to which an antibody mimetic of the disclosure specifically binds may be an antigen. Exemplary antibody mimetics include, but are not limited to, an affibody, an afflilin, an affimer, an affitin, an alphabody, an anticalin, an avimer (also known as avidity multimer), a DARPin (Designed Ankyrin Repeat Protein), a Fynomer, a Kunitz domain peptide, a monobody, and a centyrin.

The term “functional variant,” as used herein, refers to a polypeptide, or a protein having substantial or significant sequence identity or similarity to the reference polypeptide, and retains the biological activity of the reference polypeptide of which it is a variant. In reference to a nucleic acid sequence encoding the protein, a nucleic acid sequence encoding a functional variant of the protein can be for example, at least about 10% identical, at least about 25% identical, at least about 30% identical, at least about 50% identical, at least about 65% identical, at least about 70% identical, at least about 75% identical, at least about 80% identical, at least about 85% identical, at least about 90% identical, at least about 95% identical, or at least about 99% identical to the nucleic acid sequence encoding the reference polypeptide.

The phrases “pharmaceutically acceptable” or “pharmacologically acceptable” refers to molecular entities and compositions that do not produce an adverse, allergic, or other untoward reaction when administered to an animal, such as a human, as appropriate. For animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., saline solutions, phosphate buffered saline, parenteral vehicles, such as sodium chloride, Ringer's dextrose, etc.), antioxidants, preservatives (e.g., antibacterial or antifungal agents, anti-oxidants, chelating agents, and inert gases), isotonic agents, such like materials and combinations thereof, as would be known to one of ordinary skill in the art. The pH and exact concentration of the various components in a pharmaceutical composition are adjusted according to well-known parameters.

The term “T cell” refers to T lymphocytes, and includes, but is not limited to, γ:δ⁺ T cells, NK T cells, CD4⁺ T cells and CD8⁺ T cells. CD4⁺ T cells include THO, Tₕ1 and TH2 cells, as well as regulatory T cells (Tᵣₑₛ). There are at least three types of regulatory T cells: CD4⁺ CD25⁺ T_{reg}, CD25 T_{H}3 T_{reg}, and CD25 T_{R} 1 T_{reg}. “Cytotoxic T cell” refers to a T cell that can kill another cell. The majority of cytotoxic T cells are CD8⁺ MHC class I-restricted T cells, however some cytotoxic T cells are CD4⁺. In some embodiments, the T cell of the present disclosure is CD4⁺ or CD8⁺.

The activation state of a T cell defines whether the T cell is “resting” (i.e., in the G₀ phase of the cell cycle) or “activated” to proliferate after an appropriate stimulus such as the recognition of its specific antigen, or by stimulation with OKT3 antibody, PHA or PMA, etc. The “phenotype” of the T cell (e.g., naive, central memory, effector memory, lytic effectors, help effectors (TH1 and TH2 cells), and regulatory effectors), describes the function the cell exerts when activated. A healthy donor has T cells of each of these phenotypes, and which are predominately in the resting state. A naive T cell will proliferate upon activation, and then differentiate into a memory T cell or an effector T cell. The cell can then assume the resting state again, until it gets activated the next time, to exert its new function and may change its phenotype again. An effector T cell will divide upon activation and antigen-specific effector function.

“Natural killer T cells” (NKT cells; not to be confused with natural killer cells of the innate immune system) bridge the adaptive immune system with the innate immune system. Unlike conventional T cells that recognize peptide antigens presented by major histocompatibility complex (WIC) molecules, NKT cells recognize glycolipid antigen presented by a molecule called CD1d. Once activated, these cells can perform functions ascribed to both Th and Tc cells (i.e., cytokine production and release of cytolytic/cell killing molecules). They are also able to recognize and eliminate some tumor cells and cells infected with herpes viruses.

“Natural killer cells” (“NK cells”) are a type of cytotoxic lymphocyte of the innate immune system. In some embodiments, NK cells provide a first line defense against viral infections and/or tumor formation. NK cells can detect MHC presented on infected or cancerous cells, triggering cytokine release, and subsequently induce lysis and apoptosis. NK cells can further detect stressed cells in the absence of antibodies and/or MHC, thereby allowing a rapid immune response.

An “artificial cell” is an engineered particle that mimics one or many functions of a biological cell. Artificial cells can comprise artificial structures where biologically active components, for example, proteins, genes, enzymes, or other cellular structures, are encapsulated in artificial membranes.

“AML,” as used herein, refers to acute myelogenous leukemia, also known as acute myelocytic leukemia, acute granulocytic leukemia, and acute non-lymphocytic leukemia. The term “AML” refers to all subtypes, including myeloblastic (MO) on special analysis, myeloblastic (MI) without maturation, myeloblastic (M2) with maturation, promyeloctic (M3), myelomonocytic (M4), monocytic (M5), erythroleukemia (M6) and megakaryocytic (M7).

“Relapsed AML” refers to subjects (e.g., patients) who have experienced a recurrence following an interval of remission of AML.

“Refractory AML” refers to subjects (e.g., patients) whose disease does not respond to the first cycle of initial standard induction therapy (e.g., anthracycline and/or cytarabine-based therapy). In some embodiments, “refractory AML” refers to subjects (e.g., patients) who lack remission following initial therapy. In some embodiments, “refractory AML” refers to subjects whose disease does not respond to one or two or more cycles of standard induction therapy.

The term “culturing” refers to the in vitro maintenance, differentiation, and/or propagation of cells in suitable media. By “enriched” is meant a composition comprising cells present in a greater percentage of total cells than is found in the tissues where they are present in an organism.

An “anti-cancer” agent is capable of negatively affecting a cancer cell/tumor in a subject, for example, by promoting killing of cancer cells, inducing apoptosis in cancer cells, reducing the growth rate of cancer cells, reducing the incidence, number, and/or rate of development of metastases, reducing solid tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

As used herein, “click reaction” refers to a range of reactions used to covalently link a first and a second moiety, for convenient production of linked products. It typically has one or more of the following characteristics: it is fast, is specific, is high-yield, is efficient, is spontaneous, does not significantly alter biocompatibility of the linked entities, has a high reaction rate, produces a stable product, favors production of a single reaction product, has high atom economy, is chemoselective, is modular, is stereoselective, is insensitive to oxygen, is insensitive to water, is high purity, generates only inoffensive or relatively non-toxic by-products that can be removed by nonchromatographic methods (e.g., crystallization or distillation), needs no solvent or can be performed in a solvent that is benign or physiologically compatible, e.g., water, stable under physiological conditions. Examples include an alkyne/azide reaction, a diene/dienophile reaction, or a thiol/alkene reaction. Other reactions can be used. In some embodiments, the click reaction is fast, specific, and high yield.

As used herein, “click handle” refers to a chemical moiety that is capable of reacting with a second click handle in a click reaction to produce a click signature. In embodiments, a click handle is comprised by a coupling reagent, and the coupling reagent may further comprise a substrate reactive moiety.

As used herein, “sortase” refers to an enzyme which catalyzes a transpeptidation reaction between a sortase recognition motif and a sortase acceptor motif. Various sortases from prokaryotic organisms have been identified. In some embodiments, the sortase catalyzes a reaction to conjugate the C-terminus of a first moiety containing a sortase recognition motif to the N-terminus of a second moiety containing a sortase acceptor motif by a peptide bond. In some embodiments, the sortase catalyzes a reaction to couple a first moiety to a second moiety by a peptide bond. In some embodiments, sortase mediated transfer is used to couple the N terminus of a first polypeptide, e.g., an extracellular binding domain of a protein on an NK cell to the N terminus of a second polypeptide, e.g., an antigen-binding domain, to the N terminus of a second polypeptide. In some embodiments, sortase mediated transfer is used to attach a coupling moiety, e.g., a “click” handle, to the N-terminus of each polypeptide, wherein the coupling moieties mediate coupling of the polypeptides. In some embodiments, the first polypeptide is an extracellular binding domain, e.g., an antigen-binding domain, comprising a sortase acceptor motif, and the second polypeptide is a transmembrane polypeptide comprising an extracellular N-terminal sortase acceptor motif, a transmembrane domain, and an intracellular domain. Sortase-mediated transfer is used to attach a coupling moiety, e.g., a click handle, to each polypeptide.

“Sortase acceptor motif,” as used herein, refers to a moiety that acts as an acceptor for the sortase-mediated transfer of a polypeptide. In some embodiments, the sortase acceptor motif is located at the N-terminus of a polypeptide. In some embodiments, the transferred polypeptide is linked by a peptide bond at its C-terminus to the N-terminal residue of the sortase acceptor motif. N-terminal acceptor motifs include Gly-[Gly] n- (SEQ ID NO: 1), wherein n=0-5 and Ala-[Ala] n- (SEQ ID NO: 2), wherein n=0-5.

“Sortase recognition motif,” as the term is used herein, refers to polypeptide which, upon cleavage by a sortase molecule, e.g., a, forms a thioester bond with the sortase molecule. In some embodiments, sortase cleavage occurs between T and G/A. In some embodiments, the peptide bond between T and G/A is replaced with an ester bond to the sortase molecule.

“Sortase transfer signature,” as the term is used herein, refers to the portion of a sortase recognition motif and the portion of a sortase acceptor motif remaining after the reaction that couples the former to the latter. In some embodiments, wherein the sortase recognition motif is LPXTG/A (SEQ ID NO: 3) and wherein the sortase acceptor motif is GG, the resultant sortase transfer signature after sortase-mediated reaction comprises LPXTGG (SEQ ID NO: 4).

As used herein, “signal transduction pathway” refers to the biochemical relationship between a variety of signal transduction molecules that play a role in the transmission of a signal from one region of a cell to another region of a cell.

As used herein, “signaling receptor” refers to a receptor that interacts with a ligand to trigger a biochemical chain of events inside the cell, creating a response, such as signal transduction, protein interaction, enzymatic activity, gene transcription, or a combination thereof. Exemplary examples of signaling receptors include, but are not limited to, TGF-BR, interleukin-2 receptor (IL-2R), interleukin-12 receptor (IL-12R), CD3, CD28, and CD137.

### II. Cells

Provided herein are cells engineered to comprise (e.g., to express) any of the proteins described herein. The engineered cells may be, e.g., immune cells (e.g., autologous or allogeneic T cells (e.g., regulatory T cells, type 1 regulatory T cells (Tr1), CD4⁺ T cells, CD8⁺ T cells, or gamma-delta T cells), NK cells (e.g., autologous or allogeneic NK cells), NKT cells (e.g., invariant NKT cells), stem cells (e.g., iPS cells), type 1 innate lymphoid cells (ILC1), intraepithelial type 1 innate lymphoid cells (ieILC1), type 2 innate lymphoid cells (ILC2), type 3 innate lymphoid cells (ILC3), lymphoid tissue inducer cells (LTi), monocytes, macrophages, dendritic cells (DC), platelets, marrow-infiltrating lymphocytes (MIL), or B cells), fibrocytes, mesenchymal stem cells, induced neural stem cells, induced pluripotent stem cell (iPSC)-derived cells, platelets, or erythrocytes. In some embodiments, the engineered cells are tumor infiltrating lymphocytes (TILs).

In some embodiments, the cells are immune cells. Cells of the immune system include lymphocytes, monocytes/macrophages, dendritic cells, the closely related Langerhans cells, natural killer (NK) cells, mast cells, basophils, and other members of the myeloid lineage of cells.

Provided herein are modified cells, e.g., immune cells that have been engineered to comprise (e.g., express) any of the engineered proteins (e.g., chimeric proteins) described herein. The engineered immune cells may be T cells (e.g., regulatory T cells, CD4⁺ T cells, CD8⁺ T cells, or gamma-delta T cells), NK cells, invariant NK cells, NKT cells, and may be derived from stem cells (e.g., induced pluripotent stem (iPSC) cells). In some embodiments, the engineered immune cells are monocytes or granulocytes, e.g., myeloid cells, macrophages, neutrophils, dendritic cells, mast cells, eosinophils, and/or basophils. Also provided herein are methods of producing and engineering the engineered immune cells. Further provided are methods of using and administering the engineered immune cells, e.g., for adoptive cell therapy, in which case the cells may be autologous or allogeneic. Thus, the engineered immune cells provided herein may be used as an immunotherapy, such as to target cancer cells.

Engineered cells, e.g., immune cells such as NK cells, of the present disclosure are produced by engineering a cell to comprise, e.g., to express, any of the proteins described herein. The cells may be isolated from subjects, particularly human subjects. The cells may be obtained from a subject of interest, such as a subject suspected of having a particular disease or condition, a subject suspected of having a predisposition to a particular disease or condition, or a subject who is undergoing therapy for a particular disease or condition. In some embodiments, the cells, e.g., immune cells such as NK cells, may be obtained from a subject in need of immunotherapy. In some embodiments, the cells, e.g., immune cells such as NK cells, can be obtained from a normal, healthy subject. In some embodiments, the cells, e.g., immune cells such as NK cells, are allogeneic to a subject in need of treatment. In some embodiments, the cells, e.g., immune cells such as NK cells, are autologous to a subject in need of treatment.

The cells, e.g., immune cells, such as NK cells, may be enriched and/or purified from any tissue where they reside including, but not limited to, blood (including blood collected by blood banks or cord blood banks), spleen, bone marrow, tissues removed and/or exposed during surgical procedures, and tissues obtained via biopsy procedures. Tissues/organs from which the cells are enriched, isolated, and/or purified may be isolated from living or non-living subjects, where the non-living subjects are organ donors. The isolated cells may be used directly, or they can be stored for a period of time, such as by freezing. In some embodiments, the cells are isolated from blood, such as peripheral blood or cord blood. In some embodiments, the cells, e.g., immune cells, such as NK cells, are isolated from cord blood have enhanced immunomodulation capacity, such as measured by CD4- or CD8-positive T cell suppression. In some embodiments, the cells, e.g., immune cells, such as NK cells, are isolated from pooled blood, particularly pooled cord blood, for enhanced immunomodulation capacity. The pooled blood may be from 2 or more sources, such as 3, 4, 5, 6, 7, 8, 9, 10 or more sources (e.g., donor subjects).

When the population of cells, e.g., immune cells, such as NK cells, is obtained from a donor distinct from the subject to be treated, the donor is preferably allogeneic, provided the cells obtained are subject-compatible in that they can be introduced into the subject. Allogeneic donor cells may or may not be human-leukocyte-antigen (HLA)-compatible. To be rendered subject-compatible, allogeneic cells can be treated to reduce immunogenicity.

In some embodiments, the modified immune cells of the present disclosure are NK cells. NK cells differentiate and mature in the bone marrow, lymph nodes, spleen, tonsils, and thymus. NK cells can be detected by specific surface markers, canonically as CD56⁺ and CD3⁻, as well as CD2, CD11a, CD11b, CD18, and CD18 in humans. In the blood, human NK cells are commonly divided into CD56-high/CD16-low and CD56-low/CD16-high subsets. NK cells do not express T cell antigen receptors, the pan T marker CD3, surface immunoglobulin B cell receptors. NK cells may be distinguished from rare CD56⁺ CD3⁻ monocytes and dendritic cells as CD7 and low/no expression of CD14, HLA-DR, CD33, and other myeloid markers.

Stimulation of NK cells is achieved through a crosstalk of signals derived from cell surface activating and inhibitory receptors. The activation status of NK cells is regulated by a balance of intracellular signals received from an array of germ-line-encoded activating and inhibitory receptors (Campbell, Curr. Top. Microbiol. Immunol. 298:23-57, 2006; the entire contents of which are incorporated herein by reference). When NK cells encounter an abnormal cell (e.g., tumor or virus-infected cell) and activating signals predominate, the NK cells can rapidly induce apoptosis of the target cell through directed secretion of cytolytic granules containing perforin and granzymes or engagement of death domain-containing receptors. Activated NK cells can also secrete type I cytokines, such as interferon-γ, tumor necrosis factor-α and granulocyte-macrophage colony-stimulating factor (GM-CSF), which activate both innate and adaptive immune cells, as well as other cytokines and chemokines (Wu et al., Adv. Cancer Res. 90:127-56, 2003; the entire contents of which are incorporated herein by reference). Production of these soluble factors by NK cells in early innate immune responses significantly influences the recruitment and function of other hematopoietic cells. Also, through physical contacts and production of cytokines, NK cells are central players in a regulatory crosstalk network with dendritic cells and neutrophils to promote or restrain immune responses.

In some embodiments, NK cells are derived from human peripheral blood mononuclear cells (PBMCs), unstimulated leukapheresis products (PBSC), human embryonic stem cells (hESCs), induced pluripotent stem cells (iPSCs), mesenchymal stem cells (MSCs), hematopoietic stem cells (HSCs), bone marrow, CD34⁺ cells, or umbilical cord blood (CB) by using methods well known in the art. In some embodiments, NK cells are isolated from PBMCs. In some embodiments, NK cells are derived from umbilical CB. In some embodiments, the NK cells are of an NK cell lines, e.g., NK-92, NK101, KHYG-1, YT, NK-YS, YTS, HANK-1, NKL, and NK3.3 cell lines.

NK cells can be differentiated from stem cells by various methods known in the art. In some instances, NK cells can be differentiated from induced pluripotent stem cells (iPSCs), human embryonic stem cells (hESCs), mesenchymal stem cells (MSCs), or hematopoietic stem cells (HSCs). Protocols for the differentiation of NK cells from iPSCs and hESCs are described, for example, in Bock et al. J. Vis. Exp. (74):e50337, 2013; Knorr et al. Stem Cells Transl. Med. 2(4):274-83, 2013; Ni et al. Methods Mol. Biol. 1029:33-41, 2013; Zhu and Kaufman (Methods Mol. Biol. 2048:107-19, 2019). In order to differentiate iPSCs to CD34⁺CD45⁺ HPCs, embryonic bodies (EB) can be generated using different approaches, such as spinning of single cell iPSCs in round-shaped wells (spin EBs), culture on murine stroma cells, or direct induction of iPSC monolayer fragments in media with cytokines inducing differentiation towards the hematopoietic lineage. HPCs can be enriched by cell sorting or cell separation of CD34⁺ and/or CD45⁺ cells, and subsequently placed on murine feeder cells (e.g., AFT024, OP9, MS-5, EL08-1D2) in medium containing IL-3 (during the first week), IL-7, IL-15, SCF, IL-2, and Flt3L. NK-cells can also be differentiated without usage of xenogeneic stromal feeder cells, as described, e.g., by Knorr et al. Stem Cells Transl. Med. 2(4):274-83, 2013. CD3⁻CD56^{bright}CD16^{+/-} NK cells can be differentiated from hiPSC up to stage 4b (NKp80⁺) on OP9-DL1 stroma cells and are highly functional in terms of degranulation, cytokine production and cytotoxicity including antibody-dependent cellular cytotoxicity (ADCC). NK cell yield can be considerably increased through inactivation of feeder cells with mitomycin-C (MMC) without impacting on maturation or functional properties.

Additionally or in alternative, CD56⁺CD16⁺CD3⁻ NK cells can be differentiated from human iPSCs and NK-cell development can be characterized by surface expression of NK-lineage markers, as described, e.g., by Euchner et al. Front. Immunol. 12:640672, 2021. Hematopoietic priming of human iPSCs can result in CD34⁺CD45⁺ hematopoietic progenitor cells (HPC) that do not require enrichment for NK lymphocyte propagation. HPC can be further differentiated into NK cells on OP9-DL1 feeder cells resulting in high purity of CD56^{bright}CD16⁻ and CD56^{bright}CD16⁺ NK cells. The output of generated NK cells can be increased by inactivating OP9-DL1 feeder cells with MMC. CD7 expression can be detected from the first week of differentiation indicating priming towards the lymphoid lineage. Differentiation of NK cells up to stage 4b can be confirmed by assessing the expression of NKp80 on NK cells, and by a perforin and granzyme B phenotype. Differentiation of NK cells can also be confirmed by assessing killer cell immunoglobulin-like receptor KIR2DL2/DL3 and KIR3DL1 on NK cells.

In some instances, CD3 CD56 NK cells can be differentiated from CD34 hematopoietic progenitors cells (HPCs), as described, e.g., by Cichocki et al. Front Immunol, 10:2078, 2019. NK cell development can occur along a continuum whereby common lymphocyte progenitors (CLPs) gradually downregulate CD34 and upregulate CD56. Acquisition of CD94 marks commitment to the CD56^{bright} stage, and CD56^{bright} NK cells subsequently differentiate into CD56^{dim} NK cells that upregulate CD16 and killer immunoglobulin-like receptors (KIR). Support for this linear model comes from analyses of cell populations in secondary lymphoid tissues and in vitro studies of NK cell development from HPCs.

CD3⁻ CD56⁺ NK cells with cytotoxic function can be differentiated in vitro after long-term culture of CD34⁺ cells isolated from cord blood, bone marrow, fetal liver, thymus, or secondary lymphoid tissue with IL-2 or IL-15, as described, e.g., by Mrozek et al. Blood 87:2632-40, 1996; Jaleco et al. J. Immunol. 159:694-702, 1997; Sanchez et al. J. Exp. Med. 178:1857-66, 1993; and Freud et al. Immunity 22:295-304, 2005.

In some embodiments, the NK cells are isolated and expanded using a previously described method of ex vivo expansion of NK cells (Shah et al., PLOS One 8(10):e76781, 2013; the entire contents of which are incorporated herein by reference). In this method, CB mononuclear cells are isolated by Ficoll density gradient centrifugation and cultured in a bioreactor with IL-2 and artificial antigen presenting cells (aAPCs). After 7 days, the cell culture is depleted of any cells expressing CD3 and re-cultured for an additional 7 days. The cells are again CD3-depleted and characterized to determine the percentage of CD56⁺/CD3⁺ cells or NK cells. In some embodiments, NK cells are derived from umbilical CB by the isolation of CD34⁺ cells and differentiation into CD56⁺/CD3⁺ cells by culturing in medium containing SCF, IL-7, IL-15, and IL-2.

In some embodiments, NK cells can be expanded or enriched from large volumes of peripheral blood, such as an apheresis products (e.g., mobilized PBSCs or unmobilized PBSCs). In other instances, NK cells can be expanded or enriched from smaller number of blood or stem cells. Expansion of NK cells from apharesis products are described, for example, in Lapteva et al. Crit. Rev. Oncog. 19:121-132, 2014; Miller et al. Blood 105(8):3051-7, 2005; Lapteva et al. Cytotherapy 14(9):1131-43, 2012; Spanholtz et al. PLOS One 6(6):e20740, 2011; Knorr et al. Stem Cells Transl. Med. 2(4):274-83, 2013; Pfeiffer et al. Leukemia 26(11):2435-9, 2012; Shi et al. Br. J. Haematol. 143(5):641-53, 2008; Passweg et al. Leukemia 18(11):1835-8, 2004; Koehl et al. Klin. Padiatr. 217(6):345-50, 2005; and Klingemann et al. Transfusion 53(2):412-8, 2013. In some embodiments, NK cells in peripheral blood and apheresis products can be detected by flow cytometry as CD45⁺CD56⁺CD3⁻ cells. In some instances, NK cells can be enriched from apheresis products by one or two rounds of depletion of CD3⁺ T cells using magnetic beads (e.g., CLINIMACS magnetic beads) coated with anti-CD3 antibody (e.g., CLINIMACS CD3 reagent) with or without overnight activation using IL-2 or IL-15. Additional depletion of CD19⁺ B cells with anti-CD19 antibody-coated magnetic beads (e.g., CliniMACS CD19 reagent) can further improve the purity of the NK cells. Alternatively, NK cells can be enriched by isolating CD56⁺ cells using anti-CD56 monoclonal antibody (e.g., CLINIMACS CD56 reagent) with or without CD3⁺ T cell depletion.

In some embodiments, NK cells can be expanded using feeder cell-based technology. Such methods are described, for example, in Berg et al. Cytotherapy 11(3):341-55, 2009; Lapteva et al. 2012, supra; and Lapteva et al. Crit. Rev. Oncog. 19:121-132, 2014. Feeder-cell methods generally require cytokines as well as irradiated feeder cells, such as EBV-LCLs or genetically modified K562 cells, to produce large numbers of CD3⁻56⁺ NK cells with greater than 70% purity from peripheral blood mononuclear cells (PBMCs). CD3-depleted, CD56-enriched PBMCs can be cultured in the presence of EBV-LCL feeders and X-VIVO 20 medium supplemented with 10% heat inactivated human AB serum, 500 U/mL IL-2 and 2 mM L-alanyl-L-glutamine (Berg et al. Cytotherapy, 11(3):341-55, 2009).

In some embodiments, NK cells can be expanded using a genetically modified feeder cell expansion system, as described, for example, in Yang et al. (Mol. Therapy 18:428-445, 2020). In such expansion methods, human primary NK cells can be expanded directly from PBMCs and cord blood (CB), as well as tumor tissue, using an irradiated, genetically engineered cell line that expresses membrane-bound interleukin 21 (IL-21), optionally in combination in the presence of IL-15 and IL-2. Other methods of NK expansion are described in Becker et al., Cancer Immunol. Immunother. 65(4):477-84, 2016, Phan et al., Methods Mol. Biol. 1441:167-74, 2016, each of which are incorporated herein in reference in their entireties. Commercially available kits for expanding NK cells, such as CELLXVIVO Human NK Cell Expansion Kit (R&D Systems; Cat. No. CDK015) and NK Cell Activation/Expansion Kit, human (MILTENYI BIOTEC; Cat No. 130-094-483) can also be used with the methods described herein.

In some embodiments, the modified NK cells of the present disclosure are prepared directly from NK cells, e.g., by engineering the NK cells to comprise (e.g., express) a chimeric protein disclosed herein. In some embodiments, the modified NK cells are prepared by engineering an NK precursor cell to comprise (e.g., express) a chimeric protein disclosed herein, and the modified NK cell is then produced from the engineered precursor cell.

In some embodiments, the modified immune cells of the present disclosure are T cells. In some embodiments, the immune cells are alpha beta T cells and gamma delta T cells). In some embodiments, the immune cells are T cells that are one or more of CD3⁺, CD28⁺, CD4⁺, CD8⁺, CD45RA⁺, CD25⁺ and CD45RO⁺. In some embodiments, the T cells are isolated tumor infiltrating lymphocytes (TIL). In some embodiments, the T cells are CD4⁺ T cells. In some embodiments, the T cells are CD8⁺ T cells. In some embodiments, the T cells are regulatory T cell (e.g., a CD4⁺, CD25⁺, CD62L^{hi}, GITR⁺ and FoxP3⁺ T cells). In some embodiments, the T cells are memory T cells (TCM) (e.g., CD62L⁺, CCR7⁺, CD45RO⁻ and CD45RA⁻ T cells). In some embodiments, the T cells are stem cell memory T cells. In some embodiments, the T cells are naïve T cells. In some embodiments, the T cells are a mixed population of CD4⁺ T cells, CD8 T cells, stem cell memory T cells and naïve T cells.

The immune cells provided herein may be expanding using methods known in the art (see e.g., Gregory et al. Methods Mol. Biol. 380:83-105, 2007; Tricket and Kwan, J. Immunol. Methods 275(1-2):251-5, 2003; Schluck et al. Front Immunol. 10:931; Peters et al. Methods Enzymol. 631:223-37, 2020; Andrews et al. Cytotherapy 22(5):276-90; Exley et al. Curr. Protoc. Immunol. 119:14.11.1-14.11.20, 2017; and Becker et al. Cancer Immunol Immunother. 65(4): 477-84). For example, T cells can be expanding by contacting them with a surface having attached thereto an agent that stimulates a CD3/TCR complex-associated signal and a ligand that stimulates a costimulatory molecule on the surface of the T cells, including but not limited to an anti-CD3 antibody or antigen-binding fragment thereof, an anti-CD2 antibody immobilized on a surface, a protein kinase C activator (e.g., bryostatin) in conjunction with a calcium ionophore. In addition, the T cells may also be contacted with a ligand that binds to an accessory molecule on the surface of the T cells (e.g., an anti-CD3 antibody and an anti-CD28 antibody under conditions suitable for the stimulation and proliferation of the T cells.

In some embodiments, the modified immune cells of the present disclosure are natural killer T (NKT) cells. NKT cells are a heterogeneous group of T cells that share properties of both T cells and natural killer cells. Many of these cells recognize the non-polymorphic CD1d molecule, an antigen-presenting molecule that binds self and foreign lipids and glycolipids.

In some embodiments, the modified immune cells are invariant NKT (INKT) cells. In some embodiments, the modified immune cells are type 2 NKT cells.

In some embodiments, the modified immune cells of the present disclosure are macrophages. In some embodiments, the modified immune cells are M1 macrophages. In some embodiments, the modified immune cells are M2 macrophages. Macrophages can be identified using flow cytometry or immunohistochemical staining by their specific expression of proteins such as CD14, CD40, CD11b, CD64, F4/80 (mice)/EMRI (human), lysozyme M, MAC-1/MAC-3 and CD68 (Khazen et al., FEBS Letters. 579(25):5631-4, 2005).

In some embodiments, the cells, e.g., modified immune cells, of the present disclosure are stem cells, such as induced pluripotent stem cells (PSCs), mesenchymal stem cells (MSCs), or hematopoietic stem cells (HSCs). The pluripotent stem cells used herein may be induced pluripotent stem (iPS) cells, commonly abbreviated iPS cells, iPscs, or iPSCs. With the exception of germ cells, any cell can be used as a starting point for iPSCs. For example, cell types could be keratinocytes, fibroblasts, hematopoietic cells, mesenchymal cells, liver cells, or stomach cells. There is no limitation on the degree of cell differentiation or the age of an animal from which cells are collected. For example, undifferentiated progenitor cells (including somatic stem cells) and finally differentiated mature cells can be used as sources of somatic cells in the methods disclosed herein. Somatic cells can be reprogrammed to produce iPSCs using methods known to one of skill in the art (U.S. Patent Application Publication Nos. 2009/0246875, 2010/0210014, and 2011/0104125; 2012/0276636, U.S. Pat. Nos. 8,058,065, 8,129,187, 8,268,620, 8,546,140, 9,175,268, 8,741,648, and 8,691,574, and PCT Publication No. WO 2007/069666 A1, the entire contents of each of which are incorporated herein by reference). Generally, nuclear reprogramming factors are used to produce pluripotent stem cells from a somatic cell. In some embodiments, at least three, or at least four of Klf4, c-Myc, Oct3/4, Sox2, Nanog, and Lin28 are utilized. In other embodiments, Oct3/4, Sox2, c-Myc and Klf4 are utilized or Oct3/4, Sox2, Nanog, and Lin28. Methods for introducing one or more reprogramming substances, or nucleic acids encoding these reprogramming substances, are known in the art, and disclosed for example, in U.S. Pat. Nos. 8,268,620, 8,691,574, 8,741,648, 8,546,140, 8,900,871, and 8,071,369, which are incorporated herein by reference.

Once derived, iPSCs can be cultured in a medium sufficient to maintain pluripotency. iPSCs may be used with various media and techniques developed to culture pluripotent stem cells, more specifically, embryonic stem cells, as described in U.S. Pat. No. 7,442,548 and U.S. Patent Application Publication No. 2003/0211603, the entire contents of each of which are incorporated by reference herein. For example, pluripotent cells may be cultured and maintained in an essentially undifferentiated state using a defined, feeder-independent culture system, such as a TESR™ medium or E8™/Essential 8™ medium.

### III. Proteins of the Disclosure

According to the present disclosure, cells, e.g., immune cells (e.g., autologous or allogeneic T cells (e.g., regulatory T cells, type 1 regulatory T cells (Tr1), CD4⁺ T cells, CD8⁺ T cells, or gamma-delta T cells), NK cells, NKT cells (e.g., invariant NKT cells), stem cells (e.g., iPS cells), type 1 innate lymphoid cells (ILC1), intraepithelial type 1 innate lymphoid cells (ieILC1), type 2 innate lymphoid cells (ILC2), type 3 innate lymphoid cells (ILC3), lymphoid tissue inducer cells (LTi), monocytes, macrophages, dendritic cells (DC), platelets, marrow-infiltrating lymphocytes (MIL), or B cells), fibrocytes, mesenchymal stem cells, induced neural stem cells, induced pluripotent stem cell (iPSC)-derived cells, platelets or erythrocytes) may be engineered to comprise (e.g., express) one or more (e.g., two, three, four, or five) engineered proteins (e.g., chimeric proteins) disclosed herein. The engineered proteins (e.g., chimeric proteins) included in a modified cell, e.g., immune cell, such as an NK cell, may take the form of several different modalities. These modalities include, but are not limited to, a sink, a dominant negative receptor, and a signal inverter. In some embodiments, an engineered protein (e.g., chimeric protein) described herein may have one or more characteristics of these modalities.

### Sinks

A sink or sink protein, as used herein, refers to a protein comprising an extracellular domain and a transmembrane domain, wherein the extracellular domain binds to a negative signal (e.g., an exogenous ligand that inhibits the activation of an immune response), and wherein the protein lacks a fully functional intracellular domain. In some embodiments, the intracellular domain is fully non-functional. In some embodiments, the intracellular domain is naturally multi-functional, and the intracellular domain in the sink protein lacks an inhibitory function but retains other functions, e.g., a stimulatory function. In some embodiments, the sink protein lacks an intracellular domain. In some embodiments, the sink protein comprises a transmembrane domain and an extracellular domain from the same protein, e.g., the sink protein is a truncated protein lacking its intracellular domain. In some embodiments, a sink protein comprises a transmembrane domain that is derived from a different protein than the extracellular domain, i.e., the sink protein is a chimeric protein. In some embodiments, the extracellular domain of the sink protein is cleaved from the cell membrane.

In some embodiments, a sink protein may function by competing with an endogenously expressed protein for access and binding to a negative signal. However, because the sink protein lacks a fully functional intracellular domain, it is unable to induce downstream signaling that leads to an inhibitory function upon binding the negative signal. While not wishing to be bound by theory, the ability of a sink protein to interfere or block a negative signal may occur through passive interference and so may depend on the ratio of the sink protein to the endogenously expressed wild-type protein (e.g., a wild-type protein having the same extracellular domain as the sink protein) on the cell, as well as the availability of the negative signal.

### Dominant Negative Receptors (DNRs)

A dominant negative receptor (DNR), as used herein, refers to a dominant negative isoform of a protein, wherein the dominant negative isoform of the protein competes with a wild-type isoform of the protein for binding a negative signal (e.g., an exogenous ligand that inhibits the activation of an immune response). Therefore, a DNR impairs the function of an endogenously expressed protein either by forming non-functional complexes, by sequestering adaptor and/or co-receptor proteins, and/or by other mechanisms that prevent the endogenous wild-type receptors from conveying a negative signal regardless of whether or not binding to their ligand(s) occurs. Unlike a sink, which acts on and directly binds to the negative signal, a DNR inhibits the activity of a negative signal by acting on, e.g., binding to, the endogenously expressed wild-type receptor that naturally conveys the negative signal. While not wishing to be bound by theory, a true dominant negative complex may occur through active interference (unlike a sink, whose interference may be passive) and so would not be expected to be overwhelmed by high concentrations of the negative signal. The mechanism of action of a DNR can be demonstrated by using any number of protein structure-function studies, which would be apparent to those of skill in the art.

In some embodiments, truncating the intracellular domain of a receptor protein can create both a sink and a DNR. For example, truncating the intracellular domain of any one of TGF-BR2, TGF-BR1, IL-10RA, and TIGIT could, in some embodiments, create a truncated protein that functions as both a sink and a DNR modality of the disclosure.

### Signal Inverters

A signal inverter refers to a chimeric protein of the disclosure which comprises an extracellular domain, a transmembrane domain, and an intracellular domain, wherein the extracellular domain is capable of binding a negative signal (e.g., an exogenous ligand that inhibits activation of an immune response), and wherein the intracellular domain comprises at least a portion of the intracellular domain of a stimulatory polypeptide that is associated with a positive signal that promotes activation of an immune response/activates an immune cell.

A signal inverter may comprise an extracellular domain (or a portion thereof) of a natural isoform or an engineered variant of a protein that binds a negative signal. A signal inverter may comprise an intracellular domain (or a portion thereof) of a natural isoform of a protein that preferentially or exclusively interacts with pro-activation partners, or a protein altered to do so. For example, an exemplary signal inverter of the present disclosure is a chimeric protein comprising a TGF-B-binding extracellular domain fused to a DAP12 intracellular domain. Additional examples of signal inverters are described herein.

### Signal Transformers

A signal transformer, as used herein, refers to an engineered genomic locus of an immune cell (e.g., NK cell) that encodes a positive signal, wherein the genomic expression of the positive signal is induced by a negative signal, i.e., the signal transduction pathway associated with a negative signal. The signal transformer may be engineered at a genomic locus that is independent of an endogenous genomic locus naturally targeted by the negative signal. In such embodiments, the negative signal will induce expression of both the signal transformer as well as the endogenous genomic locus (referred to as a “knock-in” signal transformer). Alternately, the signal transformer may be engineered into an endogenous genomic locus naturally targeted by the negative signal, and thereby negate the expression from the endogenous genomic locus (referred to as a “knock-in, knock-out” signal transformer).

In some embodiments, a signal transformer comprises an engineered genomic locus of an immune cell (e.g., NK cell) that encodes a DAP12 signal, whose expression is induced by a negative signal (e.g., TGF-B). In some embodiments, DAP12 signal is engineered into an endogenous PD-1 genomic locus naturally targeted by the negative signal (e.g., TGF-B induced Smad2 activation).

### Override

An override refers to a natural isoform or an engineered variant of a protein that generates a positive signal in an immune cell (e.g., NK cell), that is capable of enhancing anti-tumor activity despite the negative signals received by an immune cell (e.g., NK cell). A protein of the override modality does not influence a negative signal, or a signal transduction pathway associated therewith, and functions despite the immune cell (e.g., NK cell) also experiencing one or more negative signals.

In some embodiments, an engineered protein (e.g., chimeric protein) of the override modality comprises a DAP12 protein that is capable of being constitutively expressed on the surface of a cell, e.g., an immune cell (e.g., an NK cell).

### Protein Domains

The present disclosure provides engineered proteins (e.g., chimeric proteins) and cells, e.g., immune cells, e.g., NK cells, that have been engineered to comprise (e.g., express) the engineered proteins (e.g., chimeric proteins). In some embodiments, the engineered proteins (e.g., chimeric proteins) comprise one or more of a) an extracellular domain, b) a transmembrane domain, and c) an intracellular domain. In some embodiments, the engineered proteins (e.g., chimeric proteins) of the disclosure comprise an extracellular domain and a transmembrane domain. In some embodiments, the engineered proteins (e.g., chimeric proteins) of the disclosure comprise an extracellular domain, a transmembrane domain, and one or more intracellular domains. In some embodiments, the engineered proteins (e.g., chimeric proteins) further include one or more linkers (e.g., disposed between an extracellular domain and a transmembrane domain, between a transmembrane domain and an intracellular domain, and/or between two or more intracellular domains). In some embodiments, the extracullular domain is linked to one or more additional domains (e.g., a transmembrane domain and/or intracellular domain) via a linker. For example, in some embodiments, once an extracellular domain engages a negative signal (e.g., binds its corresponding ligand), the intracellular domain transmits an activation signal to the cell, e.g., NK cell, that promotes an immune response, e.g., induces the NK cell to destroy a targeted tumor cell.

### A. Extracellular Domains

In some embodiments, an extracellular domain that may be comprised in an engineered protein (e.g., chimeric protein) of the disclosure comprises at least a portion of the extracellular domain of an inhibitory polypeptide (receptor) that associates with a negative signal (ligand). In some embodiments, the inhibitory polypeptide from which the extracellular domain is derived is selected from the inhibitory polypeptides presented in Table 1. In some embodiments, the extracellular domain of an engineered protein (e.g., chimeric protein) provided herein comprises or consists of the extracellular domain of an inhibitory polypeptide presented in Table 1.

TABLE 1
Inhibitory Polypeptides

| Inhibitory Polypeptide | | Negative Signal | Other ligand-binding |
|---|---|---|---|
| (receptor) | UNIPROT ID | (ligand) | substitutes |
| Adenosine receptor A2A | P29274 | Adenosine | A1R or A3 |
| Adenosine receptor A2B | P29275 | Adenosine | A1R or A3 |
| Prostaglandin receptor EP2 | P43116 | Prostaglandins | EP1 or EP3 |
| Prostaglandin receptor EP4 | P35408 | Prostaglandins | EP1 or EP3 |
| TGF-BR1 | P36897 | TGF-β | TGF-BR2 |
| TGF-BR2 | P37173 | TGF-β | TGF-BR1 |
| IL-10RA | Q13651 | IL-10 | |
| IL-10RB | Q08334 | IL-10 | |
| IL-18BP | O95998 | IL-18 | |
| IL-1R8 | A0A291NLA3 | IL-1 family | Non-cleavable IL-1R2 |
| IL-6RA | P08887 | IL-6 | |
| IL-6RB ( also known as | P40189 | IL-6 family | |
| gp130 and CD130) | | | |
| PD-1 | Q15116 | PD-L1/2 | B7-1 |
| CTLA-4 | P16410 | B7-1/2 | |
| TIM-3 | Q8TDQ0 | Multiple, disparate | TIM-1/4, RAGE, others |
| Lag3 | P18627 | MHCII and others | |
| BTLA | Q7Z6A9 | HVEM | LIGHT, CD160 |
| CD160 | O95971 | HVEM | LIGHT, BTLA |
| TIGIT | Q495A1 | CD155 and CD112 | DNAM-1, CD96, |
| | | | TACTILE, PVRIG, |
| | | | KIR2DL5A/B |
| TACTILE (also | P40200 | CD155 and CD111 | As for TIGIT |
| known as CD96) | | | |
| CD200R | Q8TD46 | CD200 | |
| NKp30c | O14931-2 | B7-H6, HS-GAGs | |
| KIR2DL1 | P43626 | Multiple HLA | Other KIRs, LILRs |
| KIR2DL2 | P43627 | Multiple HLA | Other KIRs, LILRs |
| KIR2DL3 | P43628 | Multiple HLA | Other KIRs, LILRs |
| KIR2DL5A | Q8N109 | CD155 | As for TIGIT |
| KIR2DL5B | Q8NHK3 | CD155 | As for TIGIT |
| KIR3DL1 | P43629 | Multiple HLA | Other KIRs, LILRs |
| KIR3DL2 | P43630 | Multiple HLA | Other KIRs, LILRs |
| KIR3DL3 | Q8N743 | Not validated | |
| LILRB1 | Q8NHL6 | Multiple HLA | Other LILRs, KIRs |
| LILRB2 | Q8N423 | Multiple HLA | Other LILRs, KIRs |
| LILRB3 | O75022 | Likely multiple | Other LILRs, KIRs |
| | | HLA | |
| LILRB4 | Q8NHJ6 | Multiple HLA | Other LILRs, KIRs |
| LILRB5 | O75023 | Multiple HLA | Other LILRs, KIRs |
| CEACAM-1 (CD66a) | P13688 | Multiple, disparate | Isoforms with other |
| | | | extracellular sequences |
| NKG2A | P26715 | HLA-E | CD94 |
| CD94 | Q13241 | HLA-E | NKG2A |
| KLRB1 (NKR-P1A) | Q12918 | Multiple, | |
| | | carbohydrates | |
| KLRG1 | Q96E93 | N- and E-cadherin | |
| CD33 | P20138 | Multiple, sialic | |
| | | glycans | |
| Siglec-7 | Q9Y286 | Multiple, sialic | |
| | | glycans | |
| Siglec-9 | Q9Y336 | Multiple, sialic | |
| | | glycans | |
| Siglec-10 | Q96LC7 | Multiple, sialic | |
| | | glycans | |
| Fas | P25445 | FasL | |
| FCRL6 | Q6DN72 | MHCII | |

In some embodiments, the extracellular domain comprises at least a portion of the extracellular domain of an inhibitory polypeptide that binds to a small molecule ligand. In some embodiments, the extracellular domain comprises at least a portion of the extracellular domain of an inhibitory polypeptide that binds to a soluble ligand, e.g., a cytokine. In some embodiments, the extracellular domain comprises at least a portion of the extracellular domain of an inhibitory polypeptide that binds to a cell surface ligand. In some embodiments, the extracellular domain comprises the extracellular domain, or a portion thereof, of one or more inhibitory polypeptides presented in Table 1.

In some embodiments, the extracellular domain comprises the extracellular domain, or a portion thereof, of: an adenosine receptor A2A that associates with an adenosine; an adenosine receptor A2B that associates with an adenosine; a prostaglandin receptor EP2 that associates with a prostaglandin; a prostaglandin receptor EP4 that associates with a prostaglandin; a TGF-BR1 that associates with a TGF-β polypeptide (also referred to herein as a “TGF-B polypeptide”); a TGF-BR2 that associates with a TGF-β polypeptide; an IL-10RA that associates with an IL-10 polypeptide; an IL-10RB that associates with an IL-10 polypeptide; an IL-18BP that associates with an IL-18 polypeptide; an IL-1R8 that associates with an IL-1 family polypeptide; an IL-6RA that associates with an IL-6 polypeptide; an IL-6RB (also known as gp130 and CD130) that associates with an IL-6 family polypeptide; a PD-1 that associates with a PD-L1 polypeptide; a PD-1 that associates with a PD-L2 polypeptide; a CTLA-4 that associates with a B7-1 polypeptide; a CTLA-4 that associates with a B7-2 polypeptide; a TIM-3; a Lag3 that associates with an MHCII polypeptide; a BTLA that associates with a HVEM polypeptide; a CD160 that associates with an HVEM polypeptide; a TIGIT that associates with a CD155 polypeptide and/or a CD112 polypeptide; a TACTILE that associates with a CD155 polypeptide and/or a CD111 polypeptide; a CD200R that associates with a CD200 polypeptide; an NKp30c that associates with a B7-H6 polypeptide and/or an HS-GAG polypeptide; a KIR2DL 1 that associates with an HLA polypeptide; a KIR2DL2 that associates with an HLA polypeptide; a KIR2DL3 that associates with an HLA polypeptide; a KIR2DLSA that associates with a CD155 polypeptide; a KIR2DL5B that associates with a CD155 polypeptide; a KIR3DL1 that associates with an HLA polypeptide; a KIR3DL2 that associates with an HLA polypeptide; a KIR3DL3; a LILRB1 that associates with an HLA polypeptide; a LILRB2 that associates with an HLA polypeptide; an HLA polypeptide; a LILRB4 that associates with an HLA polypeptide; a LILRB5 that associates with an HLA polypeptide; a CEACAM-1 (also known as CD66a); an NKG2A that associates with an HLA-E polypeptide; a CD94 that associates with an HLA-E polypeptide; a KLRB1 (NKR-PIA) that associates with a carbohydrate; a KLRG1 that associates with an N-cadherin polypeptide; a KLRG1 that associates with an E-cadherin polypeptide; a CD33 that associates with a sialic glycan; a Siglec-7 that associates with a sialic glycan; a Siglec-9 that associates with a sialic glycan; a Siglec-10 that associates with a sialic glycan; a Fas that associates with a FasL polypeptide; or an FCRL6 that associates with an MHCII polypeptide.

In some embodiments, the extracellular domain comprises the extracellular domain of one or more inhibitory polypeptides presented in Table 1.1. In some embodiments, the extracellular domain comprises an extracellular domain comprising an amino acid sequence having at least 90%, at least 91%, at least 92%, at least 93%, at least 94%, at least 95%, at least 96%, at least 97%, at least 98%, at least 99%, or 100% identity with the amino acid sequence of any one of SEQ ID NOs: 5-45.

TABLE 1.1
Examples of extracelluar domains

| Inhibitory | UNIPROT | | | Amino acid | SEQ |
|---|---|---|---|---|---|
| receptor | ID | Start | End | length | ID NO: |
| BTLA | Q7Z6A9 | 31 | 157 | 127 | 5 |
| CD160 | O95971 | 25 | 162 | 138 | 6 |
| CD200R | Q8TD46 | 29 | 243 | 215 | 7 |
| CD33 | P20138 | 18 | 259 | 242 | 8 |
| CEACAM-1 (also | P13688 | 35 | 428 | 394 | 9 |
| known as CD66a) | | | | | |
| CTLA-4 | P16410 | 36 | 161 | 126 | 10 |
| Fas | P25445 | 26 | 173 | 148 | 11 |
| FCRL6 | Q6DN72 | 20 | 307 | 288 | 12 |
| IL-10RA | Q13651 | 22 | 235 | 214 | 13 |
| IL-10RB | Q08334 | 20 | 220 | 201 | 14 |
| IL-1R8 | A0A291NLA3 | 1 | 118 | 118 | 15 |
| IL-6RA | P08887 | 20 | 365 | 346 | 16 |
| IL-6RB (also known | P40189 | 23 | 619 | 597 | 17 |
| as gp130 and CD130) | | | | | |
| KIR2DL1 | P43626 | 22 | 245 | 224 | 18 |
| KIR2DL2 | P43627 | 22 | 245 | 224 | 19 |
| KIR2DL3 | P43628 | 22 | 245 | 224 | 20 |
| KIR2DL5A | Q8N109 | 22 | 238 | 217 | 21 |
| KIR2DL5B | Q8NHK3 | 22 | 238 | 217 | 22 |
| KIR3DL1 | P43629 | 22 | 340 | 319 | 23 |
| KIR3DL2 | P43630 | 22 | 340 | 319 | 24 |
| KIR3DL3 | Q8N743 | 26 | 322 | 297 | 25 |
| Lag3 | P18627 | 23 | 450 | 428 | 26 |
| LILRB1 | Q8NHL6 | 24 | 461 | 438 | 27 |
| LILRB2 | Q8N423 | 22 | 461 | 440 | 28 |
| LILRB3 | O75022 | 24 | 443 | 420 | 29 |
| LILRB4 | Q8NHJ6 | 22 | 259 | 238 | 30 |
| LILRB5 | O75023 | 24 | 458 | 435 | 31 |
| NKp30c | O14931-2 | 18 | 135 | 118 | 32 |
| PD-1 | Q15116 | 24 | 170 | 147 | 33 |
| Siglec-10 | Q96LC7 | 17 | 550 | 534 | 34 |
| Siglec-7 | Q9Y286 | 19 | 353 | 335 | 35 |
| Siglec-9 | Q9Y336 | 18 | 348 | 331 | 36 |
| TACTILE (also | P40200 | 22 | 519 | 498 | 37 |
| known as CD96) | | | | | |
| TGF-BR1 (also | P36897 | 34 | 126 | 93 | 38 |
| referred to herein as | | | | | |
| TGF-βR1) | | | | | |
| TGF-BR2 (also | P37173 | 23 | 166 | 144 | 39 |
| referred to herein as | | | | | |
| TGF-βR2) | | | | | |
| TIGIT | Q495A1 | 22 | 141 | 120 | 40 |
| TIM-3 | Q8TDQ0 | 22 | 202 | 181 | 41 |
| CD94 | Q13241 | 1 | 10 | 10 | 42 |
| KLRB1 (also known | Q12918 | 1 | 45 | 45 | 43 |
| as NKR-P1A) | | | | | |
| KLRG1 | Q96E93 | 1 | 38 | 38 | 44 |
| NKG2A | P26715 | 1 | 70 | 70 | 45 |

### 1. Antigen-Binding Domains

In some embodiments, the engineered protein (e.g., chimeric protein) comprises an antigen-binding domain that specifically binds to a negative signal. In some embodiments, the antigen-binding domain specifically binds to a negative signal selected from the group consisting of transforming growth factor-beta (TGF-β), interleukin (IL) 10 (IL-10), IL-1, IL-6, programmed death-ligand 1 (PD-L1), programmed death-ligand 2 (PD-L2), B7-1, B7-2, MHCI, herpes virus entry mediator (HVEM), cluster of differentiation (CD) 155 (CD155), CD112, CD111, CD200, B7 homolog 6 (B7-H6), heparin and heparan sulfate (collectively referred to as HS-GAG), human leukocyte antigen (HLA) (e.g., HLA-E), N-cadherin, E-cadherin, and Fas ligand (FasL), and major histocompatibility MHCII. In some embodiments, the antigen-binding domain may recognize an epitope comprising the shared space between one or more antigens.

In some embodiments of any of the antigen-binding domains described herein, the antigen-binding domain can comprise an antibody or an antigen-binding fragment thereof. In some embodiments of any of the antigen-binding domains described herein, the antigen-binding domain comprises a single-chain antibody fragment (scFv) comprising a light chain variable domain (VL) and heavy chain variable domain (VH) of a monoclonal antibody. In some embodiments of any of the antigen-binding domains described herein, the scFv is human or humanized. In some embodiments of any of the antigen-binding domains described herein, the antigen-binding domain may comprise VH and VL that are directionally linked, for example, from N- to C-terminus, VH-linker-VL or VL-linker-VH. In some embodiments, the antigen-binding domain comprises complementary determining regions of a monoclonal antibody, variable regions of a monoclonal antibody, an scFv, a single domain antibody (e.g., a camelid single domain antibody), an antibody mimetic and/or antigen-binding fragments thereof. In some embodiments, the antigen-binding domain comprises an aptamer. In some embodiments, the antigen-binding domain comprises a T cell receptor (TCR)-like antibody. In some embodiments, the antigen-binding domain comprises a humanized amino acid sequence. Almost anything that binds a given negative signal with high affinity can be used as the antigen-binding domain. The arrangement of the extracellular domain can be multimeric, such as a diabody or multimeric (e.g., multimers). In some embodiments, the multimers can be formed by cross pairing of the variable portion of the light and heavy chains into a diabody.

Additional examples of extracellular domains that may be included in the engineered proteins (e.g., chimeric proteins) described herein are provided below:

### 2. Extracellular Domains Capable of Binding TGF-β

In some embodiments, the extracellular domain of an engineered protein (e.g., chimeric protein) described herein is capable of binding a TGF-polypeptide.

In some embodiments, an engineered protein (e.g., chimeric protein) provided herein comprises an extracellular domain of a TGF-β receptor 2 (TGF-BR2) polypeptide, or a fragment or portion thereof. In some embodiments, an engineered protein (e.g., chimeric protein) provided herein comprises an extracellular domain of a TGF-BR2 polypeptide comprising the amino acid sequence of SEQ ID NO: 46 or an amino acid sequence that is at least 80%, at least 85%, at least 90%, at least 91%, at least 92%, at least 93%, at least 94%, at least 95%, at least 96%, at least 97%, at least 98%, or at least 99% to the amino acid sequence of SEQ ID NO: 46. In some embodiments, an engineered protein (e.g., chimeric protein) provided herein comprises an extracellular domain of a TGF-BR2 polypeptide comprising a fragment or portion of an amino acid sequence that is at least 80%, at least 85%, at least 90%, at least 91%, at least 92%, at least 93%, at least 94%, at least 95%, at least 96%, at least 97%, at least 98%, at least 99% or at least 100% identical to the amino acid sequence of SEQ ID NO: 46.

In some embodiments, an engineered protein (e.g., chimeric protein) provided herein comprises an extracellular domain of a TGF-β receptor 1 (TGF-BR1) polypeptide, or a fragment or portion thereof. In some embodiments, an engineered protein (e.g., chimeric protein) provided herein comprises an extracellular domain of a TGF-BR1 polypeptide comprising the amino acid sequence of SEQ ID NO: 47, or an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 47. In some embodiments, an engineered protein (e.g., chimeric protein) provided herein comprises an extracellular domain of a TGF-BR1 polypeptide comprising a fragment or portion of an amino acid sequence that is at least 80%, at least 85%, at least 90%, at least 91%, at least 92%, at least 93%, at least 94%, at least 95%, at least 96%, at least 97%, at least 98%, at least 99% or at least 100% identical to the amino acid sequence of SEQ ID NO: 47.

In some embodiments, an engineered protein (e.g., chimeric protein) provided herein comprises an extracellular domain comprising an antigen-binding domain that specifically binds TGF-β. The antigen-binding domain can comprise a fragment of the VH and VL chains of a single-chain variable fragment (scFv) that specifically bind a TGF-β polypeptide such as those described in WO 2005/097832, WO 2012/167143, WO 2006/086469, WO 2007/076391, and WO 2014/164709; or U.S. Pat. Nos. 10,035,851; 5,772,998; and 8,597,646, each of which is incorporated herein by reference in its entirety.

### 3. Extracellular Domains Capable of Binding IL-10

In some embodiments, the extracellular domain of an engineered protein (e.g., chimeric protein) described herein is capable of binding an IL-10 polypeptide. In some embodiments, the extracellular domain comprises the extracellular domain, or a fragment or portion thereof, of an IL-10RA polypeptide. In some embodiments, the extracellular domain comprises an antigen-binding domain that specifically binds IL-10.

### 4. Extracellular Domains Capable of Binding HLA

In some embodiments, the extracellular domain of an engineered protein (e.g., chimeric protein) described herein is capable of binding an HLA polypeptide. In some embodiments, the extracellular domain comprises the extracellular domain, or a portion thereof, of an inhibitory KIR polypeptide (e.g., KIR2DL1, KIR2DL2, KIR2DL3, KIR2DLSA, KIR2DL55, or KIR3DL1). In some embodiments, the extracellular domain comprises the extracellular domain, or a portion thereof, of LILRB1, LILRB2, LILRB3, LILRB4, or LILRB5. In some embodiments, the extracellular domain comprises an antigen-binding domain that specifically binds HLA CD155.

### 5. Extracellular Domains Capable of Binding CD112 and or CD155

In some embodiments, the extracellular domain of an engineered protein (e.g., chimeric protein) described herein is capable of binding a CD112 and/or CD155 polypeptide. In some embodiments, the extracellular domain comprises the extracellular domain, or a portion thereof, of a TIGIT polypeptide.

In some embodiments, the extracellular domain comprises an antigen-binding domain that specifically binds CD112 and/or CD155.

### 6. Extracellular Domains Capable of Binding HLA-E

In some embodiments, the extracellular domain of an engineered protein (e.g., chimeric protein) described herein is capable of binding an HLA-E polypeptide. In some embodiments, the extracellular domain comprises the extracellular domain, or a portion thereof, of an NKG2A polypeptide. In some embodiments, the extracellular domain comprises an antigen-binding domain that specifically binds HLA-E.

### 7. Extracellular Domains Capable of Binding N-Cadherin and or E-Cadherin

In some embodiments, the extracellular domain of an engineered protein (e.g., chimeric protein) described herein is capable of binding an N-cadherin and/or E-cadherin polypeptide. In some embodiments, the extracellular domain comprises the extracellular domain, or a portion thereof, of a KLRG1 polypeptide.

In some embodiments, the extracellular domain comprises an antigen-binding domain that specifically binds N-cadherin and/or E-cadherin.

### 8. Extracellular Domains Capable of Binding IL-18

In some embodiments, the extracellular domain of an engineered protein (e.g., chimeric protein) described herein is capable of binding an IL-18 polypeptide. In some embodiments, the extracellular domain comprises the extracellular domain, or a portion thereof, of an IL-18BP polypeptide.

In some embodiments, the extracellular domain comprises an antigen-binding domain that specifically binds IL-18.

### B. Intracellular Domains

In some embodiments, an intracellular domain comprised in an engineered protein (e.g., chimeric protein) of the disclosure comprises the intracellular domain, or a portion thereof, of a stimulatory polypeptide. In some embodiments, the engineered protein (e.g., chimeric protein) comprises the intracellular domain, or a portion thereof, of two or more different stimulatory polypeptides. In some embodiments, the engineered protein (e.g., chimeric protein) comprises the intracellular domain, or a portion thereof, of three or more different stimulatory polypeptides. In some embodiments, the intracellular domain comprises the intracellular domain, or a portion thereof, of a stimulatory polypeptide selected from the stimulatory polypeptides presented in Table 2.

TABLE 2
Examples of Intracellular Domains

| Stimulatory Polypeptide | UNIPROT ID | Isoform(s) | Class |
|---|---|---|---|
| Caspase recruitment domain-containing protein 11 | Q9BXL7 | 1 | Adaptor |
| (also known as CARD11 and Carma 1) | | | |
| Hematopoietic cell signal transducer (also known as | Q9UBK5 | 1 | Adaptor |
| DNAX-Activation Protein 10 and DAP10) | | | |
| Linker for activation of T-cells family member 1 | O43561 | 1, 2, 3, 4, 5 | Adaptor |
| (LAT) | | | |
| Linker for activation of T-cells family member 2 | Q9GZY6 | 1 | Adaptor |
| (also known as LAT2, NTAL, and LAB) | | | |
| Lymphocyte cytosolic protein 2 (also known as | Q13094 | 1 | Adaptor |
| SLP76) | | | |
| Lymphocyte transmembrane adapter 1 (also known | Q8IWV1 | 1 | Adaptor |
| as LAX) | | | |
| Myeloid differentiation primary response protein | Q99836 | 1, 2, 4, 6, 8 | Adaptor |
| MyD88 (also known as MyD88) | | | |
| Phosphoprotein associated with glycosphingolipid- | Q9NWQ8 | 1 | Adaptor |
| enriched microdomains 1 (also known as PAG and | | | |
| CBP) | | | |
| Protein GAPT (also known as GAPT, and Growth | Q8N292 | 1 | Adaptor |
| Factor Receptor-Bound Protein 2-Binding Adapter | | | |
| Protein, Transmembrane) | | | |
| SH2 domain-containing protein 1A (also known as | O60880 | A, B, C, D, | Adaptor |
| SAP) | | E, F | |
| SH2 domain-containing protein 1B (also known as | O14796 | 1, 2 | Adaptor |
| EAT-2) | | | |
| TYRO protein tyrosine kinase-binding protein (also | O43914 | 1 | Adaptor |
| known as DNAX-Activation Protein 12 and DAP12 | | | |
| High affinity immunoglobulin epsilon receptor | P30273 | 1 | Antibody |
| subunit gamma (also known as FcRgamma and | | | receptor |
| FceRI gamma) | | | |
| High affinity immunoglobulin gamma Fc receptor I | P12314 | 1 | Antibody |
| (also known as FcRI, Fc-gamma RI, and CD64A) | | | receptor |
| Low affinity immunoglobulin gamma Fc region | P12318 | 1, 3 | Antibody |
| receptor II-a (also known as FcRII-a, Fc-gamma | | | receptor |
| RIIa, and CD32A) | | | |
| Low affinity immunoglobulin gamma Fc region | P31995 | 1, 2, 3, 4, 5 | Antibody |
| receptor II-c (also known as FcRII-c, Fc-gamma | | | receptor |
| RIIc, and CD32C) | | | |
| Low affinity immunoglobulin gamma Fc region | P08637 | 1 | Antibody |
| receptor III-A (also known as FcRIIIa, Fc-gamma | | | receptor |
| RIIIa, and CD16A) | | | |
| Lymphocyte function-associated antigen 3 (also | P19256 | 1 | CD2 family |
| known as LFA-3 and CD58) | | | receptor |
| Natural killer cell receptor 2B4 (also known as 2B4, | Q9BZW8 | 1, 3 | CD2 family |
| SLAMF4, and CD244) | | | receptor |
| Signaling lymphocytic activation molecule (also | Q13291 | 1, 2, 4 | CD2 family |
| known as SLAM, SLAMF1 and CD150) | | | receptor |
| SLAM family member 5 (also known as SLAMF5 | Q9UIB8 | 1, 2, 3, 4, | CD2 family |
| and CD84) | | 5, 7 | receptor |
| SLAM family member 6 (also known as SLAMF6, | Q96DU3 | 1 | CD2 family |
| NTB-A, and CD352) | | | receptor |
| SLAM family member 7 (also known as SLAMF7 | Q9NQ25 | 1, 3, 5 | CD2 family |
| and CD319) | | | receptor |
| T-cell surface antigen CD2 (also known as LFA-2 | P06729 | 1 | CD2 family |
| and CD2) | | | receptor |
| T-lymphocyte surface antigen Ly-9 (also known as | Q9HBG7 | 1, 2, 3 | CD2 family |
| SLAMF3, Ly-9, and CD229) | | | receptor |
| Carcinoembryonic antigen-related cell adhesion | P40198 | 1, 2, 3 | CEACAM |
| molecule 3 (also known as CEACAM-3 and | | | family |
| CD66D) | | | |
| CD209 antigen (also known as DC-SIGN, CLEC- | Q9NNX6 | 1, 5 | C-type lectin |
| 4L, and CD209) | | | family receptor |
| C-type lectin domain family 1 member B (also | Q9P126 | 1 | C-type lectin |
| known as CLEC-2) | | | family receptor |
| C-type lectin domain family 7 member A (also | Q9BXN2 | 1 | C-type lectin |
| known as Dectin-1 and CLEC-7A) | | | family receptor |
| C-type lectin domain family 9 member A (also | Q6UXN8 | 1 | C-type lectin |
| known as DNGR-1 and CD370) | | | family receptor |
| Killer cell lectin-like receptor subfamily F member | Q9NZS2 | 1 | C-type lectin |
| 1 (also known as NKp80, KLRF1 and CLEC5C) | | | family receptor |
| Killer cell lectin-like receptor subfamily F member | D3W0D1 | 1 | C-type lectin |
| 2 (also known as NKp65 and KLRF2) | | | family receptor |
| NKG2-C type II integral membrane protein (also | P26717 | 1 | C-type lectin |
| known as NKG2C, KLRC2, CD159C) | | | family receptor |
| NKG2-D type II integral membrane protein (also | P26718 | 1 | C-type lectin |
| known as NKG2D and CD314) | | | family receptor |
| NKG2-E type II integral membrane protein (also | Q07444 | 1 (E) | C-type lectin |
| known as NKG2E and KLRC3) | | | family receptor |
| Cytokine receptor common subunit beta (also | P32927 | 1 | Cytokine |
| known as CD131) | | | receptor |
| Cytokine receptor common subunit gamma (also | P31785 | 1 | Cytokine |
| known as IL-2RG and CD132) | | | receptor |
| Cytokine receptor-like factor 2 (also known as | Q9HC73 | 1 | Cytokine |
| TSLP-R) | | | receptor |
| Erythropoietin receptor (also known as EPO-R or | P19235 | 1 | Cytokine |
| EPOR) | | | receptor |
| Granulocyte colony-stimulating factor receptor (also | Q99062 | 1, 2, 3, 4 | Cytokine |
| known as G-CSF-R, GCSFR, and CD114) | | | receptor |
| Granulocyte-macrophage colony-stimulating factor | P15509 | 1, 2 | Cytokine |
| receptor subunit alpha (also known as GM-CSF-R- | | | receptor |
| alpha and CD116) | | | |
| Interferon alpha/beta receptor 1 (also known as IFN- | P17181 | 1 | Cytokine |
| R1 and IFNA/B-R1) | | | receptor |
| Interferon alpha/beta receptor 2 (also known as IFN- | P48551 | 1 | Cytokine |
| R2 and IFNA/B-R2) | | | receptor |
| Interferon lambda receptor 1 (also known as IL- | Q8IU57 | 1, 2 | Cytokine |
| 28RA and IFN-lambda-R1 | | | receptor |
| Interleukin-1 receptor accessory protein (also | Q9NPH3 | 1, 4 | Cytokine |
| known as IL-1R3 and IL-1RAP) | | | receptor |
| Interleukin-1 receptor type 1 (also known as IL- | P14778 | 1 | Cytokine |
| 1R1, IL-1RA, and CD121A) | | | receptor |
| Interleukin-1 receptor-like 1 (also known as ST2 | Q01638 | 1 | Cytokine |
| and IL-1RL1) | | | receptor |
| Interleukin-1 receptor-like 2 (also known as IL-36R | Q9HB29 | 1 | Cytokine |
| and IL-1RL2 | | | receptor |
| Interleukin-11 receptor subunit alpha (also known | Q14626 | 1 | Cytokine |
| as IL-11RA) | | | receptor |
| Interleukin-12 receptor subunit beta-1 (also known | P42701 | 1 | Cytokine |
| as IL-12RB1 and CD212) | | | receptor |
| Interleukin-12 receptor subunit beta-2 (also known | Q99665 | 1 | Cytokine |
| as IL-12RB2) | | | receptor |
| Interleukin-17 receptor A (also known as IL-17RA | Q96F46 | 1 | Cytokine |
| and CD217) | | | receptor |
| Interleukin-17 receptor B (also known as IL-17RB) | Q9NRM6 | 1 | Cytokine |
| | | | receptor |
| Interleukin-17 receptor C (also known as IL-17RC) | Q8NAC3 | 1 | Cytokine |
| | | | receptor |
| Interleukin-17 receptor E (also known as IL-17RE) | Q8NFR9 | 1 | Cytokine |
| | | | receptor |
| Interleukin-18 receptor 1 (also known as IL-18R1, | Q13478 | 1 | Cytokine |
| IL-1RRP and CD218A) | | | receptor |
| Interleukin-18 receptor accessory protein (also | O95256 | 1 | Cytokine |
| known as IL-18RB, IL-1-R7, and CD218B) | | | receptor |
| Interleukin-2 receptor subunit beta (also known as | P14784 | 1 | Cytokine |
| IL-2RB, IL-15RB, and CD122) | | | receptor |
| Interleukin-21 receptor (also known as IL-21R and | Q9HBE5 | 1 | Cytokine |
| CD360) | | | receptor |
| Interleukin-22 receptor subunit alpha-1 (also known | Q8N6P7 | 1 | Cytokine |
| as IL-22RA1) | | | receptor |
| Interleukin-23 receptor (also known as IL-23R) | Q5VWK5 | 1 | Cytokine |
| | | | receptor |
| Interleukin-27 receptor subunit alpha (also known | Q6UWB1 | 1 | Cytokine |
| as IL-27RA and WSX-1) | | | receptor |
| Interleukin-3 receptor subunit alpha (also known as | P26951 | 1 | Cytokine |
| IL-3RA and CD123) | | | receptor |
| Interleukin-6 receptor subunit beta (also known as | P40189 | 1 | Cytokine |
| IL-6RB, gp130, and CD130) | | | receptor |
| Interleukin-7 receptor subunit alpha (also known as | P16871 | 1 | Cytokine |
| IL-7RA and CD127) | | | receptor |
| Leukemia inhibitory factor receptor (also known as | P42702 | 1 | Cytokine |
| LIF-R and CD118) | | | receptor |
| Macrophage colony-stimulating factor 1 receptor | P07333 | 1 | Cytokine |
| (also known as M-CSF-R, CSF-1R, CSF1R, and | | | receptor |
| CD115) | | | |
| Oncostatin-M-specific receptor subunit beta (also | Q99650 | 1 | Cytokine |
| known as OSM-RB and IL-31RB) | | | receptor |
| Epidermal growth factor receptor (also known as | P00533 | 1 | Growth factor |
| EGFR and Hen) | | | receptor |
| Growth hormone receptor (also known as GHR and | P10912 | 1 | Growth factor |
| GH receptor) | | | receptor |
| Insulin receptor (also known as IR and CD220) | P06213 | Beta chain | Growth factor |
| | | | receptor |
| Leptin receptor (also known as LEP-R, OB-R and | P48357 | a, b, c, d, f | Growth factor |
| CD295) | | | receptor |
| Prolactin receptor (also known as PRL-R) | P16471 | 1 | Growth factor |
| | | | receptor |
| Thrombopoietin receptor (also known as TPO-R, c- | P40238 | 1, 2 | Growth factor |
| Mpl, and CD110) | | | receptor |
| B-cell antigen receptor complex-associated protein | P11912 | 1 | Ig family |
| alpha chain (also known as Ig-alpha and CD79A) | | | receptor |
| B-cell antigen receptor complex-associated protein | P40259 | 1 | Ig family |
| beta chain (also known as Ig-beta and CD79B) | | | receptor |
| CD226 antigen (also known as DNAM-1 and | Q15762 | 1 | Ig family |
| CD226) | | | receptor |
| CD83 antigen (also known as CD83) | Q01151 | 1 | Ig family |
| | | | receptor |
| Inducible T-cell costimulator (also known as ICOS | Q9Y6W8 | 1 | Ig family |
| and CD278) | | | receptor |
| Intercellular adhesion molecule 1 (also known as | P05362 | 1 | Ig family |
| ICAM-1 and CD54) | | | receptor |
| Intercellular adhesion molecule 2 (also known as | P13598 | 1 | Ig family |
| ICAM-2 and CD102) | | | receptor |
| Intercellular adhesion molecule 3 (also known as | P32942 | 1 | Ig family |
| ICAM-3 and CD50) | | | receptor |
| Killer cell immunoglobulin-like receptor 2DL4 (also | Q99706 | 1 | Ig family |
| known as KIR2DL4 and CD158D) | | | receptor |
| Killer cell immunoglobulin-like receptor 2DS1 (also | Q14954 | 1 | Ig family |
| known as KIR2DS1 and CD158H) | | | receptor |
| Killer cell immunoglobulin-like receptor 2DS2 (also | P43631 | 1 | Ig family |
| known as KIR2DS2 and CD158J) | | | receptor |
| Killer cell immunoglobulin-like receptor 2DS3 (also | Q14952 | 1 | Ig family |
| known as KIR2DS3) | | | receptor |
| Killer cell immunoglobulin-like receptor 2DS4 (also | P43632 | 1 | Ig family |
| known as KIR2DS4 and CD158I) | | | receptor |
| Killer cell immunoglobulin-like receptor 2DS50 | Q14953 | 1 | Ig family |
| (also known as KIR2DS5 and CD158G) | | | receptor |
| Killer cell immunoglobulin-like receptor 3DS1 (also | Q14943 | 1 | Ig family |
| known as KIR3DS1) | | | receptor |
| Natural cytotoxicity triggering receptor 1 (also | O76036 | 1 | Ig family |
| known as NKp46, Ly94 and CD335) | | | receptor |
| Natural cytotoxicity triggering receptor 2 (also | O95944 | 1, 2, 3 | Ig family |
| known as NKp44 and CD336) | | | receptor |
| Natural cytotoxicity triggering receptor 3 (also | O14931 | 1, 2, 3 | Ig family |
| known as NKp30 and CD337) | | | receptor |
| T-cell antigen CD7 (also known as CD7) | P09564 | 1 | Ig family |
| | | | receptor |
| T-cell surface glycoprotein CD4 (also known as | P01730 | 1 | Ig family |
| CD4) | | | receptor |
| T-cell-specific surface glycoprotein CD28 (also | P10747 | 1 | Ig family |
| known as CD28) | | | receptor |
| Transmembrane and immunoglobulin domain- | Q96BF3 | 1, 2 | Ig family |
| containing protein 2 (also known as TMIGD2, | | | receptor |
| CD28H, and IGPR-1) | | | |
| Integrin alpha-L (also known as LFA-1A and | P20701 | 1 | Integrin |
| CD11A) | | | |
| Integrin beta-2 (also known as LFA-1B and CD18) | P05107 | 1 | Integrin |
| Cytotoxic and regulatory T-cell molecule (also | O95727 | 1 | Nectin family |
| known as CRTAM and CD355) | | | receptor |
| B-cell receptor CD22 (also known as Siglec-2 and | P20273 | 1, 4 | Siglec lectin |
| CD22) | | | family receptor |
| T-cell surface glycoprotein CD3 epsilon chain (also | P07766 | 1 | Src family |
| known as CD3E) | | | tyrosine kinase |
| T-cell surface glycoprotein CD3 gamma chain (also | P09693 | 1 | Src family |
| known as CD3G) | | | tyrosine kinase |
| T-cell surface glycoprotein CD3 zeta chain (also | P20963 | 1 | Src family |
| known as CD3Z, CD3ζ, and CD247) | | | tyrosine kinase |
| Tyrosine-protein kinase Lck (also known as Lck, | P06239 | 1, 2, 3 | Src family |
| p56Lck, and LSK) | | | tyrosine kinase |
| Tyrosine-protein kinase ZAP-70 (also known as | P43403 | 1, 2, 3 | Syk family |
| ZAP70) | | | tyrosine kinase |
| Hepatitis A virus cellular receptor 1 (also known as | Q96D42 | 1 | TIM receptor |
| TIM-1, KIM-1, and CD365) | | | family |
| Toll-like receptor 1 (also known as TLR1 and | Q15399 | 1 | Toll-like |
| CD281) | | | receptor (TLR) |
| | | | family |
| Toll-like receptor 10 (also known as TLR10 and | Q9BXR5 | 1 | TLR family |
| CD290) | | | |
| Toll-like receptor 2 (also known as TLR2 and | O60603 | 1 | TLR family |
| CD282) | | | |
| Toll-like receptor 3 (also known as TLR3 and | O15455 | 1 | TLR family |
| CD283) | | | |
| Toll-like receptor 4 (also known as TLR4 and | O00206 | 1 | TLR family |
| CD284) | | | |
| Toll-like receptor 5 (also known as TLR5 and) | O60602 | 1 | TLR family |
| CD285 | | | |
| Toll-like receptor 6 (also known as TLR6 and | Q9Y2C9 | 1 | TLR family |
| CD286) | | | |
| Toll-like receptor 7 (also known as TLR7 and | Q9NYK1 | 1 | TLR family |
| CD287) | | | |
| Toll-like receptor 8 (also known as TLR8 and | Q9NR97 | 1 | TLR family |
| CD288) | | | |
| Toll-like receptor 9 (also known as TLR9 and | Q9NR96 | 1 | TLR family |
| CD289) | | | |
| CD27 antigen (also known as CD27) | P26842 | 1 | TNF family |
| | | | receptor |
| CD70 antigen (also known as CD70) | P32970 | 1 | TNF family |
| | | | receptor |
| Tumor necrosis factor ligand superfamily member | O43557 | 1 | TNF family |
| 14 (also known as LIGHT and CD258) | | | receptor |
| Tumor necrosis factor ligand superfamily member 8 | P32971 | 1 | TNF family |
| (also known as CD30L and CD153) | | | receptor |
| Tumor necrosis factor receptor superfamily member | Q9Y6Q6 | 1, 2, 3, 4, 5 | TNF family |
| 11A (also known as TNFRSF11A, RANK, and | | | receptor |
| CD265) | | | |
| Tumor necrosis factor receptor superfamily member | Q9NP84 | 1 | TNF family |
| 12A (also known as TNFRSF12A, TweakR, FN14, | | | receptor |
| and CD266) | | | |
| Tumor necrosis factor receptor superfamily member | O14836 | 1 | TNF family |
| 13B (also known as TNFRSF13B, TACI, and | | | receptor |
| CD267) | | | |
| Tumor necrosis factor receptor superfamily member | Q96RJ3 | 1 | TNF family |
| 13C (also known as TNFRSF13C, BAFF-R, and | | | receptor |
| CD268) | | | |
| Tumor necrosis factor receptor superfamily member | Q92956 | 1 | TNF family |
| 14 (also known as TNFRSF14, HVEM, and CD270) | | | receptor |
| Tumor necrosis factor receptor superfamily member | P08138 | 1 | TNF family |
| 16 (also known as TNFRSF16, NGF-R, p75NTR, | | | receptor |
| and CD271) | | | |
| Tumor necrosis factor receptor superfamily member | Q02223 | 1 | TNF family |
| 17 (also known as TNFRSF17, BCMA, and CD269) | | | receptor |
| Tumor necrosis factor receptor superfamily member | Q9Y5U5 | 1, 2, 3 | TNF family |
| 18 (also known as TNFRSF18, GITR, and CD357) | | | receptor |
| Tumor necrosis factor receptor superfamily member | Q9NS68 | 1, 2 | TNF family |
| 19 (also known as TNFRSF19, TROY, and | | | receptor |
| TRADE) | | | |
| Tumor necrosis factor receptor superfamily member | Q969Z4 | 1 | TNF family |
| 19L (also known as TNFRSF19L and RELT) | | | receptor |
| Tumor necrosis factor receptor superfamily member | P19438 | 1 | TNF family |
| lA (also known as TNFRS1A, TNF-RI, and | | | receptor |
| CD120A) | | | |
| Tumor necrosis factor receptor superfamily member | P20333 | 1 | TNF family |
| 1B (also known as TNFRSF1B, TNF-RII, and | | | receptor |
| CD120B) | | | |
| Tumor necrosis factor receptor superfamily member | Q93038 | 1 | TNF family |
| 25 (also known as TNFRSF25, DR3, and TRAMP) | | | receptor |
| Tumor necrosis factor receptor superfamily member | Q9HAV5 | 1, 2, 3 | TNF family |
| 27 (also known as TNFRSF27, XEDAR, and EDA- | | | receptor |
| A2 receptor) | | | |
| Tumor necrosis factor receptor superfamily member | P36941 | 1 | TNF family |
| 3 (also known as TNFRSF3, LTB-R, and TNF-RIII) | | | receptor |
| Tumor necrosis factor receptor superfamily member | P43489 | 1 | TNF family |
| 4 (also known as TNFRSF4, OX-40, and CD134) | | | receptor |
| Tumor necrosis factor receptor superfamily member | P25942 | 1 | TNF family |
| 5 (also known as TNFRSF5 and CD40) | | | receptor |
| Tumor necrosis factor receptor superfamily member | P28908 | 1 | TNF family |
| 8 (also known as TNFRSF8 and CD30) | | | receptor |
| Tumor necrosis factor receptor superfamily member | Q07011 | 1 | TNF family |
| 9 (also known as TNFRSF9, 4-1BB and CD137) | | | receptor |
| Tumor necrosis factor receptor superfamily member | Q9UNE0 | 1 | TNF family |
| EDAR (also known as EDAR) | | | receptor |
| Paired immunoglobulin-like type 2 receptor beta | Q9UKJ0 | 1, 2, 3 | Ig family |
| (also known as PILRB) | | | receptor |

In some embodiments, an engineered protein (e.g., chimeric protein) provided herein includes an intracellular domain comprising an intracellular domain, or a portion thereof, of one or more isoforms of the stimulatory polypeptides listed in Table 2.

In some embodiments, an engineered protein (e.g., chimeric protein) provided herein includes an intracellular domain comprising an intracellular domain, or a portion thereof, of a class of stimulatory polypeptide (e.g., as described in Table 2). For example, in some embodiments, the intracellular domain comprises at least a portion of the intracellular domain of: an adaptor polypeptide, an antibody receptor polypeptide, a CD2 family receptor polypeptide, a CEACAM family polypeptide, a C-type lectin family receptor polypeptide, a cytokine receptor polypeptide, a growth factor receptor polypeptide, an Ig family receptor polypeptide, an integrin polypeptide, a nectin family receptor polypeptide, a siglec lectin family receptor polypeptide, a src family tyrosine kinase polypeptide, a syk family tyrosine kinase polypeptide, a TIM receptor family polypeptide, a TLR family polypeptide, or a TNF family receptor polypeptide. Non-limiting examples of these polypeptides are listed in Table 2.

In some embodiments, the intracellular domain of an engineered protein (e.g., chimeric protein) disclosed herein is responsible for activation of at least one of the normal effector functions of the immune cell (e.g., NK cell) in which the engineered protein (e.g., chimeric protein) has been expressed. In some embodiments, the intracellular domain comprises a signaling domain for NK cell activation. In some embodiments, an engineered protein (e.g., chimeric protein) provided herein includes at least one (e.g., one, two, three, four, or five) intracellular domain of one or more of the polypeptides selected from the group consisting of: caspase recruitment domain-containing protein 11, hematopoietic cell signal transducer, linker for activation of T-cells family member 1, linker for activation of T-cells family member 2, lymphocyte cytosolic protein 2, lymphocyte transmembrane adapter 1, myeloid differentiation primary response protein MyD88, phosphoprotein associated with glycosphingolipid-enriched microdomains 1, protein GAPT, SH2 domain-containing protein 1A, SH2 domain-containing protein 1B. TYRO protein tyrosine kinase-binding protein, high affinity immunoglobulin epsilon receptor subunit gamma, high affinity immunoglobulin gamma Fc receptor I, low affinity immunoglobulin gamma Fc region receptor II-a, low affinity immunoglobulin gamma Fc region receptor II-c, low affinity immunoglobulin gamma Fc region receptor III-A, lymphocyte function-associated antigen 3, natural killer cell receptor 2B4, signaling lymphocytic activation molecule, SLAM family member 5, SLAM family member 6, SLAM family member 7, T-cell surface antigen CD2, T-lymphocyte surface antigen Ly-9, carcinoembryonic antigen-related cell adhesion molecule 3, CD209 antigen, C-type lectin domain family 1 member B, C-type lectin domain family 7 member A, C-type lectin domain family 9 member A, killer cell lectin-like receptor subfamily F member 1, killer cell lectin-like receptor subfamily F member 2, NKG2-C type II integral membrane protein, NKG2-D type II integral membrane protein, NKG2-E type II integral membrane protein, cytokine receptor common subunit beta, cytokine receptor common subunit gamma, cytokine receptor-like factor 2, erythropoietin receptor, granulocyte colony-stimulating factor receptor, granulocyte-macrophage colony-stimulating factor receptor subunit alpha, interferon alpha/beta receptor 1, interferon alpha/beta receptor 2, interferon lambda receptor 1, interleukin-1 receptor accessory protein, interleukin-1 receptor type 1, interleukin-1 receptor-like 1, interleukin-1 receptor-like 2, interleukin-11 receptor subunit alpha, interleukin-12 receptor subunit beta-1, interleukin-12 receptor subunit beta-2, interleukin-17 receptor A, interleukin-17 receptor B, interleukin-17 receptor C, interleukin-17 receptor E, interleukin-18 receptor 1, interleukin-18 receptor accessory protein, interleukin-2 receptor subunit beta, interleukin-21 receptor, interleukin-22 receptor subunit alpha-1, interleukin-23 receptor, interleukin-27 receptor subunit alpha, interleukin-3 receptor subunit alpha, interleukin-6 receptor subunit beta, interleukin-7 receptor subunit alpha, leukemia inhibitory factor receptor, macrophage colony-stimulating factor 1 receptor, oncostatin-M-specific receptor subunit beta, epidermal growth factor receptor, growth hormone receptor, insulin receptor, leptin receptor, prolactin receptor, thrombopoietin receptor, B-cell antigen receptor complex-associated protein alpha chain, B-cell antigen receptor complex-associated protein beta chain, CD226 antigen, CD83 antigen, inducible T-cell costimulatory, intercellular adhesion molecule 1, intercellular adhesion molecule 2, intercellular adhesion molecule 3, killer cell immunoglobulin-like receptor 2DL4, killer cell immunoglobulin-like receptor 2DS1, killer cell immunoglobulin-like receptor 2DS2, killer cell immunoglobulin-like receptor 2DS3, killer cell immunoglobulin-like receptor 2DS4, killer cell immunoglobulin-like receptor 2DS50, killer cell immunoglobulin-like receptor 3DS1, natural cytotoxicity triggering receptor 1, natural cytotoxicity triggering receptor 2, natural cytotoxicity triggering receptor 3, T-cell antigen CD7, T-cell surface glycoprotein CD4, T-cell-specific surface glycoprotein CD28, transmembrane and immunoglobulin domain-containing protein 2, integrin alpha-L, integrin beta-2, cytotoxic and regulatory T-cell molecule, B-cell receptor CD22, T-cell surface glycoprotein CD3 epsilon chain, T-cell surface glycoprotein CD3 gamma chain, T-cell surface glycoprotein CD3 zeta chain, tyrosine-protein kinase Lck, tyrosine-protein kinase ZAP-70, Hepatitis A virus cellular receptor 1, Toll-like receptor 1, Toll-like receptor 10, Toll-like receptor 2, Toll-like receptor 3, Toll-like receptor 4, Toll-like receptor 5, Toll-like receptor 6, Toll-like receptor 7, Toll-like receptor 8, Toll-like receptor 9, CD27 antigen, CD70 antigen, tumor necrosis factor ligand superfamily member 14, tumor necrosis factor ligand superfamily member 8, tumor necrosis factor receptor superfamily member 11A, tumor necrosis factor receptor superfamily member 12A, tumor necrosis factor receptor superfamily member 13B, tumor necrosis factor receptor superfamily member 13C, tumor necrosis factor receptor superfamily member 14, tumor necrosis factor receptor superfamily member 16, tumor necrosis factor receptor superfamily member 17, tumor necrosis factor receptor superfamily member 18, tumor necrosis factor receptor superfamily member 19, tumor necrosis factor receptor superfamily member 19L, tumor necrosis factor receptor superfamily member 1A, tumor necrosis factor receptor superfamily member 1B, tumor necrosis factor receptor superfamily member 25, tumor necrosis factor receptor superfamily member 27, tumor necrosis factor receptor superfamily member 3, tumor necrosis factor receptor superfamily member 4, tumor necrosis factor receptor superfamily member 5, tumor necrosis factor receptor superfamily member 8, tumor necrosis factor receptor superfamily member 9, and tumor necrosis factor receptor superfamily member EDAR, or a portion of any of the foregoing.

In some embodiments, an engineered protein (e.g., chimeric protein) provided herein comprises an intracellular domain, or a portion thereof, of: a CD226 polypeptide, a natural cytotoxicity triggering receptor 1 polypeptide, a CD160 polypeptide, a hematopoietic cell signal transducer polypeptide, a TYRO protein tyrosine kinase-binding protein polypeptide, a myeloid differentiation primary response protein MyD88 polypeptide, a granulocyte colony-stimulating factor receptor polypeptide, a macrophage colony-stimulating factor 1 receptor polypeptide, an erythropoietin receptor polypeptide, an inducible T-cell costimulatory polypeptide, a T-cell-specific surface glycoprotein CD28 polypeptide, a transmembrane and immunoglobulin domain-containing protein 2 polypeptide, a tumor necrosis factor receptor superfamily member 9 polypeptide, a tumor necrosis factor receptor superfamily member 25 polypeptide, a tumor necrosis factor receptor superfamily member 4 polypeptide, a low affinity immunoglobulin gamma Fc region receptor III-A polypeptide, a low affinity immunoglobulin gamma Fc region receptor II-c polypeptide, a high affinity immunoglobulin epsilon receptor subunit gamma polypeptide, a T-cell surface antigen CD2 polypeptide, a natural killer cell receptor 2B4 polypeptide, a SLAM family member 7 polypeptide, a T-cell surface glycoprotein CD3 epsilon chain polypeptide, a T-cell surface glycoprotein CD3 gamma chain polypeptide, a T-cell surface glycoprotein CD3 zeta chain polypeptide, a carcinoembryonic antigen-related cell adhesion molecule 3 polypeptide, 1a macrophage mannose receptor 1 polypeptide, an intercellular adhesion molecule 1 polypeptide, an intercellular adhesion molecule 2 polypeptide, an intercellular adhesion molecule 3 polypeptide, an interleukin-1 receptor-associated kinase 1 polypeptide, an interleukin-1 receptor-associated kinase-like 2 polypeptide, an interleukin-1 receptor-associated kinase 4 polypeptide, a B-cell receptor CD22 polypeptide, a sialic acid-binding Ig-like lectin 14 polypeptide, a sialic acid-binding Ig-like lectin 15 polypeptide, a hepatitis A virus cellular receptor I polypeptide, a toll-like receptor 3 polypeptide, a toll-like receptor 4 polypeptide, a toll-like receptor 9 polypeptide, a tyrosine-protein kinase SYK polypeptide, a proto-oncogene tyrosine-protein kinase Src polypeptide, a tyrosine-protein kinase ZAP-70 polypeptide, a killer cell lectin-like receptor subfamily F member 2 polypeptide, a killer cell lectin-like receptor subfamily F member 1 polypeptide, a NKG2-D type II integral membrane protein polypeptide, a C-type lectin domain family 7 member A polypeptide, a tumor necrosis factor ligand superfamily member 9 polypeptide, a tumor necrosis factor ligand superfamily member 14 polypeptide, a tumor necrosis factor ligand superfamily member 13B polypeptide, or a paired immunoglobulin-like type 2 receptor beta (PILRB).

In some embodiments, an engineered protein (e.g., chimeric protein) provided herein includes an comprising the intracellular domain of one or more stimulatory polypeptides presented in Table 2.1. In some embodiments, an engineered protein (e.g., chimeric protein) provided herein includes an intracellular domain selected from the intracellular domains presented in Table 2.1.

TABLE 2.1
Exemplary IntraceHular Domains

| | | | | Amino | SEQ |
|---|---|---|---|---|---|
| | UNIPROT | | | acid | ID |
| Intracellular Polypeptide | ID | Start | End | length | NO: |
| Hematopoietic cell signal transducer (also known as | Q9UBK5 | 70 | 93 | 24 | 48 |
| DAP10) | | | | | |
| Protein GAPT (also known as GAPT) | Q8N292 | 32 | 157 | 126 | 49 |
| TYRO protein tyrosine kinase-binding protein (also | O43914 | 62 | 113 | 52 | 50 |
| known as DAP12) | | | | | |
| High affinity immunoglobulin epsilon receptor | P30273 | 45 | 86 | 42 | 51 |
| subunit gamma (also known as FcRgamma and | | | | | |
| FceRI gamma) | | | | | |
| High affinity immunoglobulin gamma Fc receptor I | P12314 | 314 | 374 | 61 | 52 |
| (also known as FcRI, Fc-gamma RI, and CD64A) | | | | | |
| Low affinity immunoglobulin gamma Fc region | P12318 | 241 | 317 | 77 | 53 |
| receptor II-a (also known as FcRII-a, Fc-gamma | | | | | |
| RIIa, and CD32A) | | | | | |
| Low affinity immunoglobulin gamma Fc region | P31995 | 247 | 323 | 77 | 54 |
| receptor II-c (also known as FcRII-c, Fc-gamma | | | | | |
| RIIc, and CD32C) | | | | | |
| Low affinity immunoglobulin gamma Fc region | P08637 | 230 | 254 | 25 | 55 |
| receptor III-A (also known as FcRIIIa, Fc-gamma | | | | | |
| RIIIa, and CD16A) | | | | | |
| Immunoglobulin alpha Fc receptor (also known as | P24071 | 247 | 287 | 41 | 56 |
| FCARI and CD89) | | | | | |
| Lymphocyte function-associated antigen 3 (also | P19256 | 239 | 250 | 12 | 57 |
| known as LFA-3 and CD58) | | | | | |
| Natural killer cell receptor 2B4 (also known as 2B4, | Q9BZW8 | 251 | 370 | 120 | 58 |
| SLAMF4, and CD244) | | | | | |
| Signaling lymphocytic activation molecule (also | Q13291 | 259 | 335 | 77 | 59 |
| known as SLAM, SLAMF1, and CD150) | | | | | |
| SLAM family member 5 (also known as SLAMF5 | Q9UIB8 | 247 | 345 | 99 | 60 |
| and CD84) | | | | | |
| SLAM family member 6 (also known as SLAMF6, | Q96DU3 | 248 | 331 | 84 | 61 |
| NTB-A, and CD352) | | | | | |
| SLAM family member 7 (also known as SLAMF7 | Q9NQ25 | 248 | 335 | 88 | 62 |
| and CD319) | | | | | |
| T-cell surface antigen CD2 (also known as LFA-2 | P06729 | 236 | 351 | 116 | 63 |
| and CD2) | | | | | |
| T-lymphocyte surface antigen Ly-9 (also known as | Q9HBG7 | 477 | 655 | 179 | 64 |
| SLAMF3, Ly-9 and CD229) | | | | | |
| T-cell surface glycoprotein CD3 epsilon chain (also | P07766 | 153 | 207 | 55 | 65 |
| known as CD3E) | | | | | |
| T-cell surface glycoprotein CD3 gamma chain (also | P09693 | 138 | 182 | 45 | 66 |
| known as CD3G) | | | | | |
| T-cell surface glycoprotein CD3 zeta chain (also | P20963 | 52 | 164 | 113 | 67 |
| known as CD3Z and CD247) | | | | | |
| Carcinoembryonic antigen-related cell adhesion | P40198 | 177 | 252 | 76 | 68 |
| molecule 3 (also known as CEACAM-3 and | | | | | |
| CD66D) | | | | | |
| Complement receptor type 1 (also known as CR1, | P17927 | 1997 | 2039 | 43 | 69 |
| C3B/C4b receptor, and CD35) | | | | | |
| Membrane cofactor protein (also known as MCP | P15529 | 367 | 392 | 26 | 70 |
| and CD46) | | | | | |
| Macrophage mannose receptor 1 (also known as | P22897 | 1411 | 1456 | 46 | 71 |
| MMR and CD206) | | | | | |
| Cytokine receptor common subunit beta (also | P32927 | 461 | 897 | 437 | 72 |
| known as CD131) | | | | | |
| Cytokine receptor common subunit gamma (also | P31785 | 284 | 369 | 86 | 73 |
| known as IL-2RG and CD132) | | | | | |
| Cytokine receptor-like factor 2 (also known as | Q9HC73 | 253 | 371 | 119 | 74 |
| TSLP-R) | | | | | |
| Erythropoietin receptor (also known as EPO-R and | P19235 | 274 | 508 | 235 | 75 |
| EPOR) | | | | | |
| Granulocyte colony-stimulating factor receptor (also | Q99062 | 651 | 836 | 186 | 76 |
| known as G-CSF-R, GCSFR, and CD114) | | | | | |
| Granulocyte-macrophage colony-stimulating factor | P15509 | 347 | 400 | 54 | 77 |
| receptor subunit alpha (also known as GM-CSF-R- | | | | | |
| alpha and CD116) | | | | | |
| Interferon alpha/beta receptor 1 (also known as IFN- | P17181 | 458 | 557 | 100 | 78 |
| R1 and IFNA/B-R1) | | | | | |
| Interferon alpha/beta receptor 2 (also known as IFN- | P48551 | 265 | 515 | 251 | 79 |
| R2 and IFNA/B-R2) | | | | | |
| Interferon lambda receptor 1 (also known as IL- | Q8IU57 | 250 | 520 | 271 | 80 |
| 28RA and IFN-lambda-R1) | | | | | |
| Interleukin-1 receptor accessory protein (also | Q9NPH3 | 389 | 570 | 182 | 81 |
| known as IL-1R3 and IL-1RAP) | | | | | |
| Interleukin-1 receptor type 1 (also known as IL- | P14778 | 357 | 569 | 213 | 82 |
| 1R1, IL-1RA, and CD121A) | | | | | |
| Interleukin-1 receptor-like 1 (also known as ST2 | Q01638 | 350 | 556 | 207 | 83 |
| and IL-1RL1) | | | | | |
| Interleukin-1 receptor-like 2 (also known as IL-36R | Q9HB29 | 357 | 575 | 219 | 84 |
| and IL-1RL2) | | | | | |
| Interleukin-11 receptor subunit alpha (also known | Q14626 | 392 | 422 | 31 | 85 |
| as IL-11RA) | | | | | |
| Interleukin-12 receptor subunit beta-1 (also known | P42701 | 571 | 662 | 92 | 86 |
| as IL-12RB1 and CD212) | | | | | |
| Interleukin-12 receptor subunit beta-2 (also known | Q99665 | 644 | 862 | 219 | 87 |
| as IL-12RB2) | | | | | |
| Interleukin-17 receptor A (also known as IL-17RA | Q96F46 | 342 | 866 | 525 | 88 |
| and CD217) | | | | | |
| Interleukin-17 receptor B (also known as IL-17RB) | Q9NRM6 | 314 | 502 | 189 | 89 |
| Interleukin-17 receptor C (also known as IL-17RC) | Q8NAC3 | 560 | 791 | 232 | 90 |
| Interleukin-17 receptor E (also known as IL-17RE) | Q8NFR9 | 476 | 667 | 192 | 91 |
| Interleukin-18 receptor 1 (also known as IL-18R1, | Q13478 | 351 | 541 | 191 | 92 |
| IL-1RRP, and CD218A) | | | | | |
| Interleukin-18 receptor accessory protein (also | O95256 | 378 | 599 | 222 | 93 |
| known as IL-18RB, IL1-R7, and CD218B) | | | | | |
| Interleukin-2 receptor subunit beta (also known as | P14784 | 266 | 551 | 286 | 94 |
| IL-2RB, IL-15RB, and CD122) | | | | | |
| Interleukin-21 receptor (also known as IL-21R and | Q9HBE5 | 254 | 538 | 285 | 95 |
| CD360) | | | | | |
| Interleukin-22 receptor subunit alpha-1 (also known | Q8N6P7 | 250 | 574 | 325 | 96 |
| as IL-22RA1) | | | | | |
| Interleukin-23 receptor (also known as IL-23R) | Q5VWK5 | 377 | 629 | 253 | 97 |
| Interleukin-27 receptor subunit alpha (also known | Q6UWB1 | 538 | 636 | 99 | 98 |
| as IL-27RA and WSX-1) | | | | | |
| Interleukin-3 receptor subunit alpha (also known as | P26951 | 326 | 378 | 53 | 99 |
| IL-3RA and CD123) | | | | | |
| Interleukin-6 receptor subunit beta (also known as | P40189 | 642 | 918 | 277 | 100 |
| IL-6RB, gp130, and CD130) | | | | | |
| Interleukin-7 receptor subunit alpha (also known as | P16871 | 265 | 459 | 195 | 101 |
| IL-7RA and CD127) | | | | | |
| Leukemia inhibitory factor receptor (also known as | P42702 | 859 | 1097 | 239 | 102 |
| LIF-R and CD118) | | | | | |
| Macrophage colony-stimulating factor 1 receptor | P07333 | 539 | 972 | 434 | 103 |
| (also known as M-CSF-R, CSF-1R, CSF1R, and | | | | | |
| CD115) | | | | | |
| Oncostatin-M-specific receptor subunit beta (also | Q99650 | 762 | 979 | 218 | 104 |
| known as OSM-RB and IL-31RB) | | | | | |
| Epidermal growth factor receptor (also known as | P00533 | 669 | 1210 | 542 | 105 |
| EGFR and Her1) | | | | | |
| Growth hormone receptor (also known as GHR and | P10912 | 289 | 638 | 350 | 106 |
| GH receptor) | | | | | |
| Insulin receptor (also known as IR and CD220) | P06213 | 980 | 1382 | 403 | 107 |
| Leptin receptor (also known as LEP-R, OB-R, and | P48357 | 863 | 1165 | 303 | 108 |
| CD295) | | | | | |
| Prolactin receptor (also known as PRL-R) | P16471 | 259 | 622 | 364 | 109 |
| Thrombopoietin receptor (also known as TPO-R, c- | P40238 | 514 | 635 | 122 | 110 |
| Mpl, and CD110) | | | | | |
| Epidermal growth factor receptor (also known as | P00533 | 669 | 1210 | 542 | 111 |
| EGFR and ErbB1) | | | | | |
| Receptor tyrosine-protein kinase erbB-2 (also | P04626 | 676 | 1255 | 580 | 112 |
| known as HER2, Neu, and ErbB2) | | | | | |
| Hepatocyte growth factor receptor (also known as | P08581 | 956 | 1390 | 435 | 113 |
| HGFR and c-Met) | | | | | |
| Fibroblast growth factor receptor 1 (also known as | P11362 | 398 | 822 | 425 | 114 |
| FGFR1, and CD331) | | | | | |
| Fibroblast growth factor receptor 2 (also known as | P21802 | 399 | 821 | 423 | 115 |
| FGFR2 and CD332) | | | | | |
| Fibroblast growth factor receptor 3 (also known as | P22607 | 397 | 806 | 410 | 116 |
| FGFR3 and CD333) | | | | | |
| Fibroblast growth factor receptor 4 (also known as | P22455 | 391 | 802 | 412 | 117 |
| FGFR4 and CD334) | | | | | |
| Vascular endothelial growth factor receptor 2 (also | P35968 | 786 | 1356 | 571 | 118 |
| known as VEGFR-2 and CD309) | | | | | |
| Vascular endothelial growth factor receptor 3 (also | P35916 | 797 | 1363 | 567 | 119 |
| known as VEGFR-3) | | | | | |
| Ephrin type-A receptor 1 (also known as EPHAl | P21709 | 569 | 976 | 408 | 120 |
| and EPH) | | | | | |
| Ephrin type-B receptor 1 (also known as EPHB1, | P54762 | 564 | 984 | 421 | 121 |
| EK6, and ELK) | | | | | |
| Platelet-derived growth factor receptor alpha (also | P16234 | 550 | 1089 | 540 | 122 |
| known as PDGFRA and CD140a) | | | | | |
| Platelet-derived growth factor receptor beta (also | P09619 | 554 | 1106 | 553 | 123 |
| known as PDGFRB and CD140b) | | | | | |
| B-cell antigen receptor complex-associated protein | P11912 | 166 | 226 | 61 | 124 |
| alpha chain (also known as Ig-alpha and CD79A) | | | | | |
| B-cell antigen receptor complex-associated protein | P40259 | 181 | 229 | 49 | 125 |
| beta chain (also known as Ig-beta and CD79B) | | | | | |
| CD160 antigen (also known as CD160 and CD160) | O95971 | 183 | 234 | 52 | 126 |
| CD226 antigen (also known as DNAM-1 and | Q15762 | 276 | 336 | 61 | 127 |
| CD226) | | | | | |
| CD83 antigen (also known as CD83) | Q01151 | 167 | 205 | 39 | 128 |
| Inducible T-cell costimulator (also known as ICOS | Q9Y6W8 | 162 | 199 | 38 | 129 |
| and CD278) | | | | | |
| Intercellular adhesion molecule 1 (also known as | P05362 | 504 | 532 | 29 | 130 |
| ICAM-1 and CD54) | | | | | |
| Intercellular adhesion molecule 2 (also known as | P13598 | 249 | 275 | 27 | 131 |
| ICAM-2 and CD102) | | | | | |
| Intercellular adhesion molecule 3 (also known as | P32942 | 511 | 547 | 37 | 132 |
| ICAM-3 and CD50) | | | | | |
| Killer cell immunoglobulin-like receptor 2DL4 (also | Q99706 | 264 | 377 | 114 | 133 |
| known as KIR2DL4 and CD158D) | | | | | |
| Killer cell immunoglobulin-like receptor 2DS1 (also | Q14954 | 265 | 304 | 40 | 134 |
| known as KIR2DS1 and CD158H) | | | | | |
| Killer cell immunoglobulin-like receptor 2DS2 (also | P43631 | 266 | 304 | 39 | 135 |
| known as KIR2DS2 and CD158J) | | | | | |
| Killer cell immunoglobulin-like receptor 2DS3 (also | Q14952 | 265 | 304 | 40 | 136 |
| known as KIR2DS3) | | | | | |
| Killer cell immunoglobulin-like receptor 2DS4 (also | P43632 | 266 | 304 | 39 | 137 |
| known as KIR2DS4 and CD158I) | | | | | |
| Killer cell immunoglobulin-like receptor 2DS50 | Q14953 | 265 | 304 | 40 | 138 |
| (also known as KIR2DS5 and CD158G) | | | | | |
| Killer cell immunoglobulin-like receptor 3DS1 (also | Q14943 | 361 | 382 | 22 | 139 |
| known as KIR3DS1) | | | | | |
| Natural cytotoxicity triggering receptor 1 (also | O76036 | 280 | 304 | 25 | 140 |
| known as NKp46, Ly94, and CD335) | | | | | |
| Natural cytotoxicity triggering receptor 2 (also | O95944 | 214 | 276 | 63 | 141 |
| known as NKp44 and CD336) | | | | | |
| Natural cytotoxicity triggering receptor 3 (also | O14931 | 157 | 201 | 45 | 142 |
| known as NKp30 and CD337) | | | | | |
| T-cell antigen CD7 (also known as CD7) | P09564 | 202 | 240 | 39 | 143 |
| T-cell surface glycoprotein CD4 (also known as | P01730 | 419 | 458 | 40 | 144 |
| CD4) | | | | | |
| T-cell-specific surface glycoprotein CD28 (also | P10747 | 180 | 220 | 41 | 145 |
| known as CD28) | | | | | |
| Transmembrane and immunoglobulin domain- | Q96BF3 | 172 | 282 | 111 | 146 |
| containing protein 2 (also known as TMIGD2, | | | | | |
| CD28H, and IGPR-1) | | | | | |
| Intercellular adhesion molecule 5 (also known as | Q9UMF0 | 857 | 924 | 68 | 147 |
| ICAM-5 and telencephalin) | | | | | |
| Integrin alpha-L (also known as LFA-1A and | P20701 | 1112 | 1170 | 59 | 148 |
| CD11A) | | | | | |
| Integrin beta-2 (also known as LFA-1B and CD18) | P05107 | 724 | 769 | 46 | 149 |
| Cytotoxic and regulatory T-cell molecule (also | O95727 | 309 | 393 | 85 | 150 |
| known as CRTAM and CD355) | | | | | |
| B-cell receptor CD22 (also known as Siglec-2 and | P20273 | 707 | 847 | 141 | 151 |
| CD22) | | | | | |
| Sialic acid-binding Ig-like lectin 14 (also known as | Q08ET2 | 382 | 396 | 15 | 152 |
| Siglec-14) | | | | | |
| Sialic acid-binding Ig-like lectin 15 (also known as | Q6ZMC9 | 285 | 328 | 44 | 153 |
| Siglec-15) | | | | | |
| Sialic acid-binding Ig-like lectin 16 (also known as | A6NMB1 | 456 | 481 | 26 | 154 |
| Siglec-16) | | | | | |
| Hepatitis A virus cellular receptor 1 (also known as | Q96D42 | 317 | 364 | 48 | 155 |
| TIM-1, KIM-1, and CD365) | | | | | |
| Toll-like receptor 1 (also known as TLR1 and | Q15399 | 602 | 786 | 185 | 156 |
| CD281) | | | | | |
| Toll-like receptor 10 (also known as TLR10 and | Q9BXR5 | 598 | 811 | 214 | 157 |
| CD290) | | | | | |
| Toll-like receptor 2 (also known as TLR2 and | O60603 | 610 | 784 | 175 | 158 |
| CD282) | | | | | |
| Toll-like receptor 3 (also known as TLR3 and | O15455 | 726 | 904 | 179 | 159 |
| CD283) | | | | | |
| Toll-like receptor 4 (also known as TLR4 and | O00206 | 653 | 839 | 187 | 160 |
| CD284) | | | | | |
| Toll-like receptor 5 (also known as TLR5 and | O60602 | 661 | 858 | 198 | 161 |
| CD285) | | | | | |
| Toll-like receptor 6 (also known as TLR6 and | Q9Y2C9 | 608 | 796 | 189 | 162 |
| CD286) | | | | | |
| Toll-like receptor 7 (also known as TLR7 and | Q9NYK1 | 861 | 1049 | 189 | 163 |
| CD287) | | | | | |
| Toll-like receptor 8 (also known as TLR8 and | Q9NR97 | 849 | 1041 | 193 | 164 |
| CD288) | | | | | |
| Toll-like receptor 9 (also known as TLR9 and | Q9NR96 | 840 | 1032 | 193 | 165 |
| CD289) | | | | | |
| CD27 antigen (also known as CD27) | P26842 | 213 | 260 | 48 | 166 |
| Tumor necrosis factor receptor superfamily member | Q9Y6Q6 | 234 | 616 | 383 | 167 |
| 11A (also known as RANK and CD265) | | | | | |
| Tumor necrosis factor receptor superfamily member | Q9NP84 | 102 | 129 | 28 | 168 |
| 12A (also known as TweakR, FN14, and CD266) | | | | | |
| Tumor necrosis factor receptor superfamily member | O14836 | 187 | 293 | 107 | 169 |
| 13B (also known as TACI and CD267) | | | | | |
| Tumor necrosis factor receptor superfamily member | Q96RJ3 | 100 | 184 | 85 | 170 |
| 13C (also known as BAFF-R and CD268) | | | | | |
| Tumor necrosis factor receptor superfamily member | Q92956 | 224 | 283 | 60 | 171 |
| 14 (also known as HVEM and CD270) | | | | | |
| Tumor necrosis factor receptor superfamily member | P08138 | 273 | 427 | 155 | 172 |
| 16 (also known as NGF-R, p75NTR, and CD271) | | | | | |
| Tumor necrosis factor receptor superfamily member | Q02223 | 78 | 184 | 107 | 173 |
| 17 (also known as BCMA and CD269) | | | | | |
| Tumor necrosis factor receptor superfamily member | Q9Y5U5 | 184 | 241 | 58 | 174 |
| 18 (also known as GITR and CD357) | | | | | |
| Tumor necrosis factor receptor superfamily member | Q9NS68 | 192 | 423 | 232 | 175 |
| 19 (also known as TROY and TRADE) | | | | | |
| Tumor necrosis factor receptor superfamily member | Q969Z4 | 184 | 430 | 247 | 176 |
| 19L (also known as RELT) | | | | | |
| Tumor necrosis factor receptor superfamily member | P19438 | 233 | 455 | 223 | 177 |
| lA (also known as TNF-RI and CD120A) | | | | | |
| Tumor necrosis factor receptor superfamily member | P20333 | 288 | 461 | 174 | 178 |
| 1B (also known as TNF-RII and CD120B) | | | | | |
| Tumor necrosis factor receptor superfamily member | Q93038 | 221 | 417 | 197 | 179 |
| 25 (also known as DR3 and TRAMP) | | | | | |
| Tumor necrosis factor receptor superfamily member | Q9HAV5 | 160 | 297 | 138 | 180 |
| 27 (also known as XEDAR and EDA-A2 receptor) | | | | | |
| Tumor necrosis factor receptor superfamily member | P36941 | 249 | 435 | 187 | 181 |
| 3 (also known as LTB-R and TNF-RIII) | | | | | |
| Tumor necrosis factor receptor superfamily member | P43489 | 236 | 277 | 42 | 182 |
| 4 (also known as OX-40 and CD134) | | | | | |
| Tumor necrosis factor receptor superfamily member | P25942 | 216 | 277 | 62 | 183 |
| 5 (also known as CD40) | | | | | |
| Tumor necrosis factor receptor superfamily member | P28908 | 407 | 595 | 189 | 184 |
| 8 (also known as CD30) | | | | | |
| Tumor necrosis factor receptor superfamily member | Q07011 | 214 | 255 | 42 | 185 |
| 9 (also known as 4-1BB and CD137) | | | | | |
| Tumor necrosis factor receptor superfamily member | Q9UNE0 | 209 | 448 | 240 | 186 |
| EDAR (also known as EDAR) | | | | | |
| Linker for activation of T-cells family member 1 | O43561 | 28 | 262 | 235 | 187 |
| (also known as LAT) | | | | | |
| Linker for activation of T-cells family member 2 | Q9GZY6 | 27 | 243 | 217 | 188 |
| (also known as LAT2, NTAL, and LAB) | | | | | |
| Lymphocyte transmembrane adapter 1 (also known | Q8IWV1 | 59 | 398 | 340 | 189 |
| as LAX) | | | | | |
| Phosphoprotein associated with glycosphingolipid- | Q9NWQ8 | 38 | 432 | 395 | 190 |
| enriched microdomains 1 (also known as PAG and | | | | | |
| CBP) | | | | | |
| Linker for activation of T-cells family member 1 | O43561 | 28 | 262 | 235 | 191 |
| (also known as LAT) | | | | | |
| Low affinity immunoglobulin epsilon Fc receptor | P06734 | 1 | 21 | 21 | 192 |
| (also known as FCERII and CD23) | | | | | |
| CD209 antigen (also known as DC-SIGN, CLEC- | Q9NNX6 | 1 | 37 | 37 | 193 |
| 4L and CD209) | | | | | |
| C-type lectin domain family 1 member B (also | Q9P126 | 1 | 33 | 33 | 194 |
| known as CLEC-2) | | | | | |
| C-type lectin domain family 7 member A (also | Q9BXN2 | 1 | 44 | 44 | 195 |
| known as Dectin-1 and CLEC-7A) | | | | | |
| C-type lectin domain family 9 member A (also | Q6UXN8 | 1 | 35 | 35 | 196 |
| known as DNGR-1 and CD370) | | | | | |
| Killer cell lectin-like receptor subfamily F member | Q9NZS2 | 1 | 38 | 38 | 197 |
| 1 (also known as NKp80, KLRF1, and CLEC5C) | | | | | |
| Killer cell lectin-like receptor subfamily F member | D3W0D1 | 1 | 30 | 30 | 198 |
| 2 (also known as NKp65 and KLRF2) | | | | | |
| NKG2-C type II integral membrane protein (also | P26717 | 1 | 70 | 70 | 199 |
| known as NKG2C, KLRC2, and CD159C) | | | | | |
| NKG2-D type II integral membrane protein (also | P26718 | 1 | 51 | 51 | 200 |
| known as NKG2D, and CD314) | | | | | |
| NKG2-E type II integral membrane protein (also | Q07444 | 1 | 70 | 70 | 201 |
| known as NKG2E and KLRC3) | | | | | |
| C-type lectin domain family 4 member E (also | Q9ULY5 | 1 | 19 | 19 | 202 |
| known as CLEC-4E and MINCLE) | | | | | |
| C-type lectin domain family 6 member A (also | Q6EIG7 | 1 | 20 | 20 | 203 |
| known as CLEC-6A and Dectin-2) | | | | | |
| C-type lectin domain family 10 member A (also | Q8IUN9 | 1 | 39 | 39 | 204 |
| known as CLEC-10A, MGL, and CD301) | | | | | |
| C-type lectin domain family 4 member D (also | Q8WXI8 | 1 | 17 | 17 | 205 |
| known as CLEC-4D, CLEC-6, Dectin-3, and | | | | | |
| CD368) | | | | | |
| C-type lectin domain family 4 member C (also | Q8WTT0 | 1 | 21 | 21 | 206 |
| known as CLEC-4C, BDCA-2, and CD303) | | | | | |
| C-type lectin domain family 17, member A (also | Q6ZS10 | 1 | 172 | 172 | 207 |
| known as CLEC-17A, and prolectin) | | | | | |
| CD70 antigen (also known as CD70) | P32970 | 1 | 17 | 17 | 208 |
| Tumor necrosis factor ligand superfamily member | O43557 | 1 | 37 | 37 | 209 |
| 14 (also known as LIGHT and CD258) | | | | | |
| Tumor necrosis factor ligand superfamily member 8 | P32971 | 1 | 37 | 37 | 210 |
| (also known as CD30L and CD153) | | | | | |
| Tumor necrosis factor (also known as tumor | P01375 | 1 | 35 | 35 | 211 |
| necrosis factor, TNFa and TNFSF1A) | | | | | |
| Tumor necrosis factor ligand superfamily member 4 | P23510 | 1 | 23 | 23 | 212 |
| (also known as OX40L, CD252, CD134L, and | | | | | |
| CD252) | | | | | |
| CD40 ligand (also known as CD40L, CD154, and | P29965 | 1 | 22 | 22 | 213 |
| CD154) | | | | | |
| Tumor necrosis factor ligand superfamily member 6 | P48023 | 1 | 80 | 80 | 214 |
| (also known as FasL, CD178, CD95L and CD178) | | | | | |
| Tumor necrosis factor ligand superfamily member 9 | P41273 | 1 | 28 | 28 | 215 |
| (also known as 4-1BBL and CD137L) | | | | | |
| Tumor necrosis factor ligand superfamily member | P50591 | 1 | 17 | 17 | 216 |
| 10 (also known as TRAIL, TNF-related apoptosis- | | | | | |
| inducing ligand, CD253, APO-2L, and CD253) | | | | | |
| Tumor necrosis factor ligand superfamily member | O14788 | 1 | 47 | 47 | 217 |
| 11 (also known as TRANCE, RANKL, CD254, | | | | | |
| OPGL, and CD254) | | | | | |
| Tumor necrosis factor ligand superfamily member | O43508 | 1 | 21 | 21 | 218 |
| 12 (also known as TWEAK, APO-3L, and DR3L) | | | | | |
| Tumor necrosis factor ligand superfamily member | O75888 | 1 | 28 | 28 | 219 |
| 13 (also known as APRIL, CD256, TALL-2, | | | | | |
| TRDL1, and CD256) | | | | | |
| Tumor necrosis factor ligand superfamily member | Q9Y275 | 1 | 46 | 46 | 220 |
| 13B (also known as BAFF, B-Cell Activating | | | | | |
| Factor, CD257, TALL-1, and CD257) | | | | | |
| Tumor necrosis factor ligand superfamily member | Q9UNG2 | 23 | 50 | 28 | 221 |
| 18 (also known as TNFSF18, GITRL, TL-6) | | | | | |
| Paired immunoglobulin-like type 2 receptor beta | Q9UKJ0 | 213 | 227 | 15 | 535 |
| (also known as PILRB) | | | | | |

In some embodiments, an engineered protein (e.g., chimeric protein) described herein comprises an intracellular domain comprising an amino acid sequence having at least 90%, at least 91%, at least 92%, at least 93%, at least 94%, at least 95%, at least 96%, at least 97%, at least 98%, at least 99% or 100% identity with the amino acid sequence of any one of SEQ ID NOs: 48-221 and 535.

In some embodiments, an engineered protein (e.g., chimeric protein) described herein includes an intracellular domain of a DAP10 polypeptide, or a portion thereof. In some embodiments, an engineered protein (e.g., chimeric protein) described herein includes an intracellular domain derived from a human DAP10 polypeptide or a portion thereof which comprises an amino acid sequence having at least 90%, at least 91%, at least 92%, at least 93%, at least 94%, at least 95%, at least 96%, at least 97%, at least 98%, at least 99%, or 100% identity with the amino acid sequence of SEQ ID NO: 48.

In some embodiments, an engineered protein (e.g., chimeric protein) described herein includes an intracellular domain of a DAP12 polypeptide or a portion thereof. In some embodiments, an engineered protein (e.g., chimeric protein) provided herein includes an intracellular domain derived from a human DAP12 polypeptide or a portion thereof which comprises an amino acid sequence having at least 90%, at least 91%, at least 92%, at least 93%, at least 94%, at least 95%, at least 96%, at least 97%, at least 98%, at least 99%, or 100% identity with the amino acid sequence of SEQ ID NO: 50.

In some embodiments, an engineered protein (e.g., chimeric protein) described herein includes both an intracellular domain of a DAP10 polypeptide or a portion thereof, and an intracellular domain of a TGF-BR2 polypeptide or a portion thereof. In some embodiments, an engineered protein (e.g., chimeric protein) provided herein includes an intracellular domain derived from a human DAP10 polypeptide or a portion thereof, and an intracellular domain derived form a human TGF-BR2 polypeptide or a portion thereof, and the intracellular domain comprises an amino acid sequence having at least 90%, at least 91%, at least 92%, at least 93%, at least 94%, at least 95%, at least 96%, at least 97%, at least 98%, at least 99%, or 100% identity with the amino acid sequence of SEQ ID NO: 222.

In some embodiments, an engineered protein (e.g., chimeric protein) provided herein includes an intracellular domain of a CD3ζ (CD3zeta) polypeptide or a portion thereof. In some embodiments, an engineered protein (e.g., chimeric protein) provided herein includes an intracellular domain derived from human CD3zeta or a portion thereof, which comprises an amino acid sequence having at least 90%, at least 91%, at least 92%, at least 93%, at least 94%, at least 95%, at least 96%, at least 97%, at least 98%, at least 99% or 100% identity with the amino acid sequence of SEQ ID NO: 67. In some embodiments, the CD3zeta from which the intracellular domain is derived comprises a mutation in an ITAM domain.

In some embodiments, an engineered protein described herein includes an intracellular domain of PILRB, or a portion thereof. In some embodiments, an engineered protein described herein includes an intracellular domain derived from PILRB (e.g., SEQ ID NO: 533) or a portion thereof which comprises an amino acid sequence having at least 90%, at least 91%, at least 92%, at least 93%, at least 94%, at least 95%, at least 96%, at least 97%, at least 98%, at least 99%, or 100% identity with the amino acid sequence of SEQ ID NO: 535.

The intracellular domain (ICD) of an engineered protein (e.g., chimeric protein) derived herein may provide a signal that activates the cell expressing the protein. In immune cells, including NK cells, diverse upstream signals converge on four transcription factor pathways: nuclear factor kappa B (NF-κB), activator protein 1 (AP-1), nuclear factor of activated T-cells (NFAT), and signal transducer and activator of transcription proteins (STATs), crucial to cellular functions including survival, proliferation, cytokine production, and cytotoxic activity. Therefore, the activity of an intracellular domain included in an engineered protein (e.g., chimeric protein) described herein may be assessed by testing the activation of one of these four pathways using methods known in the art. For example, engineered proteins (e.g., chimeric proteins) including an intracellular domain from CARD11, DAP10, LAT, LAT2, SLP76, LAX, MyD88, PAG, GAPT, SAP, EAT-2, or DAP12 may be tested for NF-κB, AP-1, and/or NFAT activity; engineered proteins (e.g., chimeric proteins) including an intracellular domain from TLR1, TLR10, TLR2, TLR3, TLR4, TLR5, TLR6, TLR7, TLR8, and TLR9 may be tested for NF-κB and/or AP-1 activity; engineered proteins (e.g., chimeric proteins) including an intracellular domain from CD131, EGFR, EPO-R, G-CSF-R, GM-CSF-R-alpha, IL-2RB, TSLP-R may be tested for NF-κB, AP-1, STAT5, and/or STAT3 activity; engineered proteins (e.g., chimeric proteins) including an intracellular domain from IL-17RA, IL-17RB, IL-17RC, IL-17RE, IL-18R1, IL-18RB, IL-1R1, IL-1R3, IL-36R, M-CSF-R, ST2, IL-22RA1, IL-21R, GHR, IFN-R1, IFN-R2, IL-27RA, IL-11RA, IL-6RB, LIF-R, OSM-RB, LEP-R, TPO-R, IL-2RG, PRL-KR, IL-3RA, IL-23R, IL-12RB1, IL-12RB2, IL-28RA, and IL-7RA may be tested for NF—B, AP-1, STAT1, STAT 2, STAT3 and/or STAT5 signaling using methods known in the art. For example, NK-κB activity may be assessed by detecting and/or analyzing phosphorylated RelA/p65 levels, AP-1 activity may be assessed by detecting and/or analyzing phosphorylated c-Jun levels, NFAT activity may be assessed by detecting and/or analyzing dephosphorylated NFAT1 levels, STAT activity may be assessed by detecting and/or analyzing phosphorylated STAT5A levels, and phosphatidylinositol 3-kinase (PI3K) activity may be assessed by detecting phosphorylated Akt levels, each in a cell or population of cells expressing an engineered protein (e.g., chimeric protein) provided herein (in the presence and/or absence of exposure of the cells to a ligand of the engineered protein (e.g., chimeric protein) (e.g., a negative signal)).

### C. Transmembrane Domains

Suitable transmembrane domains of an engineered protein (e.g., chimeric protein) disclosed herein have the ability to: (a) be expressed at the surface of a cell, which is in some embodiments an immune cell (e.g., a NK cell), and/or (b) interact with the extracellular domain and intracellular domain for directing cellular response of the cell. The transmembrane domain can be a transmembrane domain of any membrane-bound or transmembrane protein.

In some embodiments, the transmembrane domain of an engineered protein (e.g., chimeric protein) provided herein is a transmembrane domain, or a portion thereof, of an inhibitory polypeptide. In some embodiments, the transmembrane domain and the extracellular domain are derived from the same inhibitory polypeptide. In some embodiments, the transmembrane domain and the extracellular domain are derived from different polypeptides (e.g., different inhibitory polypeptides). In some embodiments, the transmembrane domain of an engineered protein (e.g., chimeric protein) provided herein comprises or consists of a transmembrane domain, or a portion thereof, of an inhibitory polypeptide presented in Table 1. In some embodiments, the transmembrane domain of an engineered protein (e.g., chimeric protein) provided herein comprises or consists of the transmembrane domain of an inhibitory polypeptide presented in Table 1.1. In some embodiments, the transmembrane domain of the engineered protein (e.g., chimeric protein) provided herein comprises or consists of a transmembrane domain of an inhibitory polypeptide listed in Table 1.2.

TABLE 1.2
Exemplary Transmembrane Domains

| | | | | Amino | SEQ |
|---|---|---|---|---|---|
| | | | | acid | ID |
| Inhibitory polypeptide | UNIPROT ID | Start | End | length | NO: |
| BTLA | Q7Z6A9 | 158 | 178 | 21 | 223 |
| CD160 | O95971 | 163 | 182 | 20 | 224 |
| CD200R | Q8TD46 | 244 | 264 | 21 | 225 |
| CD33 | P20138 | 260 | 282 | 23 | 226 |
| CEACAM-1 | P13688 | 429 | 452 | 24 | 227 |
| (also known as CD66a) | | | | | |
| CTLA-4 | P16410 | 162 | 182 | 21 | 228 |
| Fas | P25445 | 174 | 190 | 17 | 229 |
| FCRL6 | Q6DN72 | 308 | 328 | 21 | 230 |
| IL-10RA | Q13651 | 236 | 256 | 21 | 231 |
| IL-10RB | Q08334 | 221 | 242 | 22 | 232 |
| IL-1R8 | A0A291NLA3 | 119 | 140 | 22 | 233 |
| IL-6RA | P08887 | 366 | 386 | 21 | 234 |
| IL-6RB (also known | P40189 | 620 | 641 | 22 | 235 |
| as gp130 and CD130) | | | | | |
| KIR2DL1 | P43626 | 246 | 264 | 19 | 236 |
| KIR2DL2 | P43627 | 246 | 264 | 19 | 237 |
| KIR2DL3 | P43628 | 246 | 265 | 20 | 238 |
| KIR2DL5A | Q8N109 | 239 | 259 | 21 | 239 |
| KIR2DL5B | Q8NHK3 | 239 | 259 | 21 | 240 |
| KIR3DL1 | P43629 | 341 | 360 | 20 | 241 |
| KIR3DL2 | P43630 | 341 | 360 | 20 | 242 |
| KIR3DL3 | Q8N743 | 323 | 343 | 21 | 243 |
| Lag3 | P18627 | 451 | 471 | 21 | 244 |
| LILRB1 | Q8NHL6 | 462 | 482 | 21 | 245 |
| LILRB2 | Q8N423 | 462 | 482 | 21 | 246 |
| LILRB3 | O75022 | 444 | 464 | 21 | 247 |
| LILRB4 | Q8NHJ6 | 260 | 280 | 21 | 248 |
| LILRB5 | O75023 | 459 | 479 | 21 | 249 |
| NKp30c | O14931-2 | 136 | 156 | 21 | 250 |
| PD-1 | Q15116 | 171 | 191 | 21 | 251 |
| Siglec-10 | Q96LC7 | 551 | 571 | 21 | 252 |
| Siglec-7 | Q9Y286 | 354 | 376 | 23 | 253 |
| Siglec-9 | Q9Y336 | 349 | 369 | 21 | 254 |
| TACTILE | P40200 | 520 | 540 | 21 | 255 |
| (also known as CD96) | | | | | |
| TGF-BR1 | P36897 | 127 | 147 | 21 | 256 |
| TGF-BR2 | P37173 | 167 | 187 | 21 | 257 |
| TIGIT | Q495A1 | 142 | 162 | 21 | 258 |
| TIM-3 | Q8TDQ0 | 203 | 223 | 21 | 259 |
| CD94 | Q13241 | 11 | 31 | 21 | 260 |
| KLRB1 (NKR-P1A) | Q12918 | 46 | 66 | 21 | 261 |
| KLRG1 | Q96E93 | 39 | 59 | 21 | 262 |
| NKG2A | P26715 | 71 | 93 | 23 | 263 |

In some embodiments, an engineered protein (e.g., chimeric protein) provided herein includes a transmembrane domain of a cluster of differentiation 4 (CD4) protein (e.g., a human CD4 protein) or a transmembrane domain of a cluster of differentiation 8 (CD8) protein (e.g., a human CD8 protein).

In some embodiments, an engineered protein (e.g., chimeric protein) provided herein includes a transmembrane domain comprises or consists of an amino acid sequence having at least 90%, at least 91%, at least 92%, at least 93%, at least 94%, at least 95%, at least 96%, at least 97%, at least 98%, at least 99% or 100% identity with the amino acid sequence of any one of SEQ ID NOs: 223-263.

In some embodiments, an engineered protein (e.g., chimeric protein) provided herein includes a transmembrane domain of a stimulatory polypeptide, or portion thereof. In some embodiments, an engineered protein (e.g., chimeric protein) provided herein includes a transmembrane domain and an intracellular domain, and both domains are derived from the same stimulatory polypeptide. In some embodiments, an engineered protein (e.g., chimeric protein) provided herein includes a transmembrane domain and an intracellular domain, and both domains are derived from different polypeptides (e.g., different stimulatory polypeptides or one stimulatory polypeptide and one inhibitory polypeptide). In some embodiments, an engineered protein (e.g., chimeric protein) provided herein includes a transmembrane domain of a stimulatory polypeptide presented in Table 2. In some embodiments, an engineered protein (e.g., chimeric protein) provided herein includes a transmembrane domain of a stimulatory polypeptide is selected from the stimulatory polypeptides presented in Table 2.1.

In some embodiments, the transmembrane domain of an engineered protein (e.g., chimeric protein) provided herein is a transmembrane domain of a stimulatory polypeptide listed in Table 2.2.

TABLE 2.2
Examples of Transmembrane Domains

| | | | | Amino | |
|---|---|---|---|---|---|
| | UNIPROT | | | acid | SEQ ID |
| Stimulatory Polypeptide | ID | Start | End | length | NO: |
| Hematopoietic cell signal transducer (also | Q9UBK5 | 49 | 69 | 21 | 264 |
| known as DAP10) | | | | | |
| Protein GAPT (also known as GAPT) | Q8N292 | 11 | 31 | 21 | 265 |
| TYRO protein tyrosine kinase-binding | O43914 | 41 | 61 | 21 | 266 |
| protein (also known as DAP12) | | | | | |
| High affinity immunoglobulin epsilon | P30273 | 24 | 44 | 21 | 267 |
| receptor subunit gamma (also known as | | | | | |
| FcRgamma and FceRI gamma) | | | | | |
| High affinity immunoglobulin gamma Fc | P12314 | 293 | 313 | 21 | 268 |
| receptor I (also known as FcRI, Fc-gamma | | | | | |
| RI, and CD64A) | | | | | |
| Low affinity immunoglobulin gamma Fc | P12318 | 218 | 240 | 23 | 269 |
| region receptor II-a (also known as FcRII-a, | | | | | |
| Fc-gamma RIIa, and CD32A) | | | | | |
| Low affinity immunoglobulin gamma Fc | P31995 | 224 | 246 | 23 | 270 |
| region receptor II-c (also known as FcRII-c, | | | | | |
| Fc-gamma RIIc, and CD32C) | | | | | |
| Low affinity immunoglobulin gamma Fc | P08637 | 209 | 229 | 21 | 271 |
| region receptor III-A (also known as | | | | | |
| FcRIIIa, Fc-gamma RIIIa, and CD16A) | | | | | |
| Immunoglobulin alpha Fc receptor (also | P24071 | 228 | 246 | 19 | 272 |
| known as FCARI, and CD89) | | | | | |
| Lymphocyte function-associated antigen 3 | P19256 | 216 | 238 | 23 | 273 |
| (also known as LFA-3, and CD58) | | | | | |
| Natural killer cell receptor 2B4 (also known | Q9BZW8 | 230 | 250 | 21 | 274 |
| as 2B4, SLAMF4, and CD244) | | | | | |
| Signaling lymphocytic activation molecule | Q13291 | 238 | 258 | 21 | 275 |
| (also known as SLAM, SLAMF1, and | | | | | |
| CD150) | | | | | |
| SLAM family member 5 (also known as | Q9UIB8 | 226 | 246 | 21 | 276 |
| SLAMF5 and CD84) | | | | | |
| SLAM family member 6 (also known as | Q96DU3 | 227 | 247 | 21 | 277 |
| SLAMF6, NTB-A, and CD352) | | | | | |
| SLAM family member 7 (also known as | Q9NQ25 | 227 | 247 | 21 | 278 |
| SLAMF7 and CD319) | | | | | |
| T-cell surface antigen CD2 (also known as | P06729 | 210 | 235 | 26 | 279 |
| LFA-2 and CD2) | | | | | |
| T-lymphocyte surface antigen Ly-9 (also | Q9HBG7 | 455 | 476 | 22 | 280 |
| known as SLAMF3, Ly-9, and CD229) | | | | | |
| T-cell surface glycoprotein CD3 epsilon | P07766 | 127 | 152 | 26 | 281 |
| chain (also known as CD3E) | | | | | |
| T-cell surface glycoprotein CD3 gamma | P09693 | 117 | 137 | 21 | 282 |
| chain (also known as CD3G) | | | | | |
| T-cell surface glycoprotein CD3 zeta chain | P20963 | 31 | 51 | 21 | 283 |
| (also known as CD3Z and CD247) | | | | | |
| Carcinoembryonic antigen-related cell | P40198 | 156 | 176 | 21 | 284 |
| adhesion molecule 3 (also known as | | | | | |
| CEACAM-3 and CD66D) | | | | | |
| Complement receptor type 1 (also known as | P17927 | 1972 | 1996 | 25 | 285 |
| CR1, C3B/C4b receptor and CD35) | | | | | |
| Membrane cofactor protein (also known as | P15529 | 344 | 366 | 23 | 286 |
| MCP and CD46) | | | | | |
| Macrophage mannose receptor 1 (also | P22897 | 1390 | 1410 | 21 | 287 |
| known as MMR and CD206) | | | | | |
| Cytokine receptor common subunit beta | P32927 | 444 | 460 | 17 | 288 |
| (also known as CD131) | | | | | |
| Cytokine receptor common subunit gamma | P31785 | 263 | 283 | 21 | 289 |
| (also known as IL-2RG and CD132) | | | | | |
| Cytokine receptor-like factor 2 (also known | Q9HC73 | 232 | 252 | 21 | 290 |
| as TSLP-R) | | | | | |
| Erythropoietin receptor (also known as EPO- | P19235 | 251 | 273 | 23 | 291 |
| R and EPOR) | | | | | |
| Granulocyte colony-stimulating factor | Q99062 | 628 | 650 | 23 | 292 |
| receptor (also known as G-CSF-R, GCSFR, | | | | | |
| and CD114) | | | | | |
| Granulocyte-macrophage colony-stimulating | P15509 | 321 | 346 | 26 | 293 |
| factor receptor subunit alpha (also known as | | | | | |
| GM-CSF-R-alpha and CD116) | | | | | |
| Interferon alpha/beta receptor 1 (also known | P17181 | 437 | 457 | 21 | 294 |
| as IFN-R1 and IFNA/B-R1) | | | | | |
| Interferon alpha/beta receptor 2 (also known | P48551 | 244 | 264 | 21 | 295 |
| as IFN-R2 and IFNA/B-R2) | | | | | |
| Interferon lambda receptor 1 (also known as | Q8IU57 | 229 | 249 | 21 | 296 |
| IL-28RA and IFN-lambda-R1) | | | | | |
| Interleukin-1 receptor accessory protein (also | Q9NPH3 | 368 | 388 | 21 | 297 |
| known as IL-1R3 and IL-1RAP) | | | | | |
| Interleukin-1 receptor type 1 (also known as | P14778 | 337 | 356 | 20 | 298 |
| IL-1R1, IL-1RA and CD121A) | | | | | |
| Interleukin-1 receptor-like 1 (also known as | Q01638 | 329 | 349 | 21 | 299 |
| 5T2 and IL-1RL1) | | | | | |
| Interleukin-1 receptor-like 2 (also known as | Q9HB29 | 336 | 356 | 21 | 300 |
| IL-36R and IL-1RL2) | | | | | |
| Interleukin-11 receptor subunit alpha (also | Q14626 | 371 | 391 | 21 | 301 |
| known as IL-11RA) | | | | | |
| Interleukin-12 receptor subunit beta-1 (also | P42701 | 546 | 570 | 25 | 302 |
| known as IL-12RB1 and CD212) | | | | | |
| Interleukin-12 receptor subunit beta-2 (also | Q99665 | 623 | 643 | 21 | 303 |
| known as IL-12RB2) | | | | | |
| Interleukin-17 receptor A (also known as IL- | Q96F46 | 321 | 341 | 21 | 304 |
| 17RA and CD217) | | | | | |
| Interleukin-17 receptor B (also known as IL- | Q9NRM6 | 293 | 313 | 21 | 305 |
| 17RB) | | | | | |
| Interleukin-17 receptor C (also known as IL- | Q8NAC3 | 539 | 559 | 21 | 306 |
| 17RC) | | | | | |
| Interleukin-17 receptor E (also known as IL- | Q8NFR9 | 455 | 475 | 21 | 307 |
| 17RE) | | | | | |
| Interleukin-18 receptor 1 (also known as IL- | Q13478 | 330 | 350 | 21 | 308 |
| 18R1, IL-1RRP, and CD218A) | | | | | |
| Interleukin-18 receptor accessory protein | O95256 | 357 | 377 | 21 | 309 |
| (also known as IL-18RB, IL-1-R7, and | | | | | |
| CD218B) | | | | | |
| Interleukin-2 receptor subunit beta (also | P14784 | 241 | 265 | 25 | 310 |
| known as IL-2RB, IL-15RB, and CD122) | | | | | |
| Interleukin-21 receptor (also known as IL- | Q9HBE5 | 233 | 253 | 21 | 311 |
| 21R and CD360) | | | | | |
| Interleukin-22 receptor subunit alpha-1 (also | Q8N6P7 | 229 | 249 | 21 | 312 |
| known as IL-22RA1) | | | | | |
| Interleukin-23 receptor (also known as IL- | Q5VWK5 | 356 | 376 | 21 | 313 |
| 23R) | | | | | |
| Interleukin-27 receptor subunit alpha (also | Q6UWB1 | 517 | 537 | 21 | 314 |
| known as IL-27RA and WSX-1) | | | | | |
| Interleukin-3 receptor subunit alpha (also | P26951 | 306 | 325 | 20 | 315 |
| known as IL-3RA and CD123) | | | | | |
| Interleukin-6 receptor subunit beta (also | P40189 | 620 | 641 | 22 | 316 |
| known as IL-6RB, gp130, and CD130) | | | | | |
| Interleukin-7 receptor subunit alpha (also | P16871 | 240 | 264 | 25 | 317 |
| known as IL-7RA and CD127) | | | | | |
| Leukemia inhibitory factor receptor (also | P42702 | 834 | 858 | 25 | 318 |
| known as LIF-R and CD118) | | | | | |
| Macrophage colony-stimulating factor 1 | P07333 | 518 | 538 | 21 | 319 |
| receptor (also known as M-CSF-R, CSF-1R, | | | | | |
| CSF1R, and CD115) | | | | | |
| Oncostatin-M-specific receptor subunit beta | Q99650 | 741 | 761 | 21 | 320 |
| (also known as OSM-RB and IL-31RB) | | | | | |
| Epidermal growth factor receptor (also | P00533 | 646 | 668 | 23 | 321 |
| known as EGFR and Her1) | | | | | |
| Growth hormone receptor (also known as | P10912 | 265 | 288 | 24 | 322 |
| GHR and GH receptor) | | | | | |
| Insulin receptor (also known as IR and | P06213 | 957 | 979 | 23 | 323 |
| CD220) | | | | | |
| Leptin receptor (also known as LEP-R, OB- | P48357 | 840 | 862 | 23 | 324 |
| R, and CD295) | | | | | |
| Prolactin receptor (also known as PRL-R) | P16471 | 235 | 258 | 24 | 325 |
| Thrombopoietin receptor (also known as | P40238 | 492 | 513 | 22 | 326 |
| TPO-R, c-Mpl, and CD110) | | | | | |
| Epidermal growth factor receptor (also | P00533 | 646 | 668 | 23 | 327 |
| known as EGFR and ErbB1) | | | | | |
| Receptor tyrosine-protein kinase erbB-2 | P04626 | 653 | 675 | 23 | 328 |
| (also known as HER2, Neu and ErbB2) | | | | | |
| Hepatocyte growth factor receptor (also | P08581 | 933 | 955 | 23 | 329 |
| known as HGFR and c-Met) | | | | | |
| Fibroblast growth factor receptor 1 (also | P11362 | 377 | 397 | 21 | 330 |
| known as FGFR1 and CD331) | | | | | |
| Fibroblast growth factor receptor 2 (also | P21802 | 378 | 398 | 21 | 331 |
| known as FGFR2 and CD332) | | | | | |
| Fibroblast growth factor receptor 3 (also | P22607 | 376 | 396 | 21 | 332 |
| known as FGFR3 and CD333) | | | | | |
| Fibroblast growth factor receptor 4 (also | P22455 | 370 | 390 | 21 | 333 |
| known as FGFR4 and CD334) | | | | | |
| Vascular endothelial growth factor receptor 2 | P35968 | 765 | 785 | 21 | 334 |
| (also known as VEGFR-2 and CD309) | | | | | |
| Vascular endothelial growth factor receptor 3 | P35916 | 776 | 796 | 21 | 335 |
| (also known as VEGFR-3) | | | | | |
| Ephrin type-A receptor 1 (also known as | P21709 | 548 | 568 | 21 | 336 |
| EPHA1 and EPH) | | | | | |
| Ephrin type-B receptor 1 (also known as | P54762 | 541 | 563 | 23 | 337 |
| EPHB1, EK6, and ELK) | | | | | |
| Platelet-derived growth factor receptor alpha | P16234 | 529 | 549 | 21 | 338 |
| (also known as PDGFRA and CD140a) | | | | | |
| Platelet-derived growth factor receptor beta | P09619 | 533 | 553 | 21 | 339 |
| (also known as PDGFRB and CD140b) | | | | | |
| B-cell antigen receptor complex-associated | P11912 | 144 | 165 | 22 | 340 |
| protein alpha chain (also known as Ig-alpha | | | | | |
| and CD79A) | | | | | |
| B-cell antigen receptor complex-associated | P40259 | 160 | 180 | 21 | 341 |
| protein beta chain (also known as Ig-beta and | | | | | |
| CD79B) | | | | | |
| CD160 antigen (also known as CD160 and | O95971 | 163 | 182 | 20 | 342 |
| CD160) | | | | | |
| CD226 antigen (also known as DNAM-1 and | Q15762 | 255 | 275 | 21 | 343 |
| CD226) | | | | | |
| CD83 antigen (also known as CD83) | Q01151 | 145 | 166 | 22 | 344 |
| Inducible T-cell costimulator (also known as | Q9Y6W8 | 141 | 161 | 21 | 345 |
| ICOS and CD278) | | | | | |
| Intercellular adhesion molecule 1 (also | P05362 | 481 | 503 | 23 | 346 |
| known as ICAM-1 and CD54) | | | | | |
| Intercellular adhesion molecule 2 (also | P13598 | 224 | 248 | 25 | 347 |
| known as ICAM-2 and CD102) | | | | | |
| Intercellular adhesion molecule 3 (also | P32942 | 486 | 510 | 25 | 348 |
| known as ICAM-3 and CD50) | | | | | |
| Killer cell immunoglobulin-like receptor | Q99706 | 243 | 263 | 21 | 349 |
| 2DL4 (also known as KIR2DL, and | | | | | |
| CD158D) | | | | | |
| Killer cell immunoglobulin-like receptor | Q14954 | 246 | 264 | 19 | 350 |
| 2DS1 (also known as KIR2DS1 and | | | | | |
| CD158H) | | | | | |
| Killer cell immunoglobulin-like receptor | P43631 | 246 | 265 | 20 | 351 |
| 2DS2 (also known as KIR2DS2 and | | | | | |
| CD158J) | | | | | |
| Killer cell immunoglobulin-like receptor | Q14952 | 246 | 264 | 19 | 352 |
| 2DS3 (also known as KIR2DS3) | | | | | |
| Killer cell immunoglobulin-like receptor | P43632 | 246 | 265 | 20 | 353 |
| 2DS4 (also known as KIR2DS4 and | | | | | |
| CD158I) | | | | | |
| Killer cell immunoglobulin-like receptor | Q14953 | 246 | 264 | 19 | 354 |
| 2DS50 (also known as KIR2DS5 and | | | | | |
| CD158G) | | | | | |
| Killer cell immunoglobulin-like receptor | Q14943 | 341 | 360 | 20 | 355 |
| 3DS1 (also known as KIR3DS1) | | | | | |
| Natural cytotoxicity triggering receptor 1 | O76036 | 259 | 279 | 21 | 356 |
| (also known as NKp46, Ly94, and CD335) | | | | | |
| Natural cytotoxicity triggering receptor 2 | O95944 | 193 | 213 | 21 | 357 |
| (also known as NKp44 and CD336) | | | | | |
| Natural cytotoxicity triggering receptor 3 | O14931 | 136 | 156 | 21 | 358 |
| (also known as NKp30 and CD337) | | | | | |
| T-cell antigen CD7 (also known as CD7) | P09564 | 181 | 201 | 21 | 359 |
| T-cell surface glycoprotein CD4 (also known | P01730 | 397 | 418 | 22 | 360 |
| as CD4) | | | | | |
| T-cell-specific surface glycoprotein CD28 | P10747 | 153 | 179 | 27 | 361 |
| (also known as CD28) | | | | | |
| Transmembrane and immunoglobulin | Q96BF3 | 151 | 171 | 21 | 362 |
| domain-containing protein 2 (also known as | | | | | |
| TMIGD2, CD28H, IGPR-1) | | | | | |
| Intercellular adhesion molecule 5 (also | Q9UMF0 | 836 | 856 | 21 | 363 |
| known as ICAM-5, telencephalin) | | | | | |
| Integrin alpha-L (also known as LFA-1A and | P20701 | 1091 | 1111 | 21 | 364 |
| CD11A) | | | | | |
| Integrin beta-2 (also known as LFA-1B and | P05107 | 701 | 723 | 23 | 365 |
| CD18) | | | | | |
| Cytotoxic and regulatory T-cell molecule | O95727 | 288 | 308 | 21 | 366 |
| (also known as CRTAM and CD355) | | | | | |
| B-cell receptor CD22 (also known as Siglec- | P20273 | 688 | 706 | 19 | 367 |
| 2 and CD22) | | | | | |
| Sialic acid-binding Ig-like lectin 14 (also | Q08ET2 | 359 | 381 | 23 | 368 |
| known as Siglec-14) | | | | | |
| Sialic acid-binding Ig-like lectin 15 (also | Q6ZMC9 | 264 | 284 | 21 | 369 |
| known as Siglec-15) | | | | | |
| Sialic acid-binding Ig-like lectin 16 (also | A6NMB1 | 435 | 455 | 21 | 370 |
| known as Siglec-16) | | | | | |
| Hepatitis A virus cellular receptor 1 (also | Q96D42 | 296 | 316 | 21 | 371 |
| known as TIM-1, KIM-1, and CD365) | | | | | |
| Toll-like receptor 1 (also known as TLR1 | Q15399 | 581 | 601 | 21 | 372 |
| and CD281) | | | | | |
| Toll-like receptor 10 (also known as TLR10 | Q9BXR5 | 577 | 597 | 21 | 373 |
| and CD290) | | | | | |
| Toll-like receptor 2 (also known as TLR2 | O60603 | 589 | 609 | 21 | 374 |
| and CD282) | | | | | |
| Toll-like receptor 3 (also known as TLR3 | O15455 | 705 | 725 | 21 | 375 |
| and CD283) | | | | | |
| Toll-like receptor 4 (also known as TLR4 | O00206 | 632 | 652 | 21 | 376 |
| and CD284) | | | | | |
| Toll-like receptor 5 (also known as TLR5 | O60602 | 640 | 660 | 21 | 377 |
| and CD285) | | | | | |
| Toll-like receptor 6 (also known as TLR6 | Q9Y2C9 | 587 | 607 | 21 | 378 |
| and CD286) | | | | | |
| Toll-like receptor 7 (also known as TLR7 | Q9NYK1 | 840 | 860 | 21 | 379 |
| and CD287) | | | | | |
| Toll-like receptor 8 (also known as TLR8 | Q9NR97 | 828 | 848 | 21 | 380 |
| and CD288) | | | | | |
| Toll-like receptor 9 (also known as TLR9 | Q9NR96 | 819 | 839 | 21 | 381 |
| and CD289) | | | | | |
| CD27 antigen (also known as CD27) | P26842 | 192 | 212 | 21 | 382 |
| Tumor necrosis factor receptor superfamily | Q9Y6Q6 | 213 | 233 | 21 | 383 |
| member 11A (also known as RANK and | | | | | |
| CD265) | | | | | |
| Tumor necrosis factor receptor superfamily | Q9NP84 | 81 | 101 | 21 | 384 |
| member 12A (also known as TweakR, FN14 | | | | | |
| and CD266) | | | | | |
| Tumor necrosis factor receptor superfamily | O14836 | 166 | 186 | 21 | 385 |
| member 13B (also known as TACI and | | | | | |
| CD267) | | | | | |
| Tumor necrosis factor receptor superfamily | Q96RJ3 | 79 | 99 | 21 | 386 |
| member 13C (also known as BAFF-R and | | | | | |
| CD268) | | | | | |
| Tumor necrosis factor receptor superfamily | Q92956 | 203 | 223 | 21 | 387 |
| member 14 (also known as HVEM and | | | | | |
| CD270) | | | | | |
| Tumor necrosis factor receptor superfamily | P08138 | 251 | 272 | 22 | 388 |
| member 16 (also known as NGF-R, | | | | | |
| p75NTR, and CD271) | | | | | |
| Tumor necrosis factor receptor superfamily | Q02223 | 55 | 77 | 23 | 389 |
| member 17 (also known as BCMA and | | | | | |
| CD269) | | | | | |
| Tumor necrosis factor receptor superfamily | Q9Y5U5 | 163 | 183 | 21 | 390 |
| member 18 (also known as GITR and | | | | | |
| CD3 57) | | | | | |
| Tumor necrosis factor receptor superfamily | Q9NS68 | 171 | 191 | 21 | 391 |
| member 19 (also known as TROY and | | | | | |
| TRADE) | | | | | |
| Tumor necrosis factor receptor superfamily | Q969Z4 | 163 | 183 | 21 | 392 |
| member 19L (also known as RELT) | | | | | |
| Tumor necrosis factor receptor superfamily | P19438 | 212 | 232 | 21 | 393 |
| member lA (also known as TNF-RI and | | | | | |
| CD120A) | | | | | |
| Tumor necrosis factor receptor superfamily | P20333 | 258 | 287 | 30 | 394 |
| member 1B (also known as TNF-RII and | | | | | |
| CD120B) | | | | | |
| Tumor necrosis factor receptor superfamily | Q93038 | 200 | 220 | 21 | 395 |
| member 25 (also known as DR3 and | | | | | |
| TRAMP) | | | | | |
| Tumor necrosis factor receptor superfamily | Q9HAV5 | 139 | 159 | 21 | 396 |
| member 27 (also known as XEDAR and | | | | | |
| EDA-A2 receptor) | | | | | |
| Tumor necrosis factor receptor superfamily | P36941 | 228 | 248 | 21 | 397 |
| member 3 (also known as LTB-R and TNF- | | | | | |
| RIII) | | | | | |
| Tumor necrosis factor receptor superfamily | P43489 | 215 | 235 | 21 | 398 |
| member 4 (also known as OX-40 and | | | | | |
| CD134) | | | | | |
| Tumor necrosis factor receptor superfamily | P25942 | 194 | 215 | 22 | 399 |
| member 5 (also known as CD40) | | | | | |
| Tumor necrosis factor receptor superfamily | P28908 | 386 | 406 | 21 | 400 |
| member 8 (also known as CD30) | | | | | |
| Tumor necrosis factor receptor superfamily | Q07011 | 187 | 213 | 27 | 401 |
| member 9 (also known as 4-1BB and | | | | | |
| CD137) | | | | | |
| Tumor necrosis factor receptor superfamily | Q9UNE0 | 188 | 208 | 21 | 402 |
| member EDAR (also known as EDAR) | | | | | |
| Linker for activation of T-cells family | O43561 | 5 | 27 | 23 | 403 |
| member 1 (also known as LAT) | | | | | |
| Linker for activation of T-cells family | Q9GZY6 | 6 | 26 | 21 | 404 |
| member 2 (also known as LAT2, NTAL, and | | | | | |
| LAB) | | | | | |
| Lymphocyte transmembrane adapter 1 (also | Q8IWV1 | 38 | 58 | 21 | 405 |
| known as LAX) | | | | | |
| Phosphoprotein associated with | Q9NWQ8 | 17 | 37 | 21 | 406 |
| glycosphingolipid-enriched microdomains 1 | | | | | |
| (also known as PAG and CBP) | | | | | |
| Linker for activation of T-cells family | O43561 | 5 | 27 | 23 | 407 |
| member 1 (also known as LAT) | | | | | |
| Low affinity immunoglobulin epsilon Fc | P06734 | 22 | 47 | 26 | 408 |
| receptor (also known as FCERII and CD23) | | | | | |
| CD209 antigen (also known as DC-SIGN, | Q9NNX6 | 38 | 58 | 21 | 409 |
| CLEC-4L, and CD209) | | | | | |
| C-type lectin domain family 1 member B | Q9P126 | 34 | 54 | 21 | 410 |
| (also known as CLEC-2) | | | | | |
| C-type lectin domain family 7 member A | Q9BXN2 | 45 | 65 | 21 | 411 |
| (also known as Dectin-1 and CLEC-7A) | | | | | |
| C-type lectin domain family 9 member A | Q6UXN8 | 36 | 56 | 21 | 412 |
| (also known as DNGR-1 and CD370) | | | | | |
| Killer cell lectin-like receptor subfamily F | Q9NZS2 | 39 | 59 | 21 | 413 |
| member 1 (also known as NKp80, KLRF1, | | | | | |
| and CLEC5C) | | | | | |
| Killer cell lectin-like receptor subfamily F | D3W0D1 | 31 | 51 | 21 | 414 |
| member 2 (also known as NKp65 and | | | | | |
| KLRF2) | | | | | |
| NKG2-C type II integral membrane protein | P26717 | 71 | 93 | 23 | 415 |
| (also known as NKG2C, KLRC2, and | | | | | |
| CD159C) | | | | | |
| NKG2-D type II integral membrane protein | P26718 | 52 | 72 | 21 | 416 |
| (also known as NKG2D and CD314) | | | | | |
| NKG2-E type II integral membrane protein | Q07444 | 71 | 93 | 23 | 417 |
| (also known as NKG2E and KLRC3) | | | | | |
| C-type lectin domain family 4 member E | Q9ULY5 | 20 | 40 | 21 | 418 |
| (also known as CLEC-4E and MINCLE) | | | | | |
| C-type lectin domain family 6 member A | Q6EIG7 | 21 | 41 | 21 | 419 |
| (also known as CLEC-6A and Dectin-2) | | | | | |
| C-type lectin domain family 10 member A | Q8IUN9 | 40 | 60 | 21 | 420 |
| (also known as CLEC-10A, MGL, and | | | | | |
| CD301) | | | | | |
| C-type lectin domain family 4 member D | Q8WXI8 | 18 | 38 | 21 | 421 |
| (also known as CLEC-4D, CLEC-6, Dectin- | | | | | |
| 3, and CD368) | | | | | |
| C-type lectin domain family 4 member C | Q8WTT0 | 22 | 44 | 23 | 422 |
| (also known as CLEC-4C, BDCA-2, and | | | | | |
| CD303) | | | | | |
| C-type lectin domain family 17, member A | Q6ZS10 | 173 | 193 | 21 | 423 |
| (also known as CLEC-17A and Prolectin) | | | | | |
| CD70 antigen (also known as CD70) | P32970 | 18 | 38 | 21 | 424 |
| Tumor necrosis factor ligand superfamily | O43557 | 38 | 58 | 21 | 425 |
| member 14 (also known as LIGHT and | | | | | |
| CD258) | | | | | |
| Tumor necrosis factor ligand superfamily | P32971 | 38 | 62 | 25 | 426 |
| member 8 (also known as CD30L and | | | | | |
| CD153) | | | | | |
| Tumor necrosis factor (also known as tumor | P01375 | 36 | 56 | 21 | 427 |
| necrosis factor, TNFa, and TNFSF1A) | | | | | |
| Tumor necrosis factor ligand superfamily | P23510 | 24 | 50 | 27 | 428 |
| member 4 (also known as OX40L, CD252, | | | | | |
| CD134L, and CD252) | | | | | |
| CD40 ligand (also known as CD40L, | P29965 | 23 | 46 | 24 | 429 |
| CD154, and CD154) | | | | | |
| Tumor necrosis factor ligand superfamily | P48023 | 81 | 102 | 22 | 430 |
| member 6 (also known as FasL, CD178, | | | | | |
| CD95L, and CD178) | | | | | |
| Tumor necrosis factor ligand superfamily | P41273 | 29 | 49 | 21 | 431 |
| member 9 (also known as 4-1BBL and | | | | | |
| CD137L) | | | | | |
| Tumor necrosis factor ligand superfamily | P50591 | 18 | 38 | 21 | 432 |
| member 10 (also known as TRAIL, TNF- | | | | | |
| related apoptosis-inducing ligand, CD253, | | | | | |
| APO-2L, and CD253) | | | | | |
| Tumor necrosis factor ligand superfamily | O14788 | 48 | 68 | 21 | 433 |
| member 11 (also known as TRANCE, | | | | | |
| RANKL, CD254, OPGL, and CD254) | | | | | |
| Tumor necrosis factor ligand superfamily | O43508 | 22 | 42 | 21 | 434 |
| member 12 (also known as TWEAK, APO- | | | | | |
| 3L, and DR3L) | | | | | |
| Tumor necrosis factor ligand superfamily | O75888 | 29 | 49 | 21 | 435 |
| member 13 (also known as APRIL, CD256, | | | | | |
| TALL-2, TRDL1, and CD256) | | | | | |
| Tumor necrosis factor ligand superfamily | Q9Y275 | 47 | 67 | 21 | 436 |
| member 13B (also known as BAFF, B-Cell | | | | | |
| Activating Factor, CD257, TALL-1, and | | | | | |
| CD257) | | | | | |
| Tumor necrosis factor ligand superfamily | Q9UNG2 | 51 | 71 | 21 | 437 |
| member 18 (also known as TNFSF18, | | | | | |
| GITRL, and TL-6) | | | | | |
| Paired immunoglobulin-like type 2 receptor | Q9UKJ0 | 192 | 212 | 21 | 534 |
| beta (also known as PILRB) | | | | | |

In some embodiments, an engineered protein (e.g., chimeric protein) provided herein includes a transmembrane domain comprising or consisting of an amino acid sequence having at least 90%, at least 91%, at least 92%, at least 93%, at least 94%, at least 95%, at least 96%, at least 97%, at least 98%, at least 99% or 100% identity with the amino acid sequence of any one of SEQ ID NOs: 264-437 and 534.

In some embodiments, an engineered protein (e.g., chimeric protein) provided herein includes a transmembrane domain selected from a human CD8alpha transmembrane domain, a human CD16 transmembrane domain, a human CD28 transmembrane domain, a human NKG2D transmembrane domain, a human NKp44 transmembrane domain, a human NKp46 transmembrane domain, a human CD27 transmembrane domain, a human DAP10 transmembrane domain, a PILRB transmembrane domain, and a human DAP12 transmembrane domain, or a portion of any of the foregoing.

Alternatively, the transmembrane domain of an engineered protein (e.g., chimeric protein) provided herein can be synthetic, and can comprise hydrophobic residues such as, e.g., leucine and valine. In some embodiments, a triplet of phenylalanine, tryptophan, and valine is found at one or both termini of a synthetic transmembrane domain of an engineered protein (e.g., chimeric protein) provided herein.

In some embodiments, a short polypeptide linker, e.g., between 2 and 10 amino acids in length, may form a linkage between the transmembrane domain and the intracellular domain of an engineered protein (e.g., chimeric protein) provided herein. In some embodiments, the linker is a glycine-serine linker. Any of the linkers described herein may be included in the engineered protein (e.g., chimeric protein).

In some embodiments, an engineered protein (e.g., chimeric protein) provided herein includes a transmembrane domain derived from human DAP10 comprising an amino acid sequence having at least 90%, at least 91%, at least 92%, at least 93%, at least 94%, at least 95%, at least 96%, at least 97%, at least 98%, at least 99%, or 100% identity with the amino acid sequence of SEQ ID NO: 264.

In some embodiments, an engineered protein (e.g., chimeric protein) provided herein includes a transmembrane domain derived from a human DAP12 comprising an amino acid sequence having at least 90%, at least 91%, at least 92%, at least 93%, at least 94%, at least 95%, at least 96%, at least 97%, at least 98%, at least 99%, or 100% identity with the amino acid sequence of SEQ ID NO: 266.

### D. Linkers

The term “linker” as used herein refers to any polypeptide that functions to link one or more domains of an engineered protein (e.g., chimeric protein) provided herein (e.g., a transmembrane domain to an extracellular domain and/or an intracellular domain in an engineered protein (e.g., chimeric protein) of the disclosure). In particular, linkers may be used to provide more flexibility and accessibility for the functioning of the extracellular domain, the transmembrane domain, and/or the intracellular domain. A linker can also be used to separate two different intracellular domains.

A linker useful in the engineered proteins (e.g., chimeric proteins) herein may comprise from about 1 to about 200 amino acids, from about 1 to about 10 amino acids, from about 10 to about 100 amino acids, from about 100 to about 200 amino acids, from about 10 to about 20 amino acids, from about 20 to about 30 amino acids, from about 30 to about 40 amino acids, from about 40 to about 50 amino acids, from about 50 to about 70 amino acids, from about 70 to about 90 amino acids, from about 90 to about 120 amino acids, from about 100 to about 150 amino acids, or from about 150 to about 200 amino acids, in length.

In some embodiments, the transmembrane domain and the extracellular domain are connected by a linker. In some embodiments, the linker establishes an optimal distance to facilitate the functioning of the extracellular domain. In some embodiments, the linker provides flexibility for the extracellular domain to bind to a negative signal.

In some embodiments, the transmembrane domain and the intracellular domain are connected by a linker. In some embodiments, the linker establishes an optimal distance to facilitate the functioning of the intracellular domain. In some embodiments, the linker provides flexibility for the intracellular domain to transduce a effector function signal in the cell, which in some embodiments is to induce a positive signal that activates an immune cell.

In some embodiments, the linker is selected from the linkers presented in Table 3.

TABLE 3
Exemplary Linkers

| | | SEQ |
|---|---|---|
| | | ID |
| Linker name | Amino acid sequence | NO: |
| CD8a hinge | TTTPAPRPPTPAPTIASQPLSLRPEACRPAAGGAVHTRGLDFACD | 438 |
| short | | |
| CD8a hinge | FVPVFLPAKPTTTPAPRPPTPAPTIASQPLSLRPEACRPAAGGAVH | 439 |
| long | TRGLDFACD | |
| IgG1 hinge | EPKSCDKTHTCPPCPAPELLGGPSVFLFPPKPKDTLMISRTPEVTC | 440 |
| | VVVDVSHEDPEVKFNWYVDGVEVHNAKTKPREEQYNSTYRVVS | |
| | VLTVLHQDWLNGKEYKCKVSNKALPAPIEKTISKAKGQPREPQV | |
| | YTLPPSRDELTKNQVSLTCLVKGFYPSDIAVEWESNGQPENNYKT | |
| | TPPVLDSDGSFFLYSKLTVDKSRWQQGNVFSCSVMHEALHNHYT | |
| | QKSLSLSPGK | |
| IgG1 hinge | EPKSPDKTHTCPPCPAPELLGGPSVFLFPPKPKDTLMISRTPEVTC | 441 |
| v2 | VVVDVSHEDPEVKFNWYVDGVEVHNAKTKPREEQYNSTYRVVS | |
| | VLTVLHQDWLNGKEYKCKVSNKALPAPIEKTISKAKGQPREPQV | |
| | YTLPPSRDELTKNQVSLTCLVKGFYPSDIAVEWESNGQPENNYKT | |
| | TPPVLDSDGSFFLYSKLTVDKSRWQQGNVESCSVMHEALHNHYT | |
| | QKSLSLSPGKKDPK | |
| CD28 hinge | IEVMYPPPYLDNEKSNGTIIHVKGKHLCPSPLFPGPSKP | 442 |
| KIR2DS2 | SPTEPSSKTGNPRHLH | 443 |
| hinge | | |
| IgG4 hinge | ESKYGPPCPSCP | 444 |
| short | | |
| IgG4 hinge | ESKYGPPCPSCPAPEFLGGPSVFLFPPKPKDTLMISRTPEVTCVVV | 445 |
| | DVSQEDPEVQFNWYVDGVEVHNAKTKPREEQFNSTYRVVSVLT | |
| | VLHQDWLNGKEYKCKVSNKGLPSSIEKTISKAKGQPREPQVYTL | |
| | PPSQEEMTKNQVSLTCLVKGFYPSDIAVEWESNGQPENNYKTTPP | |
| | VLDSDGSFFLYSRLTVDKSRWQEGNVFSCSVMHEALHNHYTQK | |
| | SLSLSLGK | |
| CD16_Hinge | GLAVSTISSFFPPGYQ | 446 |
| 651 G4S x1 | GGGGS | 447 |
| G4S x2 | GGGGSGGGGS | 448 |
| 652 G4S x3 | GGGGSGGGGSGGGGS | 449 |
| 653 | GGSGGSGGYPYDVPDYAGGGSGGGS | 450 |
| 654 | GGSGGSGGGGGSGGGSGGGSGGGS | 451 |
| 655 | GGSGGSGGGPEDEPGSGSGGGSGGGS | 452 |
| 656 | GGSGGSGGGGGSGGGSGGGSGGGSGSGSGSGSEDGSGSGSGS | 453 |
| 657 | GSGSGSGSGSEDEDEDEDGSGSGSGSGS | 454 |
| 658 | S | 455 |
| 659 | GSGSGSGSEDGSGSGSGS | 456 |
| 660 | GSGSGSGSGSGSGSGSGS | 457 |
| 661 | GCGGSGGGGSGGGGS | 458 |
| 654 | GGSGGSGGGGGSGGGSGGGSGGGS | 459 |
| 662 | SGRGGGGSGGGGSGGGGSGGGGSSPA | 460 |
| 663 | GGGGSGGGGSGGGGSGGGGSGGGG | 461 |
| 664 | SGRGASSGSSGSGSQKKPRYEIRWKVVVISAILALVVLTVISLIILI | 462 |
| | MLWGSGMQSPA | |

In some embodiments, a linker in an engineered protein (e.g., chimeric protein) provided herein may be derived from all or part of a naturally occurring molecule, such as from all or part of the extracellular region of CD8, CD8alpha, CD4, CD28, 4-1BB, or IgG (in particular, the linker region of an IgG, for example from IgG1, IgG2 or IgG4), or from all or part of an antibody heavy-chain constant region. Alternatively, the linker may be a synthetic sequence that corresponds to a naturally occurring linker sequence or may be an entirely synthetic linker sequence. In some embodiments, the linker corresponds to Fc domains of a human immunoglobulin, e.g., either the CH2 or CH3 domain. In some embodiments, the CH2 and CH3 linker region of a human immunoglobulin has been modified to improve dimerization. In some embodiments, the linker is derived from an immunoglobulin. In some embodiments, the linker comprises or consists of a CH3 region of a human immunoglobulin. In some embodiments, the linker comprises or consists of a CH2 region of a human immunoglobulin. In some embodiments, the linker comprises or consists of a CH2 and CH3 region of a human immunoglobulin. In some embodiments, the CH2 region is from a human IgG1, IgG2 or IgG4 immunoglobulin.

In some embodiments, the linker is derived from a human CD8α chain (e.g., NP_001139345.1). In some embodiments, the linker of the engineered proteins (e.g., chimeric proteins) described herein comprises a subsequence of CD8α, an IgG1, an IgG4, FcγRIIIα, or CD28. In some embodiments, the linker is derived from the stalk domain of a human CD8α, a human IgG1, a human IgG4, a human FcγRIIIα, or a human CD28.

In some embodiments, an engineered protein (e.g., chimeric protein) provided herein includes one or more (e.g., one, two, three, four, or five) linkers disposed between an extracellular domain and a transmembrane domain, between a transmembrane domain and an intracellular domain, and/or between two or more intracellular domains). In some embodiments, an engineered protein (e.g., chimeric protein) provided herein includes one or more linkers, wherein the linker comprises an amino acid sequence having at least 90%, at least 91%, at least 92%, at least 93%, at least 94%, at least 95%, at least 96%, at least 97%, at least 98%, at least 99%, or 100% identity with the amino acid sequence of any one of SEQ ID NOs: 438-462.

Included in the scope of the disclosure are nucleic acid sequences that encode functional portions, e.g., one, two, or three domains, of the engineered proteins (e.g., chimeric proteins) described herein. Functional portions encompass, for example, those parts of an engineered protein (e.g., chimeric protein) that retain the ability to recognize negative signals, or to detect, treat, or prevent a disease. In some embodiments, the engineered proteins (e.g., chimeric proteins) provided herein include additional amino acid residues at the amino or carboxy terminus of the portion, or at both termini, which additional amino acids are not found in the amino acid sequence of the inhibitory polypeptide and/or stimulatory polypeptide from which the domains (e.g., extracellular domain and/or intracellular domain) in the engineered protein (e.g., chimeric protein) are derived.

The engineered proteins (e.g., chimeric proteins) described herein (including functional portions and functional variants thereof) may be glycosylated, amidated, carboxylated, phosphorylated, esterified, N-acylated, or cyclized (via, e.g., a disulfide bridge) proteins, or converted into acid addition salts and/or optionally dimerized or polymerized.

### E. Signal Peptides

In some embodiments, an engineered protein (e.g., chimeric protein) provided herein includes a signal peptide (also known as a leader peptide). In some embodiments, the signal peptide is a type I membrane protein leader peptide. In some embodiments, the signal peptide is a type II membrane protein leader peptide. In some embodiments, an engineered protein (e.g., chimeric protein) provided herein includes a signal peptide at its amino terminus (N-terminus). In some embodiments, the engineered protein (e.g., chimeric protein) includes a signal peptide at the N-terminus of an extracellular domain. In some embodiments, the signal peptide is cleaved from the engineered protein (e.g., chimeric protein) during cellular processing and localization of the engineered protein to the cellular membrane (e.g., plasma membrane) of a cell expressing the protein. Exemplary signal peptides (e.g., derived from inhibitory polypeptides) that may be included in an engineered protein (e.g., chimeric protein) provided herein are listed in Table 18.

TABLE 18
Exemplary Signal Peptide Sequences

| | | | | Amino | SEQ |
|---|---|---|---|---|---|
| | UNIPROT | | | acid | ID |
| Inhibitory polypeptide | ID | Start | End | length | NO: |
| BTLA | Q7Z6A9 | 1 | 30 | 30 | 463 |
| CD160 | O95971 | 1 | 24 | 24 | 464 |
| CD200R | Q8TD46 | 1 | 28 | 28 | 465 |
| CD33 | P20138 | 1 | 17 | 17 | 466 |
| CEACAM-1 | P13688 | 1 | 34 | 34 | 467 |
| (also known as CD66a) | | | | | |
| CTLA-4 | P16410 | 1 | 35 | 35 | 468 |
| Fas | P25445 | 1 | 25 | 25 | 469 |
| FCRL6 | Q6DN72 | 1 | 19 | 19 | 470 |
| IL-10RA | Q13651 | 1 | 21 | 21 | 471 |
| IL-10RB | Q08334 | 1 | 19 | 19 | 472 |
| IL-6RA | P08887 | 1 | 19 | 19 | 473 |
| IL-6RB (also known as | P40189 | 1 | 22 | 22 | 474 |
| gp130 and CD130) | | | | | |
| KIR2DL1 | P43626 | 1 | 21 | 21 | 475 |
| KIR2DL2 | P43627 | 1 | 21 | 21 | 476 |
| KIR2DL3 | P43628 | 1 | 21 | 21 | 477 |
| KIR2DL5A | Q8N109 | 1 | 21 | 21 | 478 |
| KIR2DL5B | Q8NHK3 | 1 | 21 | 21 | 479 |
| KIR3DL1 | P43629 | 1 | 21 | 21 | 480 |
| KIR3DL2 | P43630 | 1 | 21 | 21 | 481 |
| KIR3DL3 | Q8N743 | 1 | 25 | 25 | 482 |
| Lag3 | P18627 | 1 | 22 | 22 | 483 |
| LILRB1 | Q8NHL6 | 1 | 23 | 23 | 484 |
| LILRB2 | Q8N423 | 1 | 21 | 21 | 485 |
| LILRB3 | O75022 | 1 | 23 | 23 | 486 |
| LILRB4 | Q8NHJ6 | 1 | 21 | 21 | 487 |
| LILRB5 | O75023 | 1 | 23 | 23 | 488 |
| NKp30c | O14931-2 | 1 | 18 | 18 | 489 |
| PD-1 | Q15116 | 1 | 23 | 23 | 490 |
| Siglec-10 | Q96LC7 | 1 | 16 | 16 | 491 |
| Siglec-7 | Q9Y286 | 1 | 18 | 18 | 492 |
| Siglec-9 | Q9Y336 | 1 | 17 | 17 | 493 |
| TACTILE | P40200 | 1 | 21 | 21 | 494 |
| (also known as CD96) | | | | | |
| TGF-BR1 | P36897 | 1 | 33 | 33 | 495 |
| TGF-BR2 | P37173 | 1 | 22 | 22 | 496 |
| TIGIT | Q495A1 | 1 | 21 | 21 | 497 |
| TIM-3 | Q8TDQ0 | 1 | 21 | 21 | 498 |
| CD8α | P01732 | 1 | 21 | 21 | 676 |

In some embodiments, an engineered protein (e.g., chimeric protein) provided herein includes a signal peptide that is a signal peptide of a CD8 protein.

In some embodiments, an engineered protein (e.g., chimeric protein) provided herein includes a signal peptide comprising an amino acid sequence having at least 90%, at least 91%, at least 92%, at least 93%, at least 94%, at least 95%, at least 96%, at least 97%, at least 98%, at least 99%, or 100% identity with the amino acid sequence of any one of SEQ ID NOs: 463-498 and 676.

### F. Exemplary Engineered Protein Constructs

### Sinks

Exemplary engineered proteins (e.g., chimeric proteins) of the sink modality, nucleic acids encoding the engineered proteins (e.g., chimeric proteins), and cells, e.g., immune cells, comprising one or more of these engineered proteins (e.g., chimeric proteins), are provided herein. In some embodiments, the disclosure provides proteins that acts as a sink, wherein the protein comprises an extracellular domain and a transmembrane domain, and wherein the protein lacks a fully functional intracellular domain. The extracellular domain of the sink binds to a negative signal (e.g., any of the exemplary negative signals described herein an exogenous ligand that inhibits the activation of an immune response).

In some embodiments, the sink protein comprises a transmembrane domain and an extracellular domain from the same protein, e.g., the sink protein is a truncated protein lacking its intracellular domain or a portion of its intracellular domain. In some embodiments, the sink protein comprises a transmembrane domain and an extracellular domain that are from different proteins, i.e., the sink protein is a chimeric protein.

In some embodiments, the sink protein comprises a polypeptide sequence extending to include, in addition to the extracellular and transmembrane domains, between 1 and 15 additional amino acids of an intracellular domain (e.g., as defined by UNIPROT) of the protein from which the sink protein is derived (e.g., a wild-type inhibitory protein). In some embodiments, the transmembrane domain of a sink protein may comprise up to 5, up to 10, or up to 15 amino acid residues of the intracellular domain, i.e., the corresponding intracellular domain of the protein from which the transmembrane domain is derived. In some embodiments, a sink protein comprises a amino acid sequence extended to include, in addition to the extracellular and transmembrane domains, additional amino acids of the intracellular domain up to and including a charged amino acid residue at the terminus that is oriented towards the cytoplasm of a cell where the protein is expressed.

In some embodiments, the extracellular domain of the sink protein comprises the extracellular domain, or a portion thereof, of an inhibitory polypeptide that binds to a negative signal. In embodiments, the extracellular domain of the sink protein comprises the extracellular domain, or a portion thereof, of an inhibitory polypeptide presented in Table 1 or Table 1.1. In some embodiments, the extracellular domain of the sink protein consists of the extracellular domain, or a portion thereof, of an inhibitory polypeptide presented in Table 1 or Table 1.1. In some embodiments, the inhibitory polypeptide is selected from the inhibitory polypeptides presented in Table 1 or Table 1.1. In some embodiments, the inhibitory polypeptide is adenosine receptor A2A, adenosine receptor A2B, prostaglandin receptor EP2, prostaglandin receptor EP4, TGF-BR1, TGF-BR2, IL-10RA, IL-10RA, IL-1R8, IL-6RA, IL-10RA, IL-6RB (also known as gp130 and CD130), IL-10RA, PD-1, CTLA-4, TIM-3, Lag3, BTLA, CD160, TIGIT, TACTILE (also known as CD96), KIR2DL1, KIR2DL2, KIR2DL3, KIR2DL5A, KIR2DL5B, KIR3DL1, KIR3DL2, KIR3DL3, LILRB1, LILRB2, LILRB3, LILRB4, LILRB5, CEACAM-1 (CD66a), NKG2A, KLRB1 (NKR-PIA), KLRG1, CD33, Siglec-7, Siglec-9, Siglec-10, Fas, or FCRL6.

In some embodiments, the sink protein comprises a transmembrane domain and an extracellular domain from the same protein. In some embodiments, the sink protein is a truncated version of any inhibitory protein disclosed herein, wherein the inhibitory protein is lacking its entire intracellular domain or a portion of the intracellular domain. In some embodiments, the sink protein comprises the extracellular domain, or portion thereof, and the transmembrane domain, or portion thereof, of an inhibitory polypeptide that binds to a negative signal. In embodiments, the sink protein comprises the extracellular domain, or portion thereof, and the transmembrane domain, or portion thereof, of an inhibitory polypeptide presented in Table 1 or Table 1.1. In some embodiments, the sink protein consists of the extracellular domain, or portion thereof, and the transmembrane domain, or portion thereof, of an inhibitory polypeptide presented in Table 1 or Table 1.1. In some embodiments, the inhibitory polypeptide is adenosine receptor A2A, adenosine receptor A2B, prostaglandin receptor EP2, prostaglandin receptor EP4, TGF-BR1, TGF-BR2, IL-10RA, IL-10RB, IL-10RA, IL-1R8, IL-10RA, IL-6RA, IL-10RA, IL-6RB (also known as gp 130 and CD130), IL-10RA, PD-1, I CTLA-4, TIM-3, Lag3, I BTLA, CD160, TIGIT, TACTILE (also known as CD96), CD200R, KIR2DL1, KIR2DL2, KIR2DL3, KIR2DLSA, KIR2DLSB, KIR3DL1, KIR3DL2, KIR3DL3, LILRB1, LILRB2, LILRB3, LILRB4, LILRB5, CEACAM-1 (CD66a), NKG2A, KLRB1 (NKR-PIA), KLRG1, CD33, Siglec-7, Siglec-9, Siglec-10, Fas, or FCRL6.

In some embodiments, the transmembrane domain and the extracellular domain of the sink protein are from different proteins, i.e., the sink protein is a chimeric protein. In some embodiments, the sink protein comprises an extracellular domain, or portion thereof, of a first inhibitory polypeptide that binds to a negative signal, and a transmembrane domain, or portion thereof, of a second inhibitory polypeptide that binds to a negative signal. In embodiments, the sink protein comprises an extracellular domain, or portion thereof, of a first inhibitory polypeptide presented in Table 1 or Table 1.1, and a transmembrane domain, or portion thereof, of a second inhibitory polypeptide presented in Table 1 or Table 1.1. In some embodiments, the sink protein consists of an extracellular domain, or portion thereof, of a first inhibitory polypeptide presented in Table 1 or Table 1.1, and a transmembrane domain, or portion thereof, of a second inhibitory polypeptide presented in Table 1 or Table 1.1. In some embodiments, the sink protein comprises an extracellular domain, or portion thereof, of an inhibitory polypeptide that binds to a negative signal, and a transmembrane domain, or portion thereof, of a stimulatory polypeptide. In some embodiments, the sink protein comprises an extracellular domain, or portion thereof, of an inhibitory polypeptide presented in Table 1 or Table 1.1, and a transmembrane domain, or portion thereof, of a stimulatory polypeptide presented in Table 2 or Table 2.1. In some embodiments, the sink protein consists of the extracellular domain, or portion thereof, of an inhibitory polypeptide presented in Table 1 or Table 1.1, and the transmembrane domain, or portion thereof, of a stimulatory polypeptide presented in Table 2 or Table 2.1.

In some embodiments, the extracellular domain of the sink protein comprises an antigen-binding domain that specifically binds to a negative signal. In some embodiments, the antigen-binding domain comprises a fragment of an antibody. In some embodiments, the antigen-binding domain comprises an scFv, a Fab, or a VHH. In some embodiments, the scFv is an scFv from a monoclonal antibody. In some embodiments, the scFv is connected to the transmembrane domain by a linker.

### Dominant Negative Receptors

Exemplary engineered proteins (e.g., chimeric proteins) of the dominant negative receptor (DNR) modality, nucleic acids encoding the engineered proteins (e.g., chimeric proteins), and cells, e.g., immune cells comprising one or more of these engineered proteins (e.g., chimeric proteins), are provided herein. In some embodiments, the disclosure provides dominant negative isoforms of a protein, wherein the dominant negative isoform of the protein competes with a wild-type isoform of the protein for binding a signal (e.g., a negative signal) that prevents the activation of an immune response. In some embodiments, the dominant negative isoform of a protein is a dominant negative isoform of an inhibitory polypeptide disclosed herein.

In some embodiments, the dominant negative isoform of a protein is an inhibitory polypeptide presented in Table 1 or Table 1.1, wherein at least one mutation or deletion has been introduced to produce a dominant negative isoform of the inhibitory polypeptide. In some embodiments, the dominant negative isoform of a protein is a dominant negative isoform of an inhibitory polypeptide presented in Table 1 or Table 1.1. In some embodiments, the inhibitory polypeptide is adenosine receptor A2A, adenosine receptor A2B, ITGF-BR1, TGF-BR2, IL-10RA, IL-1R8, IL-6RA, IL-6RB (gp130, CD130), PD-1, CTLA-4, Lag3, TACTILE (also known as CD96), or Fas.

In some embodiments, the chimeric protein comprises an extracellular domain of an inhibitory polypeptide presented in Table 1 or Table 1.1, and a transmembrane domain. In some embodiments, the chimeric protein comprises an extracellular domain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence of any one of SEQ ID NOs: 4-45, and a transmembrane domain (e.g., a transmembrane domain provided herein (e.g., a transmembrane domain of human CD28 or a transmembrane domain comprising an amino acid sequence having at least 90%, at least 91%, at least 92%, at least 93%, at least 94%, at least 95%, at least 96%, at least 97%, at least 98%, at least 99%, or 100% sequence identity to the amino acid sequence of any one of SEQ ID NOs: 223-263). In some embodiments, the chimeric protein comprises or consists of the extracellular domain and the transmembrane domain of an inhibitory polypeptide presented in Table 1 or Table 1.1. In some embodiments, the chimeric protein does not include an intracellular domain and/or an intracellular domain capable of providing a signal to a cell (e.g., an effector function signal).

Also provided herein are modified cells, e.g., immune cells (e.g., NK cells) engineered to comprise (e.g., express) a protein comprising a dominant negative isoform of TGF-BR1, wherein the dominant negative isoform of TGF-BR1 competes with a wild-type isoform of TGF-BR1 for binding a TGF-B (also known as TGF-β) signal that prevents the activation of an immune response. In some embodiments, the dominant negative isoform of TGF-BR1 is selected from the polypeptides described in Table 4.

TABLE 4
Exemplary Dominant Negative Receptors
Comprising a Dominant Negative
Isoform of TGF-BR1 (UNIPROT ID P36897)

| UNIPROT ID | Disease Phenotype | Mutation | SEQ ID NO: |
|---|---|---|---|
| P36897 | | Truncation of ICD | 538 |
| | | after amino acid | |
| | | residue 1147 | |
| P36897 | Loeys-Dietz syndrome 1 | K376E | 539 |
| P36897 | | K232R | 540 |
| P36897 | Loeys-Dietz syndrome 1 | T200I | 541 |
| P36897 | Loeys-Dietz syndrome 1 | K232E | 542 |
| P36897 | Loeys-Dietz syndrome 1 | S241L | 543 |
| P36897 | Loeys-Dietz syndrome 1 | M318R | 544 |
| P36897 | Loeys-Dietz syndrome 1 | G353V | 545 |
| P36897 | Loeys-Dietz syndrome 1 | D400G | 546 |
| P36897 | Loeys-Dietz syndrome 1 | R478P | 547 |

An exemplary polypeptide sequence of a TGF-BR1 polypeptide (also referred to herein as TGF-βR1) comprises or consists of the amino acid sequence of SEQ ID NO: 499. In some embodiments, the dominant negative isoform of TGF-BR1 comprises a truncation after an isoleucine at position 147 of SEQ ID NO: 499. In some embodiments, the dominant negative isoform of TGF-BR1 comprises a glutamate at position 376 of SEQ ID NO: 499. In some embodiments, the dominant negative isoform of TGF-BR1 comprises an arginine at position 232 of SEQ ID NO: 499. In some embodiments, the dominant negative isoform of TGF-BR1 comprises an isoleucine at position 200 of SEQ ID NO: 499. In some embodiments, the dominant negative isoform of TGF-BR1 comprises a glutamate at position 232 of SEQ ID NO: 499. In some embodiments, the dominant negative isoform of TGF-BR1 comprises a leucine at position 241 of SEQ ID NO: 499. In some embodiments, the dominant negative isoform of TGF-BR1 comprises an arginine at position 318 of SEQ ID NO: 499. In some embodiments, the dominant negative isoform of TGF-BR1 comprises a valine at position 353 of SEQ ID NO: 499. In some embodiments, the dominant negative isoform of TGF-BR1 comprises a glycine at position 400 of SEQ ID NO: 499. In some embodiments, the dominant negative isoform of TGF-BR1 comprises a proline at position 478 of SEQ ID NO: 499. In some embodiments, the dominant negative isoform of TGF-BR1 results in the development of one or more phenotypes associated with Loeys-Dietz syndrome 1 in a subject.

In some embodiments, the dominant negative isoform of TGF-BR1 comprises a polypeptide having at least 90%, at least 91%, at least 92%, at least 93%, at least 94%, at least 95%, at least 96%, at least 97%, at least 98%, at least 99%, or 100% sequence identity to the amino acid sequence of SEQ ID NO: 499, wherein the polypeptide comprises: a truncation after isoleucine at the amino acid position corresponding to position 147 of SEQ ID NO: 499, an arginine at the amino acid position corresponding to position 232 of SEQ ID NO: 499, an isoleucine at the amino acid position corresponding to position 200 of SEQ ID NO: 499, a glutamate at the amino acid position corresponding to position 232 of SEQ ID NO: 499, a leucine at the amino acid position corresponding to position 241 of SEQ ID NO: 499, an arginine at the amino acid position corresponding to position 318 of SEQ ID NO: 499, a valine at the amino acid position corresponding to position 353 of SEQ ID NO: 499, a glycine at the amino acid position corresponding to position 400 of SEQ ID NO: 499, or a proline at the amino acid position corresponding to position 478 of SEQ ID NO: 499. In some embodiments, the dominant negative isoform of TGF-BR1 results in the development of one or more phenotypes associated with Loeys-Dietz syndrome 1 in a subject.

Also provided herein are modified cells, e.g., immune cells engineered to express a protein comprising a dominant negative isoform of TGF-BR2, wherein the dominant negative isoform of TGF-BR2 competes with a wild-type isoform of TGF-BR2 for binding a TGF-β signal that prevents the activation of an immune response. In some embodiments, the dominant negative isoform of TGF-BR2 is selected from the polypeptides described in Table 5.

TABLE 5
Exemplary Dominant Negative Receptors
Comprising a Dominant Negative
Isoforms of TGF-BR2 (UNIPROT P37173)

| | | | OMIM ID/ | SEQ |
|---|---|---|---|---|
| | | | Ensembl SNP/ | ID |
| UNIPROT ID | Disease Phenotype | Mutation | ClinVar VCV | NO: |
| P37173 | None | Truncation | | 548 |
| | | of ICD | | |
| | | after Q194 | | |
| P37173 | None | Truncation | | 549 |
| | | of ICD | | |
| | | after Y187 | | |
| P37173 | Loeys-Dietz | R537C | 190182.0007/ | 550 |
| | Syndrome 2 | | rs104893809/ | |
| | | | VCV000012507 | |
| P37173 | Loeys-Dietz | R528H | 190182.0011/ | 551 |
| | Syndrome 2; Colon | | rs104893815/ | |
| | Cancer, Hereditary | | VCV000012511 | |
| | Nonpolyposis, | | | |
| | Type 6, Somatic, | | | |
| | Included | | | |
| P37173 | Loeys-Dietz | R528C | 190182.0012/ | 552 |
| | Syndrome 2 | | rs104893810/ | |
| | | | VCV000012512 | |
| P37173 | Loeys-Dietz | R460C | 190182.0014/ | 553 |
| | Syndrome 2 | | rs104893811/ | |
| | | | VCV000012514 | |
| P37173 | Loeys-Dietz | R460H | 190182.0015/ | 554 |
| | Syndrome 2 | | rs104893816/ | |
| | | | VCV000012515 | |
| P37173 | Loeys-Dietz | R537H | —/ | 555 |
| | Syndrome 2 | | rs1057524810/ | |
| | | | VCV000393141 | |

An exemplary amino acid sequence of a TGF-BR2 polypeptide (also referred to herein as TGF-βR2 polypeptide) comprises or consists of the amino acid sequence of SEQ ID NO: 500. In some embodiments, the dominant negative isoform of TGF-BR2 comprises a truncation after the glutamine at position 194 of SEQ ID NO: 500. In some embodiments, the dominant negative isoform of TGF-BR2 comprises a truncation after the tyrosine at position 187 of SEQ ID NO: 500. In some embodiments, the dominant negative isoform of TGF-BR2 comprises a cysteine at position 537 of SEQ ID NO: 500. In some embodiments, the dominant negative isoform of TGF-BR2 comprises a histidine at position 528 of SEQ ID NO: 500. In some embodiments, the dominant negative isoform of TGF-BR2 comprises a cysteine at position 528 of SEQ ID NO: 500. In some embodiments, the dominant negative isoform of TGF-BR2 comprises a cysteine at position 460 of SEQ ID NO: 500. In some embodiments, the dominant negative isoform of TGF-BR2 comprises a histidine at position 460 of SEQ ID NO: 500. In some embodiments, the dominant negative isoform of TGF-BR2 comprises a histidine at position 537 of SEQ ID NO: 500.

In some embodiments, the dominant negative isoform of TGF-BR2 comprises a polypeptide having at least 90%, at least 91%, at least 92%, at least 93%, at least 94%, at least 95%, at least 96%, at least 97%, at least 98%, at least 99%, or 100% sequence identity to the amino acid sequence of SEQ ID NO: 500, wherein the polypeptide comprises: a truncation after the glutamine at the amino acid position corresponding to position 194 of SEQ ID NO: 500, a truncation after the tyrosine at the amino acid position corresponding to position 187 of SEQ ID NO: 500, a cysteine at the amino acid position corresponding to position 537 of SEQ ID NO: 500, a histidine at the amino acid position corresponding to position 528 of SEQ ID NO: 500, a cysteine at the amino acid position corresponding to position 528 of SEQ ID NO: 500, a cysteine at the amino acid position corresponding to position 460 of SEQ ID NO: 500, a histidine at the amino acid position corresponding to position 460 of SEQ ID NO: 500, or a histidine at the amino acid position corresponding to position 537 of SEQ ID NO: 500.

### Signal Inverters

The chimeric proteins of the signal inverter modality of the disclosure can be generated by combining one or more of an extracellular domain, a transmembrane domain, and an intracellular domain, disclosed herein, wherein the extracellular domain is capable of engaging a negative signal that prevents activation of an immune response, and wherein the intracellular domain comprises at least a portion of the intracellular domain of a stimulatory polypeptide that is associated with a positive signal that promotes activation of an immune response or activates an immune cell.

The following set of non-limiting parameters could allow one of skill in the art to identify and combine one or more of an extracellular domain, a transmembrane domain, and an intracellular domain disclosed herein, and thereby generate exemplary chimeric proteins of the signal inverter modality of the disclosure.

### 1) Orientation

In some embodiments, the chimeric protein comprises an N-terminal to C-terminal orientation relative to the cell surface, wherein the chimeric proteins comprise an N-terminal to C-terminal orientation throughout the protein, i.e., each of the extracellular domain, the transmembrane domain, and the intracellular domain comprise the same N-terminal to C-terminal orientation. Type I receptors comprise an extracellular N-terminus and an intracellular C-terminus, and they are anchored to the plasma membrane with a stop-transfer anchor sequence. Type II receptors comprise an intracellular N-terminus and an extracellular C-terminus. Type III receptors comprise the same orientation as type I receptors of an extracellular N-terminus and an intracellular C-terminus. However, unlike type I receptors, type III receptors are anchored with a signal-anchor sequence.

In some embodiments, the chimeric protein comprises an extracellular domain, or a portion thereof, of a type I receptor in combination with an intracellular domain, or a portion thereof, of a type I receptor. In some embodiments, the chimeric protein comprises an extracellular domain, or a portion thereof, of a type I receptor in combination with an intracellular domain, or a portion thereof, of a type III receptor. In some embodiments, the chimeric protein comprises an extracellular domain, or a portion thereof, of a type I receptor in combination with an intracellular domain, or a portion thereof, of a protein that is not associated with the plasma membrane. In some embodiments, the chimeric protein comprises an extracellular domain, or a portion thereof, of a type II receptor in combination with an intracellular domain, or a portion thereof, of a type II receptor. In some embodiments, the chimeric protein comprises an extracellular domain, or a portion thereof, of a type I receptor in combination with a transmembrane domain, or a portion thereof, of a type I receptor, and an intracellular domain, or a portion thereof, of a type II receptor.

In some embodiments, the transmembrane domain and the intracellular domain are connected by a linker. In some embodiments, the transmembrane domain and the extracellular domain are connected by a linker. In some embodiments, the transmembrane domain is connected to the extracellular domain by a first linker and is connected to the intracellular domain by a second linker. Suitable linkers include any linker disclosed herein.

### 2) Transmembrane Domains

In some embodiments, the transmembrane domain of the chimeric protein comprises or consists of a transmembrane domain, or a portion thereof, of an inhibitory polypeptide disclosed herein. In some embodiments, the transmembrane domain of the chimeric protein comprises or consists of a transmembrane domain, or a portion thereof, of a stimulatory polypeptide disclosed herein. In some embodiments, the transmembrane domain of the chimeric protein is not a transmembrane domain of an inhibitory polypeptide when the intracellular domain of the chimeric protein is derived from a stimulatory polypeptide having a transmembrane domain associated with a positive signal that promotes activation of an immune response or activates an immune cell. An association, or lack of association, of the transmembrane domain of exemplary stimulatory polypeptides with a positive signal that promotes activation of an immune response or activates an immune cell is described in Table 2.

### 3) Isoforms

In some embodiments, the chimeric protein comprises an intracellular domain comprising the intracellular domain, or a portion thereof, of isoform 1 (e.g., as identified by a canonical UNIPROT ID) of a stimulatory polypeptide listed in Table 2. In some embodiments, the intracellular domain comprises the intracellular domain, or a portion thereof, of one or more isoforms of a stimulatory polypeptide listed in Table 2.

4) scFv-Based Extracellular Domains

In some embodiments, the extracellular domain of a chimeric protein comprises an antigen-binding domain that specifically binds to the negative signal that prevents activation of an immune response. In embodiments, the antigen-binding domain comprises an scFv, a Fab, or a VHH. The scFv may be anchored to the plasma membrane of an immune cell by being connected to the transmembrane domain. In some embodiments, the scFv is multivalent. In some embodiments, the scFv and the transmembrane domain are connected by a linker. In embodiments, where the extracellular domain of the chimeric protein comprises an antigen-binding domain, the transmembrane domain is preferably a transmembrane domain of a stimulatory polypeptide, or a portion thereof, disclosed herein.

### 5) Combination of Extracellular Domain and Intracellular Domain

The combination of the extracellular domain and intracellular domain in a chimeric protein may be selected based on the similarities of their structural properties (e.g., capability of oligomerization) and/or functional properties (e.g., compatibility to induce a signal transduction pathway). The compatibility of the combination of the extracellular domain and intracellular domain can be determined, for example, by screening for the associated positive signal in a cell, for example, NK cell. In some embodiments, the chimeric protein comprises an extracellular domain, or a portion thereof, of an inhibitory polypeptide that is capable of forming a dimer in combination with an intracellular domain, or a portion thereof, of a stimulatory polypeptide that is capable of forming a dimer. In some embodiments, the chimeric protein comprises an extracellular domain, or a portion thereof, of an inhibitory polypeptide that is capable of forming a trimer in combination with an intracellular domain, or a portion thereof, of a stimulatory polypeptide that is capable of forming a trimer. In some embodiments, the chimeric protein comprises an extracellular domain, or a portion thereof, of an inhibitory polypeptide that is capable of inducing signal transduction in an immune cell (e.g., NK cell), in combination with an intracellular domain, or a portion thereof, of a stimulatory polypeptide. In some embodiments, the chimeric protein does not comprise an extracellular domain, or a portion thereof, of an inhibitory polypeptide that is not capable of forming an oligomer in combination with an intracellular domain, or a portion thereof, of a stimulatory polypeptide that is capable of forming an oligomer.

In one aspect, the disclosure is directed to chimeric proteins comprising an extracellular domain, a transmembrane domain, and an intracellular domain, wherein the extracellular domain engages TGF-β, and wherein the intracellular domain comprises at least a portion of the intracellular domain of a stimulatory polypeptide that is associated with a positive signal that activates an immune cell. In some embodiments, the extracellular domain comprises at least a portion of the extracellular domain of a TGF-β receptor polypeptide.

Tables 6 and 7 show exemplary chimeric protein constructs that are capable of binding a TGF-B signal, and domains thereof.

In some embodiments, the chimeric protein comprises an extracellular domain capable of binding TGF-β, a transmembrane domain, and an intracellular domain, wherein the intracellular domain comprises the intracellular domain, or a portion thereof, of a stimulatory polypeptide that is associated with a positive signal that activates an immune cell. In some embodiments, the stimulatory polypeptide is CD226, natural cytotoxicity triggering receptor 1, CD160, hematopoietic cell signal transducer, TYRO protein tyrosine kinase-binding protein, MyD88, granulocyte colony-stimulating factor receptor, macrophage colony-stimulating factor 1 receptor, erythropoietin receptor, inducible T-cell costimulator, T-cell-specific surface glycoprotein CD28, transmembrane and immunoglobulin domain-containing protein 2, tumor necrosis factor receptor superfamily member 9, tumor necrosis factor receptor superfamily member 25, tumor necrosis factor receptor superfamily member 4, low affinity immunoglobulin gamma Fc region receptor III-A, low affinity immunoglobulin gamma Fc region receptor II-c, high affinity immunoglobulin epsilon receptor subunit gamma, T-cell surface antigen CD2, natural killer cell receptor 2B4, SLAM family member 7, T-cell surface glycoprotein CD3 epsilon chain, T-cell surface glycoprotein CD3 gamma chain, T-cell surface glycoprotein CD3 zeta chain, carcinoembryonic antigen-related cell adhesion molecule 3, macrophage mannose receptor 1, intercellular adhesion molecule 1, intercellular adhesion molecule 2, intercellular adhesion molecule 3, interleukin-1 receptor-associated kinase 1, interleukin-1 receptor-associated kinase-like 2, interleukin-1 receptor-associated kinase 4, B-cell receptor CD22, sialic acid-binding Ig-like lectin 14, sialic acid-binding Ig-like lectin 15, hepatitis A virus cellular receptor 1, toll-like receptor 3, toll-like receptor 4, toll-like receptor 9, tyrosine-protein kinase SYK, proto-oncogene tyrosine-protein kinase Src, tyrosine-protein kinase ZAP-70, killer cell lectin-like receptor subfamily F member 2, killer cell lectin-like receptor subfamily F member 1, NKG2-D type II integral membrane protein, C-type lectin domain family 7 member A, tumor necrosis factor ligand superfamily member 9, tumor necrosis factor ligand superfamily member 14, or tumor necrosis factor ligand superfamily member 13B.

In some embodiments, the chimeric protein comprises an extracellular domain of a TGF-BR1 polypeptide, a transmembrane domain, and an intracellular domain, wherein the intracellular domain comprises the intracellular domain, or a portion thereof, of a stimulatory polypeptide that is associated with a positive signal that activates an immune cell. In some embodiments, the stimulatory polypeptide is CD226, natural cytotoxicity triggering receptor 1, CD160, hematopoietic cell signal transducer, TYRO protein tyrosine kinase-binding protein, myeloid differentiation primary response protein MyD88, granulocyte colony-stimulating factor receptor, macrophage colony-stimulating factor I receptor, erythropoietin receptor, inducible T-cell costimulator, T-cell-specific surface glycoprotein CD28, transmembrane and immunoglobulin domain-containing protein 2, tumor necrosis factor receptor superfamily member 9, tumor necrosis factor receptor superfamily member 25, tumor necrosis factor receptor superfamily member 4, low affinity immunoglobulin gamma Fc region receptor III-A, low affinity immunoglobulin gamma Fc region receptor II-c, high affinity immunoglobulin epsilon receptor subunit gamma, T-cell surface antigen CD2, natural killer cell receptor 2B4, SLAM family member 7, T-cell surface glycoprotein CD3 epsilon chain, T-cell surface glycoprotein CD3 gamma chain, T-cell surface glycoprotein CD3 zeta chain, carcinoembryonic antigen-related cell adhesion molecule 3, macrophage mannose receptor 1, intercellular adhesion molecule 1, intercellular adhesion molecule 2, intercellular adhesion molecule 3, interleukin-1 receptor-associated kinase 1, interleukin-1 receptor-associated kinase-like 2, interleukin-1 receptor-associated kinase 4, B-cell receptor CD22, sialic acid-binding Ig-like lectin 14, sialic acid-binding Ig-like lectin 15, hepatitis A virus cellular receptor 1, toll-like receptor 3, toll-like receptor 4, toll-like receptor 9, tyrosine-protein kinase SYK, proto-oncogene tyrosine-protein kinase Src, tyrosine-protein kinase ZAP-70, killer cell lectin-like receptor subfamily F member 2, killer cell lectin-like receptor subfamily F member 1, NKG2-D type II integral membrane protein, C-type lectin domain family 7 member A, tumor necrosis factor ligand superfamily member 9, tumor necrosis factor ligand superfamily member 14, or tumor necrosis factor ligand superfamily member 13B.

In some embodiments, the chimeric protein comprises an extracellular domain of a TGF-β R2 polypeptide, a transmembrane domain, and an intracellular domain, wherein the intracellular domain comprises the intracellular domain, or a portion thereof, of a stimulatory polypeptide that is associated with a positive signal that activates an immune cell. In some embodiments, the stimulatory polypeptide is CD226, natural cytotoxicity triggering receptor 1, CD160, hematopoietic cell signal transducer, TYRO protein tyrosine kinase-binding protein, myeloid differentiation primary response protein MyD88, granulocyte colony-stimulating factor receptor, macrophage colony-stimulating factor 1 receptor, erythropoietin receptor, inducible T-cell costimulator, T-cell-specific surface glycoprotein CD28, transmembrane and immunoglobulin domain-containing protein 2, tumor necrosis factor receptor superfamily member 9, tumor necrosis factor receptor superfamily member 25, tumor necrosis factor receptor superfamily member 4, low affinity immunoglobulin gamma Fc region receptor III-A, I low affinity immunoglobulin gamma Fc region receptor II-c, high affinity immunoglobulin epsilon receptor subunit gamma, T-cell surface antigen CD2, natural killer cell receptor 2B4, SLAM family member 7, T-cell surface glycoprotein CD3 epsilon chain, T-cell surface glycoprotein CD3 gamma chain, T-cell surface glycoprotein CD3 zeta chain, carcinoembryonic antigen-related cell adhesion molecule 3, macrophage mannose receptor 1, intercellular adhesion molecule 1, intercellular adhesion molecule 2, intercellular adhesion molecule 3, interleukin-1 receptor-associated kinase 1, interleukin-1 receptor-associated kinase-like 2, interleukin-1 receptor-associated kinase 4, B-cell receptor CD22, sialic acid-binding Ig-like lectin 14, sialic acid-binding Ig-like lectin 15, hepatitis A virus cellular receptor 1, toll-like receptor 3, toll-like receptor 4, toll-like receptor 9, tyrosine-protein kinase SYK, proto-oncogene tyrosine-protein kinase Src, tyrosine-protein kinase ZAP-70, killer cell lectin-like receptor subfamily F member 2, killer cell lectin-like receptor subfamily F member 1, NKG2-D type II integral membrane protein, C-type lectin domain family 7 member A, tumor necrosis factor ligand superfamily member 9, tumor necrosis factor ligand superfamily member 14, or tumor necrosis factor ligand superfamily member 13B.

In some embodiments, the chimeric protein comprises an extracellular domain comprising an antigen-binding domain that specifically binds TGF-β, a transmembrane domain, and an intracellular domain, wherein the intracellular domain comprises the intracellular domain, or a portion thereof, of a stimulatory polypeptide that is associated with a positive signal that activates an immune cell. In some embodiments, the antigen-binding domain comprises an scFv. In some embodiments, the stimulatory polypeptide is CD226, natural cytotoxicity triggering receptor 1, CD160, hematopoietic cell signal transducer, TYRO protein tyrosine kinase-binding protein, I myeloid differentiation primary response protein MyD88, granulocyte colony-stimulating factor receptor, macrophage colony-stimulating factor 1 receptor, erythropoietin receptor, inducible T-cell costimulator, T-cell-specific surface glycoprotein CD28, transmembrane and immunoglobulin domain-containing protein 2, tumor necrosis factor receptor superfamily member 9, tumor necrosis factor receptor superfamily member 25, or tumor necrosis factor receptor superfamily member 4.

In some embodiments, the chimeric protein comprises an extracellular domain of a TGF-βR1 polypeptide and a TGF-βR2 polypeptide, a transmembrane domain, and an intracellular domain, wherein the intracellular domain comprises the intracellular domain, or a portion thereof, of a stimulatory polypeptide that is associated with a positive signal that activates an immune cell. In some embodiments, the chimeric protein comprises the intracellular domain, or a portion thereof, of two or more different stimulatory polypeptides. In some embodiments, the two or more different stimulatory polypeptides comprise: tyrosine-protein kinase Lck and t-cell surface glycoprotein CD3 zeta chain, T-cell surface glycoprotein CD3 zeta chain and Tyrosine-protein kinase ZAP-70, tyrosine-protein kinase ZAP-70 and linker for activation of T-cells family member 1, tyrosine-protein kinase ZAP-70 and lymphocyte cytosolic protein 2, myeloid differentiation primary response protein MyD88 and interleukin-1 receptor-associated kinase 4, interleukin-1 receptor-associated kinase 4 and interleukin-1 receptor-associated kinase 1, interleukin-1 receptor-associated kinase 4 and interleukin-1 receptor-associated kinase-like 2, interleukin-1 receptor-associated kinase 1 and TNF receptor-associated factor 6, interleukin-1 receptor-associated kinase-like 2 and TNF receptor-associated factor 6, I interleukin-3 receptor subunit alpha and cytokine receptor common subunit beta, interleukin-2 receptor subunit beta and cytokine receptor common subunit gamma, interleukin-21 receptor and cytokine receptor common subunit gamma, interleukin-7 receptor subunit alpha and cytokine receptor common subunit gamma, interleukin-7 receptor subunit alpha and cytokine receptor-like factor 2, interleukin-12 receptor subunit beta-1 and interleukin-12 receptor subunit beta-2, or interleukin-18 receptor 1 and interleukin-18 receptor accessory protein.

TABLE 6
Exemplary Chimeric Protein Constructs that Bind TGF-B and Domains Thereof

| | ECD | | ICD | | | |
|---|---|---|---|---|---|---|
| | UNIPROT | | UNIPROT | ICD | | |
| ECD^{a} | ID | ICD^{b} | ID | Isoform(s) | Class | Group |
| TGF-BR1 | P36897 | CD226 antigen | Q15762 | 1 | Ig family | NK activating |
| | | | | | receptor | receptors |
| TGF-BR1 | P36897 | Natural cytotoxicity | O76036 | 1 | Ig family | NK activating |
| | | triggering receptor 1 | | | receptor | receptors |
| TGF-BR1 | P36897 | CD160 antigen | O95971 | 3 | Ig family | NK activating |
| | | | | | receptor | receptors |
| TGF-BR1 | P36897 | Hematopoietic cell | Q9UBK5 | 1 | Adaptor | Signaling |
| | | signal transducer | | | | adaptors |
| TGF-BR1 | P36897 | TYRO protein | O43914 | 1 | Adaptor | Signaling |
| | | tyrosine kinase- | | | | adaptors |
| | | binding protein | | | | |
| TGF-BR1 | P36897 | Myeloid | Q99836 | 1, 2, 4, 6, 8 | Adaptor | Signaling |
| | | differentiation | | | | adaptors |
| | | primary response | | | | |
| | | protein MyD88 | | | | |
| TGF-BR1 | P36897 | Granulocyte colony- | Q99062 | 1, 2, 3, 4 | Cytokine | Homodimerizing |
| | | stimulating factor | | | receptor | cytokines and |
| | | receptor | | | | growth factors |
| TGF-BR1 | P36897 | Macrophage colony- | P07333 | 1 | Cytokine | Homodimerizing |
| | | stimulating factor 1 | | | receptor | cytokines and |
| | | receptor | | | | growth factors |
| TGF-BR1 | P36897 | Erythropoietin | P19235 | 1 | Growth | Homodimerizing |
| | | receptor | | | factor | cytokines and |
| | | | | | receptor | growth factors |
| TGF-BR1 | P36897 | Inducible T-cell | Q9Y6W8 | 1 | Ig family | TCR |
| | | costimulator | | | receptor | costimulatory |
| | | | | | | receptors |
| TGF-BR1 | P36897 | T-cell-specific | P10747 | 1 | Ig family | TCR |
| | | surface glycoprotein | | | receptor | costimulatory |
| | | CD28 | | | | receptors |
| TGF-BR1 | P36897 | Transmembrane and | Q96BF3 | 1, 2 | Ig family | TCR |
| | | immunoglobulin | | | receptor | costimulatory |
| | | domain-containing | | | | receptors |
| | | protein 2 | | | | |
| TGF-BR1 | P36897 | Tumor necrosis | Q07011 | 1 | TNF | Tumor Necrosis |
| | | factor receptor | | | family | Family receptors |
| | | superfamily | | | receptor | |
| | | member 9 | | | | |
| TGF-BR1 | P36897 | Tumor necrosis | Q93038 | 1 | TNF | Tumor Necrosis |
| | | factor receptor | | | family | Family receptors |
| | | superfamily | | | receptor | |
| | | member 25 | | | | |
| TGF-BR1 | P36897 | Tumor necrosis | P43489 | 1 | TNF | Tumor Necrosis |
| | | factor receptor | | | family | Family receptors |
| | | superfamily | | | receptor | |
| | | member 4 | | | | |
| TGF-BR1 | P36897 | Low affinity | P08637 | 1 | Antibody | Antibody |
| | | immunoglobulin | | | receptor | receptors |
| | | gamma Fc region | | | | |
| | | receptor III-A | | | | |
| TGF-BR1 | P36897 | Low affinity | P31995 | 1, 2, 3, 4, 5 | Antibody | Antibody |
| | | immunoglobulin | | | receptor | receptors |
| | | gamma Fc region | | | | |
| | | receptor II-c | | | | |
| TGF-BR1 | P36897 | High affinity | P30273 | 1 | Antibody | Antibody |
| | | immunoglobulin | | | receptor | receptors |
| | | epsilon receptor | | | | |
| | | subunit gamma | | | | |
| TGF-BR1 | P36897 | T-cell surface | P06729 | 1 | CD2 | CD2 family |
| | | antigen CD2 | | | family | receptors |
| | | | | | receptor | |
| TGF-BR1 | P36897 | Natural killer cell | Q9BZW8 | 1, 3 | CD2 | CD2 family |
| | | receptor 2B4 | | | family | receptors |
| | | | | | receptor | |
| TGF-BR1 | P36897 | SLAM family | Q9NQ25 | 1, 3, 5 | CD2 | CD2 family |
| | | member 7 | | | family | receptors |
| | | | | | receptor | |
| TGF-BR1 | P36897 | T-cell surface | P07766 | 1 | CD3 | CD3 family |
| | | glycoprotein CD3 | | | Chain | receptors |
| | | epsilon chain | | | | |
| TGF-BR1 | P36897 | T-cell surface | P09693 | 1 | CD3 | CD3 family |
| | | glycoprotein CD3 | | | Chain | receptors |
| | | gamma chain | | | | |
| TGF-BR1 | P36897 | T-cell surface | P20963 | 1 | CD3 | CD3 family |
| | | glycoprotein CD3 | | | Chain | receptors |
| | | zeta chain | | | | |
| TGF-BR1 | P36897 | Carcinoembryonic | P40198 | 1, 2, 3 | CEACAM | Unique |
| | | antigen-related cell | | | family | |
| | | adhesion molecule 3 | | | | |
| TGF-BR1 | P36897 | Macrophage | P22897 | 1 | C-type | Unique |
| | | mannose receptor 1 | | | lectin | |
| | | | | | family | |
| | | | | | receptor | |
| TGF-BR1 | P36897 | Intercellular | P05362 | 1 | Ig family | Intercellular |
| | | adhesion molecule 1 | | | receptor | adhesion receptors |
| TGF-BR1 | P36897 | Intercellular | P13598 | 1 | Ig family | Intercellular |
| | | adhesion molecule 2 | | | receptor | adhesion receptors |
| TGF-BR1 | P36897 | Intercellular | P32942 | 1 | Ig family | Intercellular |
| | | adhesion molecule 3 | | | receptor | adhesion receptors |
| TGF-BR1 | P36897 | Interleukin-1 | P51617 | 1 | Serine/ | Innate immune |
| | | receptor-associated | | | threonine- | signaling |
| | | kinase 1 | | | protein | serine/threonine |
| | | | | | kinase | protein kinase |
| TGF-BR1 | P36897 | Interleukin-1 | O43187 | 1 | Serine/ | Innate immune |
| | | receptor-associated | | | threonine- | signaling |
| | | kinase-like 2 | | | protein | serine/threonine |
| | | | | | kinase | protein kinase |
| TGF-BR1 | P36897 | Interleukin-1 | Q9NWZ3 | 1, 2 | Serine/ | Innate immune |
| | | receptor-associated | | | threonine- | signaling |
| | | kinase 4 | | | protein | serine/threonine |
| | | | | | kinase | protein kinase |
| TGF-BR1 | P36897 | B-cell receptor | P20273 | 1, 4 | Siglec | Siglec family |
| | | CD22 | | | lectin | activating |
| | | | | | family | receptors |
| | | | | | receptor | |
| TGF-BR1 | P36897 | Sialic acid-binding | Q08ET2 | 1 | Siglec | Siglec family |
| | | Ig-like lectin 14 | | | lectin | activating |
| | | | | | family | receptors |
| | | | | | receptor | |
| TGF-BR1 | P36897 | Sialic acid-binding | Q6ZMC9 | 1 | Siglec | Siglec family |
| | | Ig-like lectin 15 | | | lectin | activating |
| | | | | | family | receptors |
| | | | | | receptor | |
| TGF-BR1 | P36897 | Hepatitis A virus | Q96D42 | 1 | TIM | Unique |
| | | cellular receptor 1 | | | receptor | |
| | | | | | family | |
| TGF-BR1 | P36897 | Toll-like receptor 3 | O15455 | 1 | TLR | Toll-like family |
| | | | | | family | receptors |
| TGF-BR1 | P36897 | Toll-like receptor 4 | O00206 | 1 | TLR | Toll-like family |
| | | | | | family | receptors |
| TGF-BR1 | P36897 | Toll-like receptor 9 | Q9NR96 | 1 | TLR | Toll-like family |
| | | | | | family | receptors |
| TGF-BR1 | P36897 | Tyrosine-protein | P43405 | 1, 2 | Tyrosine- | Immune signaling |
| | | kinase SYK | | | protein | tyrosine protein |
| | | | | | kinase | kinase |
| TGF-BR1 | P36897 | Proto-oncogene | P12931 | 1, 2 | Tyrosine- | Immune signaling |
| | | tyrosine-protein | | | protein | tyrosine protein |
| | | kinase Src | | | kinase | kinase |
| TGF-BR1 | P36897 | Tyrosine-protein | P43403 | 1, 2, 3 | Tyrosine- | Immune signaling |
| | | kinase ZAP-70 | | | protein | tyrosine protein |
| | | | | | kinase | kinase |
| TGF-BR1 | P36897 | Killer cell lectin-like | D3W0D1 | 1 | C-type | C-type lectin |
| | | receptor subfamily F | | | lectin | family receptor |
| | | member 2 | | | family | |
| | | | | | receptor | |
| TGF-BR1 | P36897 | Killer cell lectin-like | Q9NZS2 | 1 | C-type | C-type lectin |
| | | receptor subfamily F | | | lectin | family receptor |
| | | member 1 | | | family | |
| | | | | | receptor | |
| TGF-BR1 | P36897 | NKG2-D type II | P26718 | 1 | C-type | C-type lectin |
| | | integral membrane | | | lectin | family receptor |
| | | protein | | | family | |
| | | | | | receptor | |
| TGF-BR1 | P36897 | C-type lectin | Q9BXN2 | 1 | C-type | C-type lectin |
| | | domain family 7 | | | lectin | family receptor |
| | | member A | | | family | |
| | | | | | receptor | |
| TGF-BR1 | P36897 | Tumor necrosis | P41273 | 1 | TNF | TNF Family |
| | | factor ligand | | | Family | Ligand |
| | | superfamily | | | Ligand | |
| | | member 9 | | | | |
| TGF-BR1 | P36897 | Tumor necrosis | 043557 | 1 | TNF | TNF Family |
| | | factor ligand | | | Family | Ligand |
| | | superfamily | | | Ligand | |
| | | member 14 | | | | |
| TGF-BR1 | P36897 | Tumor necrosis | Q9Y275 | 1, 2, 3 | TNF | TNF Family |
| | | factor ligand | | | Family | Ligand |
| | | superfamily | | | Ligand | |
| | | member 13B | | | | |
| TGF-BR2 | P37173 | CD226 antigen | Q15762 | 1 | Ig family | NK activating |
| | | | | | receptor | receptors |
| TGF-BR2 | P37173 | Natural cytotoxicity | O76036 | 1 | Ig family | NK activating |
| | | triggering receptor 1 | | | receptor | receptors |
| TGF-BR2 | P37173 | CD160 antigen | O95971 | 3 | Ig family | NK activating |
| | | | | | receptor | receptors |
| TGF-BR2 | P37173 | Hematopoietic cell | Q9UBK5 | 1 | Adaptor | Signaling |
| | | signal transducer | | | | adaptors |
| TGF-BR2 | P37173 | TYRO protein | O43914 | 1 | Adaptor | Signaling |
| | | tyrosine kinase- | | | | adaptors |
| | | binding protein | | | | |
| TGF-BR2 | P37173 | Myeloid | Q99836 | 1, 2, 4, 6, 8 | Adaptor | Signaling |
| | | differentiation | | | | adaptors |
| | | primary response | | | | |
| | | protein MyD88 | | | | |
| TGF-BR2 | P37173 | Granulocyte colony- | Q99062 | 1, 2, 3, 4 | Cytokine | Homodimerizing |
| | | stimulating factor | | | receptor | cytokines and |
| | | receptor | | | | growth factors |
| TGF-BR2 | P37173 | Macrophage colony- | P07333 | 1 | Cytokine | Homodimerizing |
| | | stimulating factor 1 | | | receptor | cytokines and |
| | | receptor | | | | growth factors |
| TGF-BR2 | P37173 | Erythropoietin | P19235 | 1 | Growth | Homodimerizing |
| | | receptor | | | factor | cytokines and |
| | | | | | receptor | growth factors |
| TGF-BR2 | P37173 | Inducible T-cell | Q9Y6W8 | 1 | Ig family | TCR |
| | | costimulator | | | receptor | costimulatory |
| | | | | | | receptors |
| TGF-BR2 | P37173 | T-cell-specific | P10747 | 1 | Ig family | TCR |
| | | surface glycoprotein | | | receptor | costimulatory |
| | | CD28 | | | | receptors |
| TGF-BR2 | P37173 | Transmembrane and | Q96BF3 | 1, 2 | Ig family | TCR |
| | | immunoglobulin | | | receptor | costimulatory |
| | | domain-containing | | | | receptors |
| | | protein 2 | | | | |
| TGF-BR2 | P37173 | Tumor necrosis | Q07011 | 1 | TNF | Tumor Necrosis |
| | | factor receptor | | | family | Family receptors |
| | | superfamily | | | receptor | |
| | | member 9 | | | | |
| TGF-BR2 | P37173 | Tumor necrosis | Q93038 | 1 | TNF | Tumor Necrosis |
| | | factor receptor | | | family | Family receptors |
| | | superfamily | | | receptor | |
| | | member 25 | | | | |
| TGF-BR2 | P37173 | Tumor necrosis | P43489 | 1 | TNF | Tumor Necrosis |
| | | factor receptor | | | family | Family receptors |
| | | superfamily | | | receptor | |
| | | member 4 | | | | |
| TGF-BR2 | P37173 | Low affinity | P08637 | 1 | Antibody | Antibody |
| | | immunoglobulin | | | receptor | receptors |
| | | gamma Fc region | | | | |
| | | receptor III-A | | | | |
| TGF-BR2 | P37173 | Low affinity | P31995 | 1, 2, 3, 4, 5 | Antibody | Antibody |
| | | immunoglobulin | | | receptor | receptors |
| | | gamma Fc region | | | | |
| | | receptor II-c | | | | |
| TGF-BR2 | P37173 | High affinity | P30273 | 1 | Antibody | Antibody |
| | | immunoglobulin | | | receptor | receptors |
| | | epsilon receptor | | | | |
| | | subunit gamma | | | | |
| TGF-BR2 | P37173 | T-cell surface | P06729 | 1 | CD2 | CD2 family |
| | | antigen CD2 | | | family | receptors |
| | | | | | receptor | |
| TGF-BR2 | P37173 | Natural killer cell | Q9BZW8 | 1, 3 | CD2 | CD2 family |
| | | receptor 2B4 | | | family | receptors |
| | | | | | receptor | |
| TGF-BR2 | P37173 | SLAM family | Q9NQ25 | 1, 3, 5 | CD2 | CD2 family |
| | | member 7 | | | family | receptors |
| | | | | | receptor | |
| TGF-BR2 | P37173 | T-cell surface | P07766 | 1 | CD3 | CD3 family |
| | | glycoprotein CD3 | | | Chain | receptors |
| | | epsilon chain | | | | |
| TGF-BR2 | P37173 | T-cell surface | P09693 | 1 | CD3 | CD3 family |
| | | glycoprotein CD3 | | | Chain | receptors |
| | | gamma chain | | | | |
| TGF-BR2 | P37173 | T-cell surface | P20963 | 1 | CD3 | CD3 family |
| | | glycoprotein CD3 | | | Chain | receptors |
| | | zeta chain | | | | |
| TGF-BR2 | P37173 | Carcinoembryonic | P40198 | 1, 2, 3 | CEACAM | Unique |
| | | antigen-related cell | | | family | |
| | | adhesion molecule 3 | | | | |
| TGF-BR2 | P37173 | Macrophage | P22897 | 1 | C-type | Unique |
| | | mannose receptor 1 | | | lectin | |
| | | | | | family | |
| | | | | | receptor | |
| TGF-BR2 | P37173 | Intercellular | P05362 | 1 | Ig family | Intercellular |
| | | adhesion molecule 1 | | | receptor | adhesion receptors |
| TGF-BR2 | P37173 | Intercellular | P13598 | 1 | Ig family | Intercellular |
| | | adhesion molecule 2 | | | receptor | adhesion receptors |
| TGF-BR2 | P37173 | Intercellular | P32942 | 1 | Ig family | Intercellular |
| | | adhesion molecule 3 | | | receptor | adhesion receptors |
| TGF-BR2 | P37173 | Interleukin-1 | P51617 | 1 | Serine/ | Innate immune |
| | | receptor-associated | | | threonine- | signaling |
| | | kinase 1 | | | protein | serine/threonine |
| | | | | | kinase | protein kinase |
| TGF-BR2 | P37173 | Interleukin-1 | O43187 | 1 | Serine/ | Innate immune |
| | | receptor-associated | | | threonine- | signaling |
| | | kinase-like 2 | | | protein | serine/threonine |
| | | | | | kinase | protein kinase |
| TGF-BR2 | P37173 | Interleukin-1 | Q9NWZ3 | 1, 2 | Serine/ | Innate immune |
| | | receptor-associated | | | threonine- | signaling |
| | | kinase 4 | | | protein | serine/threonine |
| | | | | | kinase | protein kinase |
| TGF-BR2 | P37173 | B-cell receptor | P20273 | 1, 4 | Siglec | Siglec family |
| | | CD22 | | | lectin | activating |
| | | | | | family | receptors |
| | | | | | receptor | |
| TGF-BR2 | P37173 | Sialic acid-binding | Q08ET2 | 1 | Siglec | Siglec family |
| | | Ig-like lectin 14 | | | lectin | activating |
| | | | | | family | receptors |
| | | | | | receptor | |
| TGF-BR2 | P37173 | Sialic acid-binding | Q6ZMC9 | 1 | Siglec | Siglec family |
| | | Ig-like lectin 15 | | | lectin | activating |
| | | | | | family | receptors |
| | | | | | receptor | |
| TGF-BR2 | P37173 | Hepatitis A virus | Q96D42 | 1 | TIM | Unique |
| | | cellular receptor 1 | | | receptor | |
| | | | | | family | |
| TGF-BR2 | P37173 | Toll-like receptor 3 | O15455 | 1 | TLR | Toll-like family |
| | | | | | family | receptors |
| TGF-BR2 | P37173 | Toll-like receptor 4 | O00206 | 1 | TLR | Toll-like family |
| | | | | | family | receptors |
| TGF-BR2 | P37173 | Toll-like receptor 9 | Q9NR96 | 1 | TLR | Toll-like family |
| | | | | | family | receptors |
| TGF-BR2 | P37173 | Tyrosine-protein | P43405 | 1, 2 | Tyrosine- | Immune signaling |
| | | kinase SYK | | | protein | tyrosine protein |
| | | | | | kinase | kinase |
| TGF-BR2 | P37173 | Proto-oncogene | P12931 | 1, 2 | Tyrosine- | Immune signaling |
| | | tyrosine-protein | | | protein | tyrosine protein |
| | | kinase Src | | | kinase | kinase |
| TGF-BR2 | P37173 | Tyrosine-protein | P43403 | 1, 2, 3 | Tyrosine- | Immune signaling |
| | | kinase ZAP-70 | | | protein | tyrosine protein |
| | | | | | kinase | kinase |
| TGF-BR2 | P37173 | Killer cell lectin-like | D3W0D1 | 1 | C-type | C-type lectin |
| | | receptor subfamily F | | | lectin | family receptor |
| | | member 2 | | | family | |
| | | | | | receptor | |
| TGF-BR2 | P37173 | Killer cell lectin-like | Q9NZS2 | 1 | C-type | C-type lectin |
| | | receptor subfamily F | | | lectin | family receptor |
| | | member 1 | | | family | |
| | | | | | receptor | |
| TGF-BR2 | P37173 | NKG2-D type II | P26718 | 1 | C-type | C-type lectin |
| | | integral membrane | | | lectin | family receptor |
| | | protein | | | family | |
| | | | | | receptor | |
| TGF-BR2 | P37173 | C-type lectin | Q9BXN2 | 1 | C-type | C-type lectin |
| | | domain family 7 | | | lectin | family receptor |
| | | member A | | | family | |
| | | | | | receptor | |
| TGF-BR2 | P37173 | Tumor necrosis | P41273 | 1 | TNF | TNF Family |
| | | factor ligand | | | Family | Ligand |
| | | superfamily | | | Ligand | |
| | | member 9 | | | | |
| TGF-BR2 | P37173 | Tumor necrosis | O43557 | 1 | TNF | TNF Family |
| | | factor ligand | | | Family | Ligand |
| | | superfamily | | | Ligand | |
| | | member 14 | | | | |
| TGF-BR2 | P37173 | Tumor necrosis | Q9Y275 | 1, 2, 3 | TNF | TNF Family |
| | | factor ligand | | | Family | Ligand |
| | | superfamily | | | Ligand | |
| | | member 13B | | | | |
| ScFv | | CD226 antigen | Q15762 | 1 | Ig family | NK activating |
| specific | | | | | receptor | receptors |
| for | | | | | | |
| TGF-B1 | | | | | | |
| ScFv | | Natural | O76036 | 1 | Ig family | NK activating |
| specific | | cytotoxicity | | | receptor | receptors |
| for | | triggering | | | | |
| TGF-B1 | | receptor 1 | | | | |
| ScFv | | CD160 antigen | O95971 | 3 | Ig family | NK activating |
| specific | | | | | receptor | receptors |
| for | | | | | | |
| TGF-B1 | | | | | | |
| ScFv | | Hematopoietic cell | Q9UBK5 | 1 | Adaptor | Signaling |
| specific | | signal transducer | | | | adaptors |
| for | | | | | | |
| TGF-B1 | | | | | | |
| ScFv | | TYRO protein | O43914 | 1 | Adaptor | Signaling |
| specific | | tyrosine kinase- | | | adaptors | |
| for | | binding protein | | | | |
| TGF-B1 | | | | | | |
| ScFv | | Myeloid | Q99836 | 1, 2, 4, 6, 8 | Adaptor | Signaling |
| specific | | differentiation | | | | adaptors |
| for | | primary response | | | | |
| TGF-B1 | | protein MyD88 | | | | |
| ScFv | | Granulocyte colony- | Q99062 | 1, 2, 3, 4 | Cytokine | Homodimerizing |
| specific | | stimulating factor | | | receptor | cytokines and |
| for | | receptor | | | | growth factors |
| TGF-B1 | | | | | | |
| ScFv | | Macrophage colony- | P07333 | 1 | Cytokine | Homodimerizing |
| specific | | stimulating factor 1 | | | receptor | cytokines and |
| for | | receptor | | | | growth factors |
| TGF-B1 | | | | | | |
| ScFv | | Erythropoietin | P19235 | 1 | Growth | Homodimerizing |
| specific | | receptor | | | factor | cytokines and |
| for | | | | | receptor | growth factors |
| TGF-B1 | | | | | | |
| ScFv | | Inducible T-cell | Q9Y6W8 | 1 | Ig family | TCR |
| specific | | costimulator | | | receptor | costimulatory |
| for | | | | | | receptors |
| TGF-B1 | | | | | | |
| ScFv | | T-cell-specific | P10747 | 1 | Ig family | TCR |
| specific | | surface glycoprotein | | | receptor | costimulatory |
| for | | CD28 | | | | receptors |
| TGF-B1 | | | | | | |
| ScFv | | Transmembrane and | Q96BF3 | 1, 2 | Ig family | TCR |
| specific | | immunoglobulin | | | receptor | costimulatory |
| for | | domain-containing | | | | receptors |
| TGF-B1 | | protein 2 | | | | |
| ScFv | | Tumor necrosis | Q07011 | 1 | TNF | Tumor Necrosis |
| specific | | factor receptor | | | family | Family receptors |
| for | | superfamily | | | receptor | |
| TGF-B1 | | member 9 | | | | |
| ScFv | | Tumor necrosis | Q93038 | 1 | TNF | Tumor Necrosis |
| specific | | factor receptor | | | family | Family receptors |
| for | | superfamily | | | receptor | |
| TGF-B1 | | member 25 | | | | |
| ScFv | | Tumor necrosis | P43489 | 1 | TNF | Tumor Necrosis |
| specific | | factor receptor | | | family | Family receptors |
| for | | superfamily | | | receptor | |
| TGF-B1 | | member 4 | | | | |

^{a}The extracellular domain (ECD) refers to the ECD of an inhibitory polypeptide, or a portion thereof (e.g., TGF-BR1, or TGF-BR2), or an scFv (e.g., scFv specific for TGF-B).
^{b}The intracellular domain (ICD) refers to the ICD of a stimulatory polypeptide, or a portion thereof (e.g., CD226 antigen).

TABLE 7
Exemplary Chimeric Protein Constructs that Bind TGF-B and Domains Thereof

| | | | UNIPROT | | UNIPROT | |
|---|---|---|---|---|---|---|
| | | | ID- | ICD^{b}- | ID- | |
| | ECD | ICD^{b}- | stimulatory | stimulatory | stimulatory | |
| | UNIPROT | stimulatory | polypeptide | polypeptide | polypeptide | |
| ECD^{a} | ID | polypeptide 1 | 1 | 2 | 2 | Class |
| TGF-BR1 | P36897 | Tyrosine-protein | P06239 | T-cell | P20963 | TCR signaling |
| and | and | kinase Lck | | surface | | pathway |
| TGF-BR2 | P37173 | | | glycoprotein | | |
| | | | | CD3 zeta | | |
| | | | | chain | | |
| TGF-BR1 | P36897 | T-cell surface | P20963 | Tyrosine- | P43403 | TCR signaling |
| and | and | glycoprotein CD3 | | protein | | pathway |
| TGF-BR2 | P37173 | zeta chain | | kinase ZAP- | | |
| | | | | 70 | | |
| TGF-BR1 | P36897 | Tyrosine-protein | P43403 | Linker for | O43561 | TCR signaling |
| and | and | kinase ZAP-70 | | activation of | | pathway |
| TGF-BR2 | P37173 | | | T-cells | | |
| | | | | family | | |
| | | | | member 1 | | |
| TGF-BR1 | P36897 | Tyrosine-protein | P43403 | Lymphocyte | Q13094 | TCR signaling |
| and | and | kinase ZAP-70 | | cytosolic | | pathway |
| TGF-BR2 | P37173 | | | protein 2 | | |
| TGF-BR1 | P36897 | Myeloid | Q99836 | Interleukin- | Q9NWZ3 | MyD88 |
| and | and | differentiation | | 1 receptor- | | signaling |
| TGF-BR2 | P37173 | primary response | | associated | | pathway |
| | | protein MyD88 | | kinase 4 | | |
| TGF-BR1 | P36897 | Interleukin-1 | Q9NWZ3 | Interleukin- | P51617 | MyD88 |
| and | and | receptor- | | 1 receptor- | | signaling |
| TGF-BR2 | P37173 | associated kinase | | associated | | pathway |
| | | 4 | | kinase 1 | | |
| TGF-BR1 | P36897 | Interleukin-1 | Q9NWZ3 | Interleukin- | O43187 | MyD88 |
| and | and | receptor- | | 1 receptor- | | signaling |
| TGF-BR2 | P37173 | associated kinase | | associated | | pathway |
| | | 4 | | kinase-like | | |
| | | | | 2 | | |
| TGF-BR1 | P36897 | Interleukin-1 | P51617 | TNF | Q9Y4K3 | MyD88 |
| and | and | receptor- | | receptor- | | signaling |
| TGF-BR2 | P37173 | associated kinase | | associated | | pathway |
| | | 1 | | factor 6 | | |
| TGF-BR1 | P36897 | Interleukin-1 | O43187 | TNF | Q9Y4K3 | MyD88 |
| and TGF- | and | receptor- | | receptor- | | signaling |
| BR2 | P37173 | associated kinase- | | associated | | pathway |
| | | like 2 | | factor 6 | | |
| TGF-BR1 | P36897 | Interleukin-3 | P26951 | Cytokine | P32927 | Heterodimeric |
| and | and | receptor subunit | | receptor | | cytokine |
| TGF-BR2 | P37173 | alpha | | common | | signaling |
| | | | | subunit beta | | |
| TGF-BR1 | P36897 | Interleukin-2 | P14784 | Cytokine | P31785 | Heterodimeric |
| and | and | receptor subunit | | receptor | | cytokine |
| TGF-BR2 | P37173 | beta | | common | | signaling |
| | | | | subunit | | |
| | | | | gamma | | |
| TGF-BR1 | P36897 | Interleukin-21 | Q9HBE5 | Cytokine | P31785 | Heterodimeric |
| and | and | receptor | | receptor | | cytokine |
| TGF-BR2 | P37173 | | | common | | signaling |
| | | | | subunit | | |
| | | | | gamma | | |
| TGF-BR1 | P36897 | Interleukin-7 | P16871 | Cytokine | P31785 | Heterodimeric |
| and | and | receptor subunit | | receptor | | cytokine |
| TGF-BR2 | P37173 | alpha | | common | | signaling |
| | | | | subunit | | |
| | | | | gamma | | |
| TGF-BR1 | P36897 | Interleukin-7 | P16871 | Cytokine | Q9HC73 | Heterodimeric |
| and | and | receptor subunit | | receptor- | | cytokine |
| TGF-BR2 | P37173 | alpha | | like factor 2 | | signaling |
| TGF-BR1 | P36897 | Interleukin-12 | P42701 | Interleukin- | Q99665 | Heterodimeric |
| and | and | receptor subunit | | 12 receptor | | cytokine |
| TGF-BR2 | P37173 | beta-1 | | subunit | | signaling |
| | | | | beta-2 | | |
| TGF-BR1 | P36897 | Interleukin-18 | Q13478 | Interleukin- | O95256 | Heterodimeric |
| and | and | receptor 1 | | 18 receptor | | cytokine |
| TGF-BR2 | P37173 | | | accessory | | signaling |
| | | | | protein | | |

^{a}The extracellular domain (ECD) refers to the ECD of one or more inhibitory polypeptides, or a portion thereof (e.g., TGF-BR1 and TGF-BR2), or an ScFv (e.g., ScFv specific for TGF-B).
^{b}The intracellular domain (ICD) refers to the ICD of a stimulatory polypeptide, or a portion thereof (e.g., IL-18R).

In some embodiments, the chimeric protein comprises an extracellular domain of a TGF-BR2 polypeptide, a transmembrane domain, and an intracellular domain, wherein the intracellular domain comprises the intracellular domain, or a portion thereof, of a IL-21R polypeptide. In some embodiments, the chimeric protein comprises an extracellular domain of a TGF-BR2 polypeptide (e.g., SEQ ID NO: 39), a transmembrane domain of a TGF-BR2 polypeptide, and an intracellular domain a IL-21R polypeptide, and optionally a signal peptide of a TGF-BR2 polypeptide (e.g., SEQ ID NO: 496). In some embodiments, the chimeric protein comprises an extracellular domain of a TGF-BR2 polypeptide (e.g., SEQ ID NO: 39), a transmembrane domain of a TGF-BR2 polypeptide (e.g., SEQ ID NO: 257), and an intracellular domain of a IL-21R polypeptide (e.g., SEQ ID NO: 95). In some embodiments, the chimeric protein comprises an amino acid sequence that is at least 80%, at least 85%, at least 90%, at least 91%, at least 92%, at least 93%, at least 94%, at least 95%, at least 96%, at least 97%, at least 98%, at least 99% or at least 100% identical to the amino acid sequence of SEQ ID NOs: 680 or 681.

In some embodiments, the chimeric protein comprises an extracellular domain of a TGF-BR2 polypeptide, a transmembrane domain, and an intracellular domain, wherein the intracellular domain comprises the intracellular domain, or a portion thereof, of a IL-2RG polypeptide. In some embodiments, the chimeric protein comprises an extracellular domain of a TGF-BR2 polypeptide (e.g., SEQ ID NO: 39), a transmembrane domain of a IL-2RG polypeptide, and an intracellular domain a IL-2RG polypeptide, and optionally a signal peptide of a CD8α polypeptide (e.g., SEQ ID NO: 676). In some embodiments, the chimeric protein comprises an extracellular domain of a TGF-BR2 polypeptide (e.g., SEQ ID NO: 39), a transmembrane domain of a IL-2RG polypeptide (e.g., SEQ ID NO: 289), and an intracellular domain of a IL-2RG polypeptide (e.g., SEQ ID NO: 73). In some embodiments, the chimeric protein comprises an amino acid sequence that is at least 80%, at least 85%, at least 90%, at least 91%, at least 92%, at least 93%, at least 94%, at least 95%, at least 96%, at least 97%, at least 98%, at least 99% or at least 100% identical to the amino acid sequence of SEQ ID NOs: 682 or 683.

In some embodiments, the chimeric protein comprises an extracellular domain of a TGF-BR 1 polypeptide, a transmembrane domain, and an intracellular domain, wherein the intracellular domain comprises the intracellular domain, or a portion thereof, of a IL-2RG polypeptide. In some embodiments, the chimeric protein comprises an extracellular domain of a TGF-BR1 polypeptide (e.g., SEQ ID NO: 38), a transmembrane domain of a IL-2RG polypeptide, and an intracellular domain a IL-2RG polypeptide, and optionally a signal peptide of a CD8α polypeptide (e.g., SEQ ID NO: 676). In some embodiments, the chimeric protein comprises an extracellular domain of a TGF-BR1 polypeptide (e.g., SEQ ID NO: 38), a transmembrane domain of a IL-2RG polypeptide (e.g., SEQ ID NO: 289), and an intracellular domain of a IL-2RG polypeptide (e.g., SEQ ID NO: 73). In some embodiments, the chimeric protein comprises an amino acid sequence that is at least 80%, at least 85%, at least 90%, at least 91%, at least 92%, at least 93%, at least 94%, at least 95%, at least 96%, at least 97%, at least 98%, at least 99% or at least 100% identical to the amino acid sequence of SEQ ID NOs: 684 or 685.

In some embodiments, the chimeric protein comprises an extracellular domain of a TGF-BR1 polypeptide, a transmembrane domain, and an intracellular domain, wherein the intracellular domain comprises the intracellular domain, or a portion thereof, of a IL-21R polypeptide. In some embodiments, the chimeric protein comprises an extracellular domain of a TGF-BR1 polypeptide (e.g., SEQ ID NO: 38), a transmembrane domain of a IL-21R polypeptide, and an intracellular domain a IL-21R polypeptide, and optionally a signal peptide of a TGFBR1 polypeptide (e.g., SEQ ID NO: 495). In some embodiments, the chimeric protein comprises an extracellular domain of a TGF-BR1 polypeptide (e.g., SEQ ID NO: 38), a transmembrane domain of a IL-21R polypeptide (e.g., SEQ ID NO: 256), and an intracellular domain of a IL-21R polypeptide (e.g., SEQ ID NO: 95). In some embodiments, the chimeric protein comprises an amino acid sequence that is at least 80%, at least 85%, at least 90%, at least 91%, at least 92%, at least 93%, at least 94%, at least 95%, at least 96%, at least 97%, at least 98%, at least 99% or at least 100% identical to the amino acid sequence of SEQ ID NOs: 686 or 687.

In some embodiments, a cell provided herein (e.g., an immune cell, e.g., an NK cell) comprises (e.g., is engineered to express) a first chimeric protein and a second chimeric protein, wherein:
(a) the first chimeric protein comprises an extracellular domain of a TGF-BR2 polypeptide, a transmembrane domain (e.g., a transmembrane domain of a TGF-BR2 polypeptide), and an intracellular domain of a IL-21R polypeptide (e.g., comprises an amino acid sequence that is at least 80%, at least 85%, at least 90%, at least 95%, at least 96%, at least 97%, at least 98%, at least 99% or at least 100% identical to the amino acid sequence of SEQ ID NOs: 680 or 681), and the second polypeptide comprises an extracellular domain of a TGF-BR2 polypeptide, a transmembrane domain (e.g., a transmembrane domain of a IL-2RG polypeptide), and an intracellular domain of a IL-2RG polypeptide (e.g., comprises an amino acid sequence that is at least 80%, at least 85%, at least 90%, at least 95%, at least 96%, at least 97%, at least 98%, at least 99% or at least 100% identical to the amino acid sequence of SEQ ID NOs: 682 or 683);
(b) the first chimeric protein comprises an extracellular domain of a TGF-BR2 polypeptide, a transmembrane domain (e.g., a transmembrane domain of a TGF-BR2 polypeptide), and an intracellular domain of a IL-21R polypeptide (e.g., comprises an amino acid sequence that is at least 80%, at least 85%, at least 90%, at least 95%, at least 96%, at least 97%, at least 98%, at least 99% or at least 100% identical to the amino acid sequence of SEQ ID NOs: 680 or 681), and the second polypeptide comprises an extracellular domain of a TGF-BR1 polypeptide, a transmembrane domain (e.g., a transmembrane domain of a IL-2RG polypeptide), and an intracellular domain of a IL-2RG polypeptide (e.g., comprises an amino acid sequence that is at least 80%, at least 85%, at least 90%, at least 95%, at least 96%, at least 97%, at least 98%, at least 99% or at least 100% identical to the amino acid sequence of SEQ ID NOs: 684 or 685);
(c) the first chimeric protein comprises an extracellular domain of a TGF-BR2 polypeptide, a transmembrane domain (e.g., a transmembrane domain of a IL-2RG polypeptide), and an intracellular domain of a IL-2RG polypeptide (e.g., comprises an amino acid sequence that is at least 80%, at least 85%, at least 90%, at least 95%, at least 96%, at least 97%, at least 98%, at least 99% or at least 100% identical to the amino acid sequence of SEQ ID NOs: 682 or 683), and the second polypeptide comprises an extracellular domain of a TGF-BR1 polypeptide, a transmembrane domain (e.g., a transmembrane domain of a IL-21R polypeptide), and an intracellular domain a IL-21R polypeptide (e.g., comprises an amino acid sequence that is at least 80%, at least 85%, at least 90%, at least 95%, at least 96%, at least 97%, at least 98%, at least 99% or at least 100% identical to the amino acid sequence of SEQ ID NOs: 686 or 687); or
(d) the first chimeric protein comprises an extracellular domain of a TGF-BR1 polypeptide, a transmembrane domain (e.g., a transmembrane domain of a IL-21R polypeptide), and an intracellular domain a IL-21R polypeptide (e.g., comprises an amino acid sequence that is at least 80%, at least 85%, at least 90%, at least 95%, at least 96%, at least 97%, at least 98%, at least 99% or at least 100% identical to the amino acid sequence of SEQ ID NOs: 686 or 687), and the second chimeric protein comprises an extracellular domain of a TGF-BR1 polypeptide, a transmembrane domain (e.g., a transmembrane domain of a IL-2RG polypeptide), and an intracellular domain of a IL-2RG polypeptide (e.g., comprises an amino acid sequence that is at least 80%, at least 85%, at least 90%, at least 95%, at least 96%, at least 97%, at least 98%, at least 99% or at least 100% identical to the amino acid sequence of SEQ ID NOs: 684 or 685). Table 8 indicates exemplary chimeric protein constructs that are capable of engaging an IL-10 signal, and domains thereof.

TABLE 8
Exemplary Chimeric Protein Constructs that Bind IL-10 and Domains Thereof

| | ECD | | ICD | | | |
|---|---|---|---|---|---|---|
| | UNIPROT | | UNIPROT | ICD | | |
| ECD^{a} | ID | ICD^{b} | ID | isoform(s) | Class | Group |
| IL-10RA | Q13651 | CD226 antigen | Q15762 | 1 | Ig family | NK activating |
| | | | | | receptor | receptors |
| IL-10RA | Q13651 | Natural cytotoxicity | O76036 | 1 | | Ig family |
| | | triggering receptor 1 | | | | receptor |
| IL-10RA | Q13651 | CD160 antigen | O95971 | 3 | Ig family | NK activating |
| | | | | | receptor | receptors |
| IL-10RA | Q13651 | Hematopoietic cell | Q9UBK5 | 1 | Adaptor | Signaling |
| | | signal transducer | | | | adaptors |
| IL-10RA | Q13651 | TYRO protein tyrosine | O43914 | 1 | Adaptor | Signaling |
| | | kinase-binding protein | | | | adaptors |
| IL-10RA | Q13651 | Myeloid differentiation | Q99836 | 1, 2, 4, 6, 8 | Adaptor | Signaling |
| | | primary response | | | | adaptors |
| | | protein MyD88 | | | | |
| IL-10RA | Q13651 | Granulocyte colony- | Q99062 | 1, 2, 3, 4 | Cytokine | Homodimerizing |
| | | stimulating factor | | | receptor | cytokines and |
| | | receptor | | | | growth factors |
| IL-10RA | Q13651 | Macrophage colony- | P07333 | 1 | Cytokine | Homodimerizing |
| | | stimulating factor 1 | | | receptor | cytokines and |
| | | receptor | | | | growth factors |
| IL-10RA | Q13651 | Erythropoietin receptor | P19235 | 1 | Growth | Homodimerizing |
| | | | | | factor | cytokines and |
| | | | | | receptor | growth factors |
| IL-10RA | Q13651 | Inducible T-cell | Q9Y6W8 | 1 | Ig family | TCR |
| | | costimulator | | | receptor | costimulatory |
| | | | | | | receptors |
| IL-10RA | Q13651 | T-cell-specific surface | P10747 | 1 | Ig family | TCR |
| | | glycoprotein CD28 | | | receptor | costimulatory |
| | | | | | | receptors |
| IL-10RA | Q13651 | Transmembmne and | Q96BF3 | 1, 2 | Ig family | TCR |
| | | immunoglobulin | | | receptor | costimulatory |
| | | domain-containing | | | | receptors |
| | | protein 2 | | | | |
| IL-10RA | Q13651 | Tumor necrosis factor | Q07011 | 1 | TNF | Tumor Necrosis |
| | | receptor superfamily | | | family | Family receptors |
| | | member 9 | | | receptor | |
| IL-10RA | Q13651 | Tumor necrosis factor | Q93038 | 1 | TNF | Tumor Necrosis |
| | | receptor superfamily | | | family | Family receptors |
| | | member 25 | | | receptor | |
| IL-10RA | Q13651 | Tumor necrosis factor | P43489 | 1 | TNF | Tumor Necrosis |
| | | receptor superfamily | | | family | Family receptors |
| | | member 4 | | | receptor | |
| IL-10RA | Q13651 | Low affinity | P08637 | 1 | Antibody | Antibody |
| | | immunoglobulin | | | receptor | receptors |
| | | gamma Fc region | | | | |
| | | receptor III-A | | | | |
| IL-10RA | Q13651 | Low affinity | P31995 | 1, 2, 3, 4, 5 | Antibody | Antibody |
| | | immunoglobulin | | | receptor | receptors |
| | | gamma Fc region | | | | |
| | | receptor II-c | | | | |
| IL-10RA | Q13651 | High affinity | P30273 | 1 | Antibody | Antibody |
| | | immunoglobulin | | | receptor | receptors |
| | | epsilon receptor | | | | |
| | | subunit gamma | | | | |
| IL-10RA | Q13651 | T-cell surface antigen | P06729 | 1 | CD2 | CD2 family |
| | | CD2 | | | family | receptors |
| | | | | | receptor | |
| IL-10RA | Q13651 | Natural killer cell | Q9BZW8 | 1, 3 | CD2 | CD2 family |
| | | receptor 2B4 | | | family | receptors |
| | | | | | receptor | |
| IL-10RA | Q13651 | SLAM family member | Q9NQ25 | 1, 3, 5 | CD2 | CD2 family |
| | | 7 | | | family | receptors |
| | | | | | receptor | |

^{a}The extracellular domain (ECD) refers to the ECD of an inhibitory polypeptide, or a portion thereof (e.g., IL-10RA), or an ScFv (e.g., ScFv specific for IL-10).
^{b}The intracellular domain (ICD) refers to the ICD of a stimulatory polypeptide, or a portion thereof (e.g., CD226 antigen).

In some embodiments, the chimeric protein comprises an extracellular domain capable of binding IL-10, a transmembrane domain, and an intracellular domain, wherein the intracellular domain comprises the intracellular domain, or a portion thereof, of a stimulatory polypeptide that is associated with a positive signal that activates an immune cell. In some embodiments, the stimulatory polypeptide is CD226, natural cytotoxicity triggering receptor 1, CD160, hematopoietic cell signal transducer, TYRO protein tyrosine kinase-binding protein, myeloid differentiation primary response protein MyD88, granulocyte colony-stimulating factor receptor, macrophage colony-stimulating factor 1 receptor, erythropoietin receptor, inducible T-cell costimulator, T-cell-specific surface glycoprotein CD28, transmembrane and immunoglobulin domain-containing protein 2, tumor necrosis factor receptor superfamily member 9, tumor necrosis factor receptor superfamily member 25, tumor necrosis factor receptor superfamily member 4, low affinity immunoglobulin gamma Fc region receptor III-A, low affinity immunoglobulin gamma Fc region receptor II-c, high affinity immunoglobulin epsilon receptor subunit gamma, T-cell surface antigen CD2, natural killer cell receptor 2B4, or SLAM family member 7.

In some embodiments, the chimeric protein comprises an extracellular domain of an IL-10RA polypeptide, a transmembrane domain, and an intracellular domain, wherein the intracellular domain comprises the intracellular domain, or a portion thereof, of a stimulatory polypeptide that is associated with a positive signal that activates an immune cell. In some embodiments, the stimulatory polypeptide is CD226, natural cytotoxicity triggering receptor 1, CD160, hematopoietic cell signal transducer, TYRO protein tyrosine kinase-binding protein, myeloid differentiation primary response protein MyD88, granulocyte colony-stimulating factor receptor, macrophage colony-stimulating factor 1 receptor, erythropoietin receptor, inducible T-cell costimulator, T-cell-specific surface glycoprotein CD28, transmembrane and immunoglobulin domain-containing protein 2, tumor necrosis factor receptor superfamily member 9, tumor necrosis factor receptor superfamily member 25, tumor necrosis factor receptor superfamily member 4, low affinity immunoglobulin gamma Fc region receptor III-A, low affinity immunoglobulin gamma Fc region receptor II-c, high affinity immunoglobulin epsilon receptor subunit gamma, T-cell surface antigen CD2, natural killer cell receptor 2B4, or SLAM family member 7.

Table 9 indicates exemplary chimeric protein constructs that are capable of engaging an HLA signal, and domains thereof. In some embodiments, the extracellular domain of the chimeric protein constructs of Table 9 comprises the extracellular domain of an inhibitory KIR provided herein (e.g., e.g., KIR2DL1, KIR2DL2, KIR2DL3, KIR2DL5A, KIR2DL55, or KIR3DL1).

TABLE 9
Exemplary Chimeric Protein Constructs that Bind HLA and Domains Thereof

| Extracellular | Intracellular | ICD UNIPROT | ICD | | |
|---|---|---|---|---|---|
| domain (ECD)^{a} | domain (ICD)^{b} | ID | isoform(s) | Class | Group |
| Inhibitory KIR | CD226 antigen | Q15762 | 1 | Ig family | NK activating |
| | | | | receptor | receptors |
| Inhibitory KIR | Natural cytotoxicity | O76036 | 1 | Ig family | NK activating |
| | triggering receptor 1 | | | receptor | receptors |
| Inhibitory KIR | CD160 antigen | O95971 | 3 | Ig family | NK activating |
| | | | | receptor | receptors |
| Inhibitory KIR | Hematopoietic cell | Q9UBK5 | 1 | Adaptor | Signaling |
| | signal transducer | | | | adaptors |
| Inhibitory KIR | TYRO protein | O43914 | 1 | Adaptor | Signaling |
| | tyrosine kinase- | | | | adaptors |
| | binding protein | | | | |
| Inhibitory KIR | Myeloid | Q99836 | 1, 2, 4, 6, 8 | Adaptor | Signaling |
| | differentiation | | | | adaptors |
| | primary response | | | | |
| | protein MyD88 | | | | |
| Inhibitory KIR | Granulocyte colony- | Q99062 | 1, 2, 3, 4 | Cytokine | Homodimerizing |
| | stimulating factor | | | receptor | cytokines and |
| | receptor | | | | growth factors |
| Inhibitory KIR | Macrophage colony- | P07333 | 1 | Cytokine | Homodimerizing |
| | stimulating factor 1 | | | receptor | cytokines and |
| | receptor | | | | growth factors |
| Inhibitory KIR | Erythropoietin | P19235 | 1 | Growth | Homodimerizing |
| | receptor | | | factor | cytokines and |
| | | | | receptor | growth factors |
| Inhibitory KIR | Inducible T-cell | Q9Y6W8 | 1 | Ig family | TCR |
| | costimulator | | | receptor | costimulatory |
| | | | | | receptors |
| Inhibitory KIR | T-cell-specific | P10747 | 1 | Ig family | TCR |
| | surface glycoprotein | | | receptor | costimulatory |
| | CD28 | | | | receptors |
| Inhibitory KIR | Transmembrane and | Q96BF3 | 1, 2 | Ig family | TCR |
| | immunoglobulin | | | receptor | costimulatory |
| | domain-containing | | | | receptors |
| | protein 2 | | | | |
| Inhibitory KIR | Tumor necrosis | Q07011 | 1 | TNF | Tumor Necrosis |
| | factor receptor | | | family | Family receptors |
| | superfamily member | | | receptor | |
| | 9 | | | | |
| Inhibitory KIR | Tumor necrosis | Q93038 | 1 | TNF | Tumor Necrosis |
| | factor receptor | | | family | Family receptors |
| | superfamily member | | | receptor | |
| | 25 | | | | |
| Inhibitory KIR | Tumor necrosis | P43489 | 1 | TNF | Tumor Necrosis |
| | factor receptor | | | family | Family receptors |
| | superfamily member | | | receptor | |
| | 4 | | | | |
| Inhibitory KIR | Killer cell | Q99706 | 1 | Ig family | KIR activating |
| | immunoglobulin-like | | | receptor | receptor |
| | receptor 2DL4 | | | | |
| Inhibitory KIR | Killer cell | Q14954 | 1 | Ig family | KIR activating |
| | immunoglobulin-like | | | receptor | receptor |
| | receptor 2DS1 | | | | |
| Inhibitory KIR | Killer cell | P43631 | 1 | Ig family | KIR activating |
| | immunoglobulin-like | | | receptor | receptor |
| | receptor 2DS2 | | | | |
| Inhibitory KIR | Killer cell | Q14952 | 1 | Ig family | KIR activating |
| | immunoglobulin-like | | | receptor | receptor |
| | receptor 2DS3 | | | | |
| Inhibitory KIR | Killer cell | P43632 | 1 | Ig family | KIR activating |
| | immunoglobulin-like | | | receptor | receptor |
| | receptor 2DS4 | | | | |
| Inhibitory KIR | Killer cell | Q14953 | 1 | Ig family | KIR activating |
| | immunoglobulin-like | | | receptor | receptor |
| | receptor 2DS5 | | | | |
| Inhibitory KIR | Killer cell | Q14943 | 1 | Ig family | KIR activating |
| | immunoglobulin-like | | | receptor | receptor |
| | receptor 3DS1 | | | | |

^{a}The extracellular domain (ECD) refers to the ECD of an inhibitory polypeptide, or a portion thereof (e.g., inhibitory KIR), or an ScFv (e.g., ScFv specific for HLA).
^{b}The intracellular domain (ICD) refers to the ICD of a stimulatory polypeptide, or a portion thereof (e.g., CD226 antigen).

In some embodiments, the chimeric protein comprises an extracellular domain capable of binding HLA, a transmembrane domain, and an intracellular domain, wherein the intracellular domain comprises the intracellular domain, or a portion thereof, of a stimulatory polypeptide that is associated with a positive signal that activates an immune cell. In some embodiments, the stimulatory polypeptide is CD226, natural cytotoxicity triggering receptor 1, CD160, hematopoietic cell signal transducer, TYRO protein tyrosine kinase-binding protein, myeloid differentiation primary response protein MyD88, granulocyte colony-stimulating factor receptor, I macrophage colony-stimulating factor 1 receptor, erythropoietin receptor, inducible T-cell costimulator, T-cell-specific surface glycoprotein CD28, transmembrane and immunoglobulin domain-containing protein 2, tumor necrosis factor receptor superfamily member 9, tumor necrosis factor receptor superfamily member 25, tumor necrosis factor receptor superfamily member 4, killer cell immunoglobulin-like receptor 2DL4, killer cell immunoglobulin-like receptor 2DS1, killer cell immunoglobulin-like receptor 2DS2, killer cell immunoglobulin-like receptor 2DS3, killer cell immunoglobulin-like receptor 2DS4, killer cell immunoglobulin-like receptor 2DS5, killer cell immunoglobulin-like receptor 3DS1, or paired immunoglobulin-like type 2 receptor beta (PILRB).

In some embodiments, the chimeric protein comprises an extracellular domain of an inhibitory KIR polypeptide, a transmembrane domain, and an intracellular domain, wherein the intracellular domain comprises the intracellular domain, or a portion thereof, of a stimulatory polypeptide that is associated with a positive signal that activates an immune cell. In some embodiments, the stimulatory polypeptide is CD226, natural cytotoxicity triggering receptor 1, CD160, hematopoietic cell signal transducer, TYRO protein tyrosine kinase-binding protein, myeloid differentiation primary response protein MyD88, granulocyte colony-stimulating factor receptor, macrophage colony-stimulating factor 1 receptor, erythropoietin receptor, inducible T-cell costimulator, T-cell-specific surface glycoprotein CD28, transmembrane and immunoglobulin domain-containing protein 2, tumor necrosis factor receptor superfamily member 9, tumor necrosis factor receptor superfamily member 25, tumor necrosis factor receptor superfamily member 4, killer cell immunoglobulin-like receptor 2DL4, killer cell immunoglobulin-like receptor 2DS1, killer cell immunoglobulin-like receptor 2DS2, killer cell immunoglobulin-like receptor 2DS3, killer cell immunoglobulin-like receptor 2DS4, killer cell immunoglobulin-like receptor 2DS5, or killer cell immunoglobulin-like receptor 3DS1, or paired immunoglobulin-like type 2 receptor beta (PILRB).

In some embodiments, the chimeric protein comprises an extracellular domain of LILRB2 (e.g., SEQ ID NO: 28), a transmembrane domain, and an intracellular domain, wherein the intracellular domain comprises the intracellular domain, or a portion thereof, of a stimulatory polypeptide that is associated with a positive signal that activates an immune cell. In some embodiments, the stimulatory polypeptide is paired immunoglobulin-like type 2 receptor beta (PILRB). In some embodiments, the chimeric protein comprises an extracellular domain of LILRB2 (e.g., SEQ ID NO: 28), a transmembrane domain of PILRB (e.g., SEQ ID NO: 534), and an intracellular domain of PILRB (e.g., SEQ ID NO: 535).

Table 10 indicates exemplary chimeric protein constructs that are capable of engaging a CD155 and/or CD112 signal, and domains thereof.

TABLE 10
Exemplary Chimeric Protein Constructs that Bind CD155 and/or CD112 and
Domains Thereof

| | ECD | | ICD | ICD | | |
|---|---|---|---|---|---|---|
| | UNIPROT | | UNIPROT | iso- | | |
| ECD^{a} | ID | ICD^{b} | ID | form(s) | Class | Group |
| TIGIT | Q495A1 | CD226 antigen | Q15762 | 1 | Ig family | NK activating |
| | | | | | receptor | receptors |
| TIGIT | Q495A1 | Natural cytotoxicity | O76036 | 1 | Ig family | NK activating |
| | | triggering receptor 1 | | | receptor | receptors |
| TIGIT | Q495A1 | CD160 antigen | O95971 | 3 | Ig family | NK activating |
| | | | | | receptor | receptors |
| TIGIT | Q495A1 | Hematopoietic cell signal | Q9UBK5 | 1 | Adaptor | Signaling adaptors |
| | | transducer | | | | |
| TIGIT | Q495A1 | TYRO protein tyrosine | O43914 | 1 | Adaptor | Signaling adaptors |
| | | kinase-binding protein | | | | |
| TIGIT | Q495A1 | Myeloid differentiation | Q99836 | 1, 2, 4, | Adaptor | Signaling adaptors |
| | | primary response protein | | 6, 8 | | |
| | | MyD88 | | | | |
| TIGIT | Q495A1 | Granulocyte colony- | Q99062 | 1, 2, 3, | Cytokine | Homodimerizing |
| | | stimulating factor | | 4 | receptor | cytokines and |
| | | receptor | | | | growth factors |
| TIGIT | Q495A1 | Macrophage colony- | P07333 | 1 | Cytokine | Homodimerizing |
| | | stimulating factor 1 | | | receptor | cytokines and |
| | | receptor | | | | growth factors |
| TIGIT | Q495A1 | Erythropoietin receptor | P19235 | 1 | Growth | Homodimerizing |
| | | | | | factor | cytokines and |
| | | | | | receptor | growth factors |
| TIGIT | Q495A1 | Inducible T-cell | Q9Y6W8 | 1 | Ig family | TCR |
| | | costimulator | | | receptor | costimulatory |
| | | | | | | receptors |
| TIGIT | Q495A1 | T-cell-specific surface | P10747 | 1 | Ig family | TCR |
| | | glycoprotein CD28 | | | receptor | costimulatory |
| | | | | | | receptors |
| TIGIT | Q495A1 | Transmembrane and | Q96BF3 | 1, 2 | Ig family | TCR |
| | | immunoglobulin domain- | | | receptor | costimulatory |
| | | containing protein 2 | | | | receptors |
| TIGIT | Q495A1 | Tumor necrosis factor | Q07011 | 1 | TNF | Tumor Necrosis |
| | | receptor superfamily | | | family | Family receptors |
| | | member 9 | | | receptor | |
| TIGIT | Q495A1 | Tumor necrosis factor | Q93038 | 1 | TNF | Tumor Necrosis |
| | | receptor superfamily | | | family | Family receptors |
| | | member 25 | | | receptor | |
| TIGIT | Q495A1 | Tumor necrosis factor | P43489 | 1 | TNF | Tumor Necrosis |
| | | receptor superfamily | | | family | Family receptors |
| | | member 4 | | | receptor | |
| | | domain (ECD) | | | | |

^{a}The extracellular domain (ECD) refers to the ECD of an inhibitory polypeptide, or a portion thereof (e.g., TIGIT), or an scFv (e.g., scFv specific for CD112 and/or CD155).
^{b}The intracellular domain (ICD) refers to the ICD of a stimulatory polypeptide, or a portion thereof (e.g., CD226 antigen).

In some embodiments, the chimeric protein comprises an extracellular domain capable of binding CD155 and/or CD112, a transmembrane domain, and an intracellular domain, wherein the intracellular domain comprises the intracellular domain, or a portion thereof, of a stimulatory polypeptide that is associated with a positive signal that activates an immune cell. In some embodiments, the stimulatory polypeptide is CD226, natural cytotoxicity triggering receptor 1, CD160, hematopoietic cell signal transducer, TYRO protein tyrosine kinase-binding protein. In some embodiments, the stimulatory polypeptide is myeloid differentiation primary response protein MyD88. In some embodiments, the stimulatory polypeptide is granulocyte colony-stimulating factor receptor. In some embodiments, the stimulatory polypeptide is macrophage colony-stimulating factor 1 receptor. In some embodiments, the stimulatory polypeptide is erythropoietin receptor. In some embodiments, the stimulatory polypeptide is inducible T-cell costimulator. In some embodiments, the stimulatory polypeptide is T-cell-specific surface glycoprotein CD28. In some embodiments, the stimulatory polypeptide is Transmembrane and immunoglobulin domain-containing protein 2. In some embodiments, the stimulatory polypeptide is tumor necrosis factor receptor superfamily member 9. In some embodiments, the stimulatory polypeptide is tumor necrosis factor receptor superfamily member 25. In some embodiments, the stimulatory polypeptide is tumor necrosis factor receptor superfamily member 4.

In some embodiments, the chimeric protein comprises an extracellular domain of a TIGIT polypeptide, a transmembrane domain, and an intracellular domain, wherein the intracellular domain comprises the intracellular domain, or a portion thereof, of a stimulatory polypeptide that is associated with a positive signal that activates an immune cell. In some embodiments, the stimulatory polypeptide is CD226. In some embodiments, the stimulatory polypeptide is natural cytotoxicity triggering receptor 1. In some embodiments, the stimulatory polypeptide is CD160. In some embodiments, the stimulatory polypeptide is hematopoietic cell signal transducer. In some embodiments, the stimulatory polypeptide is TYRO protein tyrosine kinase-binding protein. In some embodiments, the stimulatory polypeptide is myeloid differentiation primary response protein MyD88. In some embodiments, the stimulatory polypeptide is granulocyte colony-stimulating factor receptor. In some embodiments, the stimulatory polypeptide is macrophage colony-stimulating factor 1 receptor. In some embodiments, the stimulatory polypeptide is erythropoietin receptor. In some embodiments, the stimulatory polypeptide is inducible T-cell costimulator. In some embodiments, the stimulatory polypeptide is T-cell-specific surface glycoprotein CD28. In some embodiments, the stimulatory polypeptide is Transmembrane and immunoglobulin domain-containing protein 2. In some embodiments, the stimulatory polypeptide is tumor necrosis factor receptor superfamily member 9. In some embodiments, the stimulatory polypeptide is tumor necrosis factor receptor superfamily member 25. In some embodiments, the stimulatory polypeptide is tumor necrosis factor receptor superfamily member 4.

Table 11 indicates exemplary chimeric protein constructs that comprise an extracellular domain, or a portion thereof, of a TIM-3 polypeptide.

TABLE 11
Exemplary chimeric protein constructs that comprise an extracellular domain, or
a portion thereof, of a TIM-3 polypeptide

| | ECD | | ICD | | | |
|---|---|---|---|---|---|---|
| | UNIPROT | | UNIPROT | ICD | | |
| ECD^{a} | ID | ICD^{b} | ID | isoform(s) | Class | Group |
| TIM-3 | Q8TDQ0 | CD226 antigen | Q15762 | 1 | Ig family | NK activating |
| | | | | | receptor | receptors |
| TIM-3 | Q8TDQ0 | Natural cytotoxicity | O76036 | 1 | Ig family | NK activating |
| | | triggering receptor 1 | | | receptor | receptors |
| TIM-3 | Q8TDQ0 | CD160 antigen | O95971 | 3 | Ig family | NK activating |
| | | | | | receptor | receptors |
| TIM-3 | Q8TDQ0 | Hematopoietic cell | Q9UBK5 | 1 | Adaptor | Signaling |
| | | signal transducer | | | | adaptors |
| TIM-3 | Q8TDQ0 | TYRO protein | O43914 | 1 | Adaptor | Signaling |
| | | tyrosine kinase- | | | | adaptors |
| | | binding protein | | | | |
| TIM-3 | Q8TDQ0 | Myeloid | Q99836 | 1, 2, 4, 6, 8 | Adaptor | Signaling |
| | | differentiation | | | | adaptors |
| | | primary response | | | | |
| | | protein MyD88 | | | | |
| TIM-3 | Q8TDQ0 | Granulocyte colony- | Q99062 | 1, 2, 3, 4 | Cytokine | Homodimerizing |
| | | stimulating factor | | | receptor | cytokines and |
| | | receptor | | | | growth factors |
| TIM-3 | Q8TDQ0 | Macrophage colony- | P07333 | 1 | Cytokine | Homodimerizing |
| | | stimulating factor 1 | | | receptor | cytokines and |
| | | receptor | | | | growth factors |
| TIM-3 | Q8TDQ0 | Erythropoietin | P19235 | 1 | Growth | Homodimerizing |
| | | receptor | | | factor | cytokines and |
| | | | | | receptor | growth factors |
| TIM-3 | Q8TDQ0 | Inducible T-cell | Q9Y6W8 | 1 | Ig family | TCR |
| | | costimulator | | | receptor | costimulatory |
| | | | | | | receptors |
| TIM-3 | Q8TDQ0 | T-cell-specific | P10747 | 1 | Ig family | TCR |
| | | surface glycoprotein | | | receptor | costimulatory |
| | | CD28 | | | | receptors |
| TIM-3 | Q8TDQ0 | Transmembrane and | Q96BF3 | 1, 2 | Ig family | TCR |
| | | immunoglobulin | | | receptor | costimulatory |
| | | domain-containing | | | | receptors |
| | | protein 2 | | | | |
| TIM-3 | Q8TDQ0 | Tumor necrosis | Q07011 | 1 | TNF | Tumor Necrosis |
| | | factor receptor | | | family | Family receptors |
| | | superfamily member | | | receptor | |
| | | 9 | | | | |
| TIM-3 | Q8TDQ0 | Tumor necrosis | Q93038 | 1 | TNF | Tumor Necrosis |
| | | factor receptor | | | family | Family receptors |
| | | superfamily member | | | receptor | |
| | | 25 | | | | |
| TIM-3 | Q8TDQ0 | Tumor necrosis | P43489 | 1 | TNF | Tumor Necrosis |
| | | factor receptor | | | family | Family receptors |
| | | superfamily member | | | receptor | |
| | | 4 | | | | |

aThe extracellular domain (ECD) refers to the ECD of an inhibitory polypeptide, or a portion thereof (e.g., TIM-3).
^{b}The intracellular domain (ICD) refers to the ICD of a stimulatory polypeptide, or a portion thereof (e.g., CD226 antigen).

In some embodiments, the chimeric protein comprises an extracellular domain of a TIM-3 polypeptide, a transmembrane domain, and an intracellular domain, wherein the intracellular domain comprises the intracellular domain, or a portion thereof, of a stimulatory polypeptide that is associated with a positive signal that activates an immune cell. In some embodiments, the stimulatory polypeptide is CD226, natural cytotoxicity triggering receptor 1, CD160, hematopoietic cell signal transducer, TYRO protein tyrosine kinase-binding protein, myeloid differentiation primary response protein MyD88, granulocyte colony-stimulating factor receptor, macrophage colony-stimulating factor 1 receptor, erythropoietin receptor, inducible T-cell costimulator, T-cell-specific surface glycoprotein CD28, transmembrane and immunoglobulin domain-containing protein 2, tumor necrosis factor receptor superfamily member 9, tumor necrosis factor receptor superfamily member 25, or tumor necrosis factor receptor superfamily member 4.

Table 12 indicates exemplary chimeric protein constructs that are capable of engaging an HLA-E signal, and domains thereof.

TABLE 12
Exemplary Chimeric Protein Constructs that Bind HLA-E, and Domains Thereof

| | ECD | | ICD | ICD | | |
|---|---|---|---|---|---|---|
| | UNIPROT | | UNIPROT | iso- | | |
| ECD^{a} | ID | ICD^{b} | ID | form(s) | Class | Group |
| NKG2A | P26715 | NKG2-D type II integral | P26718 | 1 | C-type | C-Type |
| | | membrane protein | | | lectin family | Lectins |
| | | | | | receptor | |
| NKG2A | P26715 | C-type lectin domain family | Q9BXN2 | 1 | C-type | C-Type |
| | | 7 member A | | | lectin family | Lectins |
| | | | | | receptor | |
| NKG2A | P26715 | Killer cell lectin-like | Q9NZS2 | 1 | C-type | C-Type |
| | | receptor subfamily F | | | lectin family | Lectins |
| | | member 1 | | | receptor | |
| NKG2A | P26715 | Killer cell lectin-like | D3W0D1 | 1 | C-type | C-Type |
| | | receptor subfamily F | | | lectin family | Lectins |
| | | member 2 | | | receptor | |
| NKG2A | P26715 | Tumor necrosis factor ligand | O43557 | 1 | TNF Family | TNF |
| | | superfamily member 14 | | | Ligand | Ligand |
| | | | | | | Family |
| NKG2A | P26715 | Tumor necrosis factor ligand | P41273 | 1 | TNF Family | TNF |
| | | superfamily member 9 | | | Ligand | Ligand |
| | | | | | | Family |
| NKG2A | P26715 | Tumor necrosis factor ligand | Q9Y275 | 1, 2, 3 | TNF Family | TNF |
| | | superfamily member 13B | | | Ligand | Ligand |
| | | | | | | Family |

^{a}The extracellular domain (ECD) refers to the ECD of an inhibitory polypeptide, or a portion thereof (e.g., NKG2A), or an scFv (e.g., ScFv specific for HLA-E).
^{b}The intracellular domain (ICD) refers to the ICD of a stimulatory polypeptide, or a portion thereof (e.g., NKG2-D type II integral membrane protein).

In some embodiments, the chimeric protein comprises an extracellular domain capable of binding HLA-E, a transmembrane domain, and an intracellular domain, wherein the intracellular domain comprises the intracellular domain, or a portion thereof, of a stimulatory polypeptide that is associated with a positive signal that activates an immune cell. In some embodiments, the stimulatory polypeptide is NKG2-D type II integral membrane protein, C-type lectin domain family 7 member A, killer cell lectin-like receptor subfamily F member 1, killer cell lectin-like receptor subfamily F member 2, tumor necrosis factor ligand superfamily member 14, tumor necrosis factor ligand superfamily member 9, or tumor necrosis factor ligand superfamily member 13B.

In some embodiments, the chimeric protein comprises an extracellular domain of an NKG2A polypeptide, a transmembrane domain, and an intracellular domain, wherein the intracellular domain comprises the intracellular domain, or a portion thereof, of a stimulatory polypeptide that is associated with a positive signal that activates an immune cell. In some embodiments, the stimulatory polypeptide is NKG2-D type II integral membrane protein, NKG2-C type II integral membrane protein, C-type lectin domain family 7 member A, killer cell lectin-like receptor subfamily F member 1, killer cell lectin-like receptor subfamily F member 2, tumor necrosis factor ligand superfamily member 14, tumor necrosis factor ligand superfamily member 9, or tumor necrosis factor ligand superfamily member 13B.

In some embodiments, the chimeric protein comprises an extracellular domain of a NKG2A polypeptide or a portion thereof, a transmembrane domain, and an intracellular domain, wherein the intracellular domain comprises the intracellular domain, or a portion thereof, of a NKG2C polypeptide. In some embodiments, the chimeric protein comprises an extracellular domain of a NKG2A polypeptide or a portion thereof, a transmembrane domain of a NKG2C polypeptide, and an intracellular domain, or a portion thereof, of a NKG2C polypeptide. In some embodiments, the chimeric protein comprises from N-terminus to C-terminus, an intracellular domain of a NKG2C polypeptide or a portion thereof, a transmembrane domain of a NKG2C polypeptide, and an extracellular domain of a NKG2A polypeptide or a portion thereof. In some embodiments, the chimeric protein comprises from N-terminus to C-terminus, an intracellular domain of a NKG2C polypeptide or a portion thereof, a transmembrane domain of a NKG2C polypeptide, a portion of an extracellular domain of a NKG2C polypeptide, and an extracellular domain of a NKG2A polypeptide. In some embodiments, the chimeric protein comprises an extracellular domain of a NKG2A polypeptide (e.g., SEQ ID NO: 45) or a portion thereof, a transmembrane domain of a NKG2C polypeptide (e.g., SEQ ID NO: 415), and an intracellular domain of a NKG2C polypeptide (e.g., SEQ ID NO: 199) or a portion thereof. In some embodiments, the chimeric protein comprises an amino acid sequence that is at least 80%, at least 85%, at least 90%, at least 91%, at least 92%, at least 93%, at least 94%, at least 95%, at least 96%, at least 97%, at least 98%, at least 99% or at least 100% identical to the amino acid sequence of SEQ ID NO: 677.

Table 13 indicates exemplary chimeric protein constructs that are capable of engaging an N-cadherin and/or E-cadherin signal, and domains thereof.

TABLE 13
Exemplary Chimeric Protein Constructs that Bind N-Cadherin and/or E-
Cadherin, and Domains Thereof

| | ECD | | ICD | ICD | | |
|---|---|---|---|---|---|---|
| | UNIPROT | | UNIPROT | iso- | | |
| ECD^{a} | ID | ICD^{b} | ID | form(s) | Class | Group |
| KLRG1 | Q96E93 | C-type lectin | Q9BXN2 | 1 | C-type lectin | C-Type |
| | | domain family 7 | | | family | Lectins |
| | | member A | | | receptor | |
| KLRG1 | Q96E93 | Killer cell lectin- | Q9NZS2 | 1 | C-type lectin | C-Type |
| | | like receptor | | | family | Lectins |
| | | subfamily F | | | receptor | |
| | | member 1 | | | | |
| KLRG1 | Q96E93 | Killer cell lectin- | D3W0D1 | 1 | C-type lectin | C-Type |
| | | like receptor | | | family | Lectins |
| | | subfamily F | | | receptor | |
| | | member 2 | | | | |
| KLRG1 | Q96E93 | Tumor necrosis | O43557 | 1 | TNF Family | TNF Ligand |
| | | factor ligand | | | Ligand | Family |
| | | superfamily | | | | |
| | | member 14 | | | | |
| KLRG1 | Q96E93 | Tumor necrosis | P41273 | 1 | TNF Family | TNF Ligand |
| | | factor ligand | | | Ligand | Family |
| | | superfamily | | | | |
| | | member 9 | | | | |
| KLRG1 | Q96E93 | Tumor necrosis | Q9Y275 | 1, 2, 3 | TNF Family | TNF Ligand |
| | | factor ligand | | | Ligand | Family |
| | | superfamily | | | | |
| | | member 13B | | | | |

^{a}The extracellular domain (ECD) refers to the ECD of an inhibitory polypeptide, or a portion thereof (e.g., KLRG1), or an scFv (e.g., scFv specific for N-cadherin and/or E-cadherin).
^{b}The intracellular domain (ICD) refers to the ICD of a stimulatory polypeptide, or a portion thereof (e.g., C-type lectin domain family 7 member A).

In some embodiments, the chimeric protein comprises an extracellular domain capable of binding N-cadherin and/or E-cadherin, a transmembrane domain, and an intracellular domain, wherein the intracellular domain comprises the intracellular domain, or a portion thereof, of a stimulatory polypeptide that is associated with a positive signal that activates an immune cell. In some embodiments, the stimulatory polypeptide is C-type lectin domain family 7 member A, killer cell lectin-like receptor subfamily F member 1, killer cell lectin-like receptor subfamily F member 2, tumor necrosis factor ligand superfamily member 14, tumor necrosis factor ligand superfamily member 9, or tumor necrosis factor ligand superfamily member 13B.

In some embodiments, the chimeric protein comprises an extracellular domain of a KLRG1 polypeptide, a transmembrane domain, and an intracellular domain, wherein the intracellular domain comprises the intracellular domain, or a portion thereof, of a stimulatory polypeptide that is associated with a positive signal that activates an immune cell. In some embodiments, the stimulatory polypeptide is C-type lectin domain family 7 member A, killer cell lectin-like receptor subfamily F member 1, killer cell lectin-like receptor subfamily F member 2, tumor necrosis factor ligand superfamily member 14, tumor necrosis factor ligand superfamily member 9, or tumor necrosis factor ligand superfamily member 13B.

Table 14 indicates exemplary chimeric protein constructs that are capable of engaging an IL-18 signal, and domains thereof.

TABLE 14
Exemplary Chimeric Protein Constructs that Bind IL-18, and Domains Thereof

| | ECD | | ICD | ICD | | |
|---|---|---|---|---|---|---|
| | UNIPROT | | UNIPROT | iso- | | |
| ECD^{a} | ID | ICD^{b} | ID | form(s) | Class | Group |
| IL-18BP | O95998 | Interleukin-18 | Q13478 | 1 | Cytokine | Enhanced |
| isoform A | | receptor 1 | | | receptor | affinity IL-18R |
| IL-18BP | O95998 | Interleukin-18 | Q13478 | 1 | Cytokine | Enhanced |
| isoform B | | receptor 1 | | | receptor | affinity IL-18R |
| IL-18BP | O95998 | Interleukin-18 | Q13478 | 1 | Cytokine | Enhanced |
| isoform C | | receptor 1 | | | receptor | affinity IL-18R |
| IL-18BP | O95998 | Interleukin-18 | Q13478 | 1 | Cytokine | Enhanced |
| isoform D | | receptor 1 | | | receptor | affinity IL-18R |

^{a}The extracellular domain (ECD) refers to the ECD of an inhibitory polypeptide, or a portion thereof (e.g., IL-18BP), or an scFv (e.g., ScFv specific for IL-18).
^{b}The intracellular domain (ICD) refers to the ICD of a stimulatory polypeptide, or a portion thereof (e.g., IL-18R1).

In some embodiments, the chimeric protein comprises an extracellular domain capable of binding IL-18, a transmembrane domain, and an intracellular domain, wherein the intracellular domain comprises the intracellular domain, or a portion thereof, of a stimulatory polypeptide that is associated with a positive signal that activates an immune cell. In some embodiments, the stimulatory polypeptide is interleukin-18 receptor 1.

In some embodiments, the chimeric protein comprises an extracellular domain of an IL-18BP polypeptide, a transmembrane domain, and an intracellular domain, wherein the intracellular domain comprises the intracellular domain, or a portion thereof, of a stimulatory polypeptide that is associated with a positive signal that activates an immune cell. In some embodiments, the IL-18BP polypeptide is selected from any one of IL-18BP isoform A, IL-18BP isoform B, IL-18BP isoform C, and IL-18BP isoform D. In some embodiments, the stimulatory polypeptide is interleukin-18 receptor 1.

In some embodiments, the chimeric protein comprises an extracellular domain of a FAS polypeptide, a transmembrane domain, and an intracellular domain, wherein the intracellular domain comprises the intracellular domain, or a portion thereof, of a DR3 polypeptide. In some embodiments, the chimeric protein comprises an extracellular domain of a FAS polypeptide (e.g., SEQ ID NO: 11), a transmembrane domain of a DR3 polypeptide, and an intracellular domain a DR3 polypeptide, and optionally a signal peptide of a FAS polypeptide (e.g., SEQ ID NO: 469). In some embodiments, the chimeric protein comprises an extracellular domain of a Fas polypeptide (e.g., SEQ ID NO: 11), a transmembrane domain of a DR3 polypeptide (e.g., SEQ ID NO: 395), and an intracellular domain of a DR3 polypeptide (e.g., SEQ ID NO: 179). In some embodiments, the chimeric protein comprises an amino acid sequence that is at least 80%, at least 85%, at least 90%, at least 91%, at least 92%, at least 93%, at least 94%, at least 95%, at least 96%, at least 97%, at least 98%, at least 99% or at least 100% identical to the amino acid sequence of SEQ ID NOs: 678 or 679.

In some embodiments, the chimeric protein comprises a combination of an extracellular domain (ECD), an intracellular domain (ICD) and an intracellular domain (ICD) of an exemplary chimeric protein described in Table 22. In some embodiments, the chimeric protein comprises a combination of an extracellular domain (ECD), an intracellular domain (ICD) and an intracellular domain (ICD) of any one of the exemplary chimeric proteins of A1-A226 (described in Table 22). Each of the domains of the exemplary chimeric proteins, including amino acid sequences for each domain, is described in the disclosure.

TABLE 22
Exemplary Chimeric Proteins

| No. | ECD | TM | ICD |
|---|---|---|---|
| A1 | PD1 | EPOR | EPOR |
| A2 | PD1 | GCSFR | GCSFR |
| A3 | PD1 | CSF1R | CSF1R |
| A4 | PD1 | NKp46 | NKp46 |
| A5 | PD1 | DR3 | DR3 |
| A6 | PD1 | YES1 | YES1 |
| A7 | TIGIT | CD28 | 2B4 |
| A8 | TIGIT | CD28 | SLAMF1 |
| A9 | TIGIT | CD28 | SLAMF5 |
| A10 | TIGIT | CD28 | SLAMF6 |
| A11 | TIGIT | CD28 | SLAMF7 |
| A12 | TIGIT | CD28 | SLAMF3 |
| A13 | TIGIT | CD28 | EPOR |
| A14 | TIGIT | CD28 | GCSFR |
| A15 | TIGIT | CD28 | CSF1R |
| A16 | TIGIT | CD28 | NKp46 |
| A17 | TIGIT | CD28 | DR3 |
| A18 | TIGIT | CD28 | YES1 |
| A19 | TIGIT | TIGIT | 2B4 |
| A20 | TIGIT | TIGIT | SLAMF1 |
| A21 | TIGIT | TIGIT | SLAMF5 |
| A22 | TIGIT | TIGIT | SLAMF6 |
| A23 | TIGIT | TIGIT | SLAMF7 |
| A24 | TIGIT | TIGIT | SLAMF3 |
| A25 | TIGIT | TIGIT | EPOR |
| A26 | TIGIT | TIGIT | GCSFR |
| A27 | TIGIT | TIGIT | CSF1R |
| A28 | TIGIT | TIGIT | NKp46 |
| A29 | TIGIT | TIGIT | DR3 |
| A30 | TIGIT | TIGIT | YES1 |
| A31 | TIGIT | 2B4 | 2B4 |
| A32 | TIGIT | SLAMF1 | SLAMF1 |
| A33 | TIGIT | SLAMF5 | SLAMF5 |
| A34 | TIGIT | SLAMF6 | SLAMF6 |
| A35 | TIGIT | SLAMF7 | SLAMF7 |
| A36 | TIGIT | SLAMF3 | SLAMF3 |
| A37 | TIGIT | EPOR | EPOR |
| A38 | TIGIT | GCSFR | GCSFR |
| A39 | TIGIT | CSF1R | CSF1R |
| A40 | TIGIT | NKp46 | NKp46 |
| A41 | TIGIT | DR3 | DR3 |
| A42 | TIGIT | YES1 | YES1 |
| A43 | TIM3 | CD28 | 2B4 |
| A44 | TIM3 | CD28 | SLAMF1 |
| A45 | TIM3 | CD28 | SLAMF5 |
| A46 | TIM3 | CD28 | SLAMF6 |
| A47 | TIM3 | CD28 | SLAMF7 |
| A48 | TIM3 | CD28 | SLAMF3 |
| A49 | TIM3 | CD28 | EPOR |
| A50 | TIM3 | CD28 | GCSFR |
| A51 | TIM3 | CD28 | CSF1R |
| A52 | TIM3 | CD28 | NKp46 |
| A53 | TIM3 | CD28 | DR3 |
| A54 | TIM3 | CD28 | YES1 |
| A55 | TIM3 | TIM3 | 2B4 |
| A56 | TIM3 | TIM3 | SLAMF1 |
| A57 | TIM3 | TIM3 | SLAMF5 |
| A58 | TIM3 | TIM3 | SLAMF6 |
| A59 | TIM3 | TIM3 | SLAMF7 |
| A60 | TIM3 | TIM3 | SLAMF3 |
| A61 | TIM3 | TIM3 | EPOR |
| A62 | TIM3 | TIM3 | GCSFR |
| A63 | TIM3 | TIM3 | CSF1R |
| A64 | TIM3 | TIM3 | NKp46 |
| A65 | TIM3 | TIM3 | DR3 |
| A66 | TIM3 | TIM3 | YES1 |
| A67 | TIM3 | 2B4 | 2B4 |
| A68 | TIM3 | SLAMF1 | SLAMF1 |
| A69 | TIM3 | SLAMF5 | SLAMF5 |
| A70 | TIM3 | SLAMF6 | SLAMF6 |
| A71 | TIM3 | SLAMF7 | SLAMF7 |
| A72 | TIM3 | SLAMF3 | SLAMF3 |
| A73 | TIM3 | EPOR | EPOR |
| A74 | TIM3 | GCSFR | GCSFR |
| A75 | TIM3 | CSF1R | CSF1R |
| A76 | TIM3 | NKp46 | NKp46 |
| A77 | TIM3 | DR3 | DR3 |
| A78 | TIM3 | YES1 | YES1 |
| A79 | FAS | CD28 | 2B4 |
| A80 | FAS | CD28 | SLAMF1 |
| A81 | FAS | CD28 | SLAMF5 |
| A82 | FAS | CD28 | SLAMF6 |
| A83 | FAS | CD28 | SLAMF7 |
| A84 | FAS | CD28 | SLAMF3 |
| A85 | FAS | CD28 | EPOR |
| A86 | FAS | CD28 | GCSFR |
| A87 | FAS | CD28 | CSF1R |
| A88 | FAS | CD28 | NKp46 |
| A89 | FAS | CD28 | DR3 |
| A90 | FAS | CD28 | YES1 |
| A91 | FAS | FAS | 2B4 |
| A92 | FAS | FAS | SLAMF1 |
| A93 | FAS | FAS | SLAMF5 |
| A94 | FAS | FAS | SLAMF6 |
| A95 | FAS | FAS | SLAMF7 |
| A96 | FAS | FAS | SLAMF3 |
| A97 | FAS | FAS | EPOR |
| A98 | FAS | FAS | GCSFR |
| A99 | FAS | FAS | CSF1R |
| A100 | FAS | FAS | NKp46 |
| A101 | FAS | FAS | DR3 |
| A102 | FAS | FAS | YES1 |
| A103 | FAS | 2B4 | 2B4 |
| A104 | FAS | SLAMF1 | SLAMF1 |
| A105 | FAS | SLAMF5 | SLAMF5 |
| A106 | FAS | SLAMF6 | SLAMF6 |
| A107 | FAS | SLAMF7 | SLAMF7 |
| A108 | FAS | SLAMF3 | SLAMF3 |
| A109 | FAS | EPOR | EPOR |
| A110 | FAS | GCSFR | GCSFR |
| A111 | FAS | CSF1R | CSF1R |
| A112 | FAS | NKp46 | NKp46 |
| A113 | FAS | DR3 | DR3 |
| A114 | FAS | YES1 | YES1 |
| A115 | NKG2A | NKG2C | NKG2C |
| A116 | FAS | DR3 | DR3 |
| A117 | TGFBR2 | TGFBR2 | IL-21R |
| A118 | TGFBR2 | IL-2RG | IL-2RG |
| A119 | TGFBR2 | TGFBR2 | IL-2RG |
| A120 | TGFBR2 | IL-21R | IL-21R |
| A121 | TGFBR1 | IL-2RG | IL-2RG |
| A122 | TGFBR1 | TGFBR1 | IL-21R |
| A123 | TGFBR1 | TGFBR1 | IL-2RG |
| A124 | TGFBR1 | IL-2RG | IL-2RG |
| A125 | BTLA | CD28 | 2B4 |
| A126 | BTLA | CD28 | SLAMF1 |
| A127 | BTLA | CD28 | SLAMF5 |
| A128 | BTLA | CD28 | SLAMF6 |
| A129 | BTLA | CD28 | SLAMF7 |
| A130 | BTLA | CD28 | SLAMF3 |
| A131 | BTLA | CD28 | EPOR |
| A132 | BTLA | CD28 | GCSFR |
| A133 | BTLA | CD28 | CSF1R |
| A134 | BTLA | CD28 | NKp46 |
| A135 | BTLA | CD28 | DR3 |
| A136 | BTLA | CD28 | YES1 |
| A137 | BTLA | BTLA | 2B4 |
| A138 | BTLA | BTLA | SLAMF1 |
| A139 | BTLA | BTLA | SLAMF5 |
| A140 | BTLA | BTLA | SLAMF6 |
| A141 | BTLA | BTLA | SLAMF7 |
| A142 | BTLA | BTLA | SLAMF3 |
| A143 | BTLA | BTLA | EPOR |
| A144 | BTLA | BTLA | GCSFR |
| A145 | BTLA | BTLA | CSF1R |
| A146 | BTLA | BTLA | NKp46 |
| A147 | BTLA | BTLA | DR3 |
| A148 | BTLA | BTLA | YES1 |
| A149 | BTLA | 2B4 | 2B4 |
| A150 | BTLA | SLAMF1 | SLAMF1 |
| A151 | BTLA | SLAMF5 | SLAMF5 |
| A152 | BTLA | SLAMF6 | SLAMF6 |
| A153 | BTLA | SLAMF7 | SLAMF7 |
| A154 | BTLA | SLAMF3 | SLAMF3 |
| A155 | BTLA | EPOR | EPOR |
| A156 | BTLA | GCSFR | GCSFR |
| A157 | BTLA | CSF1R | CSF1R |
| A158 | BTLA | NKp46 | NKp46 |
| A159 | BTLA | DR3 | DR3 |
| A160 | BTLA | YES1 | YES1 |
| A161 | LILRB2 | CD28 | 2B4 |
| A162 | LILRB2 | CD28 | SLAMF1 |
| A163 | LILRB2 | CD28 | SLAMF5 |
| A164 | LILRB2 | CD28 | SLAMF6 |
| A165 | LILRB2 | CD28 | SLAMF7 |
| A166 | LILRB2 | CD28 | SLAMF3 |
| A167 | LILRB2 | CD28 | EPOR |
| A168 | LILRB2 | CD28 | GCSFR |
| A169 | LILRB2 | CD28 | CSF1R |
| A170 | LILRB2 | CD28 | NKp46 |
| A171 | LILRB2 | CD28 | DR3 |
| A172 | LILRB2 | CD28 | YES1 |
| A173 | LILRB2 | LILRB2 | 2B4 |
| A174 | LILRB2 | LILRB2 | SLAMF1 |
| A175 | LILRB2 | LILRB2 | SLAMF5 |
| A176 | LILRB2 | LILRB2 | SLAMF6 |
| A177 | LILRB2 | LILRB2 | SLAMF7 |
| A178 | LILRB2 | LILRB2 | SLAMF3 |
| A179 | LILRB2 | LILRB2 | EPOR |
| A180 | LILRB2 | LILRB2 | GCSFR |
| A181 | LILRB2 | LILRB2 | CSF1R |
| A182 | LILRB2 | LILRB2 | NKp46 |
| A183 | LILRB2 | LILRB2 | DR3 |
| A184 | LILRB2 | LILRB2 | YES1 |
| A185 | LILRB2 | 2B4 | 2B4 |
| A186 | LILRB2 | SLAMF1 | SLAMF1 |
| A187 | LILRB2 | SLAMF5 | SLAMF5 |
| A188 | LILRB2 | SLAMF6 | SLAMF6 |
| A189 | LILRB2 | SLAMF7 | SLAMF7 |
| A190 | LILRB2 | SLAMF3 | SLAMF3 |
| A191 | LILRB2 | EPOR | EPOR |
| A192 | LILRB2 | GCSFR | GCSFR |
| A193 | LILRB2 | CSF1R | CSF1R |
| A194 | LILRB2 | NKp46 | NKp46 |
| A195 | LILRB2 | DR3 | DR3 |
| A196 | LILRB2 | YES1 | YES1 |
| A197 | PD1 | CD28 | 2B4 |
| A198 | PD1 | CD28 | SLAMF1 |
| A199 | PD1 | CD28 | SLAMF5 |
| A200 | PD1 | CD28 | SLAMF6 |
| A201 | PD1 | CD28 | SLAMF7 |
| A202 | PD1 | CD28 | SLAMF3 |
| A203 | PD1 | CD28 | EPOR |
| A204 | PD1 | CD28 | GCSFR |
| A205 | PD1 | CD28 | CSF1R |
| A206 | PD1 | CD28 | NKp46 |
| A207 | PD1 | CD28 | DR3 |
| A208 | PD1 | CD28 | YES1 |
| A209 | PD1 | PD1 | 2B4 |
| A210 | PD1 | PD1 | SLAMF1 |
| A211 | PD1 | PD1 | SLAMF5 |
| A212 | PD1 | PD1 | SLAMF6 |
| A213 | PD1 | PD1 | SLAMF7 |
| A214 | PD1 | PD1 | SLAMF3 |
| A215 | PD1 | PD1 | EPOR |
| A216 | PD1 | PD1 | GCSFR |
| A217 | PD1 | PD1 | CSF1R |
| A218 | PD1 | PD1 | NKp46 |
| A219 | PD1 | PD1 | DR3 |
| A220 | PD1 | PD1 | YES1 |
| A221 | PD1 | 2B4 | 2B4 |
| A222 | PD1 | SLAMF1 | SLAMF1 |
| A223 | PD1 | SLAMF5 | SLAMF5 |
| A224 | PD1 | SLAMF6 | SLAMF6 |
| A225 | PD1 | SLAMF7 | SLAMF7 |
| A226 | PD1 | SLAMF3 | SLAMF3 |

### Cytokines

In some aspects, the disclosure provides cells and populations of cells (e.g., immune cells such as NK cells) comprising (e.g., modified to express) a chimeric protein described above and one or more cytokines, chemokine receptors, heparanase, a therapeutic agent, or an protein that may assist the immune cell overcome immunosuppression in a tumor microenvironment. In some embodiments, a cell (e.g., immune cell) provided herein comprises (e.g., is modified to express) at least one therapeutic agent selected from p40, LIGHT, CD40L, FLT3L, 4-1BBL, FasL, and heparanase.

In some embodiments, the present disclosure provides cells, e.g., immune cells (e.g., NK cells), engineered to comprise (e.g., express) an engineered protein (e.g., chimeric protein), wherein the engineered protein (e.g., chimeric protein) comprises a) an extracellular domain, b) a transmembrane domain, c) an intracellular domain, and/or d) a linker, and which are further engineered to comprise (e.g., express) a membrane-bound cytokine. In some embodiments, the membrane-bound cytokine acts in synergy with the one or more of the engineered proteins (e.g., chimeric proteins) disclosed herein. In some embodiments, the cytokine comprises at least one chemokine, interferon, interleukin, lymphokine, tumor necrosis factor, or variant or combination thereof. In some embodiments, the cytokine is an interleukin, e.g., IL-15, IL-21, IL-2, IL-12, IL-18, IL-21, IL-1, IL-3, IL-4, IL-5, IL-6, IL-7, IL-8, IL-9, IL-10, IL-11, IL-13, IL-14, IL-15, IL-16, IL-17, IL-19, IL-20, IL-22, IL-23, IL-24, IL-25, IL-26, IL-27, IL-28, IL-29, IL-30, IL-31, IL-32, IL-33, or a functional variant, fragment, or combination thereof. In some embodiments, any of the cytokines provided above can be a membrane-bound cytokine or a secreted cytokine. In some embodiments, the cytokine is a membrane-bound cytokine comprising IL-21, or a functional variant or fragment thereof. In some embodiments, the cytokine is a membrane-bound cytokine comprising IL-18, or a functional variant of fragment thereof. In some embodiments, the cytokine is a membrane-bound cytokine comprising IL-12, or a functional variant or fragment thereof. In some embodiments, the cytokine is a membrane-bound cytokine comprising IL-15, or a functional variant or fragment thereof. In some embodiments, the cytokine is a membrane-bound cytokine comprising IL-15Ra, or a functional variant or fragment thereof. In some embodiments, the cytokine is a membrane-bound cytokine comprising an IL-15/IL-15Ra complex. In some embodiments, the cytokine comprises one or both of IL-21 and IL-21R. In some embodiments, the cytokine comprises one or both of IL-18 and IL-18Ra. In some embodiments, the cytokine comprises one or both of IL-12 and IL-12RB1. In some embodiments, the cytokine comprises one or both of IL-15 and IL-15Ra.

In some embodiments, a cell provided herein comprises (e.g., is modified to express) one or more of IL-12, membrane-bound IL-12, and a fusion protein comprising IL12 subunits p35 and p40.

In some embodiments, a cell provided herein includes a membrane-bound cytokine and cytokine receptor (e.g., the cytokine's cognate receptor).

In some embodiments, the expression of any of cytokines described above can be under the control of an inducible promoter for gene transcription within the cell. In some embodiments, the inducible promoter is an EF1α promoter. In some embodiments, the inducible promoter is a PGK promoter.

In some embodiments, a cell provided herein comprises (e.g., is engineered to express) an interleukin. In some embodiments, the interleukin can comprise soluble or secreted IL-15, membrane-bound IL-15 (mbIL-15), a IL-15 receptor alpha (mbIL-15Ra), a mbIL-15 with IL-15Ra, a fusion of IL-15 and IL-15Ra, or a soluble IL-15 with IL-15Ra. In some embodiments, the IL-15 is a soluble or secreted IL-15 that complexes with IL-15Ra (e.g., on the surface of the cell). Exemplary membrane bound IL-15 (mbIL-15) proteins and fusion proteins including IL-15 and IL-15Ra are described in U.S. Pat. Nos. 10,428,305 and 9,629,877, each of which is incorporated herein by reference in its entirety. Exemplary membrane-bound IL-15 proteins are also described in Hurton et al. Proc. Nat'l. Acad. Sci. U.S.A 113(48):E7788-97, 2016, incorporated herein by reference in its entirety.

In some embodiments, a cell provided herein comprises (e.g., is engineered to express) an cytokine. In some embodiments, the cytokine is IL-15 or a fragment or variant thereof. In some embodiments, the cytokine is a complex of IL-15 or a fragment or variant thereof, and a IL-15 receptor alpha (IL-15Ra) or a fragment or variant thereof. In some embodiments, the IL-15 or a fragment or variant thereof and the IL-15Ra or fragment or variant thereof are expressed as a fusion polypeptide. In embodiments relating to IL-15 fusion polypeptides, the IL-15 may include a full-length IL-15 (e.g., a native IL-15 polypeptide) or fragment or variant thereof, fused in frame with a full-length IL-15Ra or functional fragment or variant thereof. In some embodiments, the IL-15 is linked to the IL-15Ra through a linker.

In some embodiments, a modified cell (e.g., an NK cell) expressing an engineered protein (e.g., chimeric protein) described herein further expresses a membrane-associated IL-15/IL-15Ra. In some embodiments, the mbIL-15 comprises a fusion protein between IL-15 and IL-15Ra. In a preferred embodiment, the mbIL-15 is a IL-15 and IL-15Ra linked by a 2A sequence.

In some embodiments, the IL-15 comprises an amino acid sequence having at least 90%, at least 91%, at least 92%, at least 93%, at least 94%, at least 95%, at least 96%, at least 97%, at least 98%, at least 99%, or 100% identity with the amino acid sequence of any one of SEQ ID NOs: 501-503

In some embodiments, the mbIL-15Ra comprises an amino acid sequence having at least 90%, at least 91%, at least 92%, at least 93%, at least 94%, at least 95%, at least 96%, at least 97%, at least 98%, at least 99%, or 100% identity with the amino acid sequence of SEQ ID NOs: 504-506.

In some embodiments, a cell (e.g., an immune cell) described herein comprises (e.g., is modified to express) a IgE Leader-IL-15-SG₃-(SG₄)₅-SG₃-IL-15Ra) construct. In some embodiments, the IgE Leader-IL-15-SG₃-(SG₄)₅-SG₃-IL-15Ra) construct comprises an amino acid sequence having at least 90%, at least 91%, at least 92%, at least 93%, at least 94%, at least 95%, at least 96%, at least 97%, at least 98%, at least 99%, or 100% identity with the amino acid sequence of SEQ ID NO: 507.

In some embodiments, a cell (e.g., an immune cell) described herein comprises (e.g., is modified to express) a IgE leader-IL-15-CD8a Tm+hinge construct. In some embodiments, the IgE leader-IL-15-CD8a Tm+hinge construct comprises an amino acid sequence having at least 90%, at least 91%, at least 92%, at least 93%, at least 94%, at least 95%, at least 96%, at least 97%, at least 98%, at least 99%, or 100% identity with the amino acid sequence of SEQ ID NO: 508.

In some embodiments, a cell (e.g., an immune cell) described herein comprises (e.g., is modified to express) a IL-15-(GS) 15-IL-15Ra (206-267). In some embodiments, the IL-15-(GS) 15-IL-15Ra (206-267) construct comprises an amino acid sequence having at least 90%, at least 91%, at least 92%, at least 93%, at least 94%, at least 95%, at least 96%, at least 97%, at least 98%, at least 99%, or 100% identity with the amino acid sequence of SEQ ID NO: 509.

In some embodiments, the present disclosure is directed to co-modifying cells (e.g., NK cells) with IL-15. In addition to IL-15, other cytokines are also envisioned for expression on a modified cell of the disclosure. These include, but are not limited to, cytokines, chemokines, and other molecules that contribute to the persistence, activation and proliferation of cells used for human application. NK cells expressing IL-15 are capable of continued supportive cytokine signaling, which is critical to their survival post-infusion.

In some embodiments, an engineered protein (e.g., chimeric protein) provided herein and the cytokine are each encoded by a separate vector. In some embodiments, the cytokine is IL-15 or IL-15/IL-15Ra.

TABLE A
Exemplary Cytokines

| | | |
|---|---|---|
| IL-15 | MRISKPHLRSISIQCYLCLLLNSHFLTEAGIHVFILGCFSAGLP | 501 |
| leader, pro- | KTEANWVNVISDLKKIEDLIQSMHIDATLYTESDVHPSCK | |
| peptide, mature | VTAMKCFLLELQVISLESGDASIHDTVENLIILANNSLSSN | |
| cytokine | GNVTESGCKECEELEEKNIKEFLQSFVHIVQMFINTS | |
| IL-15 | RISKPHLRSISIQCYLCLLLNSHFLTEAGIHVFILGCFSAGLPKT | 502 |
| leader, pro- | EANWVNVISDLKKIEDLIQSMHIDATLYTESDVHPSCKVTAM | |
| peptide, mature | KCFLLELQVISLESGDASIHDTVENLIILANNSLSSNGNVTESG | |
| cytokine | CKECEELEEKNIKEFLQSFVHIVQMFINTS | |
| IL-I5 | NWVNVISDLKKIEDLIQSMHIDATLYTESDVHPSCKVTAMKC | 503 |
| | FLLELQVISLESGDASIHDTVENLIILANNSLSSNGNVTESGCK | |
| | ECEELEEKNIKEFLQSFVHIVQMFINTS | |
| mbIL-15Ra | MAPRRARGCRTLGLPALLLLLLLRPPATRGITCPPPMSVE | 504 |
| leader, | HADIWVKSYSLYSRERYICNSGFKRKAGTSSLTECVLNKATN | |
| extracellular, | VAHWTTPSLKCIRDPALVHQRPAPPSTVTTAGVTPQPESLSPS | |
| transmembrane | GKEPAASSPSSNNTAATTAAIVPGSQLMPSKSPSTGTTEISSHE | |
| domain | SSHGTPSQTTAKNWELTASASHQPPGVYPQGHSDTTVAISTS | |
| intracellular | TVLLCGLSAVSLLACYLKSRQTPPLASVEMEAMEALPVTWGTS | |
| domain | SRDEDLENCSHHL | |
| mbIL-15Ra | APRRARGCRTLGLPALLLLLLLRPPATRGITCPPPMSVEHADI | 505 |
| leader, | WVKSYSLYSRERYICNSGFKRKAGTSSLTECVLNKATNVAH | |
| extracellular, | WTTPSLKCIRDPALVHQRPAPPSTVTTAGVTPQPESLSPSGKE | |
| transmembrane | PAASSPSSNNTAATTAAIVPGSQLMPSKSPSTGTTEISSHESSH | |
| domain | GTPSQTTAKNWELTASASHQPPGVYPQGHSDTTVAISTSTVL | |
| intracellular | LCGLSAVSLLACYLKSRQTPPLASVEMEAMEALPVTWGTSS | |
| domain | RDEDLENCSHHL | |
| mbIL-15Ra | ITCPPPMSVEHADIWVKSYSLYSRERYICNSGFKRKAGTSSLT | 506 |
| | ECVLNKATNVAHWTTPSLKCIRDPALVHQRPAPPSTVTTAG | |
| | VTPQPESLSPSGKEPAASSPSSNNTAATTAAIVPGSQLMPSKS | |
| | PSTGTTEISSHESSHGTPSQTTAKNWELTASASHQPPGVYPQG | |
| | HSDTTVAISTSTVLLCGLSAVSLLACYLKSRQTPPLASVEMEA | |
| | MEALPVTWGTSSRDEDLENCSHHL | |
| IgE Leader-IL-15- | MDWTWILFLVAAATRVHSNWVNVISDLKKIEDLIQSMHID | 507 |
| SG₃-(SG₄)₅-SG₃- | ATLYTESDVHPSCKVTAMKCFLLELQVISLESGDASIHDT | |
| IL15Ra) | VENLIILANNSLSSNGNVTESGCKECEELEEKNIKEFLQSF | |
| IgE Leader, IL-15, | VHIVQMFINTSSGGGSGGGGSGGGGSGGGGSGGGGSGGGG | |
| linker, IL-15Ra | SGGGITCPPPMSVEHADIWVKSYSLYSRERYICNSGFKRKAGTSSL | |
| | TECVLNKATNVAHWTTPSLKCIRDPALVHQRPAPPSTVTTAGVTP | |
| | QPESLSPSGKEPAASSPSSNNTAATTAAIVPGSQLMPSKSPSTGTT | |
| | EISSHESSHGTPSQTTAKNWELTASASHQPPGVYPQGHSDTTVAI | |
| | STSTVLLCGLSAVSLLACYLKSRQTPPLASVEMEAMEALPVTWGT | |
| | SSRDEDLENCSHHL | |
| IgE leader-IL-15- | MDWTWILFLVAAATRVHSNWVNVISDLKKIEDLIQSMHID | 508 |
| CD8a Tm + hinge | ATLYTESDVHPSCKVTAMKCFLLELQVISLESGDASIHDT | |
| IgE Leader, IL-15, | VENLIILANNSLSSNGNVTESGCKECEELEEKNIKEFLQSF | |
| CD8TM, hinge | VHIVQMFINTSTTTPAPRPPTPAPTIASQPLSLRPEACRPAAGGA | |
| | VHTRGLDFACDIYIWAPLAGTCGVLLLSLVITLYC | |
| IL15-(GS)₁₅- | MDWTWILFLVAAATRVHSNWVNVISDLKKIEDLIQSMHID | 509 |
| IL15Ra₍₂₀₆₋₂₆₇₎ | ATLYTESDVHPSCKVTAMKCFLLELQVISLESGDASIHDTVE | |
| | NLIILANNSLSSNGNVTESGCKECEELEEKNIKEFLQSFVHIVQ | |
| | MFINTSGSGSGSGSGSGSGSGSGSGSGSGSGSGSGSVAISTSTVL | |
| | LCGLSAVSLLACYLKSRQTPPLASVEMEAMEALPVTWGTSS | |
| | RDEDLENCSHHL | |

Suitable linkers, which are known to one skilled in the art, may be used, e.g., to link two cytokine polypeptides, e.g., IL-15 and IL-15RA. In certain embodiments, the use of internal ribosome entry sites (IRES) elements are used to create multigene, or polycistronic, messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

2A sequence elements can be used to create linked- or co-expression of genes in the constructs provided in the present disclosure. For example, cleavage sequences could be used to co-express genes by linking open reading frames to form a single cistron. Exemplary cleavage sequences include but are not limited to T2A, P2A, E2A and F2A, as described in Table B. In a preferred embodiment, the cleavage sequence comprises a P2A sequence.

TABLE B
Exemplary 2A Sequences

| | | |
|---|---|---|
| T2A | GSGEGRGSLLTCGDVEENPGP | 510 |
| P2A | GSGATNFSLLKQAGDVEENPGP | 511 |
| E2A | GSGQCTNYALLKLAGDVESNPGP | 512 |
| F2A | GSGVKQTLNFDLLKLAGDVESNPGP | 513 |

In some embodiments, T2A comprises an amino acid sequence having at least 90%, 91%, 92%, 93%, 94%, 95%, 96%, 97%, 98%, 99% or 100% identity with the amino acid sequence of SEQ ID NO: 510.

In some embodiments, P2A comprises an amino acid sequence having at least 90%, 91%, at least 92%, at least 93%, at least 94%, at least 95%, at least 96%, at least 97%, at least 98%, at least 99%, or 100% identity with the amino acid sequence of SEQ ID NO: 511.

In some embodiments, E2A comprises an amino acid sequence having at least 90%, at least 91%, at least 92%, at least 93%, at least 94%, at least 95%, at least 96%, at least 97%, at least 98%, at least 99%, or 100% identity with the amino acid sequence of SEQ ID NO: 512.

In some embodiments, F2A comprises an amino acid sequence having at least 90%, at least 91%, at least 92%, at least 93%, at least 94%, at least 95%, at least 96%, at least 97%, at least 98%, at least 99%, or 100% identity with the amino acid sequence of SEQ ID NO: 513. In some embodiments, a cell (e.g., an immune cell) described herein comprises (e.g., is modified to express) a chemokine or a chemokine receptor. Chemokines are a group of proteins that regulate cell trafficking and play roles in the regulation of immune response and homing of immune cells to tumors. Transgenic expression of chemokine receptors CCR2b or CXCR2 in T cells enhances trafficking to CCL2- or CXCL1-secreting solid tumors including melanoma and neuroblastoma (Craddock et al., J. Immunother. 33(8):780-8, 2010, and Kershaw et al., Hum. Gene Ther. 13(16):1971-80, 2002) The chemokine receptor molecule can comprise a naturally occurring or recombinant chemokine receptor or a chemokine-binding fragment thereof. A chemokine receptor molecule suitable for expression in a modified cell of the disclosure (e.g., NK-cell) described herein include a CXC chemokine receptor (e.g., CXCR1, CXCR2, CXCR3, CXCR4, CXCR5, CXCR6, or CXCR7), a CC chemokine receptor (e.g., CCR1, CCR2, CCR3, CCR4, CCR5, CCR6, CCR7, CCR8, CCR9, CCR10, or CCR11), a CX3C chemokine receptor (e.g., CX3CR1), a XC chemokine receptor (e.g., XCR1), or a chemokine-binding fragment thereof. In one embodiment, the chemokine receptor molecule to be expressed with an engineered protein (e.g., chimeric protein) described herein is selected based on the chemokine(s) secreted by the tumor. In one embodiment, the engineered protein (e.g., chimeric protein)-expressing cell described herein further comprises, e.g., expresses, a CCR4 receptor. In an embodiment, the engineered protein (e.g., chimeric protein) described herein and the chemokine receptor molecule are on the same vector or are on two different vectors. In embodiments where the engineered protein (e.g., chimeric protein) described herein and the chemokine receptor molecule are on the same vector, the engineered protein (e.g., chimeric protein) and the chemokine receptor molecule are each under control of two different promoters or are under the control of the same promoter.

### Chimeric Antigen Receptors (CARs)

The present disclosure provides cells (e.g., immune cells, such as NK cells) engineered to comprise, (e.g., express) an engineered protein (e.g., chimeric protein), wherein the engineered protein (e.g., chimeric protein) comprises a) an extracellular domain, b) a transmembrane domain, c) an intracellular domain, and/or d) a linker, and which is further engineered to comprise (e.g., express) a chimeric antigen receptor (CAR). In some embodiments, the CAR acts in synergy with the one or more of the engineered proteins (e.g., chimeric proteins), and/or the one or more of the cytokines (e.g., membrane-bound cytokines) disclosed herein.

In some embodiments, the CARs are activating or stimulatory CARs, costimulatory CARs (see, e.g., International Patent Publication No. WO 2014/055668), and/or inhibitory CARs (iCARs, see, e.g., Fedorov et al., Sci. Transl. Med. 5(215):215ra172, 2013). The CARs generally include an extracellular domain comprising an antigen (or ligand)-binding domain linked to one or more intracellular domains, e.g., via linkers and/or transmembrane domain(s). Such molecules typically mimic or approximate a signal through a natural antigen receptor, a signal through such a receptor in combination with a costimulatory receptor, and/or a signal through a costimulatory receptor alone. For example, once an antigen is recognized by the antigen-binding domain, the intracellular domain transmits an activation signal to the immune cell, e.g., NK cell, that induces the cell to destroy a targeted tumor cell.

In some embodiments, the CAR comprises (a) an antigen-binding domain; (b) a linker, (c) a transmembrane domain; and (d) at least one intracellular domain (e.g., comprising a stimulatory domain). In some embodiments, the CAR comprises (a) an antigen-binding domain; (b) a linker derived from one or more of CD8a (also referred to herein as CD8α), IgG1, IgG4, or CD28; (c) a transmembrane domain derived from one or more of CD27, CD28, CD8a, DAP10, DAP12, or NKG2D; and (d) at least one (e.g., one, two, three or more) intracellular domain derived from one or more of CD28, DAP10, DAP12, CD27, 4-1BB, 2B4, OX40, CD3zeta or FCER1G.

In certain embodiments of the CAR, the antigen-specific portion of the receptor comprises a tumor-associated antigen- or a pathogen-specific antigen-binding domain. Antigens include carbohydrate antigens recognized by pattern-recognition receptors, such as Dectin-1. A tumor associated antigen may be of any kind so long as it is expressed or present on the cell surface of tumor cells. Exemplary embodiments of tumor-associated antigens include, but are not limited to, CD70. In some embodiments, the antigen-specific portion of the CAR binds to CD70.

The antigen-binding domain of the CAR can comprise a fragment of the VH and VL chains of a single-chain variable fragment (scFv) that specifically binds CD70 polypeptide such as those described in U.S. Patent Application Publication Nos. 2018/0230224A1, 2019/0233528A1, and 2019/0233529A1; U.S. Pat. Nos. 8,124,738, 8,067,546, 8,562,987, 9,428,585, 9,701,752, 7,662,387, 8,535,678, 8,609,104, 8,663,642, 9,345,785, 7,641,903, 8,337,838, 8,647,624, 9,051,372, and 7,491,390; and EP 1934261, EP 1871418, EP 1594542, EP 2100619, EP 2289559, EP 1799262, and EP 3583129 A1, each of which is incorporated herein by reference in its entirety. Exemplary CD70 antigen-binding domains include, but are not limited to, anti-CD70 antibodies reviewed in Starzer et al., ESMO Open 2020; 4: e000629. The fragment can also be any number of different antigen-binding domains of a human antigen-specific antibody. In some embodiments, the fragment is an antigen-specific scFv encoded by a sequence that is optimized for human codon usage for expression in human cells.

The arrangement of the antigen-binding domain of the CAR could be multimeric, such as a diabody or multimers. In some embodiments, the multimers can be formed by cross pairing of the variable portion of the light and heavy chains into a diabody.

In some embodiments, the antigen-binding domain of the CAR comprises an antibody or an antigen-binding fragment thereof. In some embodiments, the antigen-binding domain comprises a single chain antibody fragment (scFv) comprising a light chain variable domain (VL) and heavy chain variable domain (VH) of a target antigen specific monoclonal anti-CD70 antibody, optionally, joined by a flexible linker, such as a glycine-serine linker or a Whitlow linker. In some embodiments, the scFv is humanized. In some embodiments, the antigen-binding domain may comprise VH and VL that are directionally linked, for example, from N- to C-terminus, VH-linker-VL or VL-linker-VH.

In some embodiments, scFv affinity of the CAR for CD70 may be optimized to induce cytotoxicity of tumor cells that produce high levels of CD70 without inducing cytotoxicity of normal cell that express low or normal levels of CD70. Illustrative examples of such affinity tuning are provided in Caruso et al., Cancer Res. 75:3505-18, 2015, and Liu et al., Cancer Res. 75:3596-607, 2015.

Exemplary anti-CD70 scFvs include but are not limited to 2H5, 10B4, 8B5, 18E7, 69A7, h1F6_VHE_VLA, h1F6_VHH_VLA, h1F6_VHJ_VLA, h1F6_VHM_VLA, h1F6_VHE_VLD, c1F6, 1F6-1, and 2F2, and immunologically active and/or antigen-binding fragments thereof. Exemplary anti-CD70 scFvs include but are not limited to 8G1, 1C8, 6E9, 31H1, 63B2, 40E3, 42C3, 45F11, 64F9, 72C2, 2F10, 4F11, 10H10, 17G6, 65E11, P02B10, P07D03, P08A02, P08E02, P08F08, P08G02, P12B09, P12F02, P12G07, P13F04, P15D02, P16C05, 10A1, 10E2, 11A1, 11C1, 11D1, 11E1, 12A2, 12C4, 12C5, 12D3, 12D6, 12D7, 12F5, 12H4, 8C8, 8F7, 8F8, 9D8, 9E10, 9E5, 9F4, 9F8, 12C6, CD70-1, CD70-2, CD70-3, CD70-4, CD70-5, CD70-6, CD70-7, CD70-8, CD70-9, CD70-10, CD70-11, CD70-12, CD70-13, CD70-14, CD70-15, CD70-16, CD70-17, CD70-18, CD70-19, CD70-20, CD70-21, CD70-22, CD70-23, CD70-24, CD70-25, CD70-26, CD70-27, CD70-28, CD70-29, CD70-30, CD70-31, CD70-32, CD70-33, CD70-34, CD70-35, CD70-36, CD70-37, CD70-38, CD70-39, CD70-40, CD70-41, CD70-42, CD70-43, CD70-44, CD70-45, CD70-46, CD70-47, CD70-48, CD70-49, CD70-50, CD70-51, CD70-52, CD70-53, CD70-54, CD70-55, CD70-56, CD70-57, CD70-58, CD70-59, CD70-60, CD70-61, CD70-62, CD70-63, CD70-64, CD70-65, CD70-66, CD70-67, CD70-68, CD70-69, CD70-70, CD70-71, CD70-72, CD70-73, CD70-74, CD70-75, CD70-76, CD70-77, CD70-78, CD70-79, CD70-80, CD70-81, CD70-82, CD70-83, CD70-84, CD70-85, CD70-86, CD70-87, CD70-88, CD70-89, CD70-90, CD70-91, CD70-92, CD70-93, CD70-94, CD70-95, CD70-96, CD70-97, CD70-98, CD70-99, CD70-100, CD70-101, CD70-102, CD70-103, CD70-104, CD70-105, CD70-106, CD70-107, CD70-108, CD70-109, CD70-110, CD70-111, CD70-112, CD70-113, CD70-114, CD70-115, CD70-116, CD70-117, CD70-118, CD70-119, CD70-120, CD70-121, CD70-122, CD70-123, CD70-124, CD70-125, CD70-126, CD70-127, CD70-128, CD70-129, CD70-130, CD70-131, CD70-132, CD70-133, CD70-134, CD70-135, CD70-136, CD70-137, CD70-138, CD70-139, CD70-140, CD70-141, CD70-142, CD70-143, CD70-144, CD70-145, CD70-146, CD70-147, CD70-148, CD70-149, CD70-150, CD70-151, CD70-152, CD70-153, CD70-154, CD70-155, CD70-156, CD70-157, CD70-158, CD70-159, CD70-160, CD70-161, CD70-162, CD70-163, CD70-164, CD70-165, CD70-166, CD70-167, CD70-168, CD70-169, CD70-170, CD70-171, CD70-172, CD70-173, CD70-174, CD70-175, CD70-176, CD70-177, CD70-178, CD70-179, CD70-180, 1C2, 9D1, 8B 12, 8C12, 9.00E+01, 5F4, 5B2, 6D5, 4D2, 9A1, 9G2, 9B2, 2.40E+04, 33D8, 24F2, 24B6, 19G10, 45B12, 45D9, 45F8, 45A12, 45B6, 57B6, 59D10, 27B3, 36A9, 53F1, 36D6, 53G1, 35G3, 53C1, 35F6, 36G2, 39D5, 42D12, 35C1, 41D12, 41H8, 35G2, 40F1, 53B1, 39C3, 53D1, 53H1, 53A2, ARGX-110, CTX-130, CTX-130, and SCAR70, and immunologically active and/or antigen-binding fragments thereof.

### Exemplary Anti-CD70 CAR Constructs

Disclosed herein are a chimeric antigen receptor (CAR), wherein the CAR comprises (a) a CD70 antigen-binding domain; (b) a linker derived from one or more of CD8a, IgG1, IgG4, or CD28; (c) a transmembrane domain derived from one or more of CD27, CD28, CD8a, DAP10, DAP12, or NKG2D; and (d) an intracellular domain derived from one or more of CD28, DAP10, DAP12, CD27, 4-1BB, 2B4, OX40, CD3zeta or FCER1G.

Table C provides exemplary anti-CD70 CAR constructs disclosed herein and the domains that they comprise.

TABLE C
Exemplary anti-CD70 CAR Constructs and Domains Thereof

| | Signaling | | | | | |
|---|---|---|---|---|---|---|
| ID | Peptide | ECD | Linker | TMD | ICD 1 | ICD 2 |
| CAT-70-001 | CD8a | 1F6 | CD8a | CD28 | CD28 | CD3z |
| CAT-70-002 | CD8a | 1F6 | CD8a | NKG2D | DAP10 | CD3z |
| CAT-70-003 | CD8a | 1F6 | CD8a | NKG2D | DAP12 | CD3z |
| CAT-70-004 | CD27 | CD27 | — | CD27 | CD27 | CD3z |
| CAT-70-005 | CD27 | CD27 | — | CD28 | CD28 | CD3z |
| CAT-70-006 | CD27 | CD27 | — | NKG2D | DAP10 | CD3z |
| CAT-70-007 | CD27 | CD27 | — | NKG2D | DAP12 | CD3z |
| CAT-CD70-119 | CD27 | CD27 | — | CD27 | 4-1BB | CD3z |
| CAT-CD70-122 | CD27 | CD27 | CD8a | CD8a | 4-1BB | CD3z |
| CAT-CD70-124 | CD27 | CD27 | — | CD27 | CD28 | CD3z |
| CAT-CD70-125 | CD27 | CD27 | CD8a | CD8a | CD28 | CD3z |
| CAT-CD70-127 | CD8a | 1F6 | CD8a | CD8a | 4-1BB | CD3z |
| CAT-CD70-130 | CD8a | 1F6 | IgG1 | CD28 | CD28 | CD3z |
| CAT-CD70-133 | CD8a | 1F6 | CD28 | CD28 | CD28 | CD3z |
| CAT-CD70-135 | CD8a | 1F6 | CD8a | CD8a | CD28 | CD3z |
| CAT-CD70-136 | CD27 | CD27 | — | CD27 | CD27 | DAP12 |
| CAT-CD70-137 | CD27 | CD27 | — | CD27 | CD27 | FCER1G |
| CAT-CD70-140 | CD27 | CD27 | — | DAP10 | DAP10 | CD3z |
| CAT-CD70-141 | CD27 | CD27 | — | DAP12 | DAP12 | CD3z |
| CAT-CD70-142 | CD27 | CD27 | — | DAP12 | DAP12 | — |
| CAT-CD70-143 | CD8a | 1F6 | IgG1 | CD28 | CD28 | — |
| CAT-CD70-144 | CD8a | 1F6 | CD8a | CD8a | 4-1BB | — |
| CAT-CD70-145 | CD8a | 1F6 | CD8a | CD8a | — | CD3z |
| CAT-CD70-146 | CD8a | 1F6 | CD8a | CD8a | 4-1BB | 4-1BB |
| CAT-CD70-147 | CD8a | 1F6 | CD8a | CD8a | 2B4 | CD3z |
| CAT-CD70-148 | CD8a | 1F6 | CD8a | CD8a | DAP10 | CD3z |
| CAT-CD70-149 | CD8a | 1F6 | CD8a | CD8a | DAP12 | CD3z |
| CAT-CD70-150 | CD8a | 1F6 | CD8a | CD8a | OX40 | CD3z |
| CAT-CD70-153 | CD8a | 1F6 | CD8a | NKG2D | 2B4 | CD3z |
| CAT-CD70-154 | CD8a | 1F6 | CD8a | DAP10 | DAP10 | CD3z |
| CAT-CD70-155 | CD8a | 1F6 | CD8a | DAP12 | DAP12 | CD3z |
| CAT-CD70-156 | CD8a | 1F6 | CD8a | DAP12 | DAP12 | — |
| CAT-CD70-157 | CD8a | 1F6 | CD8a | CD28 | CD28 | DAP12 |
| CAT-CD70-158 | CD8a | 1F6 | CD8a | CD8a | 4-1BB | DAP12 |
| CAT-CD70-159 | CD8a | 1F6 | CD8a | CD8a | OX40 | DAP12 |
| CAT-CD70-160 | CD8a | 1F6 | CD8a | CD8a | DAP10 | DAP12 |
| CAT-CD70-161 | CD8a | 1F6 | CD8a | CD28 | CD28 | FCER1G |
| CAT-CD70-162 | CD8a | 1F6 | CD8a | CD8a | 4-1BB | FCER1G |
| CAT-CD70-163 | CD8a | 1F6 | CD8a | CD8a | OX40 | FCER1G |
| CAT-CD70-164 | CD8a | 1F6 | CD8a | CD8a | DAP10 | FCER1G |

### Exemplary Combinations of an Engineered Protein, a CAR and a Membrane Bound Cytokine

Also provided herein are cells (e.g., immune cells, such as NK cells) engineered to comprise, (e.g., express) a chimeric protein comprising an extracellular domain, a transmembrane domain, and an intracellular domain, wherein the extracellular domain binds to TGF-β, and wherein the intracellular domain comprises an intracellular domain, or a portion thereof, of a stimulatory polypeptide, and which is further engineered to comprise (e.g., express) a chimeric antigen receptor (CAR) and a cytokine provided herein (e.g., membrane-associated IL-15/IL-15Ra). In some embodiments, the CAR comprises a CD70 antigen-binding domain.

Also provided herein are cells (e.g., immune cells, such as NK cells) engineered to comprise, (e.g., express) a protein comprising an extracellular domain and a transmembrane domain, wherein the extracellular domain is capable of binding TGF-β, and wherein the chimeric protein lacks a fully functional intracellular domain, and which is further engineered to comprise (e.g., express) a chimeric antigen receptor (CAR) and a cytokine provided herein (e.g., membrane-associated IL-15/IL-15Ra). In some embodiments, the CAR comprises a CD70 antigen-binding domain.

Also provided herein are cells (e.g., immune cells, such as NK cells) engineered to comprise, (e.g., express) a protein comprising a dominant negative isoform of a TGF-BR1, wherein the dominant negative isoform of TGF-BR1 competes with a wild-type isoform of a TGF-BR1 for binding TGF-B, and which is further engineered to comprise (e.g., express) a chimeric antigen receptor (CAR) and a cytokine provided herein (e.g., membrane-associated IL-15/IL-15Ra). In some embodiments, the CAR comprises a CD70 antigen-binding domain.

Also provided herein are cells (e.g., immune cells, such as NK cells) engineered to comprise, (e.g., express) a protein comprising a dominant negative isoform of a TGF-BR2, wherein the dominant negative isoform of TGF-BR21 competes with a wild-type isoform of a TGF-BR2 for binding TGF-B, and which is further engineered to comprise (e.g., express) a chimeric antigen receptor (CAR) and a cytokine provided herein (e.g., membrane-associated IL-15/IL-15Ra). In some embodiments, the CAR comprises a CD70 antigen-binding domain.

### Antigens

Among the antigens targeted by the CAR are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Any suitable antigen may find use in the present method. Exemplary antigens include, but are not limited to, antigenic molecules from infectious agents, glycosylated antigens, TnAntigens, auto-/self-antigens, tumor-/cancer-associated antigens, and tumor neoantigens (Linnemann et al., Nat. Med. 21(1):81-5, 2015). In particular embodiments, the antigens can be selected from the group consisting of CD70, Her2, mesothelin, Ror1, Muc16, L1Cam, Lewis Y, B7-H3, FOLR1, PSMA, PSCA, BCMA, GPRC5D, CD138, CS1, CD19, CD20, CD22, CD79a, CD79b, CD37, CXCR5, TGF-B, CD96, CD33, CD123, CLEC12a, ADGRE2, or LILRB2. In preferred embodiments, the antigen is a CD70 antigen. In particular aspects, the antigens for targeting by two or more extracellular domains include, but are not limited to TGF-B and CD33 (e.g., for AML), TGF-B and CD123 (e.g., for AML), TGF-B and CLL1 (e.g., for AML), TGF-B and CD96 (e.g., for AML); TGF-B and CD19 (e.g., for B cell malignancies); TGF-B and CD22 (e.g., for B cell malignancies); TGF-B and CD20 (e.g., for B cell malignancies); TGF-B and CD79a (e.g., for B cell malignancies); TGF-B and CD79b (e.g., for B cell malignancies); TGF-B and BCMA (e.g., for multiple myeloma); TGF-B and GPRC5D (e.g., for multiple myeloma); TGF-B and CD138 (e.g., for multiple myeloma); TGF-B and CD96 (e.g., for RCC); TGF-B and HAVCR (e.g., for RCC); TGF-B and EGFR (e.g., for RCC). The sequences for these antigens are known in the art, for example, CD33—NM_001772.4; CD123—NC_000023.11, CLL1—NM_138337.6; CD96—NM_198196.3; CD96—NM_198196.3; HAVCR1—NM_001173393.3; EGFR—NM_005228.5; CD19-NG_007275.1; CD22—NM_001771.4; CD20—NM_152866.3; CD79a—NM_001783.4; CD79b—NM_001039933.3; CD37—NM_001774.3; CXCR5—NM_001716.5; BCMA—NM_001192.3; GPRC5D NM_018654.1; and CD138—NM_001006946.1.

Tumor-associated antigens may be derived from prostate, breast, colorectal, lung, pancreatic, renal, mesothelioma, ovarian, or melanoma cancers. Exemplary tumor-associated antigens or tumor cell-derived antigens include MAGE 1, 3, and MAGE 4 (or other MAGE antigens such as those disclosed in International Patent Publication No. WO 99/40188); PRAME; BAGE; RAGE, Lage (also known as NY ESO 1); SAGE; HAGE or GAGE; Her2; mesothelin; Ror1; Muc16; L1Cam; Lewis Y; B7-H3; FOLR1; PSMA; and PSCA. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate-specific membrane antigen (PSMA), prostate-specific antigen (PSA), prostatic acid phosphates, NKX3.1, and six-transmembrane epithelial antigen of the prostate (STEAP).

Other tumor associated antigens include Plu-1, HASH-1, HasH-2, Cripto, and Criptin. Additionally, a tumor antigen may be a self-peptide hormone, such as whole length gonadotrophin hormone releasing hormone (GnRH), a short 10 amino acid long peptide, useful in the treatment of many cancers.

Tumor antigens include tumor antigens derived from cancers that are characterized by tumor-associated antigen expression, such as HER-2/neu expression. Tumor-associated antigens of interest include lineage-specific tumor antigens such as the melanocyte-melanoma lineage antigens MART-1/Melan-A, gp100, gp75, mda-7, tyrosinase and tyrosinase-related protein. Illustrative tumor-associated antigens include, but are not limited to, tumor antigens derived from or comprising any one or more of, p53, Ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, BAGE, DAM-6, -10, GAGE-1, -2, -8, GAGE-3, -4, -5, -6, -7B, NA88-A, MART-1, MCIR, gp100, PSA, PSM, tyrosinase, TRP-1, TRP-2, ART-4, CAMEL, CEA, Cyp-B, hTERT, hTRT, ICE, MUC1, MUC2, Phosphoinositide 3-kinases (PI3Ks), TRK receptors, PRAME, P15, RU1, RU2, SART-1, SART-3, Wilms' tumor antigen (WT1), AFP, catenin/m, Caspase-8/m, CEA, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mber-abl, BCR-ABL, interferon regulatory factor 4 (IRF4), ETV6/AML, LDLR/FUT, Pml/RAR, tumor-associated calcium signal transducer 1 (TACSTD1), TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch1-4), c-Met, mammalian targets of rapamycin (mTOR), WNT, extracellular signal-regulated kinases (ERKs), and their regulatory subunits, PMSA, PR-3, MDM2, mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B 1, polysialic acid, MYCN, RhoC, GD3, fucosyl GM1, PSCA, sLe, PLAC1, GM3, BORIS, Tn, GLoboH, NY-BR-1, RGsS, SART3, STn, PAX5, OY-TES 1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, LRRN1 and idiotype.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B 1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

### Safety Switch Proteins

In some embodiments, the cells, e.g., immune cells, comprising an engineered protein (e.g., chimeric protein) described herein that have been infused into a mammalian subject, e.g., a human, can be ablated in order to regulate the effect of such cells, e.g., immune cells should toxicity arise from their use. The engineered protein (e.g., chimeric protein) of the cells, e.g., immune cells of the present disclosure may comprise one or more suicide genes or safety switch proteins (e.g., caspase-9, inducible FAS (iFAS), and inducible caspase-9 (icasp9)).

As used herein, the term “safety switch protein,” “suicide protein,” or “kill switch protein” refers to an engineered protein (e.g., chimeric protein) designed to prevent potential toxicity or otherwise adverse effects of a cell therapy. In some embodiments, the safety switch protein expression is conditionally controlled to address safety concerns for transplanted engineered cells that have permanently incorporated the gene encoding the safety switch protein into its genome. This conditional regulation could be variable and might include control through a small molecule-mediated post-translational activation and tissue-specific and/or temporal transcriptional regulation. The safety switch could mediate induction of apoptosis, inhibition of protein synthesis or DNA replication, growth arrest, transcriptional and post-transcriptional genetic regulation and/or antibody-mediated depletion. In some embodiments, the safety switch protein is activated by an exogenous molecule, e.g., a prodrug, that, when activated, triggers apoptosis and/or cell death of a therapeutic cell.

The term “suicide gene” or “kill switch gene” as used herein is defined as a gene which, upon administration of a prodrug, effects transition of a gene product to a compound which kills its host cell. Examples of suicide gene/prodrug combinations which may be used include, but are not limited to inducible caspase 9 (iCASP9) and rimiducid; RQR8 and rituximab; truncated version of EGFR variant III (EGFRv3) and cetuximab; herpes simplex virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside. The E. coli purine nucleoside phosphorylase, a suicide gene which converts the prodrug 6-methylpurine deoxyriboside to toxic purine 6-methylpurine. Other examples of suicide genes used with prodrug therapy are the E. coli cytosine deaminase gene and the HSV thymidine kinase gene.

Exemplary suicide genes include but are not limited to inducible caspase 9 (or caspase 3 or 7), CD20, CD52, EGFRt, or, thymidine kinase, cytosine deaminase, HER1 and any combination thereof. Further suicide genes known in the art that may be used in the present disclosure include purine nucleoside phosphorylase (PNP), cytochrome p450 enzymes (CYP), carboxypeptidases (CP), carboxylesterase (CE), nitroreductase (NTR), guanine ribosyltransferase (XGRTP), glycosidase enzymes, methionine-γ-lyase (MET), and thymidine phosphorylase (TP).

### Engineered Protein Expression Levels

The present disclosure provides a population of engineered cells, e.g., immune cells (e.g., NK cells), wherein a plurality of the engineered cells, e.g., immune cells (e.g., NK cells), of the population comprise an engineered protein (e.g., chimeric protein) disclosed herein. The present disclosure provides a composition comprising a population of NK cells, wherein a plurality of the NK cells of the population comprise a non-naturally occurring engineered protein (e.g., chimeric protein) comprising, consisting essentially of, or consisting of: a) an extracellular domain, b) a transmembrane domain, c) an intracellular domain, and/or d) a linker. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprise the engineered protein (e.g., chimeric protein). In some embodiments, the engineered protein (e.g., chimeric protein) is expressed at a copy number of at least 1, 2, 3, 4, 5, 6, 7, 8, 9, 10, 20, 30, 40, 50, 60, 70, 80, 90 or 100, 500, 1000, 5000, 10,000, 15,000, 20,000, 25,000, 30,000, 40,000, or 50,000 copies per cell. In some embodiments, a nucleic acid encoding an engineered protein (e.g., chimeric protein) is integrated into the genome of a cell at a copy number of at least 1, 2, 3, 4, 5, 6, 7, 8, 9, 10, 20, or 30 copies per cell.

In some embodiments, the cells, e.g., immune cells (e.g., NK cells), expressing an engineered protein (e.g., chimeric protein) are further engineered to express at least one cytokine. In some embodiments, the cells, e.g., immune cells (e.g., NK cells), expressing an engineered protein (e.g., chimeric protein) are further engineered to express a CAR.

### Assays

All of the engineered proteins (e.g., chimeric proteins) disclosed herein can be generated and tested for structural and/or functional efficacy without undue experimentation in view of the present disclosure and further in view of the common general knowledge available in the art.

In some embodiments, a population of genetically engineered NK cells as disclosed herein exhibit an NK cell function (e.g., effector function). In some embodiments, the population is cytotoxic, e.g., to cancer cells. In some embodiments, the population exhibits directed secretion of cytolytic granules or engagement of death domain-containing receptors. In some embodiments, the cytolytic granules comprise perforin and/or granzymes.

Routine experimentation and assays well-known in the art can be used to identify an NK cell function in terms of its degranulation (e.g., CD107a expression), activation (e.g., CD69 production), cytokine production (e.g., TNFalpha or IFN-gamma production), target cell line killing, and/or anti-tumor efficacy in models (e.g., mice). Illustrative assays for measuring NK cell cytotoxicity and CD107a (granule release) are provided in Li et al., Cell Stem Cell 23:181-192, 2018; incorporated in its entirety herein by reference. In some embodiments, the population exhibits one or more NK cell effector functions at a level that is least 3-4 (e.g., at least 3, 4, 5, 6, 7, 8, 9, 10, 20, 30, 40, 50, 100, 150, 200, 250, 300, 400, 500, 1000, 2,500, 5,000, 10,000, 15,000, or 25,000)-fold higher than the functions exhibited by a population of NK cells not expressing an engineered protein (e.g., chimeric protein) provided herein.

Additional assays, specific to one or more stimulatory and/or inhibitory polypeptides disclosed herein, will be apparent to one of skill in the art. For example, TGF-β signal transduction is known to trigger Smad2/3 phosphorylation. Further, expression of a dominant negative isoform of a TGF beta receptor in an NK cell has been demonstrated to block Smad2/3 phosphorylation (Burga et al., Clinical Cancer Research, 25(14):4400-12, 2019; incorporated in its entirety herein by reference). Accordingly, the activity of a dominant negative isoform of a TGF beta receptor can be determined by measuring the inhibition of Smad2/3 phosphorylation in an immune cell, for example, by performing a phopho-flow assay, a luminex kit assay, or a Western blot assay (Burga et al., 2019). Further, TGF-B signal transduction is associated with a loss of expression of NKG2D and DNAM-1 on the surface of an NK cell. The expression of a dominant negative isoform of a TGF beta receptor in an NK cell has been demonstrated to block this loss of expression of NKG2D and DNAM-1 (Burga et al., 2019). Accordingly, the activity of a dominant negative isoform of a TGF beta receptor can be determined by measuring the expression of NKG2D and DNAM-1 on the surface of an NK cell, for example, by performing flow cytometry (Burga et al., 2019) or any other cell phenotyping assays known in the art. The expression of a dominant negative isoform of a TGF beta receptor in an NK cell has been demonstrated to prevent the TGF-B mediated reduction of NK cell cytotoxicity (Burga et al., 2019). Accordingly, the activity of a dominant negative isoform of a TGF beta receptor can be determined by measuring the NK cell cytotoxicity, for example, by performing a Cr-51 release assay with SHSY5Y cells (Burga et al., 2019) or any other NK cell cytotoxicity assay known in the art (Li et al., 2018).

Additional assays comprise the use of a bead-based or a cell-based system for identifying synergy among receptors on resting NK cells for the activation of natural cytotoxicity and cytokine secretion, as described by Bryceson et al., Blood 107(1):159-66, 2006. Target-cell lysis by IL-2-activated NK cells in a redirected, antibody-dependent cytotoxicity assay is triggered by a number of receptors. In contrast, cytotoxicity by resting NK cells is induced only by CD16, and not by NKp46, NKG2D, 2B4 (CD244), DNAM-1 (CD226), or CD2. Calcium flux in resting NK cells is induced with antibodies to CD16 and, to a weaker extent, antibodies to NKp46 and 2B4. Although NKp46 does not enhance CD16-mediated calcium flux, it synergizes with all other receptors. 2B4 synergizes with 3 other receptors, NKG2D and DNAM-1 each synergize with 2 other receptors, and CD2 synergizes with NKp46 only. Resting NK cells can be induced to secrete tumor necrosis factor alpha (TNF-alpha) and interferon gamma (IFN-gamma), and to kill target cells by engagement of specific, pair-wise combinations of receptors. Therefore, natural cytotoxicity by resting NK cells is induced only by mutual co-stimulation of non-activating receptors. The function of the engineered proteins (e.g., chimeric proteins) of the sink or dominant negative receptor modalities disclosed herein may be tested in expanded transduced NK cells, and assayed for an output signal of Smad2/3 phosphorylation, as described herein (Burga et al., 2019), or upstream signaling events, for example, Syk phosphorylation. Further, the function of the chimeric proteins of the signal inverter modality disclosed herein, may be tested in transfected reporter cell lines, and assayed for an output signal of a downstream pathway (e.g., NF-κB, AP-1, and/or NFAT activity) that can detect activity from diverse upstream signals.

### IV. Methods of Gene Delivery and Cell Modification

Cells, e.g., immune cells comprising an engineered protein (e.g., chimeric protein), a cytokine and/or a CAR, as disclosed herein can be prepared using numerous methods known to one of skill in the art. For example, in some embodiments, an engineered protein (e.g., chimeric protein) may be expressed in a cell, e.g., an immune cell by introducing a vector encoding the engineered protein (e.g., chimeric protein). One of skill in the art would be well-equipped to construct a vector through standard recombinant techniques (see, for example, Sambrook et al., Molecular Cloning: A Laboratory Manual, 3rd ed., Cold Spring Harbor Press, Cold Spring Harbor, N.Y., 2001 and Ausubel et al., Current Protocols in Molecular Biology, Greene Publishing Associates and John Wiley & Sons, NY, 1994, both incorporated herein by reference) for the expression of the engineered proteins (e.g., chimeric proteins) of the present disclosure. Vectors include but are not limited to, plasmids, cosmids, viruses (bacteriophage, animal viruses, and plant viruses), and artificial chromosomes (e.g., YACs), such as retroviral vectors (e.g., derived from Moloney murine leukemia virus vectors (MoMLV), MSCV, SFFV, MPSV, SNV., etc), lentiviral vectors (e.g., derived from HIV-1, HIV-2, SIV, BIV, FIV., etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors, and group B adenovirus enadenotucirev vectors.

Numerous plasmid vectors are known in the art for inducing a nucleic acid encoding a protein. These include, but are not limited to, the vectors disclosed in U.S. Pat. Nos. 6,103,470, 7,598,364, 7,989,425, and 6,416,998, and 8,546,140, each of which is incorporated herein by reference.

An episomal gene delivery system can be a plasmid, an Epstein-Barr virus (EBV)-based episomal vector, a yeast-based vector, an adenovirus-based vector, a simian virus 40 (SV40)-based episomal vector, a bovine papilloma virus (BPV)-based vector, or a lentiviral vector. A viral gene delivery system can be an RNA-based or DNA-based viral vector.

In some embodiments, the cells, e.g., immune cells (e.g., NK cells) comprise one or more nucleic acids introduced via genetic engineering that encode one or more chimeric proteins, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g., chimeric).

### Viral Vectors

Viral vectors encoding an engineered protein (e.g., chimeric protein), a cytokine a CAR, and other proteins described herein are provided in certain aspects of the present disclosure.

In some embodiments of the methods of the disclosure, introducing a nucleic acid sequence and/or a genomic editing construct into a cell, e.g., an immune cell ex vivo, in vivo, in vitro or in situ comprises the use of a viral vector. In some embodiments, the viral vector is a non-integrating non-chromosomal vector. Exemplary non-integrating non-chromosomal vectors include, but are not limited to, adeno-associated virus (AAV), adenovirus, and herpes viruses. In some embodiments, the viral vector is an integrating chromosomal vector. Integrating chromosomal vectors include, but are not limited to, adeno-associated vectors (AAV), lentiviruses, and gamma-retroviruses.

Lentiviruses are complex retroviruses, which, in addition to the common retroviral genes gag, pol, and env, contain other genes with regulatory or structural function. Lentiviral vectors are well known in the art (see, for example, U.S. Pat. Nos. 6,013,516 and 5,994,136).

A retroviral vector may also be, e.g., a gammaretroviral vector. A gammaretroviral vector may include, e.g., a promoter, a packaging signal (w), a primer binding site (PBS), one or more (e.g., two) long terminal repeats (LTR), and a transgene of interest, e.g., a gene encoding an engineered protein (e.g., chimeric protein). A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., Viruses 3(6):677-713, 2011.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell—wherein a suitable host cell is transfected with two or more vectors carrying the packaging functions, namely gag, pol and env, as well as rev and tat—is described in U.S. Pat. No. 5,994,136, incorporated herein by reference.

In certain embodiments of the methods of the disclosure, transgene delivery can occur by a combination of vectors. Exemplary but non-limiting vector combinations can include: viral plus non-viral vectors, more than one non-viral vector, or more than one viral vector. Exemplary but non-limiting vectors combinations can include: DNA-derived plus RNA-derived vectors, RNA plus reverse transcriptase, a transposon and a transposase, a non-viral vectors plus an endonuclease, and a viral vector plus an endonuclease.

In some embodiments of the methods of the disclosure, introducing a nucleic acid sequence and/or a genomic editing construct into a cell, e.g., an immune cell ex vivo, in vivo, in vitro or in situ comprises a combination of vectors. Exemplary, non-limiting vector combinations include: viral and non-viral vectors, a plurality of non-viral vectors, or a plurality of viral vectors. Exemplary but non-limiting vectors combinations include: a combination of a DNA-derived and an RNA-derived vector, a combination of an RNA and a reverse transcriptase, a combination of a transposon and a transposase, a combination of a non-viral vector and an endonuclease, and a combination of a viral vector and an endonuclease.

In some embodiments of the methods of the disclosure, genome modification comprising introducing a nucleic acid sequence and/or a genomic editing into a cell, e.g., an immune cell ex vivo, in vivo, in vitro or in situ stably integrates a nucleic acid sequence, transiently integrates a nucleic acid sequence, produces site-specific integration a nucleic acid sequence, or produces a biased integration of a nucleic acid sequence. In some embodiments, the nucleic acid sequence is a transgene. In some embodiments, the stable chromosomal integration can be a random integration, a site-specific integration, or a biased integration. In some embodiments, the site-specific integration can be non-assisted or assisted. In some embodiments, the assisted site-specific integration is co-delivered with a site-directed nuclease. In some embodiments, the site-directed nuclease comprises a transgene with 5′ and 3′ nucleotide sequence extensions that contain a percentage homology to upstream and downstream regions of the site of genomic integration. In some embodiments, the transgene with homologous nucleotide extensions enables genomic integration by homologous recombination, microhomology-mediated end joining, or nonhomologous end-joining. In some embodiments the site-specific integration occurs at a safe harbor site. Potential genomic safe harbors include, but are not limited to, intronic sequences of the human albumin gene, the adeno-associated virus site 1 (AAVS1), a naturally occurring site of integration of AAV virus on chromosome 19, the site of the chemokine (C—C motif) receptor 5 (CCR5) gene and the site of the human ortholog of the mouse Rosa26 locus.

In some embodiments, the site-specific transgene integration occurs at a site that disrupts expression of a target gene. In some embodiments, disruption of target gene expression occurs by site-specific integration at introns, exons, promoters, genetic elements, enhancers, suppressors, start codons, stop codons, and response elements. In some embodiments, exemplary target genes targeted by site-specific integration include but are not limited to CISH, SOCS, PD1, any immunosuppressive gene, and genes involved in allo-rejection.

In some embodiments, the site-specific transgene integration occurs at a site that results in enhanced expression of a target gene. In some embodiments, enhancement of target gene expression occurs by site-specific integration at introns, exons, promoters, genetic elements, enhancers, suppressors, start codons, stop codons, and response elements.

### Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. A promoter used in the context of the present disclosure includes constitutive, inducible, and tissue-specific promoters.

The expression constructs provided herein comprise a promoter to drive expression of the engineered protein (e.g., chimeric protein), cytokine, CAR and/or other protein(s) provided herein. A promoter may or may not be used in conjunction with an “enhancer,” which refers to a cis-acting regulatory sequence involved in the transcriptional activation of a nucleic acid sequence.

The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e.g., beta actin promoter, GADPH promoter, metallothionein promoter; and concatenated response element promoters, such as cyclic AMP response element promoters (ere), serum response element promoter (sre), phorbol ester promoter (TPA) and response element promoters (tre) near a minimal TATA box. It is also possible to use human growth hormone promoter sequences (e.g., the human growth hormone minimal promoter described at GENBANK, accession no. X05244, nucleotide 283-341) or a mouse mammary tumor promoter. In certain embodiments, the promoter is EF1alpha, MND, CMV IE, dectin-1, dectin-2, human CD11c, F4/80, SM22, RSV, SV40, Ad MLP, beta-actin, MHC class I or MHC class II promoter, however any other promoter that is useful to drive expression of the therapeutic gene is applicable to the practice of the present disclosure.

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. The exogenous translational control signals and initiation codons can be either natural or synthetic.

In certain embodiments, internal ribosome entry sites (IRES) elements are used to create multigene, or polycistronic, messages. Additionally, certain 2A sequence elements could be used to create linked- or co-expression of genes in the constructs provided in the present disclosure. An exemplary cleavage sequence is the F2A (Foot-and-mouth disease virus 2A) or a “2A-like” sequence (e.g., Thosea asigna virus 2A; T2A) or a P2A (e.g., porcine teschovirus-1 2A).

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming. Alternatively, a replication origin of other extra-chromosomally replicating virus as described above or an autonomously replicating sequence (ARS) can be employed.

### Selection Markers

In some embodiments, cells containing a construct of the present disclosure may be identified in vitro or in vivo by including a marker (e.g., a positive selection marker or a negative selection marker) in the expression vector. An example of a positive selection marker is a drug resistance marker.

### Other Methods of Nucleic Acid Delivery

In addition to viral delivery of the nucleic acids encoding the engineered protein (e.g., chimeric protein), cytokine, CAR, and/or other protein, the following are additional methods of recombinant gene delivery to a given cell, e.g., an immune cell (e.g., NK cell), and are thus considered in the present disclosure.

Introduction of a nucleic acid, such as DNA or RNA, into the cells, e.g., immune cells of the current disclosure may use any suitable methods for nucleic acid delivery for transformation of a cell, as described herein or as would be known to one of ordinary skill in the art. Such methods include, but are not limited to, direct delivery of DNA such as by ex vivo transfection, by injection, including microinjection); by electroporation; by calcium phosphate precipitation; by using DEAE-dextran followed by polyethylene glycol; by direct sonic loading; by liposome mediated transfection and receptor-mediated transfection; by microprojectile bombardment; by agitation with silicon carbide fibers; by Agrobacterium-mediated transformation; by desiccation/inhibition-mediated DNA uptake, and any combination of such methods. Through the application of techniques such as these, organelle(s), cell(s), tissue(s), or organism(s) may be stably or transiently transformed.

### Transposition Based Methods of Modification

Generally, the gene transfer system can include a transposon or a viral integration system.

In some embodiments, a transposon may be present in an expression vector. In some embodiments, the expression vector can be a DNA plasmid. In some embodiments, the expression vector may be a mini-circle vector. The term “mini-circle vector” as used herein can refer to small circular plasmid derivative that is free of most, if not all, prokaryotic vector parts (e.g., control sequences or non-functional sequences of prokaryotic origin).

Exemplary transposons include, but are not limited to, piggyBac, hyperactive piggyBac, Sleeping Beauty (SB), hyperactive Sleeping Beauty (SB100x), SB11, SB110, Tn7, TcBuster™, hyperactive TcBuster, Frog Prince, IS5, TnlO, Tn903, SPIN, hAT, Hermes, Hobo, AeBuster1, AeBuster2, AeBuster3, BtBuster1, BtBuster2, CfBuster1, CfBuster2, Tol2, mini-Tol2, Tc3, Mos1, MuA, Himar I, Helitron and engineered versions of transposase family enzymes (Zhang et al., PLOS Genet. 5: e1000689, 2009; Wilson et al., J. Microbiol. Methods 71:332-5, 2007). Exemplary transposons also include the transposons described in Arensburger et al., Genetics 188(1):45-57, 2011, or a SPACE INVADERS (SPIN) transposon (see, e.g., Pace et al., Proc. Natl. Acad. Sci. U.S.A. 105(44):17023-17028, 2008). Alternatively, the gene transfer system can be integrated into the genome of a host cell using, for example, a retro-transposon, random plasmid integration, recombinase-mediated integration (e.g., using CRE recombinase), homologous recombination mediated integration, or non-homologous end joining mediated integration. More examples of transposition systems that can be used with certain embodiments of the compositions and methods provided herein include Staphylococcus aureus Tn552 (Colegio et al., J. Bacteriol. 183:2384-8, 2001; Kirby et al., Mol. Microbiol. 43:173-86, 2002), Tyl (Devine & Boeke, Nucleic Acids Res. 22:3765-72, 1994, and WO 95/23875), Transposon Tn7 (Craig, Science 271:1512, 1996; Craig, Review in: Curr. Top. Microbiol. Immunol. 204:27-48, 1996), Tn/O and IS10 (Kleckner et al., Curr. Top. Microbiol. Immunol. 204:49-82, 1996), Mariner transposase (Lampe et al., EMBO J. 15:5470-9, 1996), Tel (Plasterk, Curr. Topics Microbiol. Immunol. 204:125-43, 1996), P Element (Gloor, Methods Mol. Biol. 260:97-114, 2004), Tn3 (Ichikawa & Ohtsubo, J. Biol. Chem. 265:18829-32, 1990), bacterial insertion sequences (Ohtsubo & Sekine, Curr. Top. Microbiol. Immunol. 204:1-26, 1996), retroviruses (Brown et al., Proc. Natl. Acad. Sci. U.S.A. 86:2525-9, 1989), and retrotransposon of yeast (Boeke & Corces, Ann. Rev. Microbiol. 43:403-34, 1989).

Exemplary TcBuster family transposons include, but are not limited to Ac-like (AAC46515), Ac (CAA29005), AeBuster1 (ABF20543), AeBuster2 (ABF20544), AmBuster1 (EFB22616), AmBuster2 (EFB25016), AmBuster3 (EFB20710), AmBuster4 (EFB22020), BtBuster1 (ABF22695), BtBuster2 (ABF22700), BtBuster3 (ABF22697), CfBuster1 (ABF22696), CfBuster2 (ABF22701), CfBuster3 (XP_854762), CfBuster4 (XP_545451), CsBuster (ABF20548), Daysleeper (CAB68118), DrBuster1 (ABF20549), DrBuster2 (ABF20550), EcBuster1 (XP_001504971), EcBuster3 (XP_001503499), EcBuster4 (XP_001504928), Hermes (AAC37217), hermit (LCU22467), Herves (AAS21248), hobo (A39652), Homer (AAD03082), hopper-we (AAL93203), HsBuster1 (AAF18454), HsBuster2 (ABF22698), HsBuster3 (NP_071373), HsBuster4 (AAS01734), IpTip100 (BAA36225), MamBuster2 (XP_001108973), MamBuster3 (XP_001084430), MamBuster3 (XP_001084430), MamBuster4 (XP_001101327), MmBuster2 (AAF18453), PtBuster2 (ABF22699), PtBuster3 (XP_001142453), PtBuster4 (XP_527300), Restless (CAA93759), RnBuster2 (NP_001102151), SpBuster1 (ABF20546), SpBuster2 (ABF20547), SsBuster4 (XP_001929194), Tam3 (CAA38906), TcBuster (ABF20545), Tol2 (BAA87039), tramp (CAA76545), XtBuster (ABF20551), ENSEMBL (sequences available on the World Wide Web at ensembl.org), PtBuster1 (ENSPTRG00000003364), REPBASE (sequences available on the World Wide Web at girinst.org), Ac-like2 (hAT-7_DR), Ac-like1 (hAT-6_DR), hAT-5_DR (hAT-5_DR), MlBuster1 (hAT-4_ML), Myotis-hAT1 (Myotis-hAT1), SPIN_Et (SPIN_Et), SPIN_M1 (SPIN_M1), SPIN-Og (SPIN-Og), TEFam (sequences available on the World Wide Web at tefam.biochem.vt.edu), AeHermes1 (TF0013337), AeBuster3 (TF001186), AeBuster4 (TF001187), AeBuster5 (TF001188), AeBuster7 (TF001336), AeHermes2 (TF0013338), AeTip100-2 (TF000910), Cx-Kink2 (TF001637), Cx-Kink3 (TF001638), Cx-Kink4 (TF001639), Cx-Kink5 (TF001640), Cx-Kink7 (TF001636), Cx-Kink8 (TF001635).

Compositions and methods of the disclosure may comprise a TcBuster transposon and/or a TcBuster transposase. Compositions and methods of the disclosure may comprise a TcBuster transposon and/or a hyperactive TcBuster transposase. A hyperactive TcBuster transposase demonstrates an increased excision and/or increased insertion frequency when compared to an excision and/or insertion frequency of a wild type TcBuster transposase and/or increased transposition frequency when compared to a transposition frequency of a wild type TcBuster transposase. In some embodiments, a TcBuster transposase may comprise any of the mutations disclosed in WO 2019/246486, which is incorporated herein by reference in its entirety. In some embodiments of the compositions and methods of the disclosure, a wild type TcBuster transposase comprises or consists of the amino acid sequence of SEQ ID NO: 514. In some embodiments of the compositions and methods of the disclosure, a TcBuster transposase comprises or consists of a sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 99%, or any percentage identity in between to a wild type TcBuster transposase comprising or consisting of the amino acid sequence of SEQ ID NO: 515. In some embodiments of the compositions and methods of the disclosure, a wild type TcBuster transposase is encoded by a nucleic acid sequence comprising or consisting of SEQ ID NO: 516. In some embodiments of the compositions and methods of the disclosure, a TcBuster Transposase comprises or consists of a sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 99% or any percentage identity in between to a wild type TcBuster transposase encoded by a nucleic acid sequence comprising or consisting of SEQ ID NO: 517.

In some embodiments, an engineered cell (e.g., NK cell) produced by transposition-based methods may comprise sequences flanking the nucleotide sequence incorporated into the cell's genome by transposition. Illustrative examples of such flanking sequences (also known as excision footprints) are provided in Woodard et al., PLOS ONE 7(11): e42666, 2012.

### Other Methods of Modification

In some embodiments of the methods of the disclosure, a modified cell, e.g., immune cell, of the disclosure may be produced by introducing a transgene into a cell, e.g., an immune cell of the disclosure. The introducing step may comprise delivery of a nucleic acid sequence and/or a genomic editing construct via a non-transposition delivery system.

In some embodiments of the methods of the disclosure, introducing a nucleic acid sequence and/or a genomic editing into a cell, e.g., an immune cell ex vivo, in vivo, in vitro or in situ, comprises one or more of topical delivery, adsorption, absorption, electroporation, spin-fection, co-culture, transfection, mechanical delivery, sonic delivery, vibrational delivery, magnetofection, or by nanoparticle-mediated delivery. In some embodiments of the methods of the disclosure, introducing a nucleic acid sequence and/or a genomic editing construct into a cell, e.g., an immune cell ex vivo, in vivo, in vitro or in situ, comprises liposomal transfection, calcium phosphate transfection, fugene transfection, or dendrimer-mediated transfection. In some embodiments of the methods of the disclosure, introducing a nucleic acid sequence and/or a genomic editing construct into a cell, e.g., an immune cell ex vivo, in vivo, in vitro or in situ, by mechanical transfection comprises cell squeezing, cell bombardment, or gene gun techniques. In some embodiments of the methods of the disclosure, introducing a nucleic acid sequence and/or a genomic editing construct into a cell, e.g., an immune cell ex vivo, in vivo, in vitro or in situ, by nanoparticle-mediated transfection comprises liposomal delivery, delivery by micelles, or delivery by polymerosomes.

In some embodiments of the methods of the disclosure, introducing a nucleic acid sequence and/or a genomic editing construct into a cell, e.g., an immune cell ex vivo, in vivo, in vitro or in situ, comprises a non-viral vector. In some embodiments, the non-viral vector comprises a nucleic acid. In some embodiments, the non-viral vector comprises plasmid DNA, linear double-stranded DNA (dsDNA), linear single-stranded DNA (ssDNA), DoggyBone™ DNA, nanoplasmids, minicircle DNA, single-stranded oligodeoxynucleotides (ssODN), DDNA oligonucleotides, single-stranded mRNA (ssRNA), or double-stranded mRNA (dsRNA). In some embodiments, the non-viral vector comprises a transposon of the disclosure.

In some embodiments of the methods of the disclosure, enzymes may be used to create strand breaks in the host genome to facilitate delivery or integration of the transgene. In some embodiments, enzymes create single-strand breaks or double-strand breaks. In some embodiments, examples of break-inducing enzymes include but are not limited to: transposases, integrases, endonucleases, meganucleases, megaTALs, CRISPR-Cas9, CRISPR-CasX, transcription activator-like effector nucleases (TALEN), or zinc finger nucleases (ZFN). Other editing or break-inducing enzymes may include, without limitation, nucleases such as Cas12a (includes MAD7), Cas12b, Cas12c, Cas13, and many more. In certain instance, the Cas12a nuclease is MAD7. In some embodiments, break-inducing enzymes can be delivered to the cell encoded in DNA, encoded in mRNA, as a protein, or as a nucleoprotein complex with a guide RNA (gRNA).

In some embodiments of the methods of the disclosure, the site-specific transgene integration is controlled by a vector-mediated integration site bias, by the chosen lentiviral vector and/or by the chosen gamma-retroviral vector. In some embodiments of the methods of the disclosure, the site-specific transgene integration site is a non-stable chromosomal insertion. In some embodiments, the integrated transgene may become silenced, removed, excised, or further modified.

In some embodiments of the methods of the disclosure, the genome modification is a non-stable integration of a transgene (e.g., transient non-chromosomal integration (e.g., epi-chromosomal or cytoplasmic), a semi-stable non chromosomal integration, a semi-persistent non-chromosomal insertion, or a non-stable chromosomal insertion). In some embodiments, the transient non-chromosomal insertion of a transgene does not integrate into a chromosome and the modified genetic material is not replicated during cell division.

In some embodiments of the methods of the disclosure, the genome modification is a semi-stable or persistent non-chromosomal integration of a transgene. In some embodiments, a DNA vector encodes a Scaffold/matrix attachment region (S-MAR) module that binds to nuclear matrix proteins for episomal retention of a non-viral vector allowing for autonomous replication in the nucleus of dividing cells.

In some embodiments of the methods of the disclosure, the modification to the genome by transgene insertion can occur via host cell-directed double-strand breakage repair (homology-directed repair) by homologous recombination (HR), microhomology-mediated end joining (MMEJ), nonhomologous end joining (NHEJ), transposase enzyme-mediated modification, integrase enzyme-mediated modification, endonuclease enzyme-mediated modification, or recombinant enzyme-mediated modification.

### Nanoparticle Delivery

In some embodiments intracellular delivery of gene editing tools is enabled by complexing with poly(histidine)-based micelles, e.g., comprising triblock copolymers made of a hydrophilic block, a hydrophobic block, and a charged block. In some embodiments, the hydrophilic block may be poly(ethylene oxide) (PEO), and the charged block may be poly(L-histidine). An example tri-block copolymer that may be used in various embodiments is a PEO-b-PLA-b-PHIS, with variable numbers of repeating units in each block varying by design. Diblock copolymers that may be used as intermediates for making triblock copolymers of the micelles may have hydrophilic biocompatible poly(ethylene oxide) (PEO), which is chemically synonymous with PEG, coupled to various hydrophobic aliphatic poly(anhydrides), poly(nucleic acids), poly(esters), poly(ortho esters), poly(peptides), poly(phosphazenes) and poly(saccharides), including but not limited by poly(lactide) (PLA), poly(glycolide) (PLGA), poly(lactic-co-glycolic acid) (PLGA), poly(ε-caprolactone) (PCL), and poly(trimethylene carbonate) (PTMC).

In certain embodiments of the methods of the disclosure, a cell with an ex vivo, in vivo, in vitro or in situ genomic modification can be a germline cell or a somatic cell. In certain embodiments the modified cell can be a human, non-human, mammalian, rat, mouse, or dog cell. In certain embodiments, the modified cell can be differentiated, undifferentiated, or immortalized. In certain embodiments, the modified undifferentiated cell can be a stem cell. In certain embodiments, the modified cell can be differentiated, undifferentiated, or immortalized. In certain embodiments, the modified undifferentiated cell can be an induced pluripotent stem cell. In certain embodiments, the modified cell can be a T cell, a hematopoietic stem cell, a natural killer cell, a macrophage, a dendritic cell, a monocyte, or a megakaryocyte. In certain embodiments, the modified cell can be modified while the cell is quiescent, in an activated state, resting, in interphase, in prophase, in metaphase, in anaphase, or in telophase. In certain embodiments, the modified cell can be fresh, cryopreserved, bulk, sorted into sub-populations, from whole blood, from leukapheresis, or from an immortalized cell line.

### Click Chemistry

Engineered cells, e.g., immune cells (e.g., NK cells), described herein can also be produced using coupling reagents to link an exogenous polypeptide (cytokine, targeting moiety etc.) to a cell with the use of click chemistry reactions. Coupling reagents can be used to couple an exogenous polypeptide to a cell, for example, when the exogenous polypeptide is a complex or difficult to express polypeptide, e.g., a polypeptide, e.g., a multimeric polypeptide; large polypeptide; polypeptide derivatized in vitro; an exogenous polypeptide that may have toxicity to, or which is not expressed efficiently in, the immune cells, e.g., NK cells.

The click chemistry approach was originally conceived as a method to rapidly generate complex substances by joining small subunits together in a modular fashion. (See, e.g., Kolb et al., Angew Chem. Int. Ed. 40:3004-31, 2004; Evans, Aust. J. Chem. 60:384-95, 2007.) Various forms of click chemistry reaction are known in the art, such as the Huisgen 1,3-dipolar cycloaddition copper catalyzed reaction (Tornoe et al., J. Organic Chem. 67:3057-64, 2002), which is often referred to as the “click reaction.” Other alternatives include cycloaddition reactions such as the Diels-Alder, nucleophilic substitution reactions (especially to small strained rings like epoxy and aziridine compounds), carbonyl chemistry formation of urea compounds and reactions involving carbon-carbon double bonds, such as alkynes in thiol-yne reactions. In some embodiments, the click chemistry approach comprises copper catalyzed reaction, as described, e.g., in Rostovstev et al., 2002, Angew Chem Int Ed 41:2596, 2002; Tomoe et al., J. Org. Chem. 67:3057, 2002. In other embodiments, the click chemistry approach comprises copper-free click reaction, as described, e.g., by Agard et al. (J. Am. Chem. Soc. 126:15046-47, 2004) and Ning et al. (Angew Chem. Int. Ed. 49:3065-68, 2010).

### Enzymatic Conjugation

In some embodiments, the exogenous polypeptide can be conjugated to the surface of a cell, e.g., an immune cell (e.g., an NK cell) by various chemical and enzymatic means, including but not limited to chemical conjugation with bifunctional cross-linking agents such as, e.g., an NHS ester-maleimide heterobifunctional crosslinker to connect a primary amine group with a reduced thiol group. These methods also include enzymatic strategies such as, e.g., transpeptidase reaction mediated by a sortase enzyme.

Sortase transpeptidation, also known as “sortase labeling” or “sortagging,” can be used for bioconjugation of two proteins. Methods compositions disclosed herein can use or include a sortase from any bacterial species or strain, e.g., a sortase A, a sortase B, a sortase C, a sortase D, a sortase E, a sortase F, or a sortase from a yet unidentified class of sortase enzymes (e.g., as described in Dramsi et al., Res. Microbiol. 156(3): 289-97, 2005; Comfort and Clubb, Infect. Immun. 72(5): 2710-22, 2004; and Spirig et al., Mol. Microbiol., 2011). The methods described herein can be used to evaluate candidate sortases. The amino acid sequences of many sortases and the nucleotide sequences that encode them are known to those of skill in the art and are disclosed in many of the references cited herein. The amino acid sequence of full-length, wild-type S. aureus Sortase A is SEQ ID NO: 518.

Mutant sortase molecules can be used to form engineered protein (e.g., chimeric protein) members, e.g., in situ on cells, e.g., immune cells, that comprise a sortase acceptor motif. An exemplary sortase mutant, which is efficient, and not dependent on non-physiological reaction conditions, is S. aureus sortase A mutant [P94R/E105K/E108Q/D160N/D165A/K190E/K196T]. It lacks the N-terminal 59 amino acids of S. aureus sortase A and includes mutations that render the enzyme calcium-independent and which make the enzyme faster (the amino acid residue number herein begins with residue the first residue at the N terminal end of non-truncated S. aureus sortase A). The primary amino acid sequence of this mutant is provided below. Mutations are in bold. The primary amino acid sequence of sortase A mutant [P94R/E105K/E108Q/D160N/D165A/K190E/K196T] comprises the amino acid sequence of SEQ ID NO: 519.

In some embodiments, the sortase recognition motif is LPXTG (SEQ ID NO: 520) or LPXTA (SEQ ID NO: 521) and the sortase acceptor motif is N-terminal donor sequence GGG, resulting in the sortase transfer signature that comprises LPXTGG (SEQ ID NO: 4) after the sortase-mediated reaction. The methods also include combination methods, such as e.g., sortase-mediated conjugation of click chemistry handles or “click handles” (an azide and an alkyne) on a protein and the cell, respectively, followed by a cyclo-addition reaction to chemically bond the antigen to the cell, see e.g., Neves et al., Bioconjugate Chemistry, 2013. Sortase-mediated modification of proteins is further described in PCT/US2014/037545, PCT/US2014/037554, and WO2016014553, each of which are incorporated by reference in their entireties herein. In some embodiments, a protein is modified by the conjugation of a sortase substrate comprising an amino acid, a peptide, a protein, a polynucleotide, a carbohydrate, a tag, a metal atom, a contrast agent, a catalyst, a non-polypeptide polymer, a recognition element, a small molecule, a lipid, a linker, a label, an epitope, an antigen, a therapeutic agent, a toxin, a radioisotope, a particle, or moiety comprising a reactive chemical group, e.g., a click chemistry handle.

If desired, a catalytic bond-forming polypeptide domain can be expressed on an immune cells, e.g., an NK cell, extracellularly. Many catalytic bond-forming polypeptides exist, including transpeptidases, sortases, and isopeptidases, including those derived from Spy0128 (e.g., SpyTag and SpyCatcher), a protein isolated from Streptococcus pyogenes. The components Spy Tag and SpyCatcher can be interchanged such that a system in which molecule A is fused to SpyTag and molecule B is fused to SpyCatcher is functionally equivalent to a system in which molecule A is fused to SpyCatcher and molecule B is fused to SpyTag. For the purposes of this disclosure, when Spy Tag and SpyCatcher are used, it is to be understood that the complementary molecule could be substituted in its place.

A catalytic bond-forming polypeptide, such as a SpyTag/SpyCatcher system, can be used to attach the exogenous polypeptide to the surface of a cell, e.g., a NK cell, to make an engineered cell, e.g., NK cell. The SpyTag polypeptide sequence can be expressed on the extracellular surface of the cell, e.g., NK cell. The SpyTag polypeptide can be, for example, fused to the N-terminus of a transmembrane protein, e.g., inserted in-frame at the extracellular terminus or in an extracellular loop of a multi-pass transmembrane protein, fused to a lipid-chain-anchored polypeptide, or fused to a peripheral membrane protein. The nucleic acid sequence encoding the SpyTag fusion can be expressed within an engineered cell, e.g., NK cell. An exogenous stimulatory polypeptide can be fused to SpyCatcher. The nucleic acid sequence encoding the SpyCatcher fusion can be expressed and secreted from the same cell, e.g., a NK cell, that expresses the Spy Tag fusion. Alternatively, the nucleic acid sequence encoding the SpyCatcher fusion can be produced exogenously, for example in a bacterial, fungal, insect, mammalian, or cell-free production system. Upon reaction of the SpyTag and SpyCatcher polypeptides, a covalent bond will be formed that attaches the exogenous stimulatory polypeptide to the surface of the cell, e.g., NK cell to form an engineered cell, e.g., NK cell.

### Methods of Modified Cell Cryopreservation

In some embodiments of the present disclosure, the cells, e.g., immune cells described herein are modified at a point-of-care site. In some cases, the point-of-care site is at a hospital or at a facility (e.g., a medical facility) near a subject in need of treatment. The subject undergoes apheresis and peripheral blood mononuclear cells (PBMCs) or a sub population of PBMC can be enriched for example, by elutriation or Ficoll separation. Enriched PBMC or a subpopulation of PBMC can be cryopreserved in any appropriate cryopreservation solution prior to further processing. In one instance, the elutriation process is performed using a buffer solution containing human serum albumin. Immune cells, such as NK cells, can be isolated by selection methods described herein. In one instance, the selection method for NK cells includes beads specific for CD56 on NK cells. In one case, the beads can be paramagnetic beads. The harvested immune cells can be cryopreserved in any appropriate cryopreservation solution prior to modification. The immune cells can be thawed up to 24 hours, 36 hours, 48 hours, 72 hours, or 96 hours ahead of infusion. The thawed cells can be placed in cell culture buffer, for example in cell culture buffer (e.g., RPMI) supplemented with fetal bovine serum (FBS) or human serum AB or placed in a buffer that includes cytokines such as IL-2 and IL-21, prior to modification. In another aspect, the harvested immune cells can be modified immediately without the need for cryopreservation.

In one aspect, the population of genetically modified cells is cryopreserved prior to infusion into a subject. Genetically modified NK cells that are thawed following cryopreservation maintain their ability to bind to the negative signal. In some embodiments, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98% or at least 99% of the cryopreserved genetically modified cells maintain their ability to bind to the negative signal after thawing.

In one aspect, the population of genetically modified cells is immediately infused into a subject. In another aspect, the population of genetically modified NK cells is placed in a cytokine bath prior to infusion into a subject. In a further aspect, the population of genetically modified cells is cultured and/or stimulated for no more than 1, 2, 3, 4, 5, 6, 7, 14, 21, 28, 35, 42, 49, 56, 63, or 70 days.

In some embodiments, the modified cells, e.g., immune cells do not undergo a propagation and activation step. In some embodiments, the modified cells, e.g., immune cells do not undergo an incubation or culturing step (e.g., ex vivo propagation). In some embodiments, the immune cells are expanded in culture before administration to the subject. In certain cases, the modified immune cells are placed in a buffer that includes IL-2 and IL-21 prior to infusion. In other instances, the modified immune cells are placed or rested in cell culture buffer, for example in cell culture buffer (e.g., RPMI) supplemented with fetal bovine serum (FBS) prior to infusion. Prior to infusion, the modified immune cells can be harvested, washed, and formulated in saline buffer in preparation for infusion into the subject.

### Modification of Gene Expression

In some embodiments, the cells, e.g., immune cells of the present disclosure are modified to have altered expression of certain genes. In some embodiments, the immune cells of the present disclosure are modified to have altered expression, e.g., reduced expression, of certain genes such as glucocorticoid receptor, a TGF-beta receptor (e.g., TGF-BR2), CISH, PTEN, PD-1, SHP-1, Cbl-b, adenosine receptor A2A, adenosine receptor A2B, prostaglandin receptor EP2, prostaglandin receptor EP4, HIF-1alpha, SHP-2, c-Cbl, GRAIL, Itch, SHIP-1, SHIP-2, SOCS1, SOCS2, SOCS3, SOCS4, SOCS5, SOCS6, and/or SOC7. In some embodiments, the immune cells may be modified to express a dominant negative isoform of a TGF beta receptor I or II (e.g., as listed in Tables 6 and 7) to deplete endogenous TGF beta.

In some embodiments, SOCS family proteins encoded by the CISH gene are knocked out in immune cells to improve cytotoxicity, such as in NK cells. Exemplary SOCS family of proteins include, but are not limited to SOCS1, SOCS2, SOCS3, SOCS4, SOCS5, SOCS6, SOCS7 and CISH. This approach may be used alone or in combination with other checkpoint inhibitors to improve anti-tumor activity.

In some embodiments, the altered gene expression is carried out by effecting a disruption in the gene, such as a knock-out, insertion, missense, or frameshift mutation, such as biallelic frameshift mutation, deletion of all or part of the gene, e.g., one or more exon or portion therefore, and/or knock-in. For example, the altered gene expression can be effected by sequence-specific or targeted nucleases, including DNA-binding targeted nucleases such as zinc finger nucleases (ZFN), transcription activator-like effector nucleases (TALENs), and RNA-guided nucleases such as a CRISPR-associated nuclease (Cas), specifically designed to be targeted to the sequence of the gene or a portion thereof.

In some embodiments, the alteration of the expression, activity, and/or function of the gene is carried out by disrupting the gene. In some embodiments, the gene is modified so that its expression is reduced by at least 20%, at least 30%, or at least 40%, generally at least 50%, at least 60%, at least 70%, at least 80%, at least 90%, or at least 95% as compared to the expression in the absence of the gene modification or in the absence of the components introduced to effect the modification.

In some embodiments, the alteration is transient or reversible, such that expression of the gene is restored at a later time. In other embodiments, the alteration is not reversible or transient, e.g., is permanent.

In some embodiments, gene alteration is carried out by induction of one or more double-stranded breaks and/or one or more single-stranded breaks in the gene, typically in a targeted manner. In some embodiments, the double-stranded or single-stranded breaks are made by a nuclease, e.g., an endonuclease, such as a gene-targeted nuclease (e.g., a DNA targeting molecule described herein). In some embodiments, the breaks are induced in the coding region of the gene, e.g., in an exon. For example, in some embodiments, the induction occurs near the N-terminal portion of the coding region, e.g., in the first exon, in the second exon, or in a subsequent exon.

In some embodiments, the double-stranded or single-stranded breaks undergo repair via a cellular repair process, such as by non-homologous end-joining (NHEJ) or homology-directed repair (HDR). In some embodiments, the repair process is error-prone and results in disruption of the gene, such as a frameshift mutation, e.g., biallelic frameshift mutation, which can result in complete knockout of the gene. For example, in some embodiments, the disruption comprises inducing a deletion, mutation, and/or insertion. In some embodiments, the disruption results in the presence of an early stop codon. In some embodiments, the presence of an insertion, deletion, translocation, frameshift mutation, and/or a premature stop codon results in disruption of the expression, activity, and/or function of the gene.

In some embodiments, gene alteration is achieved using antisense techniques, such as by RNA interference (RNAi), short interfering RNA (siRNA), short hairpin (shRNA), and/or ribozymes are used to selectively suppress or repress expression of the gene. In some aspects, the siRNA is comprised in a polycistronic construct.

In some embodiments, the DNA-targeting molecule includes a DNA-binding protein such as one or more zinc finger protein (ZFP) or transcription activator-like protein (TAL), fused to an effector protein such as an endonuclease. Examples include ZFNs, TALEs, and TALENs. Many gene-specific engineered zinc fingers are available commercially.

In some embodiments, the DNA-targeting molecule comprises a naturally occurring or engineered (non-naturally occurring) transcription activator-like protein (TAL) DNA binding domain, such as in a transcription activator-like protein effector (TALE) protein, see, e.g., U.S. Patent Publication No. 2011/0301073, incorporated by reference in its entirety herein. In some embodiments, TALEs may be targeted to any gene by design of TAL arrays with specificity to the target DNA sequence. In some embodiments, the TALEN is a fusion protein comprising a DNA-binding domain derived from a TALE and a nuclease catalytic domain to cleave a nucleic acid target sequence. Exemplary molecules are described, e.g., in U.S. Patent Publication Nos. US 2014/0120622 and 2013/0315884. In some embodiments the TALENs are introduced as trans genes encoded by one or more plasmid vectors.

In certain embodiments, the nuclease comprises a meganuclease (homing endonuclease) or a portion thereof that exhibits cleavage activity. Exemplary meganucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. Their recognition sequences are known. See also U.S. Pat. Nos. 5,420,032; 6,833,252; Belfort et al., Nucleic Acids Res. 25:3379-88, 1997; Dujon et al., Gene 82:115-8, 1989; Perler et al., Nucleic Acids Res. 22:1125-7, 1994; Jasin, Trends Genet. 12:224-8, 1996; Gimble et al., J. Mol. Biol. 263:163-80, 1996; and Argast et al., J. Mol. Biol. 280:345-53, 1998.

In some embodiments, the alteration is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus. The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some embodiments, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. Typically, “target sequence” refers to a sequence to which a guide sequence is designed to have complementarity, where hybridization between the target sequence and a guide sequence promotes the formation of a CRISPR complex.

The CRISPR system can induce double stranded breaks (DSBs) at the target site, followed by disruptions or alterations as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence.” In some embodiments, an exogenous template polynucleotide may be referred to as an editing template.

The components of a CRISPR system can be implemented in any suitable manner, meaning that the components of such systems including the RNA-guided nuclease (e.g., Cas enzyme) and gRNA can be delivered, formulated, or administered in any suitable form to the cells. For example, the RNA-guided nuclease may be delivered to a cell complexed with a gRNA (e.g., as a ribonucleoprotein (RNP) complex), the RNA-guided nuclease may be delivered to a cell separate (e.g., uncomplexed) to a gRNA, the RNA-guided nuclease may be delivered to a cell as a polynucleotide (e.g., DNA or RNA) encoding the nuclease that is separate from a gRNA, or both the RNA-guided nuclease and the gRNA molecule may be delivered as polynucleotides encoding each component.

One or more vectors driving expression of one or more elements of the CRISPR system can be introduced into the cell such that expression of the elements of the CRISPR system direct formation of the CRISPR complex at one or more target sites. Components can also be delivered to cells as ribonucleoprotein complexes, proteins, DNA, and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas8a, Cas8b, Cas8c, Cas9 (also known as Csn1 and Csx12), Cas10, Cas10d, Cas12, Cas12a (Cpf1), Cas12b (C2c1), Cas12c (C2c3), Cas12d (CasY), Cas12e (CasX), Cas12f (Cas14, C2c10), Cas12g, Cas12h, Cas12i, Cas12k (C2c5), C2c4, C2c8, C2c9, Cas13, Cas13a (C2c2), Cas13b, Cas13c, Cas13d, CasX, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx11, Csx16, CsaX, Csx3, Csx1, Csx15, Csf1, Csf2, Csf3, Csf4, MAD7, GSU0054, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some instances, the CRISPR enzyme can be Cas12a nuclease, such as MAD7. MAD7 is an engineered nuclease of the Class 2 type V-A CRISPR-Cas (Cas12a/Cpf1) family with a low level of homology to canonical Cas12a nucleases. MAD7 only requires a crRNA for gene editing and allows for specific targeting of AT rich regions of the genome. MAD7 cleaves DNA with a staggered cut as compared to S. pyogenes which has blunt cutting. The PAM sequence is YTTV, wherein Y indicates a C or T base, and V indicates A, C or G. The DNA cleavage sites for MAD7 relative to the target site are 19 bases after the YTTV PAM site on the sense strand and 23 bases after the complementary PAM site of the anti-sense strand.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Exemplary gRNA sequences for NR3CS (glucocorticoid receptor) include Ex3 NR3C1 sG1 5-TGC TGT TGA GGA GCT GGA-3 (SEQ ID NO: 522) and Ex3 NR3C1 sG2 5-AGC ACA CCA GGC AGA GTT-3 (SEQ ID NO: 523). Exemplary gRNA sequences for TGF-β receptor 2 include EX3 TGF-BR2 sG1 5-CGG CTG AGG AGC GGA AGA-3 (SEQ ID NO: 524) and EX3 TGF-BR2 sG2 5-TGG-AGG-TGA-GCA-ATC-CCC-3 (SEQ ID NO: 525). The T7 promoter, target sequence, and overlap sequence may have the sequence TTAATACGACTCACTATAGG (SEQ ID NO: 526)+target sequence+gttttagagctagaaatage (SEQ ID NO: 527).

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 2011/0059502, incorporated herein by reference.

### V. Methods of Use

In some embodiments, the present disclosure relates to methods of treating a disease or pathological condition in a subject, comprising administering to the subject an effective amount of the cells, e.g., immune cells of the present disclosure. In some embodiments, the present disclosure provides methods of modulating (e.g., increasing) an immune response in a subject in need thereof, comprising administering to the subject an effective amount of the immune cells of the present disclosure. In some embodiments, the present disclosure provides methods of treating a subject in need of an altered immune response, comprising administering an effective amount of the immune cells of the present disclosure. In some embodiments, the present disclosure provides methods of treating a subject in need of an increased immune response, comprising administering an effective amount of the immune cells of the present disclosure.

In some embodiments, the present disclosure provides methods for immunotherapy comprising administering an effective amount of the immune cells of the present disclosure. In some embodiments, a medical disease or disorder is treated by transfer of an immune cell population (e.g., an immune cell population provided herein) that elicits an immune response. In certain embodiments of the present disclosure, cancer or infection is treated by transfer of an immune cell population (e.g., an immune cell population provided herein) that elicits an immune response. Provided herein are methods for treating or delaying progression of cancer in an individual comprising administering to the individual an effective amount an antigen-specific cell therapy. The present methods may be applied for the treatment of immune disorders, solid cancers, hematologic cancers, and viral infections.

Tumors for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous bistiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's; paragranuloma; malignant lymphoma, small lymphocytic; malignant lymphoma, large cell, diffuse; malignant lymphoma, follicular; mycosis fungoides; other specified non-Hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML; e.g., relapsed AML or refractory AML); myelodysplastic syndrome (MDS); chronic myeloblasts leukemia; diffuse large B-cell lymphoma (DLBCL); peripheral T-cell lymphoma (PTCL); or anaplastic large cell lymphoma (ALCL).

In certain embodiments of the present disclosure, cells, e.g., immune cells are delivered to an individual in need thereof, such as an individual that has cancer or an infection. The cells then enhance the individual's immune system to attack or directly attack the respective cancer or pathogenic cells. In some cases, the individual is provided with one or more doses of the cells, e.g., immune cells. In cases where the individual is provided with two or more doses of the cells, e.g., immune cells, the duration between the administrations should be sufficient to allow time for propagation in the individual, and in specific embodiments the duration between doses is 1, 2, 3, 4, 5, 6, 7, 8, 9, 10, 11, or 12 or more weeks.

In some embodiments, the subject can be administered nonmyeloablative lymphodepleting chemotherapy prior to the immune cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine. An exemplary route of administering cyclophosphamide and fludarabine is intravenously. Likewise, any suitable dose of cyclophosphamide and fludarabine can be administered. In particular embodiments, around 60 mg/kg of cyclophosphamide is administered for two days after which around 25 mg/m² fludarabine is administered for five days.

The nonmyeloablative lymphodepleting immunotherapy can comprise, for example, the administration of an anti-CD52 agent or anti-CD20 agent. In some embodiments, the lymphodepleting immunotherapy is an anti-CD52 antibody. In some embodiments, the anti-CD52 antibody is alemtuzumab. In some embodiments, the lymphodepleting immunotherapy is an anti-CD20 antibody. Exemplary anti-CD20 antibodies include, but are not limited to rituximab, ofatumumab, ocrelizumab, obinutuzumab, ibritumomab or iodine I-131 tositumomab. An exemplary route of administering anti-CD52 agent or anti-CD20 agent is intravenously. Likewise, any suitable dose of anti-CD52 agent or anti-agent can be administered.

In certain embodiments, a growth factor that promotes the growth and activation of the immune cells is administered to the subject either concomitantly with the immune cells or subsequently to the immune cells. The immune cell growth factor can be any suitable growth factor that promotes the growth and activation of the immune cells. Examples of suitable immune cell growth factors include IL-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

Therapeutically effective amounts of immune cells can be administered by a number of routes, including parenteral administration, for example, intravenous, intraperitoneal, intramuscular, intrasternal, or intraarticular injection, or infusion.

The therapeutically effective amount of immune cells for use in adoptive cell therapy is that amount that achieves a desired effect in a subject being treated. For instance, this can be the amount of immune cells necessary to inhibit advancement, or to cause regression of a disease, e.g., cancer, or which is capable of relieving symptoms caused by a disease, e.g., cancer. It can be the amount necessary to relieve symptoms associated with the disease, e.g., cancer.

The cell, e.g., immune cell, or a population of the cells can be administered in treatment regimens consistent with the disease, for example a single or a few doses over one to several weeks to ameliorate a disease state or periodic doses over an extended time to inhibit disease progression and prevent disease recurrence. The precise dose to be employed in the formulation will also depend on the route of administration, and the seriousness of the disease or disorder. The therapeutically effective amount of cells, e.g., immune cells, will be dependent on the subject being treated, the severity and type of the affliction, and the manner of administration. The exact amount of cells, e.g., immune cells, is readily determined by one of skill in the art based on the age, weight, sex, and physiological condition of the subject. Effective doses can be extrapolated from dose response curves derived from in vitro or animal model test systems.

The cells, e.g., immune cells, may be administered in combination with one or more other therapeutic agents for the treatment of the immune-mediated disorder. Combination therapies can include, but are not limited to, one or more anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents, such as acetyls alicylic acid, ibuprofen or naproxen sodium), cytokine antagonists (for example, anti-TNF and anti-IL-6), cytokines (for example, interleukin-10 or transforming growth factor-beta), hormones (for example, estrogen), or a vaccine. In addition, immunosuppressive or tolerogenic agents including but not limited to calcineurin inhibitors (e.g., cyclosporin and tacrolimus); mTOR inhibitors (e.g., rapamycin); mycophenolate mofetil, antibodies (e.g., recognizing CD3, CD4, CD40, CD154, CD45, IVIG, or B cells); chemotherapeutic agents (e.g., methotrexate, treosulfan, or busulfan); irradiation; or chemokines, interleukins or their inhibitors (e.g., BAFF, IL-2, anti-IL-2R, IL-4, or JAK kinase inhibitors) can be administered. Such additional pharmaceutical agents can be administered before, during, or after administration of the cells, e.g., immune cells, depending on the desired effect. This administration of the cells and the agent can be by the same route or by different routes, and either at the same site or at a different site.

### VI. Pharmaceutical Compositions

Also provided herein are pharmaceutical compositions and formulations comprising cells, e.g., immune cells (e.g., NK cells) and a pharmaceutically acceptable carrier.

In some embodiments, a pharmaceutical composition comprises a dose ranging from about 1×10⁵ immune cells (e.g., NK cells) to about 1×10⁹ immune cells (e.g., NK cells). In some embodiments, the dose is about 1×10⁵, 1×10⁶, 1×10⁷, 1×10⁸, or 1×10⁹ immune cells (e.g., NK cells). In some embodiments, a pharmaceutical composition comprises a dose ranging from about 5×10⁷ immune cells (e.g., NK cells) to about 10×10¹² immune cells (e.g., NK cells).

In some embodiments, a pharmaceutical composition is cryopreserved. In some embodiments, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% %, at least 91%, at least 92%, at least 93%, at least 94%, at least 95%, at least 96%, at least 97%, at least 98%, or at least 99% of the immune cells (e.g., NK cells) in the cryopreserved pharmaceutical composition specifically bind the cognate binding partner of the extracellular domain of the engineered protein (e.g., chimeric protein), e.g., human TGF-β, after thawing. Pharmaceutical compositions and formulations as described herein can be prepared by mixing the cells, e.g., immune cells (e.g., NK cells), with one or more optional pharmaceutically acceptable carriers (Remington's Pharmaceutical Sciences 22^{nd} edition, 2012), in the form of aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride); low molecular weight (less than about 10 residues) polypeptides; proteins, such as serum albumin or immunoglobulins; amino acids such as glycine, glutamine, asparagine, histidine, arginine, or lysine; monosaccharides, disaccharides, and other carbohydrates including glucose, mannose, or dextrins; and/or sugars such as sucrose, mannitol, trehalose or sorbitol.

### VII. Combination Therapies

In some embodiments, the compositions and methods of the present embodiments involve administration of a cell, e.g., an immune cell, or a population of the cells in combination with at least one additional therapy. The additional therapy may be radiation therapy, surgery (e.g., lumpectomy or a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is a combination of radiation therapy and surgery. In some embodiments, the additional therapy is gamma irradiation. The additional therapy may be one or more of the chemotherapeutic agents known in the art.

An immune cell therapy may be administered before, during, after, or in various combinations relative to an additional cancer therapy, such as immune checkpoint therapy. The administrations may be in intervals ranging from concurrently to minutes to days to weeks. In some embodiments where the immune cell therapy is provided to a subject separately from an additional therapeutic agent, one would generally ensure that a significant period of time did not expire between the time of each delivery, such that the two compounds would still be able to exert an advantageously combined effect on the subject. In such instances, it is contemplated that one may provide a subject with the antibody therapy and the anti-cancer therapy within about 12 to 24 or 72 hours of each other and, more particularly, within about 6-12 hours of each other. In some situations, it may be desirable to extend the time period for treatment significantly where several days (2, 3, 4, 5, 6, or 7 days) to several weeks (1, 2, 3, 4, 5, 6, 7, or 8 weeks) lapse between respective administrations.

Administration of any compound or therapy of the present embodiments to a subject will follow general protocols for the administration of such compounds, taking into account the toxicity, if any, of the agents. Therefore, in some embodiments there is a step of monitoring toxicity that is attributable to combination therapy.

### Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (e.g., bullatacin and bullatacinone); a camptothecin (including the synthetic analogue topotecan); bryostatin; callystatin; CC-1065 (including its adozelesin, carzelesin and bizelesin synthetic analogues); cryptophycins (e.g., cryptophycin 1 and cryptophycin 8); dolastatin; duocarmycin (including the synthetic analogues KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, e.g., calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-etbylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids, such as retinoic acid; capecitabine; carboplatin, procarbazine, plicomycin, gemcitabien, navelbine, farnesyl-protein transferase inhibitors, transplatinum, and pharmaceutically acceptable salts, acids, or derivatives of any of the above.

### Radiotherapy

In some embodiments, the additional therapy is radiotherapy including what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

### Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells.

In some embodiments, the immunotherapy comprises administration of an antibody-drug conjugate (e.g., brentuximab vedotin and trastuzumab emtansine).

In one aspect of immunotherapy, the tumor cell must bear some marker that is amenable to targeting, i.e., is not present on the majority of other cells. In some embodiments, the marker is CD20, carcinoembryonic antigen, tyrosinase (p97), gp68, TAG-72, HMFG, Sialyl Lewis Antigen, MucA, MucB, PLAP, laminin receptor, erb B, or p155. In some embodiments, the immunotherapy includes administration of cytokines, such as IL-2, IL-4, IL-12, GM-CSF, gamma-IFN, chemokines, such as MIP-1, MCP-1, IL-8, and growth factors, such as FLT3 ligand.

In some embodiments, the additional immunotherapy for use in combination or in conjunction with the methods described herein is an immune adjuvant, e.g., Mycobacterium bovis, Plasmodium falciparum, dinitrochlorobenzene, and aromatic compounds (U.S. Pat. Nos. 5,801,005 and 5,739,169; Hui and Hashimoto, Infect. Immun. 66(11): 5329-36, 1998; Christodoulides et al. Microbiology (Reading) 144 (Pt 11): 3027-37, 1998); a cytokine therapy, e.g., interferons α, β, and γ, IL-1, GM-CSF, and TNF (Bukowski et al. Clin. Cancer Res. 4(10): 2337-47, 1998; Davidson et al. J. Immunother. 21(5): 389-9, 1998; Hellstrand et al. Acta Oncol. 37(4): 347-53, 1998); a gene therapy, e.g., TNF, IL-1, IL-2, and p53 (Qin et al. Proc. Nat'l. Acad. Sci. USA 95(24): 14411-6, 1998; Austin-Ward and Villaseca, Rev. Med. Chil. 126(7): 838-45, 1998; U.S. Pat. Nos. 5,830,880 and 5,846,945); and a monoclonal antibody(ies), e.g., anti-CD20, anti-ganglioside GM2, and anti-p185 (Hollander Front Immunol. 3:3, 2012; Hanibuchi et al. Int. J. Cancer 78(4): 480-5, 1998; U.S. Pat. No. 5,824,311).

In some embodiments, the immunotherapy may be an immune checkpoint inhibitor. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, antibodies (e.g., pembrolizumab), such as human antibodies (e.g., WO 2015/016718; Pardoll, Nat. Rev. Cancer 12(4): 252-64, 2012; both incorporated herein by reference). Known inhibitors of the immune checkpoint proteins or analogs thereof may be used, in particular chimerized, humanized or human forms of antibodies may be used.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen-binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Application Publication No. 2014/0294898, 2014/0022021, and 2011/0008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of AMP-224, nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence)).

Another immune checkpoint that can be targeted in the methods provided herein is the cytotoxic T-lymphocyte-associated protein 4 (CTLA-4), also known as CD152. In some embodiments, the immune checkpoint inhibitor is an anti-CTLA-4 antibody (e.g., a human antibody, a humanized antibody, or a chimeric antibody), an antigen-binding fragment thereof, an immunoadhesin, a fusion protein, or oligopeptide. Anti-human-CTLA-4 antibodies (or VH and/or VL domains derived therefrom) suitable for use in the present methods include the anti-CTLA-4 antibodies disclosed in U.S. Pat. No. 8,119,129, WO 01/14424, WO 98/42752, WO 00/37504 (e.g., tremelimumab), U.S. Pat. No. 6,207,156; Hurwitz et al., Proc. Natl. Acad. Sci. U.S.A. 95(17): 10067-10071, 1998; Camacho et al., Clin. Oncology 22(145): Abstract No. 2505, 2004 (antibody CP-675206); and Mokyr et al., Cancer Res. 58:5301-5304, 1998 (incorporated herein by reference). Antibodies that compete with any of these art-recognized antibodies for binding to CTLA-4 also can be used. For example, a humanized CTLA-4 antibody is described in International Patent Application No. WO 2001/014424, WO 2000/037504, and U.S. Pat. No. 8,017,114; all incorporated herein by reference.

In some embodiments, the anti-CTLA-4 antibody is ipilimumab (also known as 10D1, MDX-010, MDX-101, and Yervoy®) or antigen binding fragments and variants thereof (see, e.g., WO 2001/14424). In some embodiments, the antibody comprises the heavy and light chain CDRs or VRs of ipilimumab. Accordingly, in some embodiment, the antibody comprises the CDR1, CDR2, and CDR3 domains of the VH region of ipilimumab, and the CDR1, CDR2 and CDR3 domains of the VL region of ipilimumab. In some embodiments, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, at least about 95%, or at least about 99% variable region identity with ipilimumab).

Other molecules for modulating CTLA-4 include CTLA-4 ligands and receptors such as described in U.S. Pat. Nos. 5,844,905, 5,885,796, and WO 1995/001994 and WO 1998/042752; all incorporated herein by reference, and immunoadhesins such as described in U.S. Pat. No. 8,329,867, incorporated herein by reference.

Examples of immunotherapies for use in treatment of kidney cancer or renal cell cancer include, but are not limited to Afinitor (Everolimus), Afinitor Disperz (Everolimus), Aldesleukin, Avastin (Bevacizumab), Avelumab, Axitinib, Bavencio (Avelumab), Bevacizumab, Cabometyx (Cabozantinib-S-Malate), Cabozantinib-S-Malate, Everolimus, IL-2 (Aldesleukin), Inlyta (Axitinib), Interleukin-2 (Aldesleukin), Ipilimumab, Keytruda (Pembrolizumab), Lenvatinib Mesylate, Lenvima (Lenvatinib Mesylate), Mvasi (Bevacizumab), Nexavar (Sorafenib Tosylate), Nivolumab, Opdivo (Nivolumab), Pazopanib Hydrochloride, pembrolizumab, proleukin (Aldesleukin), Sorafenib Tosylate, Sunitinib Malate, Sutent (Sunitinib Malate), Temsirolimus, Torisel (Temsirolimus), Votrient (Pazopanib Hydrochloride), and Yervoy (Ipilimumab).

Examples of immunotherapies for use in treatment of Acute Myeloid Leukemia (AML) include, but are not limited to Azacytidine, Arsenic Trioxide, Cerubidine (Daunorubicin Hydrochloride), Cyclophosphamide, Cytarabine, Daunorubicin Hydrochloride, Daunorubicin Hydrochloride and Cytarabine Liposome, Daurismo (Glasdegib Maleate), Dexamethasone, Doxorubicin Hydrochloride, Enasidenib Mesylate, Gemtuzumab Ozogamicin, Gilteritinib Fumarate, Glasdegib Maleate, Idamycin PFS (Idarubicin Hydrochloride), Idarubicin Hydrochloride, Idhifa (Enasidenib Mesylate), Ivosidenib, Midostaurin, Mitoxantrone Hydrochloride, Mylotarg (Gemtuzumab Ozogamicin), Rubidomycin (Daunorubicin Hydrochloride), Rydapt (Midostaurin), Tabloid (Thioguanine), Thioguanine, Tibsovo (Ivosidenib), Trisenox (Arsenic Trioxide), Venclexta (Venetoclax), Venetoclax, Vincristine Sulfate, Vyxeos (Daunorubicin Hydrochloride and Cytarabine Liposome), and Xospata (Gilteritinib Fumarate).

### Surgery

In some embodiments, the additional therapy is surgery, including preventative, diagnostic or staging, curative, and palliative surgery and tumor resection. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the byperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and lovastatin. It is further contemplated that other agents that increase the sensitivity of a hyperproliferative cell to apoptosis, such as the antibody c225, could be used in combination with certain aspects of the present embodiments to improve the treatment efficacy.

### VIII. Dosage Regimens

In some embodiments, the cells, e.g., immune cells (e.g., NK cells) are modified by engineering/introducing an engineered protein (e.g., chimeric protein) (e.g., chimeric TGF-β receptor protein) into said cells and then infused into a subject. In some embodiments, the cells, e.g., immune cells are further modified by engineering/introducing a CAR and/or a cytokine (e.g., mbIL-15/IL-15Ra complex) into the cells, in addition to the engineered protein (e.g., chimeric protein), and then infused into the subject. In some embodiments, the cells are modified and then infused within about 0 days, within about 1 day, within about 2 days, within about 3 days, within about 4 days, within about 5 days, within about 6 days or within about 7 days into a subject.

In some embodiments, an amount of modified cells, e.g., immune cells, is administered to a subject in need thereof and the amount is determined based on the efficacy and the potential of inducing cytotoxicity. In another embodiment, the modified immune cells are engineered protein⁺ (e.g., chimeric protein⁺) and CD56⁺ cells.

In some embodiments, an amount of modified cells, e.g., immune cells, comprises about 10⁴ to about 10⁹ modified cells/kg. In some embodiments, an amount of modified cells, e.g., immune cells, comprises about 10⁴ to about 10⁵ modified cells/kg. In some embodiments, an amount of modified cells, e.g., immune cells, comprises about 10⁵ to about 10⁶ modified cells/kg. In some embodiments, an amount of modified cells, e.g., immune cells, comprises about 10⁶ to about 10⁷ modified cells/kg. In some embodiments, an amount of modified cells, e.g., immune cells, comprises about 10⁷ to about 10⁸ modified cells/kg. In some embodiments, an amount of modified cells, e.g., immune cells, comprises about 10⁸ to about 10⁹ modified cells/kg. In some embodiments, an amount of modified cells, e.g., cells comprises about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, about 1×10⁸, about 2×10⁸, about 3×10⁸, about 4×10⁸, about 5×10⁸, about 6×10⁸, about 7×10⁸, about 8×10⁸, about 9×10⁸, or about 1×10⁹ modified cells/kg.

In some embodiments, the modified immune cells, e.g., immune cells are targeted to the cancer via regional delivery directly to the tumor tissue. For example, in ovarian or renal cancer, the modified cells, e.g., immune cells can be delivered intraperitoneally (IP) to the abdomen or peritoneal cavity. Such IP delivery can be performed via a port or pre-existing port placed for delivery of chemotherapy drugs. Other methods of regional delivery of modified cells, e.g., immune cells can include catheter infusion into resection cavity, ultrasound guided intratumoral injection, hepatic artery infusion or intrapleural delivery.

In some embodiments, the modified cells, e.g., immune cells are administered by intravenous (IV) administration. In some embodiments, a subject in need thereof, can begin therapy with a first dose of modified cells, e.g., immune cells delivered via IV followed by a second dose of modified cells, e.g., immune cells delivered via IV. In some embodiments, a subject in need thereof, can begin therapy with a first dose of modified cells, e.g., immune cells delivered via IP followed by a second dose of modified cells, e.g., immune cells delivered via IV. In a further embodiment, the second dose of modified cells, e.g., immune cells can be followed by subsequent doses which can be delivered via IV or IP.

### IX. Articles of Manufacture or Kits

An article of manufacture or a kit comprising engineered proteins (e.g., chimeric proteins), nucleic acids encoding said engineered proteins (e.g., chimeric proteins), and/or cells, e.g., immune cells of the present disclosure, is also provided herein. The article of manufacture or kit can further comprise a package insert comprising instructions for using the engineered proteins (e.g., chimeric proteins), nucleic acids, and/or cells, e.g., immune cells, to treat or delay progression of cancer in an individual or to enhance immune function of an individual having cancer. Any of the engineered proteins (e.g., chimeric proteins), nucleic acids and/or cells, e.g., immune cells described herein may be included in the article of manufacture or kits. Suitable containers include, for example, bottles, vials, bags, and syringes. The container may be formed from a variety of materials such as glass, plastic (such as polyvinyl chloride or poly olefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags, and syringes.

### EXAMPLES

### Example 1. Generation of a Chimeric Protein of the Signal Inverter Modality

DNA constructs are prepared for expression in immune cells, e.g., NK cells, as shown in Table 15 below:

TABLE 15
TGF-BR2 chimeric protein constructs of the signal inverter modality.

| | Extracellular | | Intracellular | SEQ |
|---|---|---|---|---|
| Signal Inverter | Domain | Transmembrane | Domain | ID |
| Chimeric Protein | (ECD) | Domain (TMD) | (ICD) | NO: |
| TGF-BR2-DAP12 | TGF-BR2 | TGF-BR2 | DAP12 | 528 |
| TGF-BR2-DAP12 | TGF-BR2 | DAP12 | DAP12 | 529 |
| TGF-BR2-DAP10 | TGF-BR2 | DAP10 | DAP10 | 530 |
| TGF-BR2-DAP10 | TGF-BR2 and | DAP10 | DAP10 | 531 |
| | DAP10 | | | |
| TGF-BR2-DAP10 | TGF-BR2 | TGF-BR2 | TGF-BR2 | 532 |
| | | | and DAP10 | |

The chimeric protein constructs described in Table 15 are cloned into the multiple cloning site of retroviral gene transfer vectors: pELNS or pES. 12-6(g)ps under control of one of the following promoters: EF-1, EFS, MND, MSCV, CMV, PGK, mCAG or RPBSA. Retrovirus is produced in 293T cells by transfecting the cells with gene transfer vectors. Cells are placed in fresh culturing medium. The virus supernatant is collected 48-72 hours post-medium change by centrifugation at 800×g for 5 minutes. The supernatant is collected, filtered, and frozen in aliquots at −80° C. Non-viral gene delivery system is based on the TCBUSTER transposon system. Transgene expression is driven by one of the following promoters: EF-1, EFS, MND, MSCV, CMV, PGK, mCAG or RPBSA. The chimeric protein constructs are cloned into transposon vectors using SpeI/NheI restriction sites. Transposon DNA and mRNA encoding TCBUSTER transposase are co-delivered into immune cells, e.g., NK cells via electroporation with either a MAXCYTE or NEON electroporation instrument. Successful integration and expression efficiency are assessed post-transduction or post-transfection by flow cytometry to characterize chimeric protein expression.

### Example 2. Generation of an Engineered Protein of the Sink Modality

DNA constructs were prepared for expression in immune cells, e.g., NK cells as shown in Table 16 below:

TABLE 16
TGF-BR1 and TGF-BR2
engineered proteins (e.g., chimeric proteins)
of the sink modality.

| Sink | Extracellular | Transmembrane |
|---|---|---|
| Engineered protein | Domain (ECD) | Domain (TMD) |
| TGF-BR1 | TGF-BR1 | TGF-BR1 |
| TGF-BR2 | TGF-BR2 | TGF-BR2 |

The engineered protein (e.g., chimeric protein) constructs described in Table 16 are cloned into the multiple cloning site of retroviral gene transfer vectors: pELNS or pES.12-6(g)ps under control of one of the following promoters: EF-1, EFS, MND, MSCV, CMV, PGK, mCAG or RPBSA. Retrovirus is produced in 293T cells by transfecting the cells with gene transfer vectors. Cells are placed in fresh culturing medium. The virus supernatant is collected 48-72 hours post-medium change by centrifugation at 800×g for 5 minutes. The supernatant is collected, filtered, and frozen in aliquots at −80° C. Non-viral gene delivery system is based on TCBUSTER transposon system. Transgene expression is driven by one of the following promoters: EF-1, EFS, MND, MSCV, CMV, PGK, mCAG or RPBSA. The engineered protein (e.g., chimeric protein) constructs are cloned into transposon vectors using SpeI/NheI restriction sites. Transposon DNA and mRNA encoding TCBUSTER transposase are co-delivered into immune cells, e.g., NK cells, via electroporation with either a MAXCYTE or a NEON electroporation instrument. Successful integration and expression efficiency are assessed post-transduction or post-transfection by flow cytometry to characterize engineered protein (e.g., chimeric protein) expression.

### Example 3. Generation of an Engineered Protein of the Dominant Negative Receptor Modality

DNA constructs were prepared for expression in immune cells, e.g., NK cells, as shown in Table 17 below:

TABLE 17
TGF-BR1 and TGF-BR2 engineered
proteins of the dominant negative receptor
modality.

| DNR | | Transmembrane | Dominant |
|---|---|---|---|
| Engineered | Extracellular | Domain | Negative |
| protein | Domain (ECD) | (TMD) | Mutation |
| TGF-BR1 | TGF-BR1 | TGF-BR1 | K232R |
| TGF-BR2 | TGF-BR2 | TGF-BR2 | R537C |

The engineered protein constructs described in Table 17 are cloned into the multiple cloning site of retroviral gene transfer vectors: pELNS or pES.12-6(g)ps under control of one of the following promoters: EF-1, EFS, MND, MSCV, CMV, PGK, mCAG or RPBSA. Retrovirus is produced in 293T cells by transfecting the cells with gene transfer vectors. Cells are placed in fresh culturing medium. The virus supernatant is collected 48-72 hours post-medium change by centrifugation at 800×g for 5 minutes. The supernatant is collected, filtered, and frozen in aliquots at −80° C. Non-viral gene delivery system is based on TcBuster Transposon. Transgene expression is driven by one of the following promoters: EF-1, EFS, MND, MSCV, CMV, PGK, mCAG or RPBSA. The engineered protein constructs are cloned into transposon vectors using SpeI/NheI restriction sites. Transposon DNA and mRNA encoding TCBUSTER transposase system are co-delivered into immune cells, e.g., NK cells, via electroporation with either a MAXCYTE or a NEON electroporation instrument. Successful integration and expression efficiency are assessed post transduction by flow cytometry to characterize engineered protein expression.

### Example 4. Isolation of NK Cells from Peripheral Blood or Cord Blood

NK cells are isolated from either human peripheral blood leukapheresis samples or cord blood units. Briefly, leukapheresis samples or cord blood units are enriched for peripheral blood mononuclear cells (PBMC). One method for PBMC enrichment is separation using a Ficoll density gradient. Next, peripheral blood NK cells are isolated from PBMC samples using immunomagnetic separation beads. Beads are conjugated to a cocktail of specific immunophenotypic antibodies to enable NK cell isolation through either positive or negative selection. Isolated NK cells are activated prior to transduction. One method for NK cell activation is co-culture with irradiated artificial antigen presenting cells (aAPCs) expressing mbIL-21 and 4-1BBL for expansion in the presence of recombinant human IL-2 (hIL-2).

### Example 5. Derivation of NK Cells from iPSCs

The derivation of NK cells from iPSCs and engineered protein (e.g., chimeric protein) transfected iPSCs have been previously described (Knorr et al., Stem Cells Transl. Med. 2(4): 274-83, 2013; Ng et al., Nat Protoc. 3:768-76, 2008; each of which is incorporated in its entirety herein by reference). Briefly, 3,000 TrypLE-adapted iPSCs are seeded in 96-well round-bottom plates with APEL culture (Ng et al., 2008, supra) containing 40 ng/ml human Stem Cell Factor (SCF), 20 ng/mL human vascular endothelial growth factor (VEGF), and 20 ng/mL recombinant human bone morphogenetic protein 4 (BMP-4). After day 11 of hematopoietic differentiation, spin embryoid bodies (EBs) are then directly transferred into each well of uncoated 24-well plates under a condition of NK cell culture. Cells are then further differentiated into NK cells as previously reported (Bachanova et al., Blood 123(25): 3855-63, 2014; Ni et al., Methods Mol. Biol. 1029:33-41, 2013) using 5 ng/mL IL-3 (first week only), 10 ng/ml IL-15, 20 ng/mL IL-7, 20 ng/mL SCF, and 10 ng/mL Flt3 ligand for 28-32 days. Half-media changes are performed weekly.

### Example 6. Methods for Testing Expression and Function of Engineered Proteins in NK Cells Quantitative RT-PCR

To test the level of engineered protein (e.g., chimeric protein) expression in modified NK cells, RNA are processed from (day 9) NK cells. For cell cycle gene analysis, transcripts are evaluated using the Human Cell Cycle RT2 Profiler PCR Array (Qiagen). The transcripts are analyzed and normalized to GAPDH.

### Immunoblot

To test the engineered protein (e.g., chimeric protein) expression in modified NK cells, suspension cells are lysed in RIPA lysis buffer with fresh protease inhibitor cocktail on ice for 20 min and sonicated for 2 seconds on ice. Membrane proteins are extracted using a Membrane Protein Extraction Kit. Sample proteins are measured by a standard bicinchoninic acid assay, size fractioned by polyacrylamide gel electrophoresis (PAGE), and are transferred to nitrocellulose membrane. Non-specific binding is blocked by incubating in TBST, 5% BSA, plus 1% Triton X-100 solution for 1 hour, followed by incubation with primary antibodies, overnight at 4° C. Species specific IRDye-conjugated secondary antibodies (1:10,000,) are applied to membranes for 1 hour at room temperature. Immunoreactive products are visualized in an Odyssey® Imaging System (LI-COR). All loading samples are normalized by staining of GAPDH.

### Cell Lysis Assay

Genetically modified peripheral blood NK cells are assessed for functionality in cell killing assays. One method to test the ability of the modified NK cells to specifically target cells for lysis is co-culture with human AML tumor cell lines expressing luciferase. Cell killing is characterized across a range of effector to target ratios (E:T). As a negative control, luciferase expressing cell lines are cultured with unmodified NK cells or NK cells expressing a non-targeting construct. An additional control is culture of luciferase expressing cell lines in the absence of NK cells. After a period of co-culture, luciferase signal is analyzed and compared to control samples. Target cell killing is observed as the decrease in luciferase signal in target cells relative to controls. Alternatively, target cell killing is observed as the release of luciferase into cell culture media.

### CD107a and Cytokine Expression

CD107a expression and cytokines such as IFNγ and TNFα by NK cells are assessed to characterize functionality. Genetically modified NK cells are co-cultured with human AML cell lines across a range of E:T ratios for a period of time. Cell surface expression of CD107a is assessed by flow cytometry with a CD107a-specific antibody. Cytokine expression is assessed by intracellular cytokine staining. Briefly, samples are treated with a protein transport inhibitor such as GolgiStop™ (BD Biosciences) for a period of time. Next, samples are treated with a fixation/permeabalization solution, stained with cytokine-specific antibodies, and assessed by flow cytometry. Alternatively, cytokine secretion into cell culture can be measured through multiplex ELISA. CD107a and cytokine expression are evaluated relative to controls including unmodified NK cells, NK cells expressing a non-engineered protein construct, and modified NK cells in the absence of target cells.

### Proliferation Assays

Proliferation of modified NK cells is assessed following co-culture with human AML tumor cell lines for a period of time. One method is covalent labeling of viable NK cells with a cell proliferation dye such as carboxyfluorescein succinimidyl ester (CFSE), where proliferation corresponds to dilution of dye. Alternatively, proliferation of modified NK cells is assessed by flow cytometry to determine NK cell counts. NK cells are labeled with NK-specific phenotypic markers and are negative for other lineage phenotypic markers. For both methods, proliferation of modified NK cells is compared to controls including unmodified NK cells, NK cells expressing a non-targeting construct, and modified NK cells in the absence of target cells.

### Example 7. In Vitro Activity Assays Using Engineered Cells Expressing Chimeric Proteins

### A. Detecting Chimeric Protein-Induced Signaling Activity in a Jurkat Cell Line

Jurkat cells or related lymphocytic cell lines are transduced to express a chimeric protein provided herein are plated at 1×10⁵ cells/well in 96-well tissue culture plates. To stimulate the chimeric protein expressed by the cells, the cells are treated with an antibody or a recombinant protein ligand that binds to the extracellular domains of the chimeric protein (e.g., at a range of from about 0.1 ng/mL to about 100 ng/ml). For example, cells expressing a chimeric protein including the extracellular domain of TGFBR2 are stimulated using anti-TGFBR2 specific antibody (e.g., clone W17055E, BIOLEGEND) or recombinant TGF-B1 cytokine (R&D SYSTEMS). As positive controls, the cells or untransduced cells are stimulated with stimuli that activate non-transduced cells, such as phorbol 12-myristate 13-acetate (PMA) plus ionomycin, TNF-α, or anti-CD3 and anti-CD28 antibodies (e.g., clones OKT3 and CD28.2, BIOLEGEND). The cells are incubated for about 4 to 48 hours with the stimuli and subsequently analyzed by staining with a CD69-specific antibody (e.g., clone FN50, BIOLEGEND) and/or IL-2 production assessed using a sandwich ELISA method (e.g., using the Human IL-2 Tissue Culture Kit (MESO SCALE DIAGNOSTICS)).

### B. Detecting Chimeric Protein-Induced NF-κB, AP-1, NFAT, or STAT Pathway Activity

Chimeric protein-induced NF-κB, AP-1, NFAT, or STAT pathway activity in cells engineered to express a chimeric protein provided herein can be assessed as follows. Reporter cell lines engineered to detect NF-κB, AP-1, NFAT, STAT1, STAT3, STAT4, STAT5, or STAT6 transcriptional activity (available from SYSTEMS BIOSCIENCES, INVIVOGEN, and PROMEGA) are transduced to express a chimeric protein provided herein and are plated at a in 96-well tissue culture plates. To stimulate the chimeric protein expressed by the cells, the cells are treated with an antibody or a recombinant protein ligand that binds to the extracellular domains of the chimeric protein (e.g., at a range of from about 0.1 ng/ml to about 100 ng/ml). For example, cells expressing a chimeric protein including the extracellular domain of TGFBR2 are stimulated using anti-TGFBR2 specific antibody (e.g., clone W17055E, BIOLEGEND) or recombinant TGF-B1 cytokine (R&D SYSTEMS). As positive controls, the cells or untransduced cells are stimulated with stimuli that activate one or more of NF-κB, AP-1, NFAT, STAT1, STAT3, STAT4, STAT5, or STAT6 transcriptional activity (e.g., TNF-α). The reporter cells are incubated for about 4 to 48 hours and subsequently analyzed by to detect reporter gene activity as recommended by the reporter cell supplier (e.g., fluorescence for cells expressing reporter GFP measured by, e.g., flow cytometry, luminescence for cells expressing reporter luciferase measured, e.g., by luminometry, or absorbance for cells expressing reporter alkaline phosphatase, measured by e.g., spectrophotometry).

### C. Detecting Chimeric Protein-Induced PI3K Activity

Chimeric protein-induced PI3K activity in cells engineered to express a chimeric protein provided herein can be assessed as follows. HEK-293T cells, Jurkat cells, or other suitable cell lines, are transduced to express a chimeric protein and then transfected to express both FOXO transcription factor and a luciferase reporter gene cassette under the control of multimers of the FOXO responsive element located upstream of a minimal promoter (e.g., FOXO Reporter Kit, BPS Bioscience). The cells are plated at a density of 1×10⁴ to 1×10⁵ cells/well in 96-well tissue culture plates. After incubation for about 6 to 24 hours, the cells are stimulated with an antibody or a recombinant protein that binds to the extracellular domains of the chimeric protein (e.g., at a range of from about 0.1 ng/mL to about 100 ng/mL). For example, cells expressing a chimeric protein including the extracellular domain of TGFBR2 are stimulated using anti-TGFBR2 specific antibody (e.g., clone W17055E, BIOLEGEND) or recombinant TGF-B1 cytokine (R&D SYSTEMS). Stimuli that activate PI3K or Akt in untransduced cells, such as ethyl 2-amino-6-chloro-4-(1-cyano-2-ethoxy-2-oxoethyl)-4H-chromene-3-carboxylate (also known as SC79; Tocris Bioscience), are used as positive control. Cells are incubated for about 4 to about 48 hours and then analyzed by detecting luciferase activity, e.g., by luminometry.

### D. Detecting Chimeric Protein-Induced Cell Signaling Activity by Phosphorylation of Downstream Signaling Proteins in Activating Pathways

Chimeric protein-induced cell signaling activity (e.g., by phosphorylation of downstream signaling proteins in activating pathways) in cells engineered to express a chimeric protein provided herein can be assessed as follows. NK cells, Jurkat cells, or other non-adherent lymphocytic cell lines, are transduced to express a chimeric protein provided herein, and the cells are plated at a density of 1×10⁵ cells/well in 96-well tissue culture plates. To stimulate the chimeric protein expressed by the cells, the cells are treated with an antibody or a recombinant protein ligand that binds to the extracellular domains of the chimeric protein (e.g., at a range of from about 0.1 ng/mL to about 100 ng/mL). For example, cells expressing a chimeric protein including the extracellular domain of TGFBR2 are stimulated using anti-TGFBR2 specific antibody (e.g., clone W17055E, BIOLEGEND) or recombinant TGF-B1 cytokine (R&D SYSTEMS). Stimuli that activate a signaling pathway in untransduced cells, such as PMA plus ionomycin (for NF-κB, AP-1, or NFAT) or IL-2 (for STAT5), are used as positive controls. The cells are incubated for 5 minutes to 3 hours then fixed, permeabilized, and analyzed by staining with antibodies specific for activating phosphorylation modifications in signaling pathway proteins downstream of the chimeric protein, e.g., as measured by flow cytometry. For example, phosphorylated levels of: RelA/p65 (using anti-phospho-NF-κB p65 (Ser536) rabbit monoclonal antibody (mAb), clone 93H1) for NF-κB activity, c-Jun (using phospho-c-Jun (Ser63) rabbit mAb, clone E617P) for AP-1 activity, STAT1 (using phospho-Stat1 (Tyr701) rabbit mAb, clone D4A7), STAT3 (using phospho-Stat3 (Tyr705) (D3A7) rabbit mAb, clone D3A7), STAT4 (using phospho-Stat4 (Tyr693) rabbit mAb, clone D2E4,) STAT5A/B (using phospho-Stat5 (Tyr694) rabbit mAb, clone C71E5), STAT6 (using phospho-Stat6 (Tyr641) rabbit mAb, clone D8S9Y), and/or Akt (using phospho-Akt (Ser473) rabbit mAb, clone D9E) for PI3K activity (each of the foregoing antibodies are available from CELL SIGNALING TECHNOLOGY).

### Example 8. In Vivo Studies Using Engineered NK Cells Expressing a Chimeric Protein Functionality of Switch Receptor In Vivo

NK cells engineered to express a chimeric protein provided herein or, as a control, an inert protein including a non-functional tag, are administered to immunodeficient NSG mice in doses of from about 1×10⁶-1×10⁷ NK cells per mouse either by intraperitoneal (IP) or intravenous (IV) injection. Ligands (e.g., a negative signal, e.g., TGF-B1) that bind to the extracellular domain of the chimeric protein may either be present in NSG mice (e.g., and be cross-reactive with the chimeric protein), or may be administered by IP or IV injection. NK cell numbers are compared after about 3 to 28 days, e.g., using flow cytometry, to detect NK cells in blood and/or peritoneal fluid. Mice treated with NK cells expressing chimeric proteins that impact the proliferation of the NK cells in vivo (e.g., in response to the ligand) may exhibit an increase in NK cell number (e.g., >25% or increase (e.g., calculated by adding >1 week to NK cell half-life compared to control NK cells)).

To assess the in vivo anti-tumor activity of NK cells expressing a chimeric protein provided herein, the following experiments may be performed. A solid tumor xenograft model is developed in NSG mice. The selected tumor cells can express combinations of endogenous and/or engineered ligands that can activate or inhibit engineered NK cells (e.g., an antigen that specifically binds to a CAR expressed by the NK cells and/or a ligand that binds to the extracellular domain of a chimeric protein expressed by the NK cells). The tumor cells may also be engineered to express luciferase. The tumor cells are implanted into the NSG mice in doses of about 5×10⁵ to about 1×10⁸ cells per mouse by subcutaneous, IP, or IV injection. NK cells engineered to express a chimeric protein provided herein or control NK cells that do not express the chimeric protein are administered to the mice in doses of 1×10⁶-1×10⁷ cells per tumor-bearing mouse by IP or IV injection. The tumor cells and NK cells may be co-administered to the mice on the same day, or the tumor cells may be administered 1 to 14 days prior to the NK cells (e.g., to allow for engraftment of the tumor cells). Tumor growth and expansion is monitored using caliper measurements, imaging of luminescence signal, or frequency of tumor cells in peripheral blood (e.g., measured by flow cytometry). Tumor clearance, tumor growth rate, the number of NK cells in tumor tissue (e.g., biopsied tumor tissue), NK cell persistence, and cytokine (e.g., IFN-γ, TNF-α, IL-8, IP-10, MCP-1, and MIP-1a/b) production levels (e.g., in vivo or in vitro, e.g., in blood, peritoneal fluid, and/or tumor tissue) is assessed using methods known in the art. For example, cytokine production in vivo is measured in blood or peritoneal fluid and detected as protein (e.g., by sandwich ELISA) or in tumor cell-containing tissue (e.g., detected as mRNA following tissue homogenization, RNA isolation, and quantitative RT-PCR). Ex vivo cytokine production and/or cytotoxicity is measured using NK cells isolated from blood, peritoneal fluid, or single cell suspensions prepared from dissociation of tumor-containing tissue. After isolation, NK cells are cultured for about 4 to about 48 hours in media without stimulation to analyze cytokines and/or cytotoxicity using methods known in the art.

### Example 9. Reporter Cells Expressing Exemplary Chimeric Proteins with Different Extracellular Domains Convert Extracellular Negative Signal Stimulation into Activating Responses

Jurkat-NF-κB-GFP reporter cells expressing either chimeric proteins including the extracellular and transmembrane domains of the inhibitory receptors: BTLA (SR-100), CD200R (SR-102), CTLA-4 (SR-104), Fas (SR-105), IL-10RA (SR-106), IL-10RB (SR-107), GP130 (also known as IL-6RB; SR-109), PD1 (SR-111), TACTILE (also known as CD96; SR-112), and TIGIT (SR-113), and the intracellular domain of CD3ζ; chimeric proteins including the extracellular domains of BTLA (SR-115), CD200R (SR-117), CTLA-4 (SR-119), Fas (SR-120), IL-10RA (SR-121), IL-10RB (SR-122), GP130 (SR-124), PD1 (SR-126), TACTILE (SR-127), and TIGIT (SR-128), the transmembrane domain of CD28 and the intracellular domain of CD3ζ; or chimeric proteins including the extracellular domain of TGF-BR2, the transmembrane domain of CD28 and the intracellular domain of CD3ζ (TGFB-021) were generated. Each chimeric protein is described in Table 21 below. The reporter cells were stimulated with increasing doses of plate-coated agonist antibodies specific for the extracellular domain of the chimeric proteins, plate-coated recombinant FasL, or soluble recombinant TGF-B1. As positive control, reporter cells transduced to express a fusion protein including truncated CD19 (CD191-319) and a tag (“dCD19”) were stimulated through activation of endogenous CD3 and CD28 receptors using increasing doses of plate coated anti-CD3 and soluble anti-CD28 antibodies. As negative control, reporter cells expressing truncated non-signaling TGF-BR2₁₋₁₉₉ were stimulated using anti-TGF-BR2 antibody or soluble recombinant TGF-B1. Following an overnight incubation period with the stimulants, the reporter cells were analyzed by flow cytometry to measure the induction of CD69, a marker of cell activation (see, e.g., Castellanos et al. Eur J. Immunol. 32(11): 3108-17, 2002), and GFP, the engineered reporter of NF-κB activation in the reporter cells. The results of these experiments are summarized in Table 19 below.

TABLE 19
Results Summary

| | | | | | | | Fold-change of MFI over no stimulation | | | | | |
|---|---|---|---|---|---|---|---|---|---|---|---|---|
| | Construct | | | | % | | GFP | | | CD69 | | |
| | ID | ECD | TM | ICD | Transduced | Stimulant | High | Med. | Low | High | Med. | Low |
| Pos. Control | CONT-101 | CD19 | CD19 | N/A | 94.7 | CD3 mAb + | 12.2 | 3.0 | 1.4 | 41.5 | 9.2 | 3.7 |
| | | | | | | CD28 mAb | | | | | | |
| Neg. Control | TGFB-007 | TGFBR2 | TGFBR2 | | 93.4 | TGFBR2 mAb | 1.0 | 0.9 | 0.8 | 2.0 | 1.5 | 1.3 |
| | TGFB-007 | TGFBR2 | TGFBR2 | | 93.4 | TGFB1 cytokine | 0.8 | 0.7 | 0.7 | 1.2 | 1.2 | 1.4 |
| Engineered | SR-100 | BTLA | BTLA | CD3z | 89.1 | BTLA mAb | 13.0 | 7.5 | 6.1 | 124.7 | 124.1 | 62.7 |
| protein design | SR-102 | CD200R | CD200R | | 86.9 | CD200R mAb | 6.8 | 1.4 | 0.8 | 29.2 | 4.9 | 1.3 |
| | SR-104 | CTLA4 | CTLA4 | | 87.5 | CTLA4 mAb | 4.3 | 3.8 | 2.2 | 7.2 | 5.6 | 3.1 |
| | SR-105 | Fas | Fas | | 87.7 | FasL | 7.3 | 4.6 | 1.0 | 70.2 | 35.7 | 8.4 |
| | | | | | | recombinant | | | | | | |
| | | | | | | protein | | | | | | |
| | SR-106 | IL10RA | IL10RA | | 83.1 | IL10RA mAb | 11.4 | 3.3 | 0.8 | 56.8 | 29.5 | 4.9 |
| | SR-107 | IL10RB | IL10RB | | 86.6 | IL10RB mAb | 13.8 | 9.3 | 2.9 | 108.3 | 86.1 | 32.2 |
| | SR-109 | GP130 | GP130 | | 90.9 | GP130 mAb | 6.3 | 2.6 | 0.5 | 122.6 | 22.0 | 1.1 |
| | SR-111 | PD1 | PD1 | | 85.1 | PD1 mAb | 5.8 | 1.4 | 1.3 | 37.6 | 1.9 | 1.2 |
| | SR-112 | TACTILE | TACTILE | | 57.5 | TACTILE mAb | 1.2 | 0.9 | 1.0 | 3.9 | 1.0 | 3.3 |
| | SR-113 | TIGIT | TIGIT | | 90.9 | TIGIT mAb | 4.6 | 2.2 | 0.9 | 17.1 | 17.1 | 7.2 |
| | SR-115 | BTLA | CD28 | | 87.1 | BTLA mAb | 3.5 | 6.9 | 5.1 | 9.2 | 14.1 | 10.8 |
| | SR-117 | CD200R | CD28 | | 83.7 | CD200R mAb | 5.9 | 0.9 | 1.1 | 8.7 | 1.1 | 0.9 |
| | SR-119 | CTLA4 | CD28 | | 92.7 | CTLA4 mAb | 5.9 | 4.6 | 2.6 | 6.2 | 4.5 | 2.7 |
| | SR-120 | Fas | CD28 | | 89.3 | FasL | 10.1 | 11.2 | 2.4 | 162.0 | 128.9 | 40.5 |
| | | | | | | recombinant | | | | | | |
| | | | | | | protein | | | | | | |
| | SR-121 | IL10RA | CD28 | | 90.6 | IL10RA mAb | 5.4 | 2.9 | 1.1 | 14.3 | 7.9 | 2.2 |
| | SR-122 | IL10RB | CD28 | | 91.9 | IL10RB mAb | 8.2 | 12.5 | 6.2 | 115.2 | 118.4 | 70.1 |
| | SR-124 | GP130 | CD28 | | 79.9 | GP130 mAb | 3.0 | 3.9 | 0.5 | 64.1 | 40.7 | 1.8 |
| | SR-126 | PD1 | CD28 | | 89.2 | PD1 mAb | 5.5 | 1.2 | 0.9 | 27.3 | 1.7 | 1.1 |
| | SR-127 | TACTILE | CD28 | | 79.5 | TACTILE mAb | 1.0 | 0.8 | 1.0 | 2.0 | 1.1 | 1.1 |
| | SR-128 | TIGIT | CD28 | | 96.4 | TIGIT mAb | 1.7 | 1.2 | 0.6 | 3.4 | 2.6 | 1.2 |
| | TGFB-021 | TGFBR2 | CD28 | | 92.5 | TGFBR2 mAb | 5.0 | 2.9 | 0.9 | 88.2 | 25.2 | 2.2 |
| | TGFB-021 | TGFBR2 | CD28 | | 92.5 | TGFB1 cytokine | 10.1 | 9.3 | 2.8 | 93.0 | 62.2 | 21.5 |

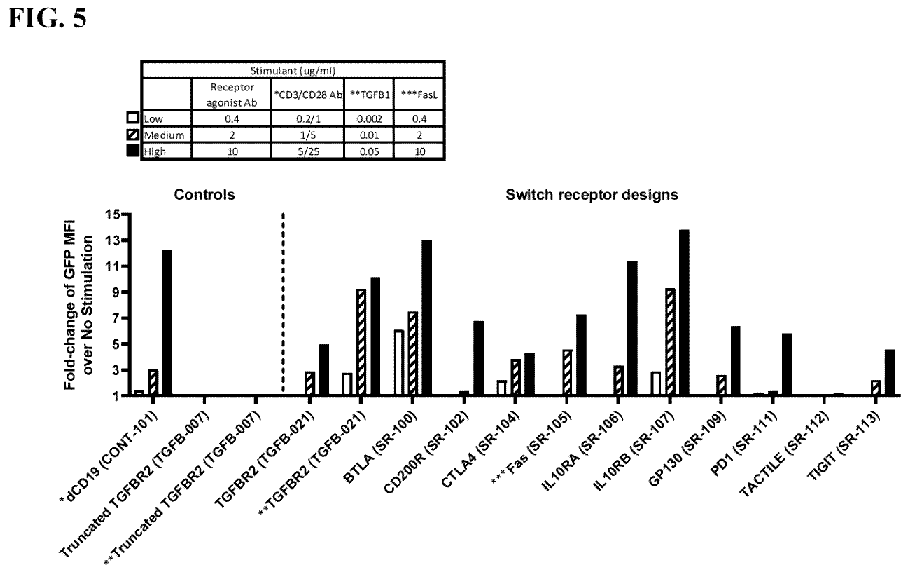

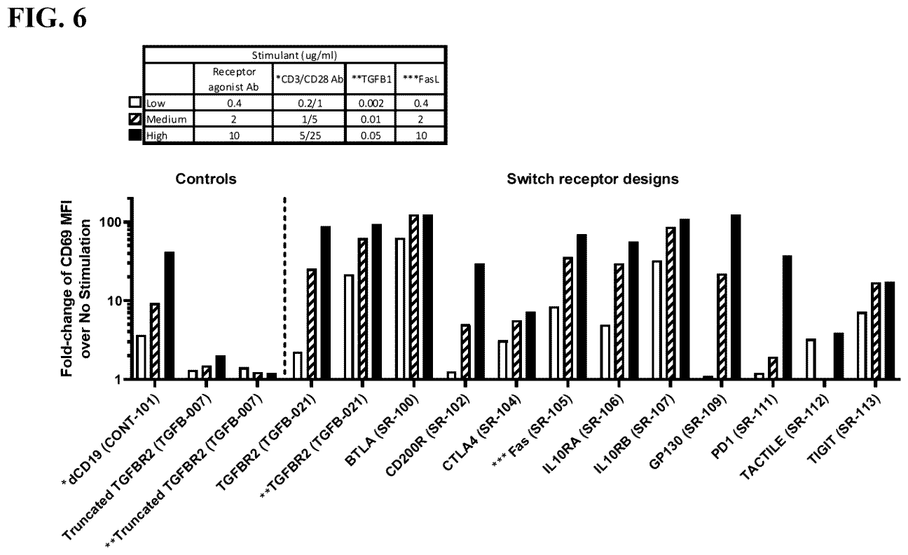

As shown in FIGS. 5 and 6, stimulation of the reporter cells expressing the chimeric proteins including the extracellular domains of inhibitory receptors induced a dose-dependent increase in NF-κB-dependent transcription (as measured by GFP expression), as compared to unstimulated transduced reporter cells (FIG. 5), as well as a dose-dependent increase in CD69 expression (FIG. 6), as compared to unstimulated transduced reporter cells. The sole exception was reporter cells expressing SR-112 (a chimeric protein that includes the extracellular domain of TACTILE) which exhibited a minimal increase in CD69 expression. The scale of the ligand (TGF-B1)-dependent increase in CD69 and NF-κB-induced GFP expression was similar among all samples, and in some cases, greater than the increase in CD69 and NK-κB-induced GFP expression induced in the control cells expressing dCD19.

These data demonstrate that the chimeric proteins including the extracellular domains of: BTLA (SR-100), CD200R (SR-102), CTLA-4 (SR-104), Fas (SR-105), IL-10RA (SR-106), IL-10RB (SR-107), GP130 (also known as IL-6RB; SR-109), PD1 (SR-111), TACTILE (SR-112), TIGIT (SR-113), and TGF-BR2 (TGFB-021) and the intracellular domain of CD3ζ, can be readily expressed. Moreover, expression of the chimeric proteins confers the cells the ability to convert negative extracellular stimuli into strong and dose-dependent activating signals. Notably, the chimeric proteins that were tested included extracellular domains of inhibitory receptors that are generally configured as monomers (e.g., PD-1, Freeman Proc. Nat'l. Acad. Sci. USA 105:10275-6, 2008), homodimers (e.g., CTLA-4, Freeman 2008, supra), heterodimers (e.g., TGF-BR2, Allendorph et al. Proc. Nat'l. Acad. Sci. U.S.A. 103(20): 7643-8, 2006), and homotrimers (e.g., Fas, Salvesen and Riedl Cell Cycle 8(17): 2723-7, 2009). Interestingly, the structural and stoichiometric requirements of the native inhibitory receptors from which the chimeric proteins were derived did not appear to impact the functioning of the chimeric proteins.

### Example 10. Expression of Chimeric Proteins Including a TGF-BR2 Extracellular Domain and Various Intracellular Domains Convert TGF-B1 Stimuli into Activating Responses

Jurkat-NFκB-GFP reporter cells transduced to express chimeric proteins including the extracellular domain of TGF-BR2 and the intracellular domain of either: 4-1BB (TGFB-052), SLP76 (TGFB-056), MYD88 (TGFB-057), DAP12 (TGFB-058), TRAF1 (TGFB-059), TRAF2 (TGFB-060), TRAF3 (TGFB-061), TIRAP (TGFB-064), BLNK (TGFB-065), FCGR3 (TGFB-068), TLR4 (TGFB-087), Syk (TGFB-091), YES1 (TGFB-095), CD28 (TGFB-019), or CD3ζ (TGFB-021) (each described in Table 21 below). Transduced cells were left unstimulated or treated with increasing concentrations of recombinant TGF-B1 (0.032 ng/ml, 0.1 ng/mL, 0.32 ng/ml, 1 ng/ml, 3.2 ng/mL, 10 ng/mL, or 32 ng/mL) to stimulate the chimeric proteins expressed by the reporter cells. After an overnight incubation period, the reporter cells were analyzed by flow cytometry to measure the induction of CD69 and GFP. As a positive control, cells transduced to express dCD19 were stimulated through the activation of endogenous CD3 and CD28 receptors with 1 μg/mL plate-coated anti-CD3 antibody plus 5 μg/mL soluble anti-CD28 agonist antibodies. As negative controls, mock transduced reporter cells and reporter cells expressing dCD19 (CONT-101) were either left unstimulated or treated with either anti-TGBR2 antibody or increasing concentrations of recombinant TGF-B1.

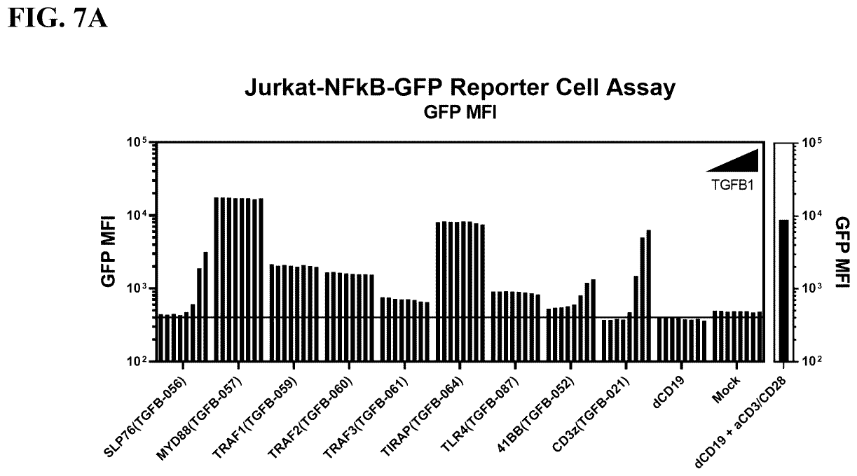

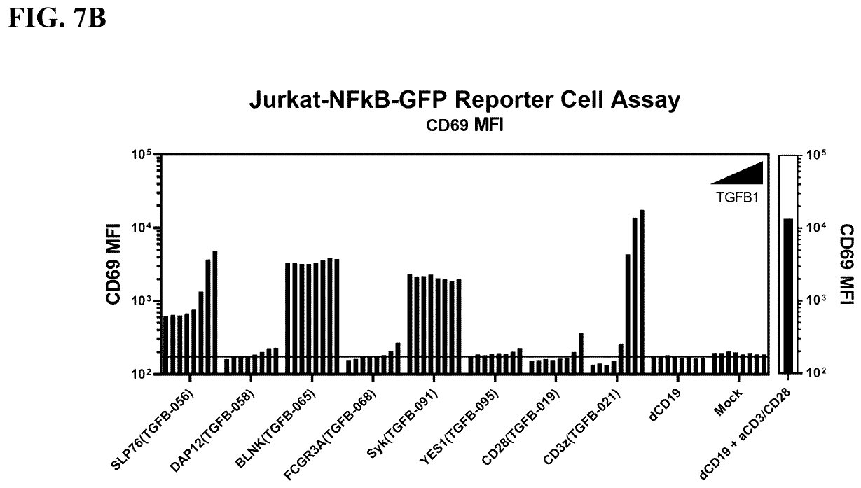

As shown in FIG. 7A, TGF-B1 stimulation of the reporter cells expressing an chimeric protein including the extracellular domain of TGF-BR2 and an intracellular domain of either: SLP76 (TGFB-056), 4-1BB (TGFB-052), or CD3ζ (TGFB-021) each induced a dose-dependent increase in NF-κB-dependent transcription. Moreover, as shown in FIG. 7B, TGF-B1 stimulation induced a dose-dependent increase in CD69 expression in reporter cells expressing the chimeric proteins including the TGF-BR2 extracellular domain and either an intracellular domain of SLP76 (TGFB-056), of DAP12 (TGFB-058), of FCGR3A (TGFB-068), YES1 (TGFB-095), CD28 (TGFB-019), and CD3ζ (TGFB-021).

Interestingly, the expression of some of the chimeric proteins in the reporter cells was sufficient to activate signaling without extracellular TGF-B1 stimuli, as compared to mock transduced reporter cells and reporter cells expressing dCD19. Specifically, reporter cells expressing chimeric proteins including the extracellular domain of TGF-BR2 and either the intracellular domain of: MYD88 (TGFB-057), TRAF1 (TGFB-059), TRAF2 (TGFB-060), TRAF3 (TGFB-061), TIRAP (TGFB-064), TLR4 (TGFB-087), BLNK (TGFB-065), and Syk (TGFB-091) induced NF-κB-dependent transcription and/or increased CD69 expression levels independent of exogenous TGF-B1 exposure, while also exhibiting minimal to no change in either activation marker following exposure to exogenous TGF-B1. In addition, reporter cells expressing chimeric proteins including the extracellular domain of TGF-BR2 and either the intracellular domain of: 4-1BB (TGFB-052), SLP76 (TGFB-056), and YES1 (TGFB-095) exhibited both constitutive and dose-dependent TGF-B1-induced signaling.

Overall, these data demonstrate that expression of chimeric proteins including the extracellular domain of TGF-BR2 and an intracellular domain of a stimulatory polypeptide in an immune cell enables signal modulation in the cells capable of either: 1) dose-dependent conversion of a negative signal, TGF-B1, to an activating signaling event(s); 2) ligand-independent constitutive signaling; and 3) a combination of negative signal (TGF-B1)-induced activating signaling and independent constitutive activating signaling.

### Example 11. NK Cells Expressing Chimeric Proteins Including an Extracellular Domain of TGBR2 and One of Various Intracellular Domains Exhibit Increased Expansion, Cytotoxicity, and INF-γ and IP-10 Secretion

### NK Cell Expansion

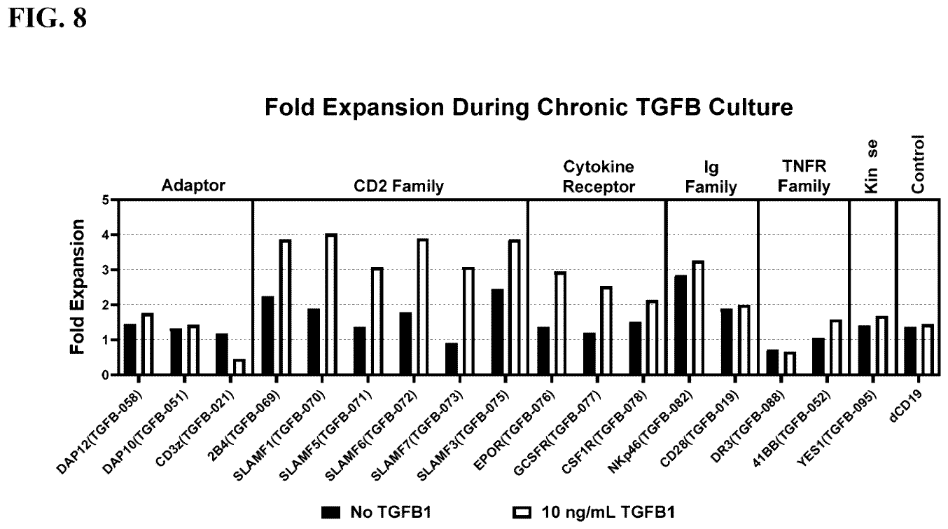

The effect of NK cell expression of chimeric proteins including an extracellular domain of TGF-BR2 and the intracellular domain of either SLP76 (TGFB-056), MYD88 (TGFB-057), DAP12 (TGFB-058), TRAF1 (TGFB-059), TRAF2 (TGFB-060), TRAF3 (TGFB-061) TRAF6 (TGFB-062), TIRAP (TGFB-064), BLNK (TGFB-065), GRB2 (TGFB-066), FCGR2A (TGFB-67), FCGR3A (TGFB-068), 2B4 (TGFB-069), SLAMF1 (TGFB-070), SLAMF5 (TGFB-071), SLAMF6 (TGFB-072), SLAMF7 (TGFB-073), LFA2 (TGFB-074), SLAMF3 (TGFB-075), EPOR (TGFB-076), GCSFR (TGFB-077), CSFIR (TGFB-078), FGFR1 (TGFB-079), DNAM1 (TGFB-080), ICOS (TGFB-081), NKp46 (TGFB-082), NKp44 (TGFB-083), NKp30 (TGFB-084), IRAK4 (TGFB-086), TLR4 (TGFB-087), DR3 (TGFB-088), Syk (TGFB-091), Lyn (TGFB-094), YES1 (TGFB-095), Fgr (TGFB-096), 4-1BB (TGFB-052), DAP10 (TGFB-051), CD28 (TGFB-019), or CD3z (TGFB-021). As negative controls, mock transduced NK cells (Mock) and NK cells expressing dCD19 (CONT-101) were used. Each chimeric protein is described in Table 21 below. As shown in FIG. 8, NK cells expressing chimeric proteins including the extracellular domain of TGF-BR2 and the intracellular domain of either: DAP12 (TGFB-058), 2B4 (TGFB-069), SLAMF1 (TGFB-070), SLAMF5 (TGFB-071), SLAMF6 (TGFB-072), SLAMF7 (TGFB-073), SLAMF3 (TGFB-075), EPOR (TGFB-076), GCSFR (TGFB-077), CSFIR (TGFB-078), NKp46 (TGFB-082), or CD28 (TGFB-019) exhibited improved NK cell expansion following 5 days of chronic exposure to TGF-B1, as compared to control NK cells expressing dCD19.

NK cells expressing chimeric proteins including the extracellular domain of TGF-BR2 and either the intracellular domain of: 2B4 (TGFB-069), SLAMF1 (TGFB-070), SLAMF6 (TGFB-072), or SLAMF3 (TGFB-075) also exhibited improved NK cell expansion following 5 days of culture in the absence of exogenous TGF-B1 stimuli, as compared to control NK cells expressing dCD19. Chronic exposure of these NK cells to TGF-B1 further improved cell expansion. In contrast, NK cells expressing chimeric proteins including the extracellular domain of TGF-BR2 and the intracellular domain of either: NKp46 (TGFB-082) or CD28 (TGFB-019) exhibited an equivalent increase in cell expansion in the absence or presence of exogenous TGF-B1 exposure. These findings demonstrated that these chimeric proteins are constitutively active in NK cells and that their expression alone may be beneficial to NK cell expansion potential.

Surprisingly, expression of chimeric proteins including the TGF-BR2 extracellular domain and either an intracellular domain of SLAMF1 (TGFB-070), SLAMF3 (TGFB-075), 2B4 (TGFB-069), SLAMF5 (TGFB-071), SLAMF6 (TGFB-072), and SLAM7 (TGFB-073) induced the greatest gain of function in NK cell expansion. All six of these chimeric proteins include intracellular domains that are derived from SLAM family members and include Immune Tyrosine Switch Motifs (ITSMs). Although all of these SLAM family members contain an ITSM, the proteins are not believed to play interchangeable functions (see, e.g., Dragovich Autoimmun Rev. 17(7): 674-82, 2018).

Overall, these data demonstrated that the chimeric proteins could convert an immunosuppressive negative signal, TGF-B1, into a gain of function in NK cells: increased expansion potential.

### NK Cell Cytokine Secretion and Cytotoxicity

To analyze the effect of chimeric protein expression on NK cell cytokine production and cytotoxic activity, assays utilizing the SKOV-3 target cells were performed using the NK cells following chronic exposure to TGF-B1 or in the absence of TGF-B1 exposure. SKOV-3 tumor cells co-express inhibitor and activating ligands of thee inhibitory signals reduce NK cell maximum cytokine production and cytotoxicity (see, e.g., Maas, Oncoimmunology 9(1): e1843247, 2020).

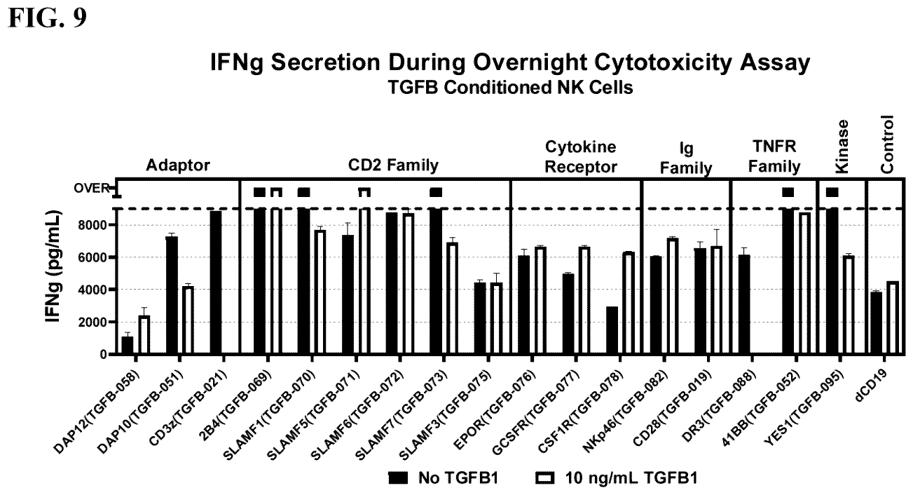

As shown in FIG. 9, NK cells expressing chimeric proteins including the extracellular domain of TGF-BR2 and either the intracellular domain of either: 2B4 (TGFB-069), SLAMF1 (TGFB-070), SLAMF5 (TGFB-071), SLAMF6 (TGFB-072), SLAMF7 (TGFB-073), EPOR (TGFB-076), GCSFR (TGFB-077), NKp46 (TGFB-082), CD28 (TGFB-019), 4-1BB (TGFB-052), or YES1 (TGFB-095) exhibited increased SKOV-3-induced interferon-gamma (IFN-γ) production both with or without chronic TGF-B1 exposure, as compared to control NK cells expressing dCD19. This data demonstrated that these chimeric proteins were constitutively active in the NK cells and that the proteins enhanced the NK cells' capacity to produce INF-γ following target cell contact.

Moreover, NK cells expressing chimeric proteins including the extracellular domain of TGF-BR2 and the intracellular domain of either: SLAMF5 (TGFB-071), CSFIR (TGFB-078), or GCSFR (TGFB-077) exhibited enhanced SKOV-3 induced IFN-γ secretion following chronic TGF-B1 exposure, indicating that these three chimeric proteins confer a gain of function phenotype to NK cells that is both constitutive and ligand (TGF-B1)-dependent. In contrast, NK cells expressing chimeric proteins including the extracellular domain of TGF-BR2 and the intracellular domain of either: DAP10 (TGFB-051), CD3z (TGFB-021), or DR3 (TGFB-088) exhibited enhanced SKOV-3-induced IFN-γ production in the absence of chronic TGF-B1 exposure but not following chronic TGF-B1 exposure. This result demonstrated that these chimeric proteins are constitutively active in NK cells that enhance their INF-γ production capacity upon contact with target cells.

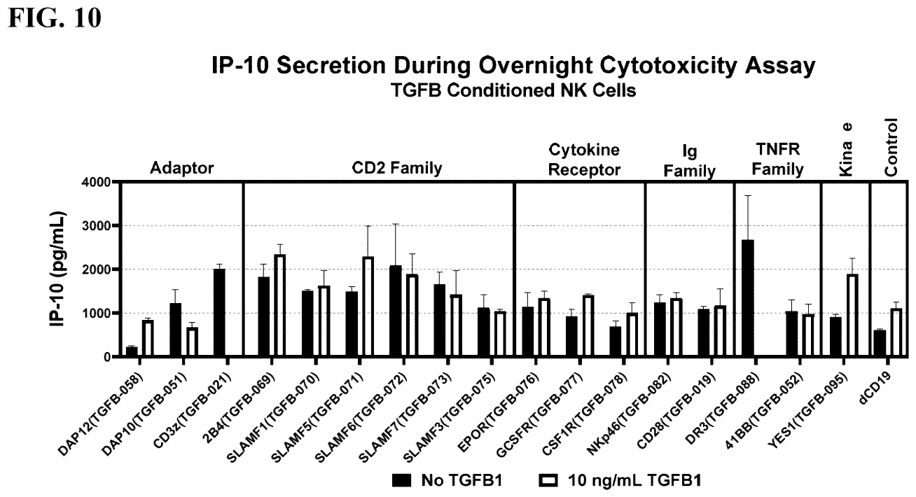

NK cells expressing chimeric proteins including the extracellular domain of TGBR2 and the intracellular domain of either: 2B4 (TGFB-069), SLAMF1 (TGFB-070), SLAMF5 (TGFB-071), SLAMF6 (TGFB-072), SLAMF7 (TGFB-073), EPOR (TGFB-076), or GCSFR (TGFB-077) exhibited increased SKOV-3-induced IP-10 production both with or without chronic TGF-B1 exposure, as compared with control NK cells expressing dCD19 (see FIG. 10). In contrast, NK cells expressing chimeric proteins including the extracellular domain of TGBR2 and the intracellular domain of either DAP10 (TGFB-051) or DR3 (TGFB-088) exhibited increased SKOV-3-induced IP-10 production in the absence of chronic TGF-B1 exposure but not following chronic TGF-B1 exposure. Overall, these results demonstrated that these chimeric proteins were constitutively active in the NK cells and enhanced the NK cells' IP-10 production capacity upon contact with target cells.

Interestingly, NK cells expressing an chimeric protein including the extracellular domain of TGF-BR2 and the intracellular domain of YES1 (TGFB-095) enhanced SKOV-3-induced IP-10 production selectively following TGF-B1 exposure, demonstrating that this chimeric protein confers a ligand-dependent gain of function to NK cells.

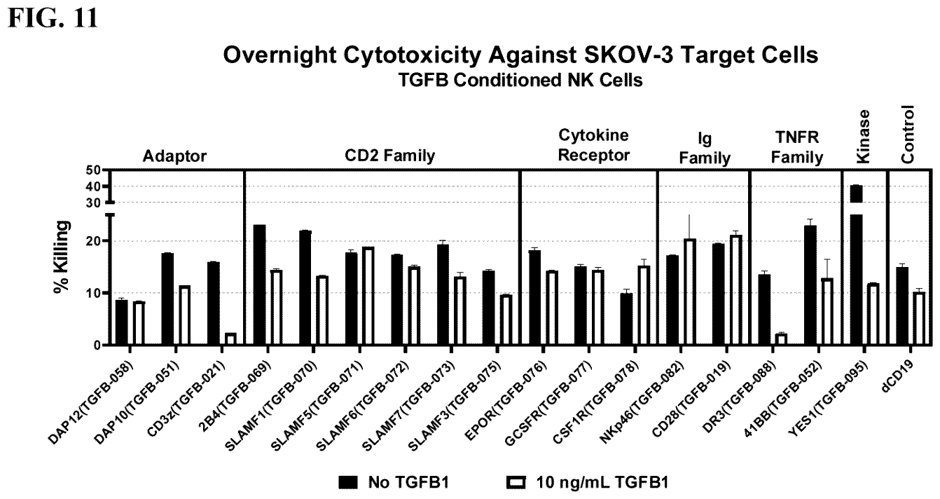

Finally, cytotoxicity assays were performed to assess the impact of the chimeric proteins on NK cells. As shown in FIG. 11, NK cells expressing chimeric proteins including the extracellular domain of TGF-BR2 and the intracellular domain of either: 2B4 (TGFB-069), SLAMF1 (TGFB-070), SLAMF5 (TGFB-071), SLAMF6 (TGFB-072), SLAMF7 (TGFB-073), EPOR (TGFB-076), GCSFR (TGFB-077), NKp46 (TGFB-082), or CD28 (TGFB-019) exhibited increased cytotoxic activity against SKOV-3 tumor cells both with or without chronic TGF-B1 pre-exposure, as compared with control NK cells expressing dCD19. This data demonstrated that these chimeric proteins were constitutively active in NK cells and enhanced their ability to kill the target tumor cells (i.e., SKOV-3 cells).

NK cells expressing an chimeric protein including the extracellular domain of TGF-BR2 and the intracellular domain of CSFIR (TGFB-078) enhanced the cytotoxicity of the NK cells selectively following chronic TGF-B1 exposure, demonstrating that this chimeric protein confers a ligand (TGF-B1)-dependent gain of function to NK cells. In contrast, NK cells expressing chimeric proteins including the extracellular domain of TGF-BR2 and the intracellular domain of either: DAP10 (TGFB-051), CD3ζ (TGFB-021), DR3 (TGFB-088), 4-1BB (TGFB-052), or YES1 (TGFB-095) increased NK cell cytotoxicity in the absence of chronic TGF-B1 exposure but not following chronic TGF-B1 exposure. These results demonstrated that these chimeric proteins were constitutively active in the NK cells and enhanced the NK cells' cytotoxic activity against target cells.

Surprisingly, NK cells expressing a chimeric protein including the extracellular domain of TFBR2 and the intracellular domain of YES1 exhibited the greatest increase in NK cell cytotoxicity in the absence of chronic TGF-B1 exposure-almost double the cytotoxicity that was observed by NK cells expressing the other chimeric proteins. YES1 is one of 8 Src-family cytoplasmic protein tyrosine kinases. In NK cells, the recruitment of Src-family kinases to cell surface receptors is critical to initiate both activating and inhibitory signaling (see Meza Guzman et al. Cancers 12(4): 952, 2020). However, NK cells expressing chimeric proteins including the intracellular domains of other Src-family kinases (e.g., Fgr (TGFB-096) and Lyn (TGFB-094)) did exhibit enhanced cytotoxicity, suggesting chimeric proteins including the intracellular domain of YES1 can have a selective advantage in priming NK cells for enhanced cytotoxic activity.

The data corresponding to Example 3 is summarized in Table 20 below.

TABLE 20
Results Summary

| | | | | | | | Average IFNg | | Average IP-10 | |
|---|---|---|---|---|---|---|---|---|---|---|
| | Transduction | Transduction | 5-Day Fold Expansion | | Average % Killing | | Secretion | | Secretion | |
| | Efficiency, | Efficiency, NK | 10 ng/mL | No | 10 ng/mL | No | 10 ng/mL | No | 10 ng/mL | No |
| Construct ID | Jurkat Cells | Cells | TGF-B1 | TGF-B1 | TGF-B1 | TGF-B1 | TGF-B1 | TGF-B1 | TGF-B1 | TGF-B1 |
| TGFB-056 | 49.4 | 9.5 | 0.41 | 0.70 | 2.07 | 16.05 | 1.68 | 5132.37 | UNDER | 1430.87 |
| TGFB-057 | 80.0 | 66.2 | 1.02 | 0.89 | 11.24 | 15.45 | 7771.60 | 5112.42 | 2758.66 | 1472.41 |
| TGFB-058 | 72.3 | 58.3 | 1.76 | 1.45 | 8.35 | 8.62 | 2364.21 | 1067.04 | 836.97 | 221.40 |
| TGFB-059 | 81.4 | 54.7 | 0.90 | 0.84 | 7.99 | 11.99 | 3057.45 | 2800.11 | 1296.50 | 816.81 |
| TGFB-060 | 70.9 | 34.8 | 1.11 | 1.40 | 8.77 | 10.55 | 3066.92 | 2146.22 | 1232.29 | 544.54 |
| TGFB-061 | 59.3 | 31.2 | 1.29 | 1.39 | 8.90 | 10.69 | 3092.46 | 2197.01 | 762.20 | 360.21 |
| TGFB-062 | 68.8 | 41.5 | 1.41 | 1.45 | 8.40 | 9.69 | 3012.76 | 2077.12 | 1369.84 | 509.05 |
| TGFB-064 | 67.0 | 48.9 | 1.04 | 1.11 | 9.89 | 9.04 | 4793.52 | 2697.08 | 1536.76 | 467.80 |
| TGFB-065 | 73.8 | 39.5 | 0.71 | 0.78 | 9.68 | 11.32 | 3134.42 | 3493.24 | 1027.76 | 650.10 |
| TGFB-066 | 34.7 | 23.7 | 1.44 | 1.37 | 8.89 | 6.86 | 3274.12 | 1127.69 | 1242.01 | 187.49 |
| TGFB-051 | 63.9 | 57.9 | 1.42 | 1.33 | 11.41 | 17.50 | 4201.64 | 7257.03 | 670.75 | 1224.37 |
| TGFB-021 | 59.1 | 61.5 | 0.45 | 1.18 | 2.26 | 15.90 | UNDER | 8855.22 | UNDER | 2002.40 |
| TGFB-067 | 56.6 | 52.7 | 1.60 | 1.26 | 11.75 | 9.94 | 4640.09 | 3562.63 | 1044.33 | 500.31 |
| TGFB-068 | 48.7 | 37.5 | 1.55 | 0.92 | 8.25 | 16.65 | 2822.77 | 5132.19 | 686.50 | 839.76 |
| TGFB-069 | 78.1 | 71.4 | 3.86 | 2.24 | 14.40 | 23.02 | OVER | OVER | 2342.69 | 1821.08 |
| TGFB-070 | 74.7 | 66.8 | 4.03 | 1.88 | 13.25 | 21.91 | 7687.75 | OVER | 1630.33 | 1513.97 |
| TGFB-071 | 81.4 | 71.5 | 3.06 | 1.37 | 18.77 | 17.75 | OVER | 7378.81 | 2296.84 | 1485.50 |
| TGFB-072 | 63.5 | 61.3 | 3.89 | 1.79 | 14.98 | 17.29 | 8702.74 | 8752.26 | 1880.88 | 2084.17 |
| TGFB-073 | 64.0 | 61.6 | 3.08 | 0.90 | 13.13 | 19.28 | 6905.27 | OVER | 1420.25 | 1657.32 |
| TGFB-074 | 41.2 | 38.8 | 2.40 | 1.44 | 11.22 | 14.58 | 5734.39 | 5516.58 | 1172.09 | 1506.55 |
| TGFB-075 | 72.7 | 58.5 | 3.86 | 2.45 | 9.64 | 14.26 | 4417.77 | 4407.46 | 1039.20 | 1114.15 |
| TGFB-076 | 72.3 | 54.9 | 2.94 | 1.37 | 14.27 | 18.15 | 6648.59 | 6104.32 | 1336.90 | 1141.19 |
| TGFB-077 | 65.1 | 52.6 | 2.52 | 1.19 | 14.40 | 15.01 | 6643.39 | 4956.00 | 1411.44 | 923.86 |
| TGFB-078 | 69.2 | 40.1 | 2.14 | 1.52 | 15.24 | 9.90 | 6322.80 | 2933.81 | 999.86 | 688.84 |
| TGFB-079 | 58.2 | 38.0 | 1.49 | 1.19 | 9.28 | 8.59 | 3578.95 | 1660.44 | 685.32 | 303.97 |
| TGFB-080 | 62.7 | 53.2 | 2.11 | 2.23 | 10.78 | 15.11 | 4381.97 | 4292.84 | 1230.70 | 1209.45 |
| TGFB-081 | 74.5 | 67.5 | 2.31 | 2.80 | 18.77 | 17.83 | OVER | 7726.72 | 2176.27 | 1885.33 |
| TGFB-082 | 68.4 | 61.9 | 3.26 | 2.85 | 20.43 | 17.07 | 7170.48 | 6053.91 | 1346.39 | 1233.19 |
| TGFB-083 | 67.0 | 55.7 | 2.08 | 2.76 | 14.07 | 16.14 | 3957.04 | 4083.78 | 968.50 | 804.85 |
| TGFB-084 | 71.8 | 63.4 | 2.19 | 2.18 | 13.72 | 16.04 | 4866.92 | 4251.35 | 1339.51 | 1040.21 |
| TGFB-019 | 52.1 | 55.0 | 1.99 | 1.88 | 21.11 | 19.48 | 6699.87 | 6549.49 | 1165.68 | 1082.83 |
| TGFB-086 | 51.8 | 13.4 | 1.76 | 2.08 | 7.83 | 14.23 | 3147.62 | 3392.39 | 938.14 | 746.35 |
| TGFB-087 | 65.1 | 49.5 | 2.51 | 1.97 | 11.95 | 16.75 | 6062.36 | 5906.26 | 1256.42 | 1092.36 |
| TGFB-088 | 14.7 | 54.7 | 0.66 | 0.72 | 2.23 | 13.51 | UNDER | 6151.66 | UNDER | 2665.41 |
| TGFB-052 | 71.2 | 64.3 | 1.57 | 1.06 | 12.76 | 22.96 | 8769.31 | OVER | 976.30 | 1045.64 |
| TGFB-091 | 49.9 | 10.5 | 0.24 | 0.43 | 1.90 | 1.74 | UNDER | UNDER | UNDER | UNDER |
| TGFB-094 | 75.2 | 44.7 | 1.20 | 1.47 | 16.51 | 10.04 | OVER | 2869.89 | 3205.68 | 733.56 |
| TGFB-095 | 78.3 | 37.2 | 1.69 | 1.41 | 11.76 | 40.40 | 6083.67 | OVER | 1892.04 | 906.32 |
| TGFB-096 | 77.3 | 47.0 | 1.00 | 0.91 | 15.99 | 23.65 | OVER | OVER | 2329.70 | 1284.39 |
| TGFB-007 | 68.3 | 67.7 | 2.00 | 1.42 | 24.06 | 14.20 | OVER | 6340.12 | 2149.84 | 1242.53 |
| dCD19 | 55.3 | 53.9 | 1.44 | 1.37 | 10.22 | 14.87 | 4498.80 | 3807.07 | 1110.72 | 605.02 |
| Mock | 1.3 | 0.4 | 1.08 | 2.18 | 6.82 | 11.32 | 2372.82 | 2716.49 | 958.59 | 483.80 |

TN = Transduction;
ND = Not detectable;
OVER = >9,000 pg/mL IFN-γ

### Materials and Methods for Examples 9, 10 and 11

Isolation and Expansion of Human NK Cells from Peripheral Blood

Human NK cells were isolated from fresh peripheral blood using the EasySep™ Human NK Cell Isolation Kit (STEMCELL TECHNOLOGIES) according to the manufacturer's instructions. Cells were activated by co-culture with engineered feeder cell line at a 2:1 (engineered feeder cell: NK) ratio and expanded prior to being frozen in cryopreservation media.

### Viral Particle Production

Viral particles encoding the chimeric proteins listed in Table 21 (below), and a fusion protein including truncated CD19 (CD191-319) and a tag (“dCD19”) were generated. Briefly, packaging cells seeded in 24-well plates in Dulbecco's modified eagle medium (DMEM) supplemented with 10% fetal bovine serum (FBS) were transfected with viral constructs using the PEIpro® transfection reagent (POLYPLUS) according to the manufacturer's instructions. 24-hours post-transfection, the culture media was changed to HyClone™ SFM4Transfx-293 serum free media (CYTIVA). Culture supernatants containing viral particles were collected 48 and 72 hours post-transfection, combined and stored at 4° C. until ready for use.

TABLE 21
Tested chimeric proteins

| | | Amino | Amino |
|---|---|---|---|
| | | acid | acid |
| | | sequence | sequence |
| | Description (extracellular | with | without |
| | domain (ECD)- | signal | signal |
| | transmembrane domain | peptide- | peptide- |
| | (TM)-intracellular domain | SEQ ID | SEQ ID |
| Construct ID | (ICD) (signal peptides excluded) | NO: | NO: |
| CONT-101 (“dCD19”) | CD19 ECD-CD19 TM | — | — |
| AA-SR-100 (SR-100) | BTLA ECD-BTLA TM-CD3z ICD | 556 | 557 |
| AA-SR-102 (SR-102) | CD200R ECD-CD200R TM-CD3z ICD | 558 | 559 |
| AA-SR-104 (SR-104) | CTLA4 ECD-CTLA4 TM-CD3z ICD | 560 | 561 |
| AA-SR-105 (SR-105) | Fas ECD-Fas TM-CD3z ICD | 562 | 563 |
| AA-SR-106 (SR-106) | IL10RA ECD-IL10RA TM-CD3z ICD | 564 | 565 |
| AA-SR-107 (SR-107) | IL10RB ECD-IL10RB TM-CD3z ICD | 566 | 567 |
| AA-SR-109 (SR-109) | GP130 ECD-GP130 TM-CD3z ICD | 568 | 569 |
| AA-SR-111 (SR-111) | PD1 ECD-PD1 TM-CD3z ICD | 570 | 571 |
| AA-SR-112 (SR-112) | TACTILE ECD-TACTILE TM-CD3z ICD | 572 | 573 |
| AA-SR-113 (SR-113) | TIGIT ECD-TIGIT TM-CD3z ICD | 574 | 575 |
| AA-SR-115 (SR-115) | BTLA ECD-CD28 TM-CD3z ICD | 576 | 577 |
| AA-SR-117 (SR-117) | CD200R ECD-CD28 TM-CD3z ICD | 578 | 579 |
| AA-SR-119 (SR-119) | CTLA4 ECD-CD28 TM-CD3z ICD | 580 | 581 |
| AA-SR-120 (SR-120) | Fas ECD-CD28 TM-CD3z ICD | 582 | 583 |
| AA-SR-121 (SR-121) | IL10RA ECD-CD28 TM-CD3z ICD | 584 | 585 |
| AA-SR-122 (SR-122) | IL10RB ECD-CD28 TM-CD3z ICD | 586 | 587 |
| AA-SR-124 (SR-124) | GP130 ECD-CD28 TM-CD3z ICD | 588 | 589 |
| AA-SR-126 (SR-126) | PD1 ECD-CD28 TM-CD3z ICD | 590 | 591 |
| AA-SR-127 (SR-127) | TACTILE ECD-CD28 TM-CD3z ICD | 592 | 593 |
| AA-SR-128 (SR-128) | TIGIT ECD-CD28 TM-CD3z ICD | 594 | 595 |
| AA-TGFB-056 (TGFB-056) | TGF-BR2 ECD-CD28 Long TM-SLP76 ICD | 596 | 597 |
| AA-TGFB-057 (TGFB-057) | TGF-BR2 ECD-CD28 Long TM-MYD88 ICD | 598 | 599 |
| AA-TGFB-058 (TGFB-058) | TGF-BR2 ECD-DAP12 TM-DAP12 ICD | 600 | 601 |
| AA-TGFB-059 (TGFB-059) | TGF-BR2 ECD-CD28 Long TM-TRAF1 ICD | 602 | 603 |
| AA-TGFB-060 (TGFB-060) | TGF-BR2 ECD-CD28 Long TM-TRAF2 ICD | 604 | 605 |
| AA-TGFB-061 (TGFB-061) | TGF-BR2 ECD-CD28 Long TM-TRAF3 ICD | 606 | 607 |
| AA-TGFB-062 (TGFB-062) | TGF-BR2 ECD-CD28 Long TM-TRAF6 ICD | 608 | 609 |
| AA-TGFB-064 (TGFB-064) | TGF-BR2 ECD-CD28 Long TM-TIRAP ICD | 610 | 611 |
| AA-TGFB-065 (TGFB-065) | TGF-BR2 ECD-CD28 Long TM-BLNK ICD | 612 | 613 |
| AA-TGFB-066 (TGFB-066) | TGF-BR2 ECD-CD28 Long TM-GRB2 ICD | 614 | 615 |
| AA-TGFB-067 (TGFB-067) | TGF-BR2 ECD-FCGR2A TM-FCGR2A ICD | 616 | 617 |
| AA-TGFB-068 (TGFB-068) | TGF-BR2 ECD-FCGR3A TM-FCGR3A ICD | 618 | 619 |
| AA-TGFB-069 (TGFB-069) | TGF-BR2 ECD-2B4 TM-2B4 ICD | 620 | 621 |
| AA-TGFB-070 (TGFB-070) | TGF-BR2 ECD-SLAMF1 TM-SLAMF1 ICD | 622 | 623 |
| AA-TGFB-071 (TGFB-071) | TGF-BR2 ECD-SLAMF5 TM-SLAMF5 ICD | 624 | 625 |
| AA-TGFB-072 (TGFB-072) | TGF-BR2 ECD-SLAMF6 TM-SLAMF6 ICD | 626 | 627 |
| AA-TGFB-073 (TGFB-073) | TGF-BR2 ECD-SLAMF7 TM-SLAMF7 ICD | 628 | 629 |
| AA-TGFB-074 (TGFB-074) | TGF-BR2 ECD-LFA2 TM-LFA2 ICD | 630 | 631 |
| AA-TGFB-075 (TGFB-075) | TGF-BR2 ECD-SLAMF3 TM-SLAMF3 ICD | 632 | 633 |
| AA-TGFB-076 (TGFB-076) | TGF-BR2 ECD-EPOR TM-EPOR ICD | 634 | 635 |
| AA-TGFB-077 (TGFB-077) | TGF-BR2 ECD-GCSFR TM-GCSFR ICD | 636 | 637 |
| AA-TGFB-078 (TGFB-078) | TGF-BR2 ECD-CSF1R TM-CSF1R ICD | 638 | 639 |
| AA-TGFB-079 (TGFB-079) | TGF-BR2 ECD-FGFR1 TM-FGFR1 ICD | 640 | 641 |
| AA-TGFB-080 (TGFB-080) | TGF-BR2 ECD-DNAM1 TM-DNAM1 ICD | 642 | 643 |
| AA-TGFB-081 (TGFB-081) | TGF-BR2 ECD-ICOS TM-ICOS ICD | 644 | 645 |
| AA-TGFB-082 (TGFB-082) | TGF-BR2 ECD-NKp46 TM-NKp46 ICD | 646 | 647 |
| AA-TGFB-083 (TGFB-083) | TGF-BR2 ECD-NKp44 TM-NKp44 ICD | 648 | 649 |
| AA-TGFB-084 (TGFB-084) | TGF-BR2 ECD-NKp30 TM-NKp30 ICD | 650 | 651 |
| AA-TGFB-086 (TGFB-086) | TGF-BR2 ECD-CD28 Long TM-IRAK4 ICD | 652 | 653 |
| AA-TGFB-087 (TGFB-087) | TGF-BR2 ECD-TLR4 TM-TLR4 ICD | 654 | 655 |
| AA-TGFB-088 (TGFB-088) | TGF-BR2 ECD-DR3 TM-DR3 ICD | 656 | 657 |
| AA-TGFB-091 (TGFB-091) | TGF-BR2 ECD-CD28 Long TM-Syk ICD | 658 | 659 |
| AA-TGFB-094 (TGFB-094) | TGF-BR2 ECD-CD28 Long TM-Lyn ICD | 660 | 661 |
| AA-TGFB-095 (TGFB-095) | TGF-BR2 ECD-CD28 Long TM-YES1 ICD | 662 | 663 |
| AA-TGFB-096 (TGFB-096) | TGF-BR2 ECD-CD28 Long TM-Fgr ICD | 664 | 665 |
| AA-TGFB-052 (TGFB-052) | TGF-BR2 ECD-CD28 TM-41BB ICD | 666 | 667 |
| AA-TGFB-051 (TGFB-051) | TGF-BR2 ECD-CD28 TM-DAP10v2 ICD | 668 | 669 |
| AA-TGFB-019 (TGFB-019) | TGF-BR2 ECD-CD28 TM-CD28 ICD | 670 | 671 |
| AA-TGFB-021 (TGFB-021) | TGF-BR2 ECD-CD28 TM-CD3z ICD | 672 | 673 |
| AA-TGFB-007 (TGFB-007) | TGF-BR2 ECD-TGFBR2 | 674 | 675 |
| | TM-TGFBR2 truncated | | |
| | ICD | | |

### Cell Transduction

Previously expanded human NK cells were thawed and cultured overnight in the presence of 100 IU/mL of recombinant human IL-2. The next day, the NK cells were activated by co-culture with engineered feeder cells at a 2:1 ratio, and the NK cells were cultured for 72 hours.

Previously collected viral supernatants were loaded into RetroNectin (TAKARA BIO)-coated 24-well tissue culture plates. Following supernatant removal, 5×10⁵ activated human NK cells or Jurkat-NFκB-GFP reporter cells (SYSTEM BIOSCIENCES) were added to the plates. After 3 days of culture, the transduced NK cells were collected and counted using an automated cell counter. Jurkat-NF-κB-GFP cells were collected following 4 days of culture and counted using an automated cell counter. Approximately 1×10⁵ cells from each transduced cell culture were collected for staining. Cells were pelleted using centrifugation and resuspended in Cell Staining Buffer (BIOLEGEND) and then stained with antibodies to detect the expression of the chimeric proteins following the manufacturer's recommended protocols. Stained cells were analyzed using a BD FACSymphony™ A3 cell analyzer (BD BIOSCIENCES).

### Jurkat-NFκB-GFP Reporter Assays

Transduced Jurkat-NF-κB-GFP reporter cells expressing a chimericprotein of interest were plated at 1×10⁵ cells per well in 96-well tissue culture plates previously coated with increasing concentrations of a monoclonal antibody (mAb) targeting the extracellular domain of the chimeric protein expressed by the cells (low: 0.4 μg/mL; medium: 2 μg/mL; or high: 10 μg/mL), soluble TGF-B1 (low: 0.002 μg/mL; medium: 0.01 μg/mL; or high: 0.05 μg/mL; PEPROTECH) or plate-coated FasL (low: 0.4 μg/mL; medium: 2 μg/mL; or high: 10 μg/mL; THERMO FISHER SCIENTIFIC) (“stimulants”). Cells were incubated with their respective treatment conditions overnight, collected and analyzed by flow cytometry for expression of GFP and CD69.

The monoclonal antibodies that were used were: anti-CD3 mAb clone OKT3, anti-CD28 mAb clone CD28.2, anti-TIM-3 mAb clone F38-2E2, anti-CD161 mAb clone HP-3G10, anti-CD96 mAb clone NK92.39, anti-TIGIT mAb clone A15153G, anti-BTLA mAb clone MIH26, anti-CD200R mAb clone OX-108, anti-CD33 mAb clone WM53, anti-PD-1 mAb clone EH12.2H7, and anti-TGF-BR2 mAb clone W17055E (each from BIOLEGEND); anti-NKG2A mAb clone 131411, anti-CTLA-4 mAb clone 1003705, anti-IL-10RA mAb clone 37607, anti-IL-10RB mAb clone 90220, anti-IL-6RA mAb clone 17506R, anti-gp130 mAb clone 28126, and anti-CD94 mAb clone 131412 (each from R&D Systems); anti-CD160 mAb clone CNX46-3, anti-LILRB2 mAb clone 42D1), and anti-KLRG1 mAb clone 13F12F2 (each from THERMO FISHER SCIENTIFIC).

### Exposure of Human NK Cells to Chronic TGF-B1 Treatment

To expose human NK cells to chronic stimulation with TGF-B1 cytokine, 5×10⁵ human NK cells transduced to express each chimeric protein were collected on day 3 post-transduction and plated in 24-well cell culture plates. The cells were cultured for 5 days in the presence of 100 IU/mL recombinant human IL-2 cytokine, with either 10 ng/mL recombinant TGF-B1 cytokine (PEPROTECH), or noTGF-B1 as a control. Fresh cytokine was added to the NK cells on days 2 and 4 of the culture. On day 5, the NK cells were collected and counted using an automated cell counter. Cell survival was assessed by flow cytometry and cells were counted to calculate fold expansion.

### Cytokine and Chemokine Analysis

To detect secreted soluble factors (i.e., IP10 and interferon-gamma (IFN-γ)), supernatant samples were collected and stored at −20° C. Subsequently, the supernatant samples were thawed and analyzed using either the V-PLEX Proinflammatory Panel 1 Human Kit (to detect IFN-γ) and the V-PLEX Chemokine Panel 1 Human 1 Human Kit (to detect IP-10) (both from MESO SCALE DIAGNOSTICS), and the MESO QuickPlex SQ 120 instrument (MESO SCALE DIAGNOSTICS).

### Evaluation of NK Cell Cytotoxicity Against Tumor Cells

For cytotoxicity assays, SKOV-3 target cells expressing NanoLuc luciferase (SKOV3-luc Cells) were seeded in 96-well plates and cultured for 24-hours. Transduced NK cells were treated with 10 ng/ml of -TGF-B1 for 5 days as described above in the “Exposure of human NK cells to chronic TGF-B1 treatment” section, prior to being collected and plated for the cytotoxicity assay. For both acute and chronic assays, NK cells were added to the assay wells to achieve a 3:1 ratio of NK cells to SKOV3-Luc target cells and incubated overnight. Control wells contained NK cells alone or target cells alone.

For each sample, 10 μL of supernatant was collected and placed in 96-well assay plates (PERKINELMER HEALTH SCIENCES). For control wells containing target cells only, supernatant was removed and 200 μL of Passive Lysis 5× buffer (PROMEGA) was added to the wells to lyse the SKOV3-Luc cells. 10 μL of lysate was then used in assays to obtain a “maximum killing luminescence” value to normalize luminescence from the remaining samples. For the remaining samples, Nano-Glo® Luciferase Assay System (PROMEGA) was added, and luminescence measured using an EnVision® multimode plate reader (PERKINELMER) according to the manufacturer's protocol. The percent of target cell killing was calculated as 100% x luminescence from supernatant/maximum killing luminescence.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit, and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope, and concept of the disclosure as defined by the appended claims.

1. A chimeric protein comprising an extracellular domain, a transmembrane domain, and an intracellular domain, wherein the extracellular domain is capable of binding a negative signal, and wherein the intracellular domain comprises an intracellular domain, or a portion thereof, of a stimulatory polypeptide, and the negative signal is selected from the group consisting of IL-10, TGF-β, IL-1, IL-6, PD-L1, PD-L2, B7-1, B7-2, MHCI, HVEM, CD155, CD112, CD111, CD200, B7-H6, HS-GAG, HLA, N-cadherin, E-cadherin, FasL, MHCII, TIM-3, IL-18, adenosine, and prostaglandin.

2. The chimeric protein of claim 1, wherein said chimeric protein is capable of activating an immune cell selected from a natural killer (NK) cell, an NKT cell, a T cell, and a macrophage.

3. (canceled)

4. The chimeric protein of claim 1, wherein the extracellular domain comprises an antigen-binding domain that specifically binds to the negative signal.

5.-8. (canceled)

9. The chimeric protein of claim 1, wherein the extracellular domain comprises the extracellular domain, or a portion thereof, of an inhibitory polypeptide that binds the negative signal.

10. The chimeric protein of claim 9, wherein the inhibitory polypeptide is an inflammatory mediator receptor, an inhibitory cytokine receptor, an immune checkpoint receptor, or a dual activator-checkpoint receptor.

11. The chimeric protein of claim 9, wherein the inhibitory polypeptide is selected from the inhibitory polypeptides presented in Table 1 or Table 1.1.

12. The chimeric protein of claim 1, wherein the transmembrane domain comprises the transmembrane domain, or a portion thereof, of an inhibitory polypeptide presented in Table 1 or Table 1.2, or a stimulatory polypeptide presented in Table 2 or Table 2.2.

13. (canceled)

14. The chimeric protein of claim 1, wherein the intracellular domain comprises the intracellular domain, or a portion thereof, of a stimulatory polypeptide selected from the stimulatory polypeptides presented in Table 2 or Table 2.1.

15.-16. (canceled)

17. The chimeric protein of claim 1, wherein:
the inhibitory polypeptide is a type I receptor, and the stimulatory polypeptide is a type I receptor;
the inhibitory polypeptide is a type I receptor, and the stimulatory polypeptide is a type III receptor;
the inhibitory polypeptide is a type II receptor, and the stimulatory polypeptide is a type II receptor;
the inhibitory polypeptide is a type I receptor, and the stimulatory polypeptide is not associated with the plasma membrane; or
the inhibitory polypeptide is a type I receptor, the stimulatory polypeptide is a type II receptor, and the transmembrane domain, or portion thereof, is from a type I receptor.

18. (canceled)

19. The chimeric protein of claim 1, wherein a combination of the extracellular domain, or a portion thereof, of an inhibitory polypeptide and the intracellular domain, or a portion thereof, of a stimulatory polypeptide is selected from the combinations presented in any one of Tables 6-14.

20. The chimeric protein of claim 1, wherein the extracellular domain and the transmembrane domain are connected by a linker.

21. The chimeric protein of claim 1, wherein the transmembrane domain and the intracellular domain are connected by a linker.

22. (canceled)

23. A modified immune cell engineered to express a chimeric protein comprising an extracellular domain, a transmembrane domain, and an intracellular domain, wherein the extracellular domain is capable of binding a negative signal, and wherein the intracellular domain comprises an intracellular domain, or a portion thereof, of a stimulatory polypeptide, and the negative signal is selected from the group consisting of IL-10, TGF-β, IL-1, IL-6, PD-L1, PD-L2, B7-1, B7-2, MHCI, HVEM, CD155, CD112, CD111, CD200, B7-H6, HS-GAG, HLA, N-cadherin, E-cadherin, FasL, MHCII, TIM-3, IL-18, adenosine, and prostaglandin.

24.-94. (canceled)

95. A protein comprising an extracellular domain and a transmembrane domain, wherein the extracellular domain is capable of binding a negative signal, and wherein the protein lacks a fully functional intracellular domain, and the negative signal is selected from the group consisting of IL-10, TGF-β, IL-1, IL-6, PD-L1, PD-L2, B7-1, B7-2, MHCI, HVEM, CD155, CD112, CD111, CD200, B7-H6, HS-GAG, HLA, N-cadherin, E-cadherin, FasL, MHCII, TIM-3, IL-18, adenosine, and prostaglandin.

96.-110. (canceled)

111. A modified cell engineered to express a protein comprising an extracellular domain and a transmembrane domain, wherein the extracellular domain is capable of binding a negative signal, and wherein the protein lacks a fully functional intracellular domain, and the negative signal is selected from the group consisting of IL-10, TGF-β, IL-1, IL-6, PD-L1, PD-L2, B7-1, B7-2, MHCI, HVEM, CD155, CD112, CD111, CD200, B7-H6, HS-GAG, HLA, N-cadherin, E-cadherin, FasL, MHCII, TIM-3, IL-18, adenosine, and prostaglandin.

112.-131. (canceled)

132. A modified cell engineered to express a protein comprising a dominant negative isoform of a protein, wherein the dominant negative isoform of the protein competes with a wild-type isoform of the protein for binding a negative signal, wherein the negative signal is selected from the group consisting of IL-10, TGF-β, IL-1, IL-6, PD-L1, PD-L2, B7-1, B7-2, MHCI, HVEM, CD155, CD112, CD111, CD200, B7-H6, HS-GAG, HLA, N-cadherin, E-cadherin, FasL, MHCII, TIM-3, IL-18, adenosine, and prostaglandin.

133.-145. (canceled)

146. A modified cell engineered to express at least two proteins selected from the group consisting of:
(a) a chimeric protein comprising an extracellular domain, a transmembrane domain, and an intracellular domain, wherein the extracellular domain is capable of binding a negative signal, and wherein the intracellular domain comprises an intracellular domain, or a portion thereof, of a stimulatory polypeptide;
(b) a protein comprising a dominant negative isoform of a protein, wherein the dominant negative isoform of the protein competes with a wild-type isoform of the protein for binding a negative signal that prevents the activation of an immune response; and
(c) a protein comprising an extracellular domain and a transmembrane domain, wherein the extracellular domain is capable of binding a negative signal, and wherein the protein lacks a fully functional intracellular domain,
wherein the negative signal is selected from the group consisting of IL-10, TGF-β, IL-1, IL-6, PD-L1, PD-L2, B7-1, B7-2, MHCI, HVEM, CD155, CD112, CD111, CD200, B7-H6, HS-GAG, HLA, N-cadherin, E-cadherin, FasL, MHCII, TIM-3, IL-18, adenosine, and prostaglandin.

147.-149. (canceled)

150. A polynucleotide comprising a nucleic acid sequence encoding a chimeric protein of claim 1.

151. A pharmaceutical composition comprising the modified cell of claim 23, and a pharmaceutically acceptable excipient.

152. A method of treating a subject in need of an altered immune response, the method comprising administering to the subject an effective amount of a composition comprising the modified cell of claim 23, thereby treating the subject in need of the altered immune response.

153. A method of treating a disease or pathological condition in a subject, comprising administering to the subject an effective amount of a composition comprising the modified cell of claim 23, thereby treating the disease or pathological condition in the subject.

154. A method of treating a cancer in a subject, comprising administering to the subject a therapeutically effective amount of a composition comprising the modified cell of claim 23, thereby treating the cancer in the subject.

155. A method of generating the modified cell of any one of the preceding claims, the method comprising:
(a) introducing a nucleic acid encoding the chimeric protein of claim 1, into a cell;
(b) culturing the cell under conditions allowing the expression of the protein in or on the cell; and
(c) recovering the cell from the culture,
thereby generating the modified cell.

156. A cell obtained by the method of claim 155.

157. A kit comprising a chimeric protein and/or a nucleic acid encoding the chimeric protein, wherein the chimeric protein is the chimeric protein of claim 1.

158. A kit comprising an engineered protein and/or a nucleic acid encoding the engineered protein, wherein the engineered protein is the engineered protein of claim 95.


SEQUENCE LISTING (687 sequences)

SEQ ID NO: 1-3 - skipped (3 entries)

SEQ ID NO: 4 - protein, 6 aa, synthetic construct
LPXTGG

SEQ ID NO: 5 - protein, 127 aa, Homo sapiens
KESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYR

SEQ ID NO: 6 - protein, 138 aa, Homo sapiens
GCINITSSASQEGTRLNLICTVWHKKEEAEGFVVFLCKDRSGDCSPETSLKQLRLKRDPGIDGVGEISSQLMFTISQVTPLHSGTYQCCARSQKSGIRLQGHFFSILFTETGNYTVTGLKQRQHLEFSHNEGTLSSGF

SEQ ID NO: 7 - protein, 215 aa, Homo sapiens
MDEKQITQNYSKVLAEVNTSWPVKMATNAVLCCPPIALRNLIIITWEIILRGQPSCTKAYRKETNETKETNCTDERITWVSRPDQNSDLQIRPVAITHDGYYRCIMVTPDGNFHRGYHLQVLVTPEVTLFQNRNRTAVCKAVAGKPAAQISWIPEGDCATKQEYWSNGTVTVKSTCHWEVHNVSTVTCHVSHLTGNKSLYIELLPVPGAKKSAKL

SEQ ID NO: 8 - protein, 242 aa, Homo sapiens
DPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVH

SEQ ID NO: 9 - protein, 394 aa, Homo sapiens
QLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPG

SEQ ID NO: 10 - protein, 126 aa, Homo sapiens [sequence omitted]

SEQ ID NO: 11 - protein, 148 aa, Homo sapiens
QVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSN

SEQ ID NO: 12 - protein, 288 aa, Homo sapiens
LYLQAWPNPVFEGDALTLRCQGWKNTPLSQVKFYRDGKFLHFSKENQTLSMGAATVQSRGQYSCSGQVMYIPQTFTQTSETAMVQVQELFPPPVLSAIPSPEPREGSLVTLRCQTKLHPLRSALRLLFSFHKDGHTLQDRGPHPELCIPGAKEGDSGLYWCEVAPEGGQVQKQSPQLEVRVQAPVSRPVLTLHHGPADPAVGDMVQLLCEAQRGSPPILYSFYLDEKIVGNHSAPCGGTTSLLFPVKSEQDAGNYSCEAENSVSRERSEPKKLSLKGSQVLFTPASNW

SEQ ID NO: 13 - protein, 214 aa, Homo sapiens
HGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTN

SEQ ID NO: 14 - protein, 201 aa, Homo sapiens
MVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPS

SEQ ID NO: 15 - protein, 118 aa, Homo sapiens
MPGVCDRAPDFLSPSEDQVLRPALGSSVALNCTAWVVSGPHCSLPSVQWLKDGLPLGIGGHYSLHEYSWVKANLSEVLVSSVLGVNVTSTEVYGAFTCSIQNISFSSFTLQRAGPTSH

SEQ ID NO: 16 - protein, 346 aa, Homo sapiens
LAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLP

SEQ ID NO: 17 - protein, 597 aa, Homo sapiens
ELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIE

SEQ ID NO: 18 - protein, 224 aa, Homo sapiens
HEGVHRKPSLLAHPGPLVKSEETVILQCWSDVMFEHFLLHREGMFNDTLRLIGEHHDGVSKANFSISRMTQDLAGTYRCYGSVTHSPYQVSAPSDPLDIVIIGLYEKPSLSAQPGPTVLAGENVTLSCSSRSSYDMYHLSREGEAHERRLPAGPKVNGTFQADFPLGPATHGGTYRCFGSFHDSPYEWSKSSDPLLVSVTGNPSNSWPSPTEPSSKTGNPRHLH

SEQ ID NO: 19 - protein, 224 aa, Homo sapiens
HEGVHRKPSLLAHPGRLVKSEETVILQCWSDVRFEHFLLHREGKFKDTLHLIGEHHDGVSKANFSIGPMMQDLAGTYRCYGSVTHSPYQLSAPSDPLDIVITGLYEKPSLSAQPGPTVLAGESVTLSCSSRSSYDMYHLSREGEAHECRFSAGPKVNGTFQADFPLGPATHGGTYRCFGSFRDSPYEWSNSSDPLLVSVIGNPSNSWPSPTEPSSKTGNPRHLH

SEQ ID NO: 20 - protein, 224 aa, Homo sapiens
HEGVHRKPSLLAHPGPLVKSEETVILQCWSDVRFQHFLLHREGKFKDTLHLIGEHHDGVSKANFSIGPMMQDLAGTYRCYGSVTHSPYQLSAPSDPLDIVITGLYEKPSLSAQPGPTVLAGESVTLSCSSRSSYDMYHLSREGEAHERRFSAGPKVNGTFQADFPLGPATHGGTYRCFGSFRDSPYEWSNSSDPLLVSVTGNPSNSWPSPTEPSSETGNPRHLH

SEQ ID NO: 21 - protein, 217 aa, Homo sapiens
HEGGQDKPLLSAWPSAVVPRGGHVTLLCRSRLGFTIFSLYKEDGVPVPELYNKIFWKSILMGPVTPAHAGTYRCRGSHPRSPIEWSAPSNPLVIVVTGLFGKPSLSAQPGPTVRTGENVTLSCSSRSSFDMYHLSREGRAHEPRLPAVPSVNGTFQADFPLGPATHGGTYTCFGSLHDSPYEWSDPSDPLLVSVTGNSSSSSSSPTEPSSKTGIRRH

SEQ ID NO: 22 - protein, 217 aa, Homo sapiens
HEGGQDKPLLSAWPSAVVPRGGHVTLLCRSRLGFTIFSLYKEDGVPVPELYNKIFWKSILMGPVTPAHAGTYRCRGSHPRSPIEWSAPSNPLVIVVTGLFGKPSLSAQPGPTVRTGENVTLSCSSRSSFDMYHLSREGRAHEPRLPAVPSVDGTFQADFPLGPATHGGTYTCFSSLHDSPYEWSDPSDPLLVSVTGNSSSSSSSPTEPSSKTGIRRH

SEQ ID NO: 23 - protein, 319 aa, Homo sapiens
HMGGQDKPFLSAWPSAVVPRGGHVTLRCHYRHRFNNFMLYKEDRIHIPIFHGRIFQESFNMSPVTTAHAGNYTCRGSHPHSPTGWSAPSNPVVIMVTGNHRKPSLLAHPGPLVKSGERVILQCWSDIMFEHFFLHKEGISKDPSRLVGQIHDGVSKANFSIGPMMLALAGTYRCYGSVTHTPYQLSAPSDPLDIVVTGPYEKPSLSAQPGPKVQAGESVTLSCSSRSSYDMYHLSREGGAHERRLPAVRKVNRTFQADFPLGPATHGGTYRCFGSFRHSPYEWSDPSDPLLVSVTGNPSSSWPSPTEPSSKSGNPRHLH

SEQ ID NO: 24 - protein, 319 aa, Homo sapiens
LMGGQDKPFLSARPSTVVPRGGHVALQCHYRRGFNNFMLYKEDRSHVPIFHGRIFQESFIMGPVTPAHAGTYRCRGSRPHSLTGWSAPSNPLVIMVTGNHRKPSLLAHPGPLLKSGETVILQCWSDVMFEHFFLHREGISEDPSRLVGQIHDGVSKANFSIGPLMPVLAGTYRCYGSVPHSPYQLSAPSDPLDIVITGLYEKPSLSAQPGPTVQAGENVTLSCSSWSSYDIYHLSREGEAHERRLRAVPKVNRTFQADFPLGPATHGGTYRCFGSFRALPCVWSNSSDPLLVSVTGNPSSSWPSPTEPSSKSGICRHLH

SEQ ID NO: 25 - protein, 297 aa, Homo sapiens
QDKPFLSAWPGTVVSEGQHVTLQCRSRLGFNEFSLSKEDGMPVPELYNRIFRNSFLMGPVTPAHAGTYRCCSSHPHSPTGWSAPSNPVVIMVTGVHRKPSLLAHPGPLVKSGETVILQCWSDVRFERFLLHREGITEDPLRLVGQLHDAGSQVNYSMGPMTPALAGTYRCFGSVTHLPYELSAPSDPLDIVVVGLYGKPSLSAQPGPTVQAGENVTLSCSSRSLFDIYHLSREAEAGELRLTAVLRVNGTFQANFPLGPVTHGGNYRCFGSFRALPHAWSDPSDPLPVSVTGNSRHL

SEQ ID NO: 26 - protein, 428 aa, Homo sapiens
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHL

SEQ ID NO: 27 - protein, 438 aa, Homo sapiens
GHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGV

SEQ ID NO: 28 - protein, 440 aa, Homo sapiens
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGV

SEQ ID NO: 29 - protein, 420 aa, Homo sapiens
GPFPKPTLWAEPGSVISWGSPVTIWCQGSQEAQEYRLHKEGSPEPLDRNNPLEPKNKARFSIPSMTEHHAGRYRCHYYSSAGWSEPSDPLEMVMTGAYSKPTLSALPSPVVASGGNMTLRCGSQKGYHHFVLMKEGEHQLPRTLDSQQLHSRGFQALFPVGPVTPSHRWRFTCYYYYTNTPWVWSHPSDPLEILPSGVSRKPSLLTLQGPVLAPGQSLTLQCGSDVGYNRFVLYKEGERDFLQRPGQQPQAGLSQANFTLGPVSPSNGGQYRCYGAHNLSSEWSAPSDPLNILMAGQIYDTVSLSAQPGPTVASGENVTLLCQSWWQFDTFLLTKEGAAHPPLRLRSMYGAHKYQAEFPMSPVTSAHAGTYRCYGSYSSNPHLLSHPSEPLELVVSGHSGGSSLPPTGPPSTPGLGRYLE

SEQ ID NO: 30 - protein, 238 aa, Homo sapiens
QAGPLPKPTLWAEPGSVISWGNSVTIWCQGTLEAREYRLDKEESPAPWDRQNPLEPKNKARFSIPSMTEDYAGRYRCYYRSPVGWSQPSDPLELVMTGAYSKPTLSALPSPLVTSGKSVTLLCQSRSPMDTFLLIKERAAHPLLHLRSEHGAQQHQAEFPMSPVTSVHGGTYRCFSSHGFSHYLLSHPSDPLELIVSGSLEDPRPSPTRSVSTAAGPEDQPLMPTGSVPHSGLRRHWE

SEQ ID NO: 31 - protein, 435 aa, Homo sapiens
GTLPKPTLWAEPASVIARGKPVTLWCQGPLETEEYRLDKEGLPWARKRQNPLEPGAKAKFHIPSTVYDSAGRYRCYYETPAGWSEPSDPLELVATGFYAEPTLLALPSPVVASGGNVTLQCDTLDGLLTFVLVEEEQKLPRTLYSQKLPKGPSQALFPVGPVTPSCRWRFRCYYYYRKNPQVWSNPSDLLEILVPGVSRKPSLLIPQGSVVARGGSLTLQCRSDVGYDIFVLYKEGEHDLVQGSGQQPQAGLSQANFTLGPVSRSHGGQYRCYGAHNLSPRWSAPSDPLDILIAGLIPDIPALSVQPGPKVASGENVTLLCQSWHQIDTFFLTKEGAAHPPLCLKSKYQSYRHQAEFSMSPVTSAQGGTYRCYSAIRSYPYLLSSPSYPQELVVSGPSGDPSLSPTGSTPTPGPEDQPLTPTGLDPQSGLGRHLG

SEQ ID NO: 32 - protein, 118 aa, Homo sapiens
ALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLG

SEQ ID NO: 33 - protein, 147 aa, Homo sapiens
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 34 - protein, 534 aa, Homo sapiens
MDGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKPDVYIPETLEPGQPVTVICVFNWAFEECPPPSFSWTGAALSSQGTKPTTSHFSVLSFTPRPQDHNTDLTCHVDFSRKGVSAQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLSVQYPPENLRVMVSQANRTVLENLGNGTSLPVLEGQSLCLVCVTHSSPPARLSWTQRGQVLSPSQPSDPGVLELPRVQVEHEGEFTCHARHPLGSQHVSLSLSVHYSPKLLGPSCSWEAEGLHCSCSSQASPAPSLRWWLGEELLEGNSSQDSFEVTPSSAGPWANSSLSLHGGLSSGLRLRCEAWNVHGAQSGSILQLPDKKGLISTAFSN

SEQ ID NO: 35 - protein, 335 aa, Homo sapiens
QKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFRMEKGNIKWNYKYDQLSVNVTALTHRPNILIPGTLESGCFQNLTCSVPWACEQGTPPMISWMGTSVSPLHPSTTRSSVLTLIPQPQHHGTSLTCQVTLPGAGVTTNRTIQLNVSYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVSGVLL

SEQ ID NO: 36 - protein, 331 aa, Homo sapiens
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQG

SEQ ID NO: 37 - protein, 498 aa, Homo sapiens
VWEKTVNTEENVYATLGSDVNLTCQTQTVGFFVQMQWSKVTNKIDLIAVYHPQYGFYCAYGRPCESLVTFTETPENGSKWTLHLRNMSCSVSGRYECMLVLYPEGIQTKIYNLLIQTHVTADEWNSNHTIEIEINQTLEIPCFQNSSSKISSEFTYAWSVENSSTDSWVLLSKGIKEDNGTQETLISQNHLISNSTLLKDRVKLGTDYRLHLSPVQIFDDGRKFSCHIRVGPNKILRSSTTVKVFAKPEIPVIVENNSTDVLVERRFTCLLKNVFPKANITWFIDGSFLHDEKEGIYITNEERKGKDGFLELKSVLTRVHSNKPAQSDNLTIWCMALSPVPGNKVWNISSEKITFLLGSEISSTDPPLSVTESTLDTQPSPASSVSPARYPATSSVTLVDVSALRPNTTPQPSNSSMTTRGFNYPWTSSGTDTKKSVSRIPSETYSSSPSGAGSTLHDNVFTSTARAFSEVPTTANGSTKTNHVHITGIVVNKPKDGM

SEQ ID NO: 38 - protein, 93 aa, Homo sapiens
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVEL

SEQ ID NO: 39 - protein, 144 aa, Homo sapiens
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 40 - protein, 120 aa, Homo sapiens
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIP

SEQ ID NO: 41 - protein, 181 aa, Homo sapiens
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIG

SEQ ID NO: 42 - protein, 10 aa, Homo sapiens
MAVFKTTLWR

SEQ ID NO: 43 - protein, 45 aa, Homo sapiens
MDQQAIYAELNLPTDSGPESSSPSSLPRDVCQGSPWHQFALKLSC

SEQ ID NO: 44 - protein, 38 aa, Homo sapiens
MTDSVIYSMLELPTATQAQNDYGPQQKSSSSRPSCSCL

SEQ ID NO: 45 - protein, 70 aa, Homo sapiens
MDNQGVIYSDLNLPPNPKRQQRKPKGNKNSILATEQEITYAELNLQKASQDFQGNDKTYHCKDLPSAPEK

SEQ ID NO: 46 - protein, 144 aa, Homo sapiens; identical to SEQ ID NO: 39

SEQ ID NO: 47 - protein, 93 aa, Homo sapiens; identical to SEQ ID NO: 38

SEQ ID NO: 48 - protein, 24 aa, Homo sapiens
LCARPRRSPAQEDGKVYINMPGRG

SEQ ID NO: 49 - protein, 126 aa, Homo sapiens
HWKHRVATRFTLPRFLQRRSSRRKVCTKTFLGPRIIGLRHEISVETQDHKSAVRGNNTHDNYENVEAGPPKAKGKTDKELYENTGQSNFEEHIYGNETSSDYYNFQKPRPSEVPQDEDIYILPDSY

SEQ ID NO: 50 - protein, 52 aa, Homo sapiens
YFLGRLVPRGRGAAEAATRKQRITETESPYQELQGQRSDVYSDLNTQRPYYK

SEQ ID NO: 51 - protein, 42 aa, Homo sapiens
RLKIQVRKAAITSYEKSDGVYTGLSTRNQETYETLKHEKPPQ

SEQ ID NO: 52 - protein, 61 aa, Homo sapiens
RKELKRKKKWDLEISLDSGHEKKVISSLQEDRHLEEELKCQEQKEEQLQEGVHRKEPQGAT

SEQ ID NO: 53 - protein, 77 aa, Homo sapiens
CRKKRISANSTDPVKAAQFEPPGRQMIAIRKRQLEETNNDYETADGGYMTLNPRAPTDDDKNIYLTLPPNDHVNSNN

SEQ ID NO: 54 - protein, 77 aa, Homo sapiens
CRKKRISANSTDPVKAAQFEPPGRQMIAIRKRQPEETNNDYETADGGYMTLNPRAPTDDDKNIYLTLPPNDHVNSNN

SEQ ID NO: 55 - protein, 25 aa, Homo sapiens
KTNIRSSTRDWKDHKFKWRKDPQDK

SEQ ID NO: 56 - protein, 41 aa, Homo sapiens
ENWHSHTALNKEASADVAEPSWSQQMCQPGLTFARTPSVCK

SEQ ID NO: 57 - protein, 12 aa, Homo sapiens
KCDRKPDRTNSN

SEQ ID NO: 58 - protein, 120 aa, Homo sapiens
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 59 - protein, 77 aa, Homo sapiens
QLRRRGKTNHYQTTVEKKSLTIYAQVQKPGPLQKKLDSFPAQDPCTTIYVAATEPVPESVQETNSITVYASVTLPES

SEQ ID NO: 60 - protein, 99 aa, Homo sapiens
RLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVI

SEQ ID NO: 61 - protein, 84 aa, Homo sapiens
LRKRRDSLSLSTQRTQGPAESARNLEYVSVSPTNNTVYASVTHSNRETEIWTPRENDTITIYSTINHSKESKPTFSRATALDNV

SEQ ID NO: 62 - protein, 88 aa, Homo sapiens
WFLKRERQEEYIEEKKRVDICRETPNICPHSGENTEYDTIPHTNRTILKEDPANTVYSTVEIPKKMENPHSLLTMPDTPRLFAYENVI

SEQ ID NO: 63 - protein, 116 aa, Homo sapiens
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 64 - protein, 179 aa, Homo sapiens
KRKGRCSVPAFCSSQAEAPADTPEPTAGHTLYSVLSQGYEKLDTPLRPARQQPTPTSDSSSDSNLTTEEDEDRPEVHKPISGRYEVFDQVTQEGAGHDPAPEGQADYDPVTPYVTEVESVVGENTMYAQVFNLQGKTPVSQKEESSATIYCSIRKPQVVPPPQQNDLEIPESPTYENFT

SEQ ID NO: 65 - protein, 55 aa, Homo sapiens
KNRKAKAKPVTRGAGAGGRQRGQNKERPPPVPNPDYEPIRKGQRDLYSGLNQRRI

SEQ ID NO: 66 - protein, 45 aa, Homo sapiens
GQDGVRQSRASDKQTLLPNDQLYQPLKDREDDQYSHLQGNQLRRN

SEQ ID NO: 67 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 68 - protein, 76 aa, Homo sapiens
AKTGRTSIQRDLKEQQPQALAPGRGPSHSSAFSMSPLSTAQAPLPNPRTAASIYEELLKHDTNIYCRMDHKAEVAS

SEQ ID NO: 69 - protein, 43 aa, Homo sapiens
KHRKGNNAHENPKEVAIHLHSQGGSSVHPRTLQTNEENSRVLP

SEQ ID NO: 70 - protein, 26 aa, Homo sapiens
RYLQRRKKKGTYLTDETHREVKFTSL

SEQ ID NO: 71 - protein, 46 aa, Homo sapiens
YKKRRVHLPQEGAFENTLYFNSQSSPGTSDMKDLVGNIEQNEHSVI

SEQ ID NO: 72 - protein, 437 aa, Homo sapiens
RFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 73 - protein, 86 aa, Homo sapiens
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 74 - protein, 119 aa, Homo sapiens
KLWRVKKFLIPSVPDPKSIFPGLFEIHQGNFQEWITDTQNVAHLHKMAGAEQESGPEEPLVVQLAKTEAESPRMLDPQTEEKEASGGSLQLPHQPLQGGDVVTIGGFTFVMNDRSYVAL

SEQ ID NO: 75 - protein, 235 aa, Homo sapiens
HRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 76 - protein, 186 aa, Homo sapiens
SPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 77 - protein, 54 aa, Homo sapiens
KRFLRIQRLFPPVPQIKDKLNDNHEVEDEIIWEEFTPEEGKGYREEVLTVKEIT

SEQ ID NO: 78 - protein, 100 aa, Homo sapiens
KVFLRCINYVFFPSLKPSSSIDEYFSEQPLKNLLLSTSEEQIEKCFIIENISTIATVEETNQTDEDHKKYSSQTSQDSGNYSNEDESESKTSEELQQDFV

SEQ ID NO: 79 - protein, 251 aa, Homo sapiens
KWIGYICLRNSLPKVLNFHNFLAWPFPNLPPLEAMDMVEVIYINRKKKVWDYNYDDESDSDTEAAPRTSGGGYTMHGLTVRPLGQASATSTESQLIDPESEEEPDLPEVDVELPTMPKDSPQQLELLSGPCERRKSPLQDPFPEEDYSSTEGSGGRITFNVDLNSVFLRVLDDEDSDDLEAPLMLSSHLEEMVDPEDPDNVQSNHLLASGEGTQPTFPSPSSEGLWSEDAPSDQSDTSESDVDLGDGYIMR

SEQ ID NO: 80 - protein, 271 aa, Homo sapiens
KTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 81 - protein, 182 aa, Homo sapiens
YRAHFGTDETILDGKEYDIYVSYARNAEEEEFVLLTLRGVLENEFGYKLCIFDRDSLPGGIVTDETLSFIQKSRRLLVVLSPNYVLQGTQALLELKAGLENMASRGNINVILVQYKAVKETKVKELKRAKTVLTVIKWKGEKSKYPQGRFWKQLQVAMPVKKSPRRSSSDEQGLSYSSLKNV

SEQ ID NO: 82 - protein, 213 aa, Homo sapiens
KIFKIDIVLWYRDSCYDFLPIKASDGKTYDAYILYPKTVGEGSTSDCDIFVFKVLPEVLEKQCGYKLFIYGRDDYVGEDIVEVINENVKKSRRLIIILVRETSGFSWLGGSSEEQIAMYNALVQDGIKVVLLELEKIQDYEKMPESIKFIKQKHGAIRWSGDFTQGPQSAKTRFWKNVRYHMPVQRRSPSSKHQLLSPATKEKLQREAHVPLG

SEQ ID NO: 83 - protein, 207 aa, Homo sapiens
LKMFWIEATLLWRDIAKPYKTRNDGKLYDAYVVYPRNYKSSTDGASRVEHFVHQILPDVLENKCGYTLCIYGRDMLPGEDVVTAVETNIRKSRRHIFILTPQITHNKEFAYEQEVALHCALIQNDAKVILIEMEALSELDMLQAEALQDSLQHLMKVQGTIKWREDHIANKRSLNSKFWKHVRYQMPVPSKIPRKASSLTPLAAQKQ

SEQ ID NO: 84 - protein, 219 aa, Homo sapiens
NIFKIDIVLWYRSAFHSTETIVDGKLYDAYVLYPKPHKESQRHAVDALVLNILPEVLERQCGYKLFIFGRDEFPGQAVANVIDENVKLCRRLIVIVVPESLGFGLLKNLSEEQIAVYSALIQDGMKVILIELEKIEDYTVMPESIQYIKQKHGAIRWHGDFTEQSQCMKTKFWKTVRYHMPPRRCRPFPPVQLLQHTPCYRTAGPELGSRRKKCTLTTG

SEQ ID NO: 85 - protein, 31 aa, Homo sapiens
RLRRGGKDGSPKPGFLASVIPVDRRPGAPNL

SEQ ID NO: 86 - protein, 92 aa, Homo sapiens
NRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 87 - protein, 219 aa, Homo sapiens
HYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 88 - protein, 525 aa, Homo sapiens
CMTWRLAGPGSEKYSDDTKYTDGLPAADLIPPPLKPRKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVRCPDWFECENLYSADDQDAPSLDEEVFEEPLLPPGTGIVKRAPLVREPGSQACLAIDPLVGEEGGAAVAKLEPHLQPRGQPAPQPLHTLVLAAEEGALVAAVEPGPLADGAAVRLALAGEGEACPLLGSPGAGRNSVLFLPVDPEDSPLGSSTPMASPDLLPEDVREHLEGLMLSLFEQSLSCQAQGGCSRPAMVLTDPHTPYEEEQRQSVQSDQGYISRSSPQPPEGLTEMEEEEEEEQDPGKPALPLSPEDLESLRSLQRQLLFRQLQKNSGWDTMGSESEGPSA

SEQ ID NO: 89 - protein, 189 aa, Homo sapiens
RHERIKKTSFSTTTLLPPIKVLVVYPSEICFHHTICYFTEFLQNHCRSEVILEKWQKKKIAEMGPVQWLATQKKAADKVVFLLSNDVNSVCDGTCGKSEGSPSENSQDLFPLAFNLFCSDLRSQIHLHKYVVVYFREIDTKDDYNALSVCPKYHLMKDATAFCAELLHVKQQVSAGKRSQACHDGCCSL

SEQ ID NO: 90 - protein, 232 aa, Homo sapiens
KKDHAKGWLRLLKQDVRSGAAARGRAALLLYSADDSGFERLVGALASALCQLPLRVAVDLWSRRELSAQGPVAWFHAQRRQTLQEGGVVVLLFSPGAVALCSEWLQDGVSGPGAHGPHDAFRASLSCVLPDFLQGRAPGSYVGACFDRLLHPDAVPALFRTVPVFTLPSQLPDFLGALQQPRAPRSGRLQERAEQVSRALQPALDSYFHPPGTPAPGRGVGPGAGPGAGDGT

SEQ ID NO: 91 - protein, 192 aa, Homo sapiens
TCRRPQSGPGPARPVLLLHAADSEAQRRLVGALAELLRAALGGGRDVIVDLWEGRHVARVGPLPWLWAARTRVAREQGTVLLLWSGADLRPVSGPDPRAAPLLALLHAAPRPLLLLAYFSRLCAKGDIPPPLRALPRYRLLRDLPRLLRALDARPFAEATSWGRLGARQRRQSRLELCSRLEREAARLADLG

SEQ ID NO: 92 - protein, 191 aa, Homo sapiens
YRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSES

SEQ ID NO: 93 - protein, 222 aa, Homo sapiens
SALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTVTWRGLKSVPPNSRFWAKMRYHMPVKNSQGFTWNQLRITSRIFQWKGLSRTETTGRSSQPKEW

SEQ ID NO: 94 - protein, 286 aa, Homo sapiens
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 95 - protein, 285 aa, Homo sapiens
SLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 96 - protein, 325 aa, Homo sapiens
SYRYVTKPPAPPNSLNVQRVLTFQPLRFIQEHVLIPVFDLSGPSSLAQPVQYSQIRVSGPREPAGAPQRHSLSEITYLGQPDISILQPSNVPPPQILSPLSYAPNAAPEVGPPSYAPQVTPEAQFPFYAPQAISKVQPSSYAPQATPDSWPPSYGVCMEGSGKDSPTGTLSSPKHLRPKGQLQKEPPAGSCMLGGLSLQEVTSLAMEESQEAKSLHQPLGICTDRTSDPNVLHSGEEGTPQYLKGQLPLLSSVQIEGHPMSLPLQPPSRPCSPSDQGPSPWGLLESLVCPKDEAKSPAPETSDLEQPTELDSLFRGLALTVQWES

SEQ ID NO: 97 - protein, 253 aa, Homo sapiens
NRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 98 - protein, 99 aa, Homo sapiens
TSGRCYHLRHKVLPRWVWEKVPDPANSSSGQPHMEQVPEAQPLGDLPILEVEEMEPPPVMESSQPAQATAPLDSGYEKHFLPTPEELGLLGPPRPQVLA

SEQ ID NO: 99 - protein, 53 aa, Homo sapiens
RRYLVMQRLFPRIPHMKDPIGDSFQNDKLVVWEAGKAGLEECLVTEVQVVQKT

SEQ ID NO: 100 - protein, 277 aa, Homo sapiens
NKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 101 - protein, 195 aa, Homo sapiens
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 102 - protein, 239 aa, Homo sapiens
YRKREWIKETFYPDIPNPENCKALQFQKSVCEGSSALKTLEMNPCTPNNVEVLETRSAFPKIEDTEIISPVAERPEDRSDAEPENHVVVSYCPPIIEEEIPNPAADEAGGTAQVIYIDVQSMYQPQAKPEEEQENDPVGGAGYKPQMHLPINSTVEDIAAEEDLDKTAGYRPQANVNTWNLVSPDSPRSIDSNSEIVSFGSPCSINSRQFLIPPKDEDSPKSNGGGWSFTNFFQNKPND

SEQ ID NO: 103 - protein, 434 aa, Homo sapiens
KYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 104 - protein, 218 aa, Homo sapiens
KSQWIKETCYPDIPDPYKSSILSLIKFKENPHLIIMNVSDCIPDAIEVVSKPEGTKIQFLGTRKSLTETELTKPNYLYLLPTEKNHSGPGPCICFENLTYNQAASDSGSCGHVPVSPKAPSMLGLMTSPENVLKALEKNYMNSLGEIPAGETSLNYVSQLASPMFGDKDSLPTNPVEAPHCSEYKMQMAVSLRLALPPPTENSSLSSITLLDPGEHYC

SEQ ID NO: 105 - protein, 542 aa, Homo sapiens
RRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 106 - protein, 350 aa, Homo sapiens
KQQRIKMLILPPVPVPKIKGIDPDLLKEGKLEEVNTILAIHDSYKPEFHSDDSWVEFIELDIDEPDEKTEESDTDRLLSSDHEKSHSNLGVKDGDSGRTSCCEPDILETDFNANDIHEGTSEVAQPQRLKGEADLLCLDQKNQNNSPYHDACPATQQPSVIQAEKNKPQPLPTEGAESTHQAAHIQLSNPSSLSNIDFYAQVSDITPAGSVVLSPGQKNKAGMSQCDMHPEMVSLCQENFLMDNAYFCEADAKKCIPVAPHIKVESHIQPSLNQEDIYITTESLTTAAGRPGTGEHVPGSEMPVPDYTSIHIVQSPQGLILNATALPLPDKEFLSSCGYVSTDQLNKIMP

SEQ ID NO: 107 - protein, 403 aa, Homo sapiens
RKRQPDGPLGPLYASSNPEYLSASDVFPCSVYVPDEWEVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIYETDYYRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENKAPESEELEMEFEDMENVPLDRSSHCQREEAGGRDGGSSLGFKRSYEEHIPYTHMNGGKKNGRILTLPRSNPS

SEQ ID NO: 108 - protein, 303 aa, Homo sapiens
SHQRMKKLFWEDVPNPKNCSWAQGLNFQKPETFEHLFIKHTASVTCGPLLLEPETISEDISVDTSWKNKDEMMPTTVVSLLSTTDLEKGSVCISDQFNSVNFSEAEGTEVTYEDESQRQPFVKYATLISNSKPSETGEEQGLINSSVTKCFSSKNSPLKDSFSNSSWEIEAQAFFILSDQHPNIISPHLTFSEGLDELLKLEGNFPEENNDKKSIYYLGVTSIKKRESGVLLTDKSRVSCPFPAPCLFTDIRVLQDSCSHFVENNINLGTSSKKTFASYMPQFQTCSTQTHKIMENKMCDLTV

SEQ ID NO: 109 - protein, 364 aa, Homo sapiens
KGYSMVTCIFPPVPGPKIKGFDAHLLEKGKSEELLSALGCQDFPPTSDYEDLLVEYLEVDDSEDQHLMSVHSKEHPSQGMKPTYLDPDTDSGRGSCDSPSLLSEKCEEPQANPSTFYDPEVIEKPENPETTHTWDPQCISMEGKIPYFHAGGSKCSTWPLPQPSQHNPRSSYHNITDVCELAVGPAGAPATLLNEAGKDALKSSQTIKSREEGKATQQREVESFHSETDQDTPWLLPQEKTPFGSAKPLDYVEIHKVNKDGALSLLPKQRENSGKPKKPGTPENNKEYAKVSGVMDNNILVLVPDPHAKNVACFEESAKEAPPSLEQNQAEKALANFTATSSKCRLQLGGLDYLDPACFTHSFH

SEQ ID NO: 110 - protein, 122 aa, Homo sapiens
RWQFPAHYRRLRHALWPSLPDLHRVLGQYLRDTAALSPPKATVSDTCEEVEPSLLEILPKSSERTPLPLCSSQAQMDYRRLQPSCLGTMPLSVCPPMAESGSCCTTHIANHSYLPLSYWQQP

SEQ ID NO: 111 - protein, 542 aa, Homo sapiens; identical to SEQ ID NO: 105

SEQ ID NO: 112 - protein, 580 aa, Homo sapiens
KRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 113 - protein, 435 aa, Homo sapiens
KKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 114 - protein, 425 aa, Homo sapiens
KMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 115 - protein, 423 aa, Homo sapiens
RMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 116 - protein, 410 aa, Homo sapiens
RLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSSSSSGDDSVFAHDLLPPAPPSSGGSRT

SEQ ID NO: 117 - protein, 412 aa, Homo sapiens
RGQALHGRHPRPPATVQKLSRFPLARQFSLESGSSGKSSSSLVRGVRLSSSGPALLAGLVSLDLPLDPLWEFPRDRLVLGKPLGEGCFGQVVRAEAFGMDPARPDQASTVAVKMLKDNASDKDLADLVSEMEVMKLIGRHKNIINLLGVCTQEGPLYVIVECAAKGNLREFLRARRPPGPDLSPDGPRSSEGPLSFPVLVSCAYQVARGMQYLESRKCIHRDLAARNVLVTEDNVMKIADFGLARGVHHIDYYKKTSNGRLPVKWMAPEALFDRVYTHQSDVWSFGILLWEIFTLGGSPYPGIPVEELFSLLREGHRMDRPPHCPPELYGLMRECWHAAPSQRPTFKQLVEALDKVLLAVSEEYLDLRLTFGPYSPSGGDASSTCSSSDSVFSHDPLPLGSSSFPFGSGVQT

SEQ ID NO: 118 - protein, 571 aa, Homo sapiens
LRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 119 - protein, 567 aa, Homo sapiens
FCNMRRPAHADIKTGYLSIIMDPGEVPLEEQCEYLSYDASQWEFPRERLHLGRVLGYGAFGKVVEASAFGIHKGSSCDTVAVKMLKEGATASEHRALMSELKILIHIGNHLNVVNLLGACTKPQGPLMVIVEFCKYGNLSNFLRAKRDAFSPCAEKSPEQRGRFRAMVELARLDRRRPGSSDRVLFARFSKTEGGARRASPDQEAEDLWLSPLTMEDLVCYSFQVARGMEFLASRKCIHRDLAARNILLSESDVVKICDFGLARDIYKDPDYVRKGSARLPLKWMAPESIFDKVYTTQSDVWSFGVLLWEIFSLGASPYPGVQINEEFCQRLRDGTRMRAPELATPAIRRIMLNCWSGDPKARPAFSELVEILGDLLQGRGLQEEEEVCMAPRSSQSSEEGSFSQVSTMALHIAQADAEDSPPSLQRHSLAARYYNWVSFPGCLARGAETRGSSRMKTFEEFPMTPTTYKGSVDNQTDSGMVLASEEFEQIESRHRQESGFSCKGPGQNVAVTRAHPDSQGRRRRPERGARGGQVFYNSEYGELSEPSEEDHCSPSARVTFFTDNSY

SEQ ID NO: 120 - protein, 408 aa, Homo sapiens
RSRRAQRQRQQRQRDRATDVDREDKLWLKPYVDLQAYEDPAQGALDFTRELDPAWLMVDTVIGEGEFGEVYRGTLRLPSQDCKTVAIKTLKDTSPGGQWWNFLREATIMGQFSHPHILHLEGVVTKRKPIMIITEFMENGALDAFLREREDQLVPGQLVAMLQGIASGMNYLSNHNYVHRDLAARNILVNQNLCCKVSDFGLTRLLDDFDGTYETQGGKIPIRWTAPEAIAHRIFTTASDVWSFGIVMWEVLSFGDKPYGEMSNQEVMKSIEDGYRLPPPVDCPAPLYELMKNCWAYDRARRPHFQKLQAHLEQLLANPHSLRTIANFDPRMTLRLPSLSGSDGIPYRTVSEWLESIRMKRYILHFHSAGLDTMECVLELTAEDLTQMGITLPGHQKRILCSIQGFKD

SEQ ID NO: 121 - protein, 421 aa, Homo sapiens
SRKRAYSKEAVYSDKLQHYSTGRGSPGMKIYIDPFTYEDPNEAVREFAKEIDVSFVKIEEVIGAGEFGEVYKGRLKLPGKREIYVAIKTLKAGYSEKQRRDFLSEASIMGQFDHPNIIRLEGVVTKSRPVMIITEFMENGALDSFLRQNDGQFTVIQLVGMLRGIAAGMKYLAEMNYVHRDLAARNILVNSNLVCKVSDFGLSRYLQDDTSDPTYTSSLGGKIPVRWTAPEAIAYRKFTSASDVWSYGIVMWEVMSFGERPYWDMSNQDVINAIEQDYRLPPPMDCPAALHQLMLDCWQKDRNSRPRFAEIVNTLDKMIRNPASLKTVATITAVPSQPLLDRSIPDFTAFTTVDDWLSAIKMVQYRDSFLTAGFTSLQLVTQMTSEDLLRIGITLAGHQKKILNSIHSMRVQISQSPTAMA

SEQ ID NO: 122 - protein, 540 aa, Homo sapiens
KQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFL

SEQ ID NO: 123 - protein, 553 aa, Homo sapiens
MLWQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVEPEPELEQLPDSGCPAPRAEAEDSFL

SEQ ID NO: 124 - protein, 61 aa, Homo sapiens
RKRWQNEKLGLDAGDEYEDENLYEGLNLDDCSMYEDISRGLQGTYQDVGSLNIGDVQLEKP

SEQ ID NO: 125 - protein, 49 aa, Homo sapiens
LDKDDSKAGMEEDHTYEGLDIDQTATYEDIVTLRTGEVKWSVGEHPGQE

SEQ ID NO: 126 - protein, 52 aa, Homo sapiens
KRAVSTPSNEGAIIFLPPWLFSRRRRLERMSRGREKCYSSPGYPQESSNQFH

SEQ ID NO: 127 - protein, 61 aa, Homo sapiens
NRRRRRERRDLFTESWDTQKAPNNYRSPISTSQPTNQSMDDTREDIYVNYPTFSRRPKTRV

SEQ ID NO: 128 - protein, 39 aa, Homo sapiens
RLQSIFPDFSKAGMERAFLPVTSPNKHLGLVTPHKTELV

SEQ ID NO: 129 - protein, 38 aa, Homo sapiens
CWLTKKKYSSSVHDPNGEYMFMRAVNTAKKSRLTDVTL

SEQ ID NO: 130 - protein, 29 aa, Homo sapiens
NRQRKIKKYRLQQAQKGTPMKPNTQATPP

SEQ ID NO: 131 - protein, 27 aa, Homo sapiens
GQHLRQQRMGTYGVRAAWRRLPQAFRP

SEQ ID NO: 132 - protein, 37 aa, Homo sapiens
REHQRSGSYHVREESTYLPLTSMQPTEAMGEEPSRAE

SEQ ID NO: 133 - protein, 114 aa, Homo sapiens
RWCSKKKDAAVMNQEPAGHRTVNREDSDEQDPQEVTYAQLDHCIFTQRKITGPSQRSKRPSTDTSVCIELPNAEPRALSPAHEHHSQALMGSSRETTALSQTQLASSNVPAAGI

SEQ ID NO: 134 - protein, 40 aa, Homo sapiens
LHRWCSDKKNAAVMDQEPAGNRTVNSEDSDEQDHQEVSYA

SEQ ID NO: 135 - protein, 39 aa, Homo sapiens
HRWCSNKKNAAVMDQEPAGNRTVNSEDSDEQDHQEVSYA

SEQ ID NO: 136 - protein, 40 aa, Homo sapiens
LHRWCSDKKNASVMDQGPAGNRTVNREDSDEQDHQEVSYA

SEQ ID NO: 137 - protein, 39 aa, Homo sapiens
HRWCSDKKNAAVMDQEPAGNRTVNSEDSDEQDHQEVSYA

SEQ ID NO: 138 - protein, 40 aa, Homo sapiens
LHRWCSNKKNASVMDQGPAGNRTVNREDSDEQDHQEVSYA

SEQ ID NO: 139 - protein, 22 aa, Homo sapiens
HRWCSNKKKCCCNGPRACREQK

SEQ ID NO: 140 - protein, 25 aa, Homo sapiens
RKRTRERASRASTWEGRRRLNTQTL

SEQ ID NO: 141 - protein, 63 aa, Homo sapiens
WWGDIWWKTMMELRSLDTQKATCHLQQVTDLPWTSVSSPVEREILYHTVARTKISDDDDEHTL

SEQ ID NO: 142 - protein, 45 aa, Homo sapiens
GSTVYYQGKCLTWKGPRRQLPAVVPAPLPPPCGSSAHLLPPVPGG

SEQ ID NO: 143 - protein, 39 aa, Homo sapiens
RTQIKKLCSWRDKNSAACVVYEDMSHSRCNTLSSPNQYQ

SEQ ID NO: 144 - protein, 40 aa, Homo sapiens
CVRCRHRRRQAERMSQIKRLLSEKKTCQCPHRFQKTCSPI

SEQ ID NO: 145 - protein, 41 aa, Homo sapiens
RSKRSRLLHSDYMNMTPRRPGPTRKHYQPYAPPRDFAAYRS

SEQ ID NO: 146 - protein, 111 aa, Homo sapiens
FWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 147 - protein, 68 aa, Homo sapiens
AFYVQSTACKKGEYNVQEAESSGEAVCLNGAGGGAGGAAGAEGGPEAAGGAAESPAEGEVFAIQLTSA

SEQ ID NO: 148 - protein, 59 aa, Homo sapiens
YKVGFFKRNLKEKMEAGRGVPNGIPAEDSEQLASGQEAGDPGCLKPLHEKDSESGGGKD

SEQ ID NO: 149 - protein, 46 aa, Homo sapiens
KALIHLSDLREYRRFEKEKLKSQWNNDNPLFKSATTTVMNPKFAES

SEQ ID NO: 150 - protein, 85 aa, Homo sapiens
IMKLRKAHVIWKKENEVSEHTLESYRSRSNNEETSSEEKNGQSSHPMRCMNYITKLYSEAKTKRKENVQHSKLEEKHIQVPESIV

SEQ ID NO: 151 - protein, 141 aa, Homo sapiens
KLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 152 - protein, 15 aa, Homo sapiens
TRCGGPQQSRAERPG

SEQ ID NO: 153 - protein, 44 aa, Homo sapiens
RAARRRPEHLDTPDTPPRSQAQESNYENLSQMNPRSPPATMCSP

SEQ ID NO: 154 - protein, 26 aa, Homo sapiens
LLRVRSCRRKAARAALGMEAADAVTD

SEQ ID NO: 155 - protein, 48 aa, Homo sapiens
KKYFFKKEVQQLSVSFSSLQIKALQNAVEKEVQAEDNIYIENSLYATD

SEQ ID NO: 156 - protein, 185 aa, Homo sapiens
SYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKK

SEQ ID NO: 157 - protein, 214 aa, Homo sapiens
CLHFDLPWYLRMLGQCTQTWHRVRKTTQEQLKRNVRFHAFISYSEHDSLWVKNELIPNLEKEDGSILICLYESYFDPGKSISENIVSFIEKSYKSIFVLSPNFVQNEWCHYEFYFAHHNLFHENSDHIILILLEPIPFYCIPTRYHKLKALLEKKAYLEWPKDRRKCGLFWANLRAAINVNVLATREMYELQTFTELNEESRGSTISLMRTDCL

SEQ ID NO: 158 - protein, 175 aa, Homo sapiens
HRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 159 - protein, 179 aa, Homo sapiens
EGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 160 - protein, 187 aa, Homo sapiens
KFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 161 - protein, 198 aa, Homo sapiens
TKFRGFCFICYKTAQRLVFKDHPQGTEPDMYKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKHQSIRGFVQKQQYLRWPEDFQDVGWFLHKLSQQILKKEKEKKKDNNIPLQTVATIS

SEQ ID NO: 162 - protein, 189 aa, Homo sapiens
YLDLPWYLRMVCQWTQTRRRARNIPLEELQRNLQFHAFISYSEHDSAWVKSELVPYLEKEDIQICLHERNFVPGKSIVENIINCIEKSYKSIFVLSPNFVQSEWCHYELYFAHHNLFHEGSNNLILILLEPIPQNSIPNKYHKLKALMTQRTYLQWPKEKSKRGLFWANIRAAFNMKLTLVTENNDVKS

SEQ ID NO: 163 - protein, 189 aa, Homo sapiens
HLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

SEQ ID NO: 164 - protein, 193 aa, Homo sapiens
HHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 165 - protein, 193 aa, Homo sapiens
GWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 166 - protein, 48 aa, Homo sapiens
QRRKYRSNKGESPVEPAEPCHYSCPREEEGSTIPIQEDYRKPEPACSP

SEQ ID NO: 167 - protein, 383 aa, Homo sapiens
CYRKKGKALTANLWHWINEACGRLSGDKESSGDSCVSTHTANFGQQGACEGVLLLTLEEKTFPEDMCYPDQGGVCQGTCVGGGPYAQGEDARMLSLVSKTEIEEDSFRQMPTEDEYMDRPSQPTDQLLFLTEPGSKSTPPFSEPLEVGENDSLSQCFTGTQSTVGSESCNCTEPLCRTDWTPMSSENYLQKEVDSGHCPHWAASPSPNWADVCTGCRNPPGEDCEPLVGSPKRGPLPQCAYGMGLPPEEEASRTEARDQPEDGADGRLPSSARAGAGSGSSPGGQSPASGNVTGNSNSTFISSGQVMNFKGDIIVVYVSQTSQEGAAAAAEPMGRPVQEETLARRDSFAGNGPRFPDPCGGPEGLREPEKASRPVQEQGGAKA

SEQ ID NO: 168 - protein, 28 aa, Homo sapiens
RRCRRREKFTTPIEETGGEGCPAVALIQ

SEQ ID NO: 169 - protein, 107 aa, Homo sapiens
KKRGDPCSCQPRSRPRQSPAKSSQDHAMEAGSPVSTSPEPVETCSFCFPECRAPTQESAVTPGTPDPTCAGRWGCHTRTTVLQPCPHIPDSGLGIVCVPAQEGGPGA

SEQ ID NO: 170 - protein, 85 aa, Homo sapiens
SWRRRQRRLRGASSAEAPDGDKDAPEPLDKVIILSPGISDATAPAWPPPGEDPGTTPPGHSVPVPATELGSTELVTTKTAGPEQQ

SEQ ID NO: 171 - protein, 60 aa, Homo sapiens
CVKRRKPRGDVVKVIVSVQRKRQEAEGEATVIEALQAPPDVTTVAVEETIPSFTGRSPNH

SEQ ID NO: 172 - protein, 155 aa, Homo sapiens
KRWNSCKQNKQGANSRPVNQTPPPEGEKLHSDSGISVDSQSLHDQQPHTQTASGQALKGDGGLYSSLPPAKREEVEKLLNGSAGDTWRHLAGELGYQPEHIDSFTHEACPVRALLASWATQDSATLDALLAALRRIQRADLVESLCSESTATSPV

SEQ ID NO: 173 - protein, 107 aa, Homo sapiens
RKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 174 - protein, 58 aa, Homo sapiens
QLGLHIWQLRSQCMWPRETQLLLEVPPSTEDARSCQFPEEERGERSAEEKGRLGDLWV

SEQ ID NO: 175 - protein, 232 aa, Homo sapiens
YCKRQFMEKKPSWSLRSQDIQYNGSELSCFDRPQLHEYAHRACCQCRRDSVQTCGPVRLLPSMCCEEACSPNPATLGCGVHSAASLQARNAGPAGEMVPTFFGSLTQSICGEFSDAWPLMQNPMGGDNISFCDSYPELTGEDIHSLNPELESSTSLDSNSSQDLVGGAVPVQSHSENFTAATDLSRYNNTLVESASTQDALTMRSQLDQESGAVIHPATQTSLQVRQRLGSL

SEQ ID NO: 176 - protein, 247 aa, Homo sapiens
NLLKRKGYHCTAHKEVGPGPGGGGSGINPAYRTEDANEDTIGVLVRLITEKKENAAALEELLKEYHSKQLVQTSHRPVSKLPPAPPNVPHICPHRHHLHTVQGLASLSGPCCSRCSQKKWPEVLLSPEAVAATTPVPSLLPNPTRVPKAGAKAGRQGEITILSVGRFRVARIPEQRTSSMVSEVKTITEAGPSWGDLPDSPQPGLPPEQQALLGSGGSRTKWLKPPAENKAEENRYVVRLSESNLVI

SEQ ID NO: 177 - protein, 223 aa, Homo sapiens
MYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 178 - protein, 174 aa, Homo sapiens
KKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 179 - protein, 197 aa, Homo sapiens
TYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 180 - protein, 138 aa, Homo sapiens
LYCKQFFNRHCQRGGLLQFEADKTAKEESLFPVPPSKETSAESQVSENIFQTQPLNPILEDDCSSTSGFPTQESFTMASCTSESHSHWVHSPIECTELDLQKFSSSASYTGAETLGGNTVESTGDRLELNVPFEVPSP

SEQ ID NO: 181 - protein, 187 aa, Homo sapiens
KSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 182 - protein, 42 aa, Homo sapiens
ALYLLRRDQRLPPDAHKPPGGGSFRTPIQEEQADAHSTLAKI

SEQ ID NO: 183 - protein, 62 aa, Homo sapiens
KKVAKKPTNKAPHPKQEPQEINFPDDLPGSNTAAPVQETLHGCQPVTQEDGKESRISVQERQ

SEQ ID NO: 184 - protein, 189 aa, Homo sapiens
CHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 185 - protein, 42 aa, Homo sapiens
KRGRKKLLYIFKQPFMRPVQTTQEEDGCSCRFPEEEEGGCEL

SEQ ID NO: 186 - protein, 240 aa, Homo sapiens
YILKTKPSAPACCTSHPGKSVEAQVSKDEEKKEAPDNVVMFSEKDEFEKLTATPAKPTKSENDASSENEQLLSRSVDSDEEPAPDKQGSPELCLLSLVHLAREKSATSNKSAGIQSRRKKILDVYANVCGVVEGLSPTELPFDCLEKTSRMLSSTYNSEKAVVKTWRHLAESFGLKRDEIGGMTDGMQLFDRISTAGYSIPELLTKLVQIERLDAVESLCADILEWAGVVPPASQPHAAS

SEQ ID NO: 187 - protein, 235 aa, Homo sapiens
HCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 188 - protein, 217 aa, Homo sapiens
RCSRPGAKRSEKIYQQRSLREDQQSFTGSRTYSLVGQAWPGPLADMAPTRKDKLLQFYPSLEDPASSRYQNFSKGSRHGSEEAYIDPIAMEYYNWGRFSKPPEDDDANSYENVLICKQKTTETGAQQEGIGGLCRGDLSLSLALKTGPTSGLCPSASPEEDEESEDYQNSASIHQWRESRKVMGQLQREASPGPVGSPDEEDGEPDYVNGEVAATEA

SEQ ID NO: 189 - protein, 340 aa, Homo sapiens
WNWNKRKKRQVPYLRVTVMPLLTLPQTRQRAKNIYDILPWRQEDLGRHESRSMRIFSTESLLSRNSESPEHVPSQAGNAFQEHTAHIHATEYAVGIYDNAMVPQMCGNLTPSAHCINVRASRDCASISSEDSHDYVNVPTAEEIAETLASTKSPSRNLFVLPSTQKLEFTEERDEGCGDAGDCTSLYSPGAEDSDSLSNGEGSSQISNDYVNMTGLDLSAIQERQLWVAFQCCRDYENVPAADPSGSQQQAEKDVPSSNIGHVEDKTDDPGTHVQCVKRTFLASGDYADFQPFTQSEDSQMKHREEMSNEDSSDYENVLTAKLGGRDSEQGPGTQLLPDE

SEQ ID NO: 190 - protein, 395 aa, Homo sapiens
SSCDREKKPRQHSGDHENLMNVPSDKEMFSRSVTSLATDAPASSEQNGALTNGDILSEDSTLTCMQHYEEVQTSASDLLDSQDSTGKPKCHQSRELPRIPPESAVDTMLTARSVDGDQGLGMEGPYEVLKDSSSQENMVEDCLYETVKEIKEVAAAAHLEKGHSGKAKSTSASKELPGPQTEGKAEFAEYASVDRNKKCRQSVNVESILGNSCDPEEEAPPPVPVKLLDENENLQEKEGGEAEESATDTTSETNKRFSSLSYKSREEDPTLTEEEISAMYSSVNKPGQLVNKSGQSLTVPESTYTSIQGDPQRSPSSCNDLYATVKDFEKTPNSTLPPAGRPSEEPEPDYEAIQTLNREEEKATLGTNGHHGLVPKENDYESISDLQQGRDITRL

SEQ ID NO: 191 - protein, 235 aa, Homo sapiens; identical to SEQ ID NO: 187

SEQ ID NO: 192 - protein, 21 aa, Homo sapiens
MEEGQYSEIEELPRRRCCRRG

SEQ ID NO: 193 - protein, 37 aa, Homo sapiens
MSDSKEPRLQQLGLLEEEQLRGLGFRQTRGYKSLAGC

SEQ ID NO: 194 - protein, 33 aa, Homo sapiens
MQDEDGYITLNIKTRKPALISVGSASSSWWRVM

SEQ ID NO: 195 - protein, 44 aa, Homo sapiens
MEYHPDLENLDEDGYTQLHFDSQSNTRIAVVSEKGSCAASPPWR

SEQ ID NO: 196 - protein, 35 aa, Homo sapiens
MHEEEIYTSLQWDSPAPDTYQKCLSSNKCSGACCL

SEQ ID NO: 197 - protein, 38 aa, Homo sapiens
MQDEERYMTLNVQSKKRSSAQTSQLTFKDYSVTLHWYK

SEQ ID NO: 198 - protein, 30 aa, Homo sapiens
MENEDGYMTLSFKNRCKSKQKSKDFSLYPQ

SEQ ID NO: 199 - protein, 70 aa, Homo sapiens
MSKQRGTFSEVSLAQDPKRQQRKPKGNKSSISGTEQEIFQVELNLQNPSLNHQGIDKIYDCQGLLPPPEK

SEQ ID NO: 200 - protein, 51 aa, Homo sapiens
MGWIRGRRSRHSWEMSEFHNYNLDLKKSDFSTRWQKQRCPVVKSKCRENAS

SEQ ID NO: 201 - protein, 70 aa, Homo sapiens
MSKQRGTFSEVSLAQDPKWQQRKPKGNKSSISGTEQEIFQVELNLQNASLNHQGIDKIYDCQGLLPPPEK

SEQ ID NO: 202 - protein, 19 aa, Homo sapiens
MNSSKSSETQCTERGCFSS

SEQ ID NO: 203 - protein, 20 aa, Homo sapiens
MMQEQQPQSTEKRGWLSLRL

SEQ ID NO: 204 - protein, 39 aa, Homo sapiens
MTRTYENFQYLENKVKVQGFKNGPLPLQSLLQRLCSGPC

SEQ ID NO: 205 - protein, 17 aa, Homo sapiens
MGLEKPQSKLEGGMHPQ

SEQ ID NO: 206 - protein, 21 aa, Homo sapiens
MVPEEEPQDREKGLWWFQLKV

SEQ ID NO: 207 - protein, 172 aa, Homo sapiens
MHNLYSITGYPDPPGTMEEEEEDDDYENSTPPYKDLPPKPGTMEEEEEDDDYENSTPPYKDLPPKPGTMEEEEEDDDYENSTPPYKDLPPKPGSSAPPRPPRAAKETEKPPLPCKPRNMTGLDLAAVTCPPPQLAVNLEPSPLQPSLAATPVPWLNQRSGGPGCCQKRWMVY

SEQ ID NO: 208 - protein, 17 aa, Homo sapiens
MPEEGSGCSVRRRPYGC

SEQ ID NO: 209 - protein, 37 aa, Homo sapiens
MEESVVRPSVFVVDGQTDIPFTRLGRSHRRQSCSVAR

SEQ ID NO: 210 - protein, 37 aa, Homo sapiens
MDPGLQQALNGMAPPGDTAMHVPAGSVASHLGTTSRS

SEQ ID NO: 211 - protein, 35 aa, Homo sapiens
MSTESMIRDVELAEEALPKKTGGPQGSRRCLFLSL

SEQ ID NO: 212 - protein, 23 aa, Homo sapiens
MERVQPLEENVGNAARPRFERNK

SEQ ID NO: 213 - protein, 22 aa, Homo sapiens
MIETYNQTSPRSAATGLPISMK

SEQ ID NO: 214 - protein, 80 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTG

SEQ ID NO: 215 - protein, 28 aa, Homo sapiens
MEYASDASLDPEAPWPPAPRARACRVLP

SEQ ID NO: 216 - protein, 17 aa, Homo sapiens
MAMMEVQGGPSLGQTCV

SEQ ID NO: 217 - protein, 47 aa, Homo sapiens
MRRASRDYTKYLRGSEEMGGGPGAPHEGPLHAPPPPAPHQPPAASRS

SEQ ID NO: 218 - protein, 21 aa, Homo sapiens
MAARRSQRRRGRRGEPGTALL

SEQ ID NO: 219 - protein, 28 aa, Homo sapiens
MPASSPFLLAPKGPPGNMGGPVREPALS

SEQ ID NO: 220 - protein, 46 aa, Homo sapiens
MDDSTEREQSRLTSCLKKREEMKLKECVSILPRKESPSVRSSKDGK

SEQ ID NO: 221 - protein, 28 aa, Homo sapiens
MCLSHLENMPLSHSRTQGAQRSSWKLWL

SEQ ID NO: 222 - protein, 36 aa, Homo sapiens
CYRVNRQQKLSSLCARPRRSPAQEDGKVYINMPGRG

SEQ ID NO: 223 - protein, 21 aa, Homo sapiens
LLPLGGLPLLITTCFCLFCCL

SEQ ID NO: 224 - protein, 20 aa, Homo sapiens
LQEKVWVMLVTSLVALQGMS

SEQ ID NO: 225 - protein, 21 aa, Homo sapiens
YIPYIILTIIILTIVGFIWLL

SEQ ID NO: 226 - protein, 23 aa, Homo sapiens
GAIGGAGVTALLALCLCLIFFIV

SEQ ID NO: 227 - protein, 24 aa, Homo sapiens
AIAGIVIGVVALVALIAVALACFL

SEQ ID NO: 228 - protein, 21 aa, Homo sapiens
FLLWILAAVSSGLFFYSFLLT

SEQ ID NO: 229 - protein, 17 aa, Homo sapiens
LGWLCLLLLPIPLIVWV

SEQ ID NO: 230 - protein, 21 aa, Homo sapiens
LVPWLPASLLGLMVIAAALLV

SEQ ID NO: 231 - protein, 21 aa, Homo sapiens
VIIFFAFVLLLSGALAYCLAL

SEQ ID NO: 232 - protein, 22 aa, Homo sapiens
WMVAVILMASVFMVCLALLGCF

SEQ ID NO: 233 - protein, 22 aa, Homo sapiens
VAAVLASLLVLLALLLAALLYV

SEQ ID NO: 234 - protein, 21 aa, Homo sapiens
TFLVAGGSLAFGTLLCIAIVL

SEQ ID NO: 235 - protein, 22 aa, Homo sapiens
AIVVPVCLAFLLTTLLGVLFCF

SEQ ID NO: 236 - protein, 19 aa, Homo sapiens
ILIGTSVVIILFILLFFLL

SEQ ID NO: 237 - protein, 19 aa, Homo sapiens
ILIGTSVVIILFILLFFLL

SEQ ID NO: 238 - protein, 20 aa, Homo sapiens
VLIGTSVVIILFILLLFFLL

SEQ ID NO: 239 - protein, 21 aa, Homo sapiens
LHILIGTSVAIILFIILFFFL

SEQ ID NO: 240 - protein, 21 aa, Homo sapiens
LHILIGTSVAIILFIILFFFL

SEQ ID NO: 241 - protein, 20 aa, Homo sapiens
ILIGTSVVIILFILLLFFLL

SEQ ID NO: 242 - protein, 20 aa, Homo sapiens
VLIGTSVVIFLFILLLFFLL

SEQ ID NO: 243 - protein, 21 aa, Homo sapiens
HVLIGTSVVIIPFAILLFFLL

SEQ ID NO: 244 - protein, 21 aa, Homo sapiens
LLFLILGVLSLLLLVTGAFGF

SEQ ID NO: 245 - protein, 21 aa, Homo sapiens
VIGILVAVILLLLLLLLLFLI

SEQ ID NO: 246 - protein, 21 aa, Homo sapiens
VIGILVAVVLLLLLLLLLFLI

SEQ ID NO: 247 - protein, 21 aa, Homo sapiens
VLIGVSVAFVLLLFLLLFLLL

SEQ ID NO: 248 - protein, 21 aa, Homo sapiens
VLIGVLVVSILLLSLLLFLLL

SEQ ID NO: 249 - protein, 21 aa, Homo sapiens
VVTGVSVAFVLLLFLLLFLLL

SEQ ID NO: 250 - protein, 21 aa, Homo sapiens
AGTVLLLRAGFYAVSFLSVAV

SEQ ID NO: 251 - protein, 21 aa, Homo sapiens
VGVVGGLLGSLVLLVWVLAVI

SEQ ID NO: 252 - protein, 21 aa, Homo sapiens
GAFLGIGITALLFLCLALIIM

SEQ ID NO: 253 - protein, 23 aa, Homo sapiens
GAVGGAGATALVFLSFCVIFIVV

SEQ ID NO: 254 - protein, 21 aa, Homo sapiens
VVGGAGATALVFLSFCVIFVV

SEQ ID NO: 255 - protein, 21 aa, Homo sapiens
SWPVIVAALLFCCMILFGLGV

SEQ ID NO: 256 - protein, 21 aa, Homo sapiens
AAVIAGPVCFVCISLMLMVYI

SEQ ID NO: 257 - protein, 21 aa, Homo sapiens
VTGISLLPPLGVAISVIIIFY

SEQ ID NO: 258 - protein, 21 aa, Homo sapiens
LLGAMAATLVVICTAVIVVVA

SEQ ID NO: 259 - protein, 21 aa, Homo sapiens
IYIGAGICAGLALALIFGALI

SEQ ID NO: 260 - protein, 21 aa, Homo sapiens
LISGTLGIICLSLMSTLGILL

SEQ ID NO: 261 - protein, 21 aa, Homo sapiens
AGIILLVLVVTGLSVSVTSLI

SEQ ID NO: 262 - protein, 21 aa, Homo sapiens
VAIALGLLTAVLLSVLLYQWI

SEQ ID NO: 263 - protein, 23 aa, Homo sapiens
LIVGILGIICLILMASVVTIVVI

SEQ ID NO: 264 - protein, 21 aa, Homo sapiens
LLAGLVAADAVASLLIVGAVF

SEQ ID NO: 265 - protein, 21 aa, Homo sapiens
AISVGISLLLLLVVCGIGCVW

SEQ ID NO: 266 - protein, 21 aa, Homo sapiens
GVLAGIVMGDLVLTVLIALAV

SEQ ID NO: 267 - protein, 21 aa, Homo sapiens
LCYILDAILFLYGIVLTLLYC

SEQ ID NO: 268 - protein, 21 aa, Homo sapiens
VLFYLAVGIMFLVNTVLWVTI

SEQ ID NO: 269 - protein, 23 aa, Homo sapiens
IIVAVVIATAVAAIVAAVVALIY

SEQ ID NO: 270 - protein, 23 aa, Homo sapiens
IIVAVVTGIAVAAIVAAVVALIY

SEQ ID NO: 271 - protein, 21 aa, Homo sapiens
VSFCLVMVLLFAVDTGLYFSV

SEQ ID NO: 272 - protein, 19 aa, Homo sapiens
LIRMAVAGLVLVALLAILV

SEQ ID NO: 273 - protein, 23 aa, Homo sapiens
YALIPIPLAVITTCIVLYMNGIL

SEQ ID NO: 274 - protein, 21 aa, Homo sapiens
FLVIIVILSALFLGTLACFCV

SEQ ID NO: 275 - protein, 21 aa, Homo sapiens
WAVYAGLLGGVIMILIMVVIL

SEQ ID NO: 276 - protein, 21 aa, Homo sapiens
LLSVLAMFFLLVLILSSVFLF

SEQ ID NO: 277 - protein, 21 aa, Homo sapiens
ILFMVSGICIVFGFIILLLLV

SEQ ID NO: 278 - protein, 21 aa, Homo sapiens
VLLCLLLVPLLLSLFVLGLFL

SEQ ID NO: 279 - protein, 26 aa, Homo sapiens
IYLIIGICGGGSLLMVFVALLVFYIT

SEQ ID NO: 280 - protein, 22 aa, Homo sapiens
LWIGLFLMVCLLCVGIFSWCIW

SEQ ID NO: 281 - protein, 26 aa, Homo sapiens
VMSVATIVIVDICITGGLLLLVYYWS

SEQ ID NO: 282 - protein, 21 aa, Homo sapiens
GFLFAEIVSIFVLAVGVYFIA

SEQ ID NO: 283 - protein, 21 aa, Homo sapiens
LCYLLDGILFIYGVILTALFL

SEQ ID NO: 284 - protein, 21 aa, Homo sapiens
IVTGVLVGVALVAALVCFLLL

SEQ ID NO: 285 - protein, 25 aa, Homo sapiens
ALIVGTLSGTIFFILLIIFLSWIIL

SEQ ID NO: 286 - protein, 23 aa, Homo sapiens
VWVIAVIVIAIVVGVAVICVVPY

SEQ ID NO: 287 - protein, 21 aa, Homo sapiens
GVVIIVILLILTGAGLAAYFF

SEQ ID NO: 288 - protein, 17 aa, Homo sapiens
VLALIVIFLTIAVLLAL

SEQ ID NO: 289 - protein, 21 aa, Homo sapiens
VVISVGSMGLIISLLCVYFWL

SEQ ID NO: 290 - protein, 21 aa, Homo sapiens
FILISSLAILLMVSLLLLSLW

SEQ ID NO: 291 - protein, 23 aa, Homo sapiens
LILTLSLILVVILVLLTVLALLS

SEQ ID NO: 292 - protein, 23 aa, Homo sapiens
IILGLFGLLLLLTCLCGTAWLCC

SEQ ID NO: 293 - protein, 26 aa, Homo sapiens
NLGSVYIYVLLIVGTLVCGIVLGFLF

SEQ ID NO: 294 - protein, 21 aa, Homo sapiens
IWLIVGICIALFALPFVIYAA

SEQ ID NO: 295 - protein, 21 aa, Homo sapiens
IGGIITVFLIALVLTSTIVTL

SEQ ID NO: 296 - protein, 21 aa, Homo sapiens
FLVLPSLLILLLVIAAGGVIW

SEQ ID NO: 297 - protein, 21 aa, Homo sapiens
VLLVVILIVVYHVYWLEMVLF

SEQ ID NO: 298 - protein, 20 aa, Homo sapiens
HMIGICVTLTVIIVCSVFIY

SEQ ID NO: 299 - protein, 21 aa, Homo sapiens
IYCIIAVCSVFLMLINVLVII

SEQ ID NO: 300 - protein, 21 aa, Homo sapiens
AYLIGGLIALVAVAVSVVYIY

SEQ ID NO: 301 - protein, 21 aa, Homo sapiens
SLGILSFLGLVAGALALGLWL

SEQ ID NO: 302 - protein, 25 aa, Homo sapiens
WLIFFASLGSFLSILLVGVLGYLGL

SEQ ID NO: 303 - protein, 21 aa, Homo sapiens
WMAFVAPSICIAIIMVGIFST

SEQ ID NO: 304 - protein, 21 aa, Homo sapiens
VYWFITGISILLVGSVILLIV

SEQ ID NO: 305 - protein, 21 aa, Homo sapiens
LLLLSLLVATWVLVAGIYLMW

SEQ ID NO: 306 - protein, 21 aa, Homo sapiens
WALVWLACLLFAAALSLILLL

SEQ ID NO: 307 - protein, 21 aa, Homo sapiens
LGLLILALLALLTLLGVVLAL

SEQ ID NO: 308 - protein, 21 aa, Homo sapiens
GMIIAVLILVAVVCLVTVCVI

SEQ ID NO: 309 - protein, 21 aa, Homo sapiens
GVVLLYILLGTIGTLVAVLAA

SEQ ID NO: 310 - protein, 25 aa, Homo sapiens
IPWLGHLLVGLSGAFGFIILVYLLI

SEQ ID NO: 311 - protein, 21 aa, Homo sapiens
GWNPHLLLLLLLVIVFIPAFW

SEQ ID NO: 312 - protein, 21 aa, Homo sapiens
YSFSGAFLFSMGFLVAVLCYL

SEQ ID NO: 313 - protein, 21 aa, Homo sapiens
LLLGMIVFAVMLSILSLIGIF

SEQ ID NO: 314 - protein, 21 aa, Homo sapiens
VLPGILFLWGLFLLGCGLSLA

SEQ ID NO: 315 - protein, 20 aa, Homo sapiens
TSLLIALGTLLALVCVFVIC

SEQ ID NO: 316 - protein, 22 aa, Homo sapiens
AIVVPVCLAFLLTTLLGVLFCF

SEQ ID NO: 317 - protein, 25 aa, Homo sapiens
PILLTISILSFFSVALLVILACVLW

SEQ ID NO: 318 - protein, 25 aa, Homo sapiens
VGLIIAILIPVAVAVIVGVVTSILC

SEQ ID NO: 319 - protein, 21 aa, Homo sapiens
VVVACMSIMALLLLLLLLLLY

SEQ ID NO: 320 - protein, 21 aa, Homo sapiens
LIHILLPMVFCVLLIMVMCYL

SEQ ID NO: 321 - protein, 23 aa, Homo sapiens
IATGMVGALLLLLVVALGIGLFM

SEQ ID NO: 322 - protein, 24 aa, Homo sapiens
FPWLLIIIFGIFGLTVMLFVFLFS

SEQ ID NO: 323 - protein, 23 aa, Homo sapiens
IIIGPLIFVFLFSVVIGSIYLFL

SEQ ID NO: 324 - protein, 23 aa, Homo sapiens
AGLYVIVPVIISSSILLLGTLLI

SEQ ID NO: 325 - protein, 24 aa, Homo sapiens
TTVWISVAVLSAVICLIIVWAVAL

SEQ ID NO: 326 - protein, 22 aa, Homo sapiens
ISLVTALHLVLGLSAVLGLLLL

SEQ ID NO: 327 - protein, 23 aa, Homo sapiens
IATGMVGALLLLLVVALGIGLFM

SEQ ID NO: 328 - protein, 23 aa, Homo sapiens
SIISAVVGILLVVVLGVVFGILI

SEQ ID NO: 329 - protein, 23 aa, Homo sapiens
GLIAGVVSISTALLLLLGFFLWL

SEQ ID NO: 330 - protein, 21 aa, Homo sapiens
IIIYCTGAFLISCMVGSVIVY

SEQ ID NO: 331 - protein, 21 aa, Homo sapiens
IAIYCIGVFLIACMVVTVILC

SEQ ID NO: 332 - protein, 21 aa, Homo sapiens
ILSYGVGFFLFILVVAAVTLC

SEQ ID NO: 333 - protein, 21 aa, Homo sapiens
IILYASGSLALAVLLLLAGLY

SEQ ID NO: 334 - protein, 21 aa, Homo sapiens
IIILVGTAVIAMFFWLLLVII

SEQ ID NO: 335 - protein, 21 aa, Homo sapiens
IVILVGTGVIAVFFWVLLLLI

SEQ ID NO: 336 - protein, 21 aa, Homo sapiens
IVAVIFGLLLGAALLLGILVF

SEQ ID NO: 337 - protein, 23 aa, Homo sapiens
LIAGSAAAGVVFVVSLVAISIVC

SEQ ID NO: 338 - protein, 21 aa, Homo sapiens
AAVLVLLVIVIISLIVLVVIW

SEQ ID NO: 339 - protein, 21 aa, Homo sapiens
VVISAILALVVLTIISLIILI

SEQ ID NO: 340 - protein, 22 aa, Homo sapiens
IITAEGIILLFCAVVPGTLLLF

SEQ ID NO: 341 - protein, 21 aa, Homo sapiens
GIIMIQTLLIILFIIVPIFLL

SEQ ID NO: 342 - protein, 20 aa, Homo sapiens
LQEKVWVMLVTSLVALQGMS

SEQ ID NO: 343 - protein, 21 aa, Homo sapiens
GGTVLLLLFVISITTIIVIFL

SEQ ID NO: 344 - protein, 22 aa, Homo sapiens
IVLLLALVIFYLTLIIFTCKFA

SEQ ID NO: 345 - protein, 21 aa, Homo sapiens
FWLPIGCAAFVVVCILGCILI

SEQ ID NO: 346 - protein, 23 aa, Homo sapiens
IVIITVVAAAVIMGTAGLSTYLY

SEQ ID NO: 347 - protein, 25 aa, Homo sapiens
MVIIVTVVSVLLSLFVTSVLLCFIF

SEQ ID NO: 348 - protein, 25 aa, Homo sapiens
FVPVFVAVLLTLGVVTIVLALMYVF

SEQ ID NO: 349 - protein, 21 aa, Homo sapiens
AVIRYSVAIILFTILPFFLLH

SEQ ID NO: 350 - protein, 19 aa, Homo sapiens
VLIGTSVVKIPFTILLFFL

SEQ ID NO: 351 - protein, 20 aa, Homo sapiens
VLIGTSVVKIPFTILLFFLL

SEQ ID NO: 352 - protein, 19 aa, Homo sapiens
VLIGTSVVKLPFTILLFFL

SEQ ID NO: 353 - protein, 20 aa, Homo sapiens
VLIGTSVVKIPFTILLFFLL

SEQ ID NO: 354 - protein, 19 aa, Homo sapiens
VLIGTSVVKLPFTILLFFL

SEQ ID NO: 355 - protein, 20 aa, Homo sapiens
ILIGTSVVKIPFTILLFFLL

SEQ ID NO: 356 - protein, 21 aa, Homo sapiens
MGLAFLVLVALVWFLVEDWLS

SEQ ID NO: 357 - protein, 21 aa, Homo sapiens
LVPVFCGLLVAKSLVLSALLV

SEQ ID NO: 358 - protein, 21 aa, Homo sapiens
AGTVLLLRAGFYAVSFLSVAV

SEQ ID NO: 359 - protein, 21 aa, Homo sapiens
AALAVISFLLGLGLGVACVLA

SEQ ID NO: 360 - protein, 22 aa, Homo sapiens
MALIVLGGVAGLLLFIGLGIFF

SEQ ID NO: 361 - protein, 27 aa, Homo sapiens
FWVLVVVGGVLACYSLLVTVAFIIFWV

SEQ ID NO: 362 - protein, 21 aa, Homo sapiens
FLFVLLGVGSMGVAAIVWGAW

SEQ ID NO: 363 - protein, 21 aa, Homo sapiens
LWVAVGGAAGGAALLAAGAGL

SEQ ID NO: 364 - protein, 21 aa, Homo sapiens
YLYVLSGIGGLLLLLLIFIVL

SEQ ID NO: 365 - protein, 23 aa, Homo sapiens
IAAIVGGTVAGIVLIGILLLVIW

SEQ ID NO: 366 - protein, 21 aa, Homo sapiens
ILLLTLVSFLIFILFIIVQLF

SEQ ID NO: 367 - protein, 19 aa, Homo sapiens
VAVGLGSCLAILILAICGL

SEQ ID NO: 368 - protein, 23 aa, Homo sapiens
TLIRGALMGAGFLLTYGLTWIYY

SEQ ID NO: 369 - protein, 21 aa, Homo sapiens
VALLLGALGFKALLLLGVLAA

SEQ ID NO: 370 - protein, 21 aa, Homo sapiens
VVLVAVGEVAMKILLLCLCLI

SEQ ID NO: 371 - protein, 21 aa, Homo sapiens
IYAGVCISVLVLLALLGVIIA

SEQ ID NO: 372 - protein, 21 aa, Homo sapiens
LLIVTIVATMLVLAVTVTSLC

SEQ ID NO: 373 - protein, 21 aa, Homo sapiens
ALLIVTIVVIMLVLGLAVAFC

SEQ ID NO: 374 - protein, 21 aa, Homo sapiens
ALVSGMCCALFLLILLTGVLC

SEQ ID NO: 375 - protein, 21 aa, Homo sapiens
FFMINTSILLIFIFIVLLIHF

SEQ ID NO: 376 - protein, 21 aa, Homo sapiens
TIIGVSVLSVLVVSVVAVLVY

SEQ ID NO: 377 - protein, 21 aa, Homo sapiens
FSLFIVCTVTLTLFLMTILTV

SEQ ID NO: 378 - protein, 21 aa, Homo sapiens
LIVTIGATMLVLAVTVTSLCI

SEQ ID NO: 379 - protein, 21 aa, Homo sapiens
LILFSLSISVSLFLMVMMTAS

SEQ ID NO: 380 - protein, 21 aa, Homo sapiens
AVILFFFTFFITTMVMLAALA

SEQ ID NO: 381 - protein, 21 aa, Homo sapiens
FALSLLAVALGLGVPMLHHLC

SEQ ID NO: 382 - protein, 21 aa, Homo sapiens
ILVIFSGMFLVFTLAGALFLH

SEQ ID NO: 383 - protein, 21 aa, Homo sapiens
GLIILLLFASVALVAAIIFGV

SEQ ID NO: 384 - protein, 21 aa, Homo sapiens
ILGGALSLTFVLGLLSGFLVW

SEQ ID NO: 385 - protein, 21 aa, Homo sapiens
TLGLCLCAVLCCFLVAVACFL

SEQ ID NO: 386 - protein, 21 aa, Homo sapiens
FGAPALLGLALVLALVLVGLV

SEQ ID NO: 387 - protein, 21 aa, Homo sapiens
WWFLSGSLVIVIVCSTVGLII

SEQ ID NO: 388 - protein, 22 aa, Homo sapiens
LIPVYCSILAAVVVGLVAYIAF

SEQ ID NO: 389 - protein, 23 aa, Homo sapiens
ILWTCLGLSLIISLAVFVLMFLL

SEQ ID NO: 390 - protein, 21 aa, Homo sapiens
LGWLTVVLLAVAACVLLLTSA

SEQ ID NO: 391 - protein, 21 aa, Homo sapiens
AAVICSALATVLLALLILCVI

SEQ ID NO: 392 - protein, 21 aa, Homo sapiens
YAVIAIVPVFCLMGLLGILVC

SEQ ID NO: 393 - protein, 21 aa, Homo sapiens
VLLPLVIFFGLCLLSLLFIGL

SEQ ID NO: 394 - protein, 30 aa, Homo sapiens
FALPVGLIVGVTALGLLIIGVVNCVIMTQV

SEQ ID NO: 395 - protein, 21 aa, Homo sapiens
MFWVQVLLAGLVVPLLLGATL

SEQ ID NO: 396 - protein, 21 aa, Homo sapiens
LVALVSSLLVVFTLAFLGLFF

SEQ ID NO: 397 - protein, 21 aa, Homo sapiens
LAVLLPLAFFLLLATVFSCIW

SEQ ID NO: 398 - protein, 21 aa, Homo sapiens
VAAILGLGLVLGLLGPLAILL

SEQ ID NO: 399 - protein, 22 aa, Homo sapiens
ALVVIPIIFGILFAILLVLVFI

SEQ ID NO: 400 - protein, 21 aa, Homo sapiens
PVLFWVILVLVVVVGSSAFLL

SEQ ID NO: 401 - protein, 27 aa, Homo sapiens
IISFFLALTSTALLFLLFFLTLRFSVV

SEQ ID NO: 402 - protein, 21 aa, Homo sapiens
LIIAMSTIFIMAIAIVLIIMF

SEQ ID NO: 403 - protein, 23 aa, Homo sapiens
ILVPCVLGLLLLPILAMLMALCV

SEQ ID NO: 404 - protein, 21 aa, Homo sapiens
ELLWPGAALLVLLGVAASLCV

SEQ ID NO: 405 - protein, 21 aa, Homo sapiens
IFSGFAGLLAILLVVAVFCIL

SEQ ID NO: 406 - protein, 21 aa, Homo sapiens
LWGSLAAVAIFFVITFLIFLC

SEQ ID NO: 407 - protein, 23 aa, Homo sapiens
ILVPCVLGLLLLPILAMLMALCV

SEQ ID NO: 408 - protein, 26 aa, Homo sapiens
TQIVLLGLVTAALWAGLLTLLLLWHW

SEQ ID NO: 409 - protein, 21 aa, Homo sapiens
LGHGPLVLQLLSFTLLAGLLV

SEQ ID NO: 410 - protein, 21 aa, Homo sapiens
ALILLILCVGMVVGLVALGIW

SEQ ID NO: 411 - protein, 21 aa, Homo sapiens
LIAVILGILCLVILVIAVVLG

SEQ ID NO: 412 - protein, 21 aa, Homo sapiens
VMVISCVFCMGLLTASIFLGV

SEQ ID NO: 413 - protein, 21 aa, Homo sapiens
ILLGISGTVNGILTLTLISLI

SEQ ID NO: 414 - protein, 21 aa, Homo sapiens
YYCLLLIFGCIVILIFIMTGI

SEQ ID NO: 415 - protein, 23 aa, Homo sapiens
LTAEVLGIICIVLMATVLKTIVL

SEQ ID NO: 416 - protein, 21 aa, Homo sapiens
PFFFCCFIAVAMGIRFIIMVA

SEQ ID NO: 417 - protein, 23 aa, Homo sapiens
LTAEVLGIICIVLMATVLKTIVL

SEQ ID NO: 418 - protein, 21 aa, Homo sapiens
QMFLWTVAGIPILFLSACFIT

SEQ ID NO: 419 - protein, 21 aa, Homo sapiens
WSVAGISIALLSACFIVSCVV

SEQ ID NO: 420 - protein, 21 aa, Homo sapiens
HLLLSLGLGLLLLVIICVVGF

SEQ ID NO: 421 - protein, 21 aa, Homo sapiens
LIPSVIAVVFILLLSVCFIAS

SEQ ID NO: 422 - protein, 23 aa, Homo sapiens
WSMAVVSILLLSVCFTVSSVVPH

SEQ ID NO: 423 - protein, 21 aa, Homo sapiens
LCLLVVTSLFLGCLGLTVTLI

SEQ ID NO: 424 - protein, 21 aa, Homo sapiens
VLRAALVPLVAGLVICLVVCI

SEQ ID NO: 425 - protein, 21 aa, Homo sapiens
VGLGLLLLLMGAGLAVQGWFL

SEQ ID NO: 426 - protein, 25 aa, Homo sapiens
YFYLTTATLALCLVFTVATIMVLVV

SEQ ID NO: 427 - protein, 21 aa, Homo sapiens
FSFLIVAGATTLFCLLHFGVI

SEQ ID NO: 428 - protein, 27 aa, Homo sapiens
LLLVASVIQGLGLLLCFTYICLHFSAL

SEQ ID NO: 429 - protein, 24 aa, Homo sapiens
IFMYLLTVFLITQMIGSALFAVYL

SEQ ID NO: 430 - protein, 22 aa, Homo sapiens
LCLLVMFFMVLVALVGLGLGMF

SEQ ID NO: 431 - protein, 21 aa, Homo sapiens
WALVAGLLLLLLLAAACAVFL

SEQ ID NO: 432 - protein, 21 aa, Homo sapiens
LIVIFTVLLQSLCVAVTYVYF

SEQ ID NO: 433 - protein, 21 aa, Homo sapiens
MFVALLGLGLGQVVCSVALFF

SEQ ID NO: 434 - protein, 21 aa, Homo sapiens
VPLALGLGLALACLGLLLAVV

SEQ ID NO: 435 - protein, 21 aa, Homo sapiens
VALWLSWGAALGAVACAMALL

SEQ ID NO: 436 - protein, 21 aa, Homo sapiens
LLAATLLLALLSCCLTVVSFY

SEQ ID NO: 437 - protein, 21 aa, Homo sapiens
FCSIVMLLFLCSFSWLIFIFL

SEQ ID NO: 438 - protein, 45 aa, Homo sapiens
TTTPAPRPPTPAPTIASQPLSLRPEACRPAAGGAVHTRGLDFACD

SEQ ID NO: 439 - protein, 55 aa, Homo sapiens
FVPVFLPAKPTTTPAPRPPTPAPTIASQPLSLRPEACRPAAGGAVHTRGLDFACD

SEQ ID NO: 440 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 441 - protein, 236 aa, Homo sapiens
EPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVESCSVMHEALHNHYTQKSLSLSPGKKDPK

SEQ ID NO: 442 - protein, 39 aa, Homo sapiens
IEVMYPPPYLDNEKSNGTIIHVKGKHLCPSPLFPGPSKP

SEQ ID NO: 443 - protein, 16 aa, Homo sapiens
SPTEPSSKTGNPRHLH

SEQ ID NO: 444 - protein, 12 aa, Homo sapiens
ESKYGPPCPSCP

SEQ ID NO: 445 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 446 - protein, 16 aa, Homo sapiens
GLAVSTISSFFPPGYQ

SEQ ID NO: 447 - protein, 5 aa, synthetic construct
GGGGS

SEQ ID NO: 448 - protein, 10 aa, synthetic construct
GGGGSGGGGS

SEQ ID NO: 449 - protein, 15 aa, synthetic construct
GGGGSGGGGSGGGGS

SEQ ID NO: 450 - protein, 25 aa, synthetic construct
GGSGGSGGYPYDVPDYAGGGSGGGS

SEQ ID NO: 451 - protein, 24 aa, synthetic construct
GGSGGSGGGGGSGGGSGGGSGGGS

SEQ ID NO: 452 - protein, 26 aa, synthetic construct
GGSGGSGGGPEDEPGSGSGGGSGGGS

SEQ ID NO: 453 - protein, 42 aa, synthetic construct
GGSGGSGGGGGSGGGSGGGSGGGSGSGSGSGSEDGSGSGSGS

SEQ ID NO: 454 - protein, 28 aa, synthetic construct
GSGSGSGSGSEDEDEDEDGSGSGSGSGS

SEQ ID NO: 455 - skipped

SEQ ID NO: 456 - protein, 18 aa, synthetic construct
GSGSGSGSEDGSGSGSGS

SEQ ID NO: 457 - protein, 18 aa, synthetic construct
GSGSGSGSGSGSGSGSGS

SEQ ID NO: 458 - protein, 15 aa, synthetic construct
GCGGSGGGGSGGGGS

SEQ ID NO: 459 - protein, 24 aa, synthetic construct
GGSGGSGGGGGSGGGSGGGSGGGS

SEQ ID NO: 460 - protein, 26 aa, synthetic construct
SGRGGGGSGGGGSGGGGSGGGGSSPA

SEQ ID NO: 461 - protein, 24 aa, synthetic construct
GGGGSGGGGSGGGGSGGGGSGGGG

SEQ ID NO: 462 - protein, 58 aa, synthetic construct
SGRGASSGSSGSGSQKKPRYEIRWKVVVISAILALVVLTVISLIILIMLWGSGMQSPA

SEQ ID NO: 463 - protein, 30 aa, Homo sapiens
MKTLPAMLGTGKLFWVFFLIPYLDIWNIHG

SEQ ID NO: 464 - protein, 24 aa, Homo sapiens
MLLEPGRGCCALAILLAIVDIQSG

SEQ ID NO: 465 - protein, 28 aa, Homo sapiens
MLCPWRTANLGLLLILTIFLVAASSSLC

SEQ ID NO: 466 - protein, 17 aa, Homo sapiens
MPLLLLLPLLWAGALAM

SEQ ID NO: 467 - protein, 34 aa, Homo sapiens
MGHLSAPLHRVRVPWQGLLLTASLLTFWNPPTTA

SEQ ID NO: 468 - protein, 35 aa, Homo sapiens
MACLGFQRHKAQLNLATRTWPCTLLFFLLFIPVFC

SEQ ID NO: 469 - protein, 25 aa, Homo sapiens
MLGIWTLLPLVLTSVARLSSKSVNA

SEQ ID NO: 470 - protein, 19 aa, Homo sapiens
MLLWTAVLLFVPCVGKTVW

SEQ ID NO: 471 - protein, 21 aa, Homo sapiens
MLPCLVVLLAALLSLRLGSDA

SEQ ID NO: 472 - protein, 19 aa, Homo sapiens
MAWSLGSWLGGCLLVSALG

SEQ ID NO: 473 - protein, 19 aa, Homo sapiens
MLAVGCALLAALLAAPGAA

SEQ ID NO: 474 - protein, 22 aa, Homo sapiens
MLTLQTWLVQALFIFLTTESTG

SEQ ID NO: 475 - protein, 21 aa, Homo sapiens
MSLLVVSMACVGFFLLQGAWP

SEQ ID NO: 476 - protein, 21 aa, Homo sapiens
MSLMVVSMACVGFFLLQGAWP

SEQ ID NO: 477 - protein, 21 aa, Homo sapiens
MSLMVVSMVCVGFFLLQGAWP

SEQ ID NO: 478 - protein, 21 aa, Homo sapiens
MSLMVISMACVGFFLLQGAWT

SEQ ID NO: 479 - protein, 21 aa, Homo sapiens
MSLMVVSMACVGFFLLQGAWT

SEQ ID NO: 480 - protein, 21 aa, Homo sapiens
MSLMVVSMACVGLFLVQRAGP

SEQ ID NO: 481 - protein, 21 aa, Homo sapiens
MSLTVVSMACVGFFLLQGAWP

SEQ ID NO: 482 - protein, 25 aa, Homo sapiens
MSLMVVSMACVGFFLLEGPWPHVGG

SEQ ID NO: 483 - protein, 22 aa, Homo sapiens
MWEAQFLGLLFLQPLWVAPVKP

SEQ ID NO: 484 - protein, 23 aa, Homo sapiens
MTPILTVLICLGLSLGPRTHVQA

SEQ ID NO: 485 - protein, 21 aa, Homo sapiens
MTPIVTVLICLGLSLGPRTHV

SEQ ID NO: 486 - protein, 23 aa, Homo sapiens
MTPALTALLCLGLSLGPRTRVQA

SEQ ID NO: 487 - protein, 21 aa, Homo sapiens
MIPTFTALLCLGLSLGPRTHM

SEQ ID NO: 488 - protein, 23 aa, Homo sapiens
MTLTLSVLICLGLSVGPRTCVQA

SEQ ID NO: 489 - protein, 18 aa, Homo sapiens
MAWMLLLILIMVHPGSCA

SEQ ID NO: 490 - protein, 23 aa, Homo sapiens
MQIPQAPWPVVWAVLQLGWRPGW

SEQ ID NO: 491 - protein, 16 aa, Homo sapiens
MLLPLLLSSLLGGSQA

SEQ ID NO: 492 - protein, 18 aa, Homo sapiens
MLLLLLLPLLWGRERVEG

SEQ ID NO: 493 - protein, 17 aa, Homo sapiens
MLLLLLPLLWGRERAEG

SEQ ID NO: 494 - protein, 21 aa, Homo sapiens
MEKKWKYCAVYYIIQIHFVKG

SEQ ID NO: 495 - protein, 33 aa, Homo sapiens
MEAAVAAPRPRLLLLVLAAAAAAAAALLPGATA

SEQ ID NO: 496 - protein, 22 aa, Homo sapiens
MGRGLLRGLWPLHIVLWTRIAS

SEQ ID NO: 497 - protein, 21 aa, Homo sapiens
MRWCLLLIWAQGLRQAPLASG

SEQ ID NO: 498 - protein, 21 aa, Homo sapiens
MFSHLPFDCVLLLLLLLLTRS

SEQ ID NO: 499 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 500 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 501 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 502 - protein, 161 aa, Homo sapiens
RISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 503 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 504 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 505 - protein, 266 aa, Homo sapiens
APRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 506 - protein, 237 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 507 - protein, 402 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGGSGGGGSGGGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 508 - protein, 201 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 509 - protein, 224 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGSGSGSGSGSGSGSGSGSGSGSGSGSGSVAISTSTVLLCGLSAVSLLACYLKSRQTPPLASVEMEAMEALPVTWGTSSRDEDLENCSHHL

SEQ ID NO: 510 - protein, 21 aa, synthetic construct
GSGEGRGSLLTCGDVEENPGP

SEQ ID NO: 511 - protein, 22 aa, synthetic construct
GSGATNFSLLKQAGDVEENPGP

SEQ ID NO: 512 - protein, 23 aa, synthetic construct
GSGQCTNYALLKLAGDVESNPGP

SEQ ID NO: 513 - protein, 25 aa, synthetic construct
GSGVKQTLNFDLLKLAGDVESNPGP

SEQ ID NO: 514 - protein, 636 aa, Tribolium castaneum
MMLNWLKSGKLESQSQEQSSCYLENSNCLPPTLDSTDIIGEENKAGTTSRKKRKYDEDYLNFGFTWTGDKDEPNGLCVICEQVVNNSSLNPAKLKRHLDTKHPTLKGKSEYFKRKCNELNQKKHTFERYVRDDNKNLLKASYLVSLRIAKQGEAYTIAEKLIKPCTKDLTTCVFGEKFASKVDLVPLSDTTISRRIEDMSYFCEAVLVNRLENAKCGFTLQMDESTDVAGLAILLVFVRYIHESSFEEDMLFCKALPTQTTGEEIFNLLNAYFEKHSIPWNLCYHICTDGAKAMVGVIKGVIARIKKLVPDIKASHCCLHRHALAVKRIPNALHEVLNDAVKMINFIKSRPLNARVFALLCDDLGSLHKNLLLHTEVRWLSRGKVLTRFWELRDEIRIFFNEREFAGKLNDTSWLQNLAYIADIFSYLNEVNLSLQGPNSTIFKVNSRINSIKSKLKLWEECITKNNTECFANLNDFLETSNTALDPNLKSNILEHLNGLKNTFLEYFPPTCNNISWVENPFNECGNVDTLPIKEREQLIDIRTDTTLKSSFVPDGIGPFWIKLMDEFPEISKRAVKELMPFVTTYLCEKSFSVYVATKTKYRNRLDAEDDMRLQLTTIHPDIDNLCNNKQAQKSH

SEQ ID NO: 515 - protein, 636 aa, Tribolium castaneum; identical to SEQ ID NO: 514

SEQ ID NO: 516 - DNA, 1911 bp, Tribolium castaneum
  source 1..1911: mol_type = genomic DNA
atgatgttgaattggctgaaaagtggaaagcttgaaagtcaatcacaggaacagagttcctgctaccttgagaactctaactgcctgccaccaacgctcgattctacagatattatcggtgaagagaacaaagctggtaccacctctcgcaagaagcggaaatatgacgaggactatctgaacttcggttttacatggactggcgacaaggatgagcccaacggactttgtgtgatttgcgagcaggtagtcaacaattcctcacttaacccggccaaactgaaacgccatttggacacaaagcatccgacgcttaaaggcaagagcgaatacttcaaaagaaaatgtaacgagctcaatcaaaagaagcatacttttgagcgatacgtaagggacgataacaagaacctcctgaaagcttcttatctcgtcagtttgagaatagctaaacagggcgaggcatataccatagcggagaagttgatcaagccttgcaccaaggatctgacaacttgcgtatttggagaaaaattcgcgagcaaagttgatctcgtccccctgtccgacacgactatttcaaggcgaatcgaagacatgagttacttctgtgaagccgtgctggtgaacaggttgaaaaatgctaaatgtgggtttacgctgcagatggacgagtcaacagatgttgccggtcttgcaatcctgcttgtgtttgttaggtacatacatgaaagctcttttgaggaggatatgttgttctgcaaagcacttcccactcagacgacaggggaggagattttcaatcttctcaatgcctatttcgaaaagcactccatcccatggaatctgtgttaccacatttgcacagacggtgccaaggcaatggtaggagttattaaaggagtcatagcgagaataaaaaaactcgtccctgatataaaagctagccactgttgcctgcatcgccacgctttggctgtaaagcgaataccgaatgcattgcacgaggtgctcaatgacgctgttaaaatgatcaacttcatcaagtctcggccgttgaatgcgcgcgtcttcgctttgctgtgtgacgatttggggagcctgcataaaaatcttcttcttcataccgaagtgaggtggctgtctagaggaaaggtgctgacccgattttgggaactgagagatgaaattagaattttcttcaacgaaagggaatttgccgggaaattgaacgacaccagttggttgcaaaatttggcatatatagctgacatattcagttatctgaatgaagttaatctttccctgcaagggccgaatagcacaatcttcaaggtaaatagccgcattaacagtattaaatcaaagttgaagttgtgggaagagtgtataacgaaaaataacactgagtgttttgcgaacctcaacgattttttggaaacttcaaacactgcgttggatccaaacctgaagtctaatattttggaacatctcaacggtcttaagaacacctttctggagtattttccacctacgtgtaataatatctcctgggtggagaatcctttcaatgaatgcggtaacgtcgatacactcccaataaaagagagggaacaattgattgacatacggactgatacgacattgaaatcttcattcgtgcctgatggtataggaccattctggatcaaactgatggacgaatttccagaaattagcaaacgagctgtcaaagagctcatgccatttgtaaccacttacctctgtgagaaatcattttccgtctatgtagccacaaaaacaaaatatcgaaatagacttgatgctgaagacgatatgcgactccaacttactactatccatccagacattgacaacctttgtaacaacaagcaggctcagaaatcccactga

SEQ ID NO: 517 - DNA, 1911 bp, Tribolium castaneum; identical to SEQ ID NO: 516
  source 1..1911: mol_type = genomic DNA

SEQ ID NO: 518 - protein, 206 aa, Staphylococcus aureus
MKKWTNRLMTIAGVVLILVAAYLFAKPHIDNYLHDKDKDEKIEQYDKNVKEQASKDNKQQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVEVLDEQKGKDKQLTLITCDDYNEKTGVWEKRKIFVATEVK

SEQ ID NO: 519 - protein, 156 aa, synthetic construct
MQAKPQIPKDKSKVAGYIEIPDADIKEPVYPGPATREQLNRGVSFAKENQSLDDQNISIAGHTFIDRPNYQFTNLKAAKKGSMVYFKVGNETRKYKMTSIRNVKPTAVEVLDEQKGKDKQLTLITCDDYNEETGVWETRKIFVATEVKLEHHHHHH

SEQ ID NO: 520 - protein, 5 aa, synthetic construct
LPXTG

SEQ ID NO: 521 - protein, 5 aa, synthetic construct
LPXTA

SEQ ID NO: 522 - DNA, 18 bp, Homo sapiens
  source 1..18: mol_type = genomic DNA
tgctgttgaggagctgga

SEQ ID NO: 523 - DNA, 18 bp, Homo sapiens
  source 1..18: mol_type = genomic DNA
agcacaccaggcagagtt

SEQ ID NO: 524 - DNA, 18 bp, Homo sapiens
  source 1..18: mol_type = genomic DNA
cggctgaggagcggaaga

SEQ ID NO: 525 - DNA, 18 bp, Homo sapiens
  source 1..18: mol_type = genomic DNA
tggaggtgagcaatcccc

SEQ ID NO: 526 - DNA, 20 bp, synthetic construct
  source 1..20: mol_type = other DNA
ttaatacgactcactatagg

SEQ ID NO: 527 - DNA, 20 bp, Homo sapiens
  source 1..20: mol_type = genomic DNA
gttttagagctagaaatagc

SEQ ID NO: 528 - protein, 240 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYVYFLGRLVPRGRGAAEAATRKQRITETESPYQELQGQRSDVYSDLNTQRPYYK

SEQ ID NO: 529 - protein, 239 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQGVLAGIVMGDLVLTVLIALAVYFLGRLVPRGRGAAEAATRKQRITETESPYQELQGQRSDVYSDLNTQRPYYK

SEQ ID NO: 530 - protein, 211 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQLLAGLVAADAVASLLIVGAVFLCARPRRSPAQEDGKVYINMPGRG

SEQ ID NO: 531 - protein, 241 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQQTTPGERSSLPAFYPGTSGSCSGCGSLSLPLLAGLVAADAVASLLIVGAVFLCARPRRSPAQEDGKVYINMPGRG

SEQ ID NO: 532 - protein, 223 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSLCARPRRSPAQEDGKVYINMPGRG

SEQ ID NO: 533 - protein, 227 aa, Homo sapiens
MGRPLLLPLLLLLQPPAFLQPGGSTGSGPSYLYGVTQPKHLSASMGGSVEIPFSFYYPWELAIVPNVRISWRRGHFHGQSFYSTRPPSIHKDYVNRLFLNWTEGQESGFLRISNLRKEDQSVYFCRVELDTRRSGRQQLQSIKGTKLTITQAVTTTTTWRPSSTTTIAGLRVTESKGHSESWHLSLDTAIRVALAVAVLKTVILGLLCLLLLWWRRRKGSRAPSSDF

SEQ ID NO: 534 - protein, 21 aa, Homo sapiens
VALAVAVLKTVILGLLCLLLL

SEQ ID NO: 535 - protein, 15 aa, Homo sapiens
WWRRRKGSRAPSSDF

SEQ ID NO: 536 - protein, 598 aa, Homo sapiens
MTPIVTVLICLGLSLGPRTHVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 537 - protein, 440 aa, Homo sapiens; identical to SEQ ID NO: 28

SEQ ID NO: 538 - protein, 147 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYI

SEQ ID NO: 539 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTERYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 540 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVRIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 541 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRIIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 542 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVEIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 543 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERLWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 544 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHREIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 545 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLVLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 546 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRAGIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 547 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYAPGAARLTALRIKKTLSQLSQQEGIKM

SEQ ID NO: 548 - protein, 194 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQ

SEQ ID NO: 549 - protein, 187 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFY

SEQ ID NO: 550 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAECFSELEHLDRLSGRSCSEEKIPEDGSLNTTK

SEQ ID NO: 551 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEAHLTAQCVAERFSELEHLDRLSGRSCSEEKIPEDGSLNTTK

SEQ ID NO: 552 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEACLTAQCVAERFSELEHLDRLSGRSCSEEKIPEDGSLNTTK

SEQ ID NO: 553 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSCCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 554 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSHCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 555 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAEHFSELEHLDRLSGRSCSEEKIPEDGSLNTTK

SEQ ID NO: 556 - protein, 295 aa, synthetic construct
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 557 - protein, 265 aa, synthetic construct
KESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 558 - protein, 382 aa, synthetic construct
MLCPWRTANLGLLLILTIFLVAASSSLCMDEKQITQNYSKVLAEVNTSWPVKMATNAVLCCPPIALRNLIIITWEIILRGQPSCTKAYRKETNETKETNCTDERITWVSRPDQNSDLQIRPVAITHDGYYRCIMVTPDGNFHRGYHLQVLVTPEVTLFQNRNRTAVCKAVAGKPAAQISWIPEGDCATKQEYWSNGTVTVKSTCHWEVHNVSTVTCHVSHLTGNKSLYIELLPVPGAKKSAKLYIPYIILTIIILTIVGFIWLLKVNGCRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 559 - protein, 354 aa, synthetic construct
MDEKQITQNYSKVLAEVNTSWPVKMATNAVLCCPPIALRNLIIITWEIILRGQPSCTKAYRKETNETKETNCTDERITWVSRPDQNSDLQIRPVAITHDGYYRCIMVTPDGNFHRGYHLQVLVTPEVTLFQNRNRTAVCKAVAGKPAAQISWIPEGDCATKQEYWSNGTVTVKSTCHWEVHNVSTVTCHVSHLTGNKSLYIELLPVPGAKKSAKLYIPYIILTIIILTIVGFIWLLKVNGCRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 560 - protein, 299 aa, synthetic construct
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 561 - protein, 264 aa, synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 562 - protein, 308 aa, synthetic construct
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 563 - protein, 283 aa, synthetic construct
QVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 564 - protein, 375 aa, synthetic construct
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNVIIFFAFVLLLSGALAYCLALQLYVRRRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 565 - protein, 354 aa, synthetic construct
HGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNVIIFFAFVLLLSGALAYCLALQLYVRRRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 566 - protein, 361 aa, synthetic construct
MAWSLGSWLGGCLLVSALGMVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSWMVAVILMASVFMVCLALLGCFALLWCVYRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 567 - protein, 342 aa, synthetic construct
MVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSWMVAVILMASVFMVCLALLGCFALLWCVYRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 568 - protein, 759 aa, synthetic construct
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 569 - protein, 737 aa, synthetic construct
ELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 570 - protein, 334 aa, synthetic construct
MQIPQAPWPVVWAVLQLGWRPGWMQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVVGVVGGLLGSLVLLVWVLAVICSRAARGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 571 - protein, 311 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVVGVVGGLLGSLVLLVWVLAVICSRAARGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 572 - protein, 657 aa, synthetic construct
MEKKWKYCAVYYIIQIHFVKGVWEKTVNTEENVYATLGSDVNLTCQTQTVGFFVQMQWSKVTNKIDLIAVYHPQYGFYCAYGRPCESLVTFTETPENGSKWTLHLRNMSCSVSGRYECMLVLYPEGIQTKIYNLLIQTHVTADEWNSNHTIEIEINQTLEIPCFQNSSSKISSEFTYAWSVENSSTDSWVLLSKGIKEDNGTQETLISQNHLISNSTLLKDRVKLGTDYRLHLSPVQIFDDGRKFSCHIRVGPNKILRSSTTVKVFAKPEIPVIVENNSTDVLVERRFTCLLKNVFPKANITWFIDGSFLHDEKEGIYITNEERKGKDGFLELKSVLTRVHSNKPAQSDNLTIWCMALSPVPGNKVWNISSEKITFLLGSEISSTDPPLSVTESTLDTQPSPASSVSPARYPATSSVTLVDVSALRPNTTPQPSNSSMTTRGFNYPWTSSGTDTKKSVSRIPSETYSSSPSGAGSTLHDNVFTSTARAFSEVPTTANGSTKTNHVHITGIVVNKPKDGMSWPVIVAALLFCCMILFGLGVRKWCQRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 573 - protein, 636 aa, synthetic construct
VWEKTVNTEENVYATLGSDVNLTCQTQTVGFFVQMQWSKVTNKIDLIAVYHPQYGFYCAYGRPCESLVTFTETPENGSKWTLHLRNMSCSVSGRYECMLVLYPEGIQTKIYNLLIQTHVTADEWNSNHTIEIEINQTLEIPCFQNSSSKISSEFTYAWSVENSSTDSWVLLSKGIKEDNGTQETLISQNHLISNSTLLKDRVKLGTDYRLHLSPVQIFDDGRKFSCHIRVGPNKILRSSTTVKVFAKPEIPVIVENNSTDVLVERRFTCLLKNVFPKANITWFIDGSFLHDEKEGIYITNEERKGKDGFLELKSVLTRVHSNKPAQSDNLTIWCMALSPVPGNKVWNISSEKITFLLGSEISSTDPPLSVTESTLDTQPSPASSVSPARYPATSSVTLVDVSALRPNTTPQPSNSSMTTRGFNYPWTSSGTDTKKSVSRIPSETYSSSPSGAGSTLHDNVFTSTARAFSEVPTTANGSTKTNHVHITGIVVNKPKDGMSWPVIVAALLFCCMILFGLGVRKWCQRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 574 - protein, 281 aa, synthetic construct
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKARVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 575 - protein, 260 aa, synthetic construct
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKARVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 576 - protein, 296 aa, synthetic construct
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 577 - protein, 266 aa, synthetic construct
KESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 578 - protein, 382 aa, synthetic construct
MLCPWRTANLGLLLILTIFLVAASSSLCMDEKQITQNYSKVLAEVNTSWPVKMATNAVLCCPPIALRNLIIITWEIILRGQPSCTKAYRKETNETKETNCTDERITWVSRPDQNSDLQIRPVAITHDGYYRCIMVTPDGNFHRGYHLQVLVTPEVTLFQNRNRTAVCKAVAGKPAAQISWIPEGDCATKQEYWSNGTVTVKSTCHWEVHNVSTVTCHVSHLTGNKSLYIELLPVPGAKKSAKLFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 579 - protein, 354 aa, synthetic construct
MDEKQITQNYSKVLAEVNTSWPVKMATNAVLCCPPIALRNLIIITWEIILRGQPSCTKAYRKETNETKETNCTDERITWVSRPDQNSDLQIRPVAITHDGYYRCIMVTPDGNFHRGYHLQVLVTPEVTLFQNRNRTAVCKAVAGKPAAQISWIPEGDCATKQEYWSNGTVTVKSTCHWEVHNVSTVTCHVSHLTGNKSLYIELLPVPGAKKSAKLFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 580 - protein, 300 aa, synthetic construct
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 581 - protein, 265 aa, synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 582 - protein, 312 aa, synthetic construct
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 583 - protein, 287 aa, synthetic construct
QVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 584 - protein, 374 aa, synthetic construct
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 585 - protein, 353 aa, synthetic construct
HGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 586 - protein, 359 aa, synthetic construct
MAWSLGSWLGGCLLVSALGMVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 587 - protein, 340 aa, synthetic construct
MVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 588 - protein, 758 aa, synthetic construct
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 589 - protein, 736 aa, synthetic construct
ELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 590 - protein, 333 aa, synthetic construct
MQIPQAPWPVVWAVLQLGWRPGWMQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 591 - protein, 310 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 592 - protein, 658 aa, synthetic construct
MEKKWKYCAVYYIIQIHFVKGVWEKTVNTEENVYATLGSDVNLTCQTQTVGFFVQMQWSKVTNKIDLIAVYHPQYGFYCAYGRPCESLVTFTETPENGSKWTLHLRNMSCSVSGRYECMLVLYPEGIQTKIYNLLIQTHVTADEWNSNHTIEIEINQTLEIPCFQNSSSKISSEFTYAWSVENSSTDSWVLLSKGIKEDNGTQETLISQNHLISNSTLLKDRVKLGTDYRLHLSPVQIFDDGRKFSCHIRVGPNKILRSSTTVKVFAKPEIPVIVENNSTDVLVERRFTCLLKNVFPKANITWFIDGSFLHDEKEGIYITNEERKGKDGFLELKSVLTRVHSNKPAQSDNLTIWCMALSPVPGNKVWNISSEKITFLLGSEISSTDPPLSVTESTLDTQPSPASSVSPARYPATSSVTLVDVSALRPNTTPQPSNSSMTTRGFNYPWTSSGTDTKKSVSRIPSETYSSSPSGAGSTLHDNVFTSTARAFSEVPTTANGSTKTNHVHITGIVVNKPKDGMFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 593 - protein, 637 aa, synthetic construct
VWEKTVNTEENVYATLGSDVNLTCQTQTVGFFVQMQWSKVTNKIDLIAVYHPQYGFYCAYGRPCESLVTFTETPENGSKWTLHLRNMSCSVSGRYECMLVLYPEGIQTKIYNLLIQTHVTADEWNSNHTIEIEINQTLEIPCFQNSSSKISSEFTYAWSVENSSTDSWVLLSKGIKEDNGTQETLISQNHLISNSTLLKDRVKLGTDYRLHLSPVQIFDDGRKFSCHIRVGPNKILRSSTTVKVFAKPEIPVIVENNSTDVLVERRFTCLLKNVFPKANITWFIDGSFLHDEKEGIYITNEERKGKDGFLELKSVLTRVHSNKPAQSDNLTIWCMALSPVPGNKVWNISSEKITFLLGSEISSTDPPLSVTESTLDTQPSPASSVSPARYPATSSVTLVDVSALRPNTTPQPSNSSMTTRGFNYPWTSSGTDTKKSVSRIPSETYSSSPSGAGSTLHDNVFTSTARAFSEVPTTANGSTKTNHVHITGIVVNKPKDGMFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 594 - protein, 280 aa, synthetic construct
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 595 - protein, 259 aa, synthetic construct
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 596 - protein, 735 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSALRNVPFRSEVLGWDPDSLADYFKKLNYKDCEKAVKKYHIDGARFLNLTENDIQKFPKLRVPILSKLSQEINKNEERRSIFTRKPQVPRFPEETESHEEDNGGWSSFEEDDYESPNDDQDGEDDGDYESPNEEEEAPVEDDADYEPPPSNDEEALQNSILPAKPFPNSNSMYIDRPPSGKTPQQPPVPPQRPMAALPPPPAGRNHSPLPPPQTNHEEPSRSRNHKTAKLPAPSIDRSTKPPLDRSLAPFDREPFTLGKKPPFSDKPSIPAGRSLGEHLPKIQKPPLPPTTERHERSSPLPGKKPPVPKHGWGPDRRENDEDDVHQRPLPQPALLPMSSNTFPSRSTKPSPMNPLPSSHMPGAFSESNSSFPQSASLPPYFSQGPSNRPPIRAEGRNFPLPLPNKPRPPSPAEEENSLNEEWYVSYITRPEAEAALRKINQDGTFLVRDSSKKTTTNPYVLMVLYKDKVYNIQIRYQKESQVYLLGTGLRGKEDFLSVSDIIDYFRKMPLLLIDGKNRGSRYQCTLTHAAGYP

SEQ ID NO: 597 - protein, 713 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSALRNVPFRSEVLGWDPDSLADYFKKLNYKDCEKAVKKYHIDGARFLNLTENDIQKFPKLRVPILSKLSQEINKNEERRSIFTRKPQVPRFPEETESHEEDNGGWSSFEEDDYESPNDDQDGEDDGDYESPNEEEEAPVEDDADYEPPPSNDEEALQNSILPAKPFPNSNSMYIDRPPSGKTPQQPPVPPQRPMAALPPPPAGRNHSPLPPPQTNHEEPSRSRNHKTAKLPAPSIDRSTKPPLDRSLAPFDREPFTLGKKPPFSDKPSIPAGRSLGEHLPKIQKPPLPPTTERHERSSPLPGKKPPVPKHGWGPDRRENDEDDVHQRPLPQPALLPMSSNTFPSRSTKPSPMNPLPSSHMPGAFSESNSSFPQSASLPPYFSQGPSNRPPIRAEGRNFPLPLPNKPRPPSPAEEENSLNEEWYVSYITRPEAEAALRKINQDGTFLVRDSSKKTTTNPYVLMVLYKDKVYNIQIRYQKESQVYLLGTGLRGKEDFLSVSDIIDYFRKMPLLLIDGKNRGSRYQCTLTHAAGYP

SEQ ID NO: 598 - protein, 498 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 599 - protein, 476 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 600 - protein, 239 aa, synthetic construct; identical to SEQ ID NO: 529

SEQ ID NO: 601 - protein, 217 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQGVLAGIVMGDLVLTVLIALAVYFLGRLVPRGRGAAEAATRKQRITETESPYQELQGQRSDVYSDLNTQRPYYK

SEQ ID NO: 602 - protein, 618 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSASSSGSSPRPAPDENEFPFGCPPTVCQDPKEPRALCCAGCLSENPRNGEDQICPKCRGEDLQSISPGSRLRTQEKAHPEVAEAGIGCPFAGVGCSFKGSPQSVQEHEVTSQTSHLNLLLGFMKQWKARLGCGLESGPMALEQNLSDLQLQAAVEVAGDLEVDCYRAPCSESQEELALQHFMKEKLLAELEGKLRVFENIVAVLNKEVEASHLALATSIHQSQLDRERILSLEQRVVELQQTLAQKDQALGKLEQSLRLMEEASFDGTFLWKITNVTRRCHESACGRTVSLFSPAFYTAKYGYKLCLRLYLNGDGTGKRTHLSLFIVIMRGEYDALLPWPFRNKVTFMLLDQNNREHAIDAFRPDLSSASFQRPQSETNVASGCPLFFPLSKLQSPKHAYVKDDTMFLKCIVETST

SEQ ID NO: 603 - protein, 596 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSASSSGSSPRPAPDENEFPFGCPPTVCQDPKEPRALCCAGCLSENPRNGEDQICPKCRGEDLQSISPGSRLRTQEKAHPEVAEAGIGCPFAGVGCSFKGSPQSVQEHEVTSQTSHLNLLLGFMKQWKARLGCGLESGPMALEQNLSDLQLQAAVEVAGDLEVDCYRAPCSESQEELALQHFMKEKLLAELEGKLRVFENIVAVLNKEVEASHLALATSIHQSQLDRERILSLEQRVVELQQTLAQKDQALGKLEQSLRLMEEASFDGTFLWKITNVTRRCHESACGRTVSLFSPAFYTAKYGYKLCLRLYLNGDGTGKRTHLSLFIVIMRGEYDALLPWPFRNKVTFMLLDQNNREHAIDAFRPDLSSASFQRPQSETNVASGCPLFFPLSKLQSPKHAYVKDDTMFLKCIVETST

SEQ ID NO: 604 - protein, 703 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEACSRQHRLDQDKIEALSSKVQQLERSIGLKDLAMADLEQKVLEMEASTYDGVFIWKISDFARKRQEAVAGRIPAIFSPAFYTSRYGYKMCLRIYLNGDGTGRGTHLSLFFVVMKGPNDALLRWPFNQKVTLMLLDQNNREHVIDAFRPDVTSSSFQRPVNDMNIASGCPLFCPVSKMEAKNSYVRDDAIFIKAIVDLTGL

SEQ ID NO: 605 - protein, 681 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEACSRQHRLDQDKIEALSSKVQQLERSIGLKDLAMADLEQKVLEMEASTYDGVFIWKISDFARKRQEAVAGRIPAIFSPAFYTSRYGYKMCLRIYLNGDGTGRGTHLSLFFVVMKGPNDALLRWPFNQKVTLMLLDQNNREHVIDAFRPDVTSSSFQRPVNDMNIASGCPLFCPVSKMEAKNSYVRDDAIFIKAIVDLTGL

SEQ ID NO: 606 - protein, 770 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSESSKKMDSPGALQTNPPLKLHTDRSAGTPVFVPEQGGYKEKFVKTVEDKYKCEKCHLVLCSPKQTECGHRFCESCMAALLSSSSPKCTACQESIVKDKVFKDNCCKREILALQIYCRNESRGCAEQLMLGHLLVHLKNDCHFEELPCVRPDCKEKVLRKDLRDHVEKACKYREATCSHCKSQVPMIALQKHEDTDCPCVVVSCPHKCSVQTLLRSELSAHLSECVNAPSTCSFKRYGCVFQGTNQQIKAHEASSAVQHVNLLKEWSNSLEKKVSLLQNESVEKNKSIQSLHNQICSFEIEIERQKEMLRNNESKILHLQRVIDSQAEKLKELDKEIRPFRQNWEEADSMKSSVESLQNRVTELESVDKSAGQVARNTGLLESQLSRHDQMLSVHDIRLADMDLRFQVLETASYNGVLIWKIRDYKRRKQEAVMGKTLSLYSQPFYTGYFGYKMCARVYLNGDGMGKGTHLSLFFVIMRGEYDALLPWPFKQKVTLMLMDQGSSRRHLGDAFKPDPNSSSFKKPTGEMNIASGCPVFVAQTVLENGTYIKDDTIFIKVIVDTSDLPDP

SEQ ID NO: 607 - protein, 748 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSESSKKMDSPGALQTNPPLKLHTDRSAGTPVFVPEQGGYKEKFVKTVEDKYKCEKCHLVLCSPKQTECGHRFCESCMAALLSSSSPKCTACQESIVKDKVFKDNCCKREILALQIYCRNESRGCAEQLMLGHLLVHLKNDCHFEELPCVRPDCKEKVLRKDLRDHVEKACKYREATCSHCKSQVPMIALQKHEDTDCPCVVVSCPHKCSVQTLLRSELSAHLSECVNAPSTCSFKRYGCVFQGTNQQIKAHEASSAVQHVNLLKEWSNSLEKKVSLLQNESVEKNKSIQSLHNQICSFEIEIERQKEMLRNNESKILHLQRVIDSQAEKLKELDKEIRPFRQNWEEADSMKSSVESLQNRVTELESVDKSAGQVARNTGLLESQLSRHDQMLSVHDIRLADMDLRFQVLETASYNGVLIWKIRDYKRRKQEAVMGKTLSLYSQPFYTGYFGYKMCARVYLNGDGMGKGTHLSLFFVIMRGEYDALLPWPFKQKVTLMLMDQGSSRRHLGDAFKPDPNSSSFKKPTGEMNIASGCPVFVAQTVLENGTYIKDDTIFIKVIVDTSDLPDP

SEQ ID NO: 608 - protein, 724 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLAQAVHSLSVIPDSGYISEVRNFQETIHQLEGRLVRQDHQIRELTAKMETQSMYVSELKRTIRTLEDKVAEIEAQQCNGIYIWKIGNFGMHLKCQEEEKPVVIHSPGFYTGKPGYKLCMRLHLQLPTAQRCANYISLFVHTMQGEYDSHLPWPFQGTIRLTILDQSEAPVRQNHEEIMDAKPELLAFQRPTIPRNPKGFGYVTFMHLEALRQRTFIKDDTLLVRCEVSTRFDMGSLRREGFQPRSTDAGV

SEQ ID NO: 609 - protein, 702 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLAQAVHSLSVIPDSGYISEVRNFQETIHQLEGRLVRQDHQIRELTAKMETQSMYVSELKRTIRTLEDKVAEIEAQQCNGIYIWKIGNFGMHLKCQEEEKPVVIHSPGFYTGKPGYKLCMRLHLQLPTAQRCANYISLFVHTMQGEYDSHLPWPFQGTIRLTILDQSEAPVRQNHEEIMDAKPELLAFQRPTIPRNPKGFGYVTFMHLEALRQRTFIKDDTLLVRCEVSTRFDMGSLRREGFQPRSTDAGV

SEQ ID NO: 610 - protein, 423 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSASSTSLPAPGSRPKKPLGKMADWFRQTLLKKPKKRPNSPESTSSDASQPTSQDSPLPPSLSSVTSPSLPPTHASDSGSSRWSKDYDVCVCHSEEDLVAAQDLVSYLEGSTASLRCFLQLRDATPGGAIVSELCQALSSSHCRVLLITPGFLQDPWCKYQMLQALTEAPGAEGCTIPLLSGLSRAAYPPELRFMYYVDGRGPDGGFRQVKEAVMRYLQTLS

SEQ ID NO: 611 - protein, 401 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSASSTSLPAPGSRPKKPLGKMADWFRQTLLKKPKKRPNSPESTSSDASQPTSQDSPLPPSLSSVTSPSLPPTHASDSGSSRWSKDYDVCVCHSEEDLVAAQDLVSYLEGSTASLRCFLQLRDATPGGAIVSELCQALSSSHCRVLLITPGFLQDPWCKYQMLQALTEAPGAEGCTIPLLSGLSRAAYPPELRFMYYVDGRGPDGGFRQVKEAVMRYLQTLS

SEQ ID NO: 612 - protein, 658 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSDKLNKITVPASQKLRQLQKMVHDIKNNEGGIMNKIKKLKVKAPPSVPRRDYASESPADEEEQWSDDFDSDYENPDEHSDSEMYVMPAEENADDSYEPPPVEQETRPVHPALPFARGEYIDNRSSQRHSPPFSKTLPSKPSWPSEKARLTSTLPALTALQKPQVPPKPKGLLEDEADYVVPVEDNDENYIHPTESSSPPPEKAPMVNRSTKPNSSTPASPPGTASGRNSGAWETKSPPPAAPSPLPRAGKKPTTPLKTTPVASQQNASSVCEEKPIPAERHRGSSHRQEAVQSPVFPPAQKQIHQKPIPLPRFTEGGNPTVDGPLPSFSSNSTISEQEAGVLCKPWYAGACDRKSAEEALHRSNKDGSFLIRKSSGHDSKQPYTLVVFFNKRVYNIPVRFIEATKQYALGRKKNGEEYFGSVAEIIRNHQHSPLVLIDSQNNTKDSTRLKYAVKVS

SEQ ID NO: 613 - protein, 636 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSDKLNKITVPASQKLRQLQKMVHDIKNNEGGIMNKIKKLKVKAPPSVPRRDYASESPADEEEQWSDDFDSDYENPDEHSDSEMYVMPAEENADDSYEPPPVEQETRPVHPALPFARGEYIDNRSSQRHSPPFSKTLPSKPSWPSEKARLTSTLPALTALQKPQVPPKPKGLLEDEADYVVPVEDNDENYIHPTESSSPPPEKAPMVNRSTKPNSSTPASPPGTASGRNSGAWETKSPPPAAPSPLPRAGKKPTTPLKTTPVASQQNASSVCEEKPIPAERHRGSSHRQEAVQSPVFPPAQKQIHQKPIPLPRFTEGGNPTVDGPLPSFSSNSTISEQEAGVLCKPWYAGACDRKSAEEALHRSNKDGSFLIRKSSGHDSKQPYTLVVFFNKRVYNIPVRFIEATKQYALGRKKNGEEYFGSVAEIIRNHQHSPLVLIDSQNNTKDSTRLKYAVKVS

SEQ ID NO: 614 - protein, 419 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSEAIAKYDFKATADDELSFKRGDILKVLNEECDQNWYKAELNGKDGFIPKNYIEMKPHPWFFGKIPRAKAEEMLSKQRHDGAFLIRESESAPGDFSLSVKFGNDVQHFKVLRDGAGKYFLWVVKFNSLNELVDYHRSTSVSRNQQIFLRDIEQVPQQPTYVQALFDFDPQEDGELGFRRGDFIHVMDNSDPNWWKGACHGQTGMFPRNYVTPVNRNV

SEQ ID NO: 615 - protein, 397 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSEAIAKYDFKATADDELSFKRGDILKVLNEECDQNWYKAELNGKDGFIPKNYIEMKPHPWFFGKIPRAKAEEMLSKQRHDGAFLIRESESAPGDFSLSVKFGNDVQHFKVLRDGAGKYFLWVVKFNSLNELVDYHRSTSVSRNQQIFLRDIEQVPQQPTYVQALFDFDPQEDGELGFRRGDFIHVMDNSDPNWWKGACHGQTGMFPRNYVTPVNRNV

SEQ ID NO: 616 - protein, 266 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 617 - protein, 244 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 618 - protein, 212 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVSFCLVMVLLFAVDTGLYFSVKTNIRSSTRDWKDHKFKWRKDPQDK

SEQ ID NO: 619 - protein, 190 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVSFCLVMVLLFAVDTGLYFSVKTNIRSSTRDWKDHKFKWRKDPQDK

SEQ ID NO: 620 - protein, 307 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 621 - protein, 285 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 622 - protein, 264 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQWAVYAGLLGGVIMILIMVVILQLRRRGKTNHYQTTVEKKSLTIYAQVQKPGPLQKKLDSFPAQDPCTTIYVAATEPVPESVQETNSITVYASVTLPES

SEQ ID NO: 623 - protein, 242 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQWAVYAGLLGGVIMILIMVVILQLRRRGKTNHYQTTVEKKSLTIYAQVQKPGPLQKKLDSFPAQDPCTTIYVAATEPVPESVQETNSITVYASVTLPES

SEQ ID NO: 624 - protein, 286 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQLLSVLAMFFLLVLILSSVFLFRLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVI

SEQ ID NO: 625 - protein, 264 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQLLSVLAMFFLLVLILSSVFLFRLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVI

SEQ ID NO: 626 - protein, 271 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQILFMVSGICIVFGFIILLLLVLRKRRDSLSLSTQRTQGPAESARNLEYVSVSPTNNTVYASVTHSNRETEIWTPRENDTITIYSTINHSKESKPTFSRATALDNV

SEQ ID NO: 627 - protein, 249 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQILFMVSGICIVFGFIILLLLVLRKRRDSLSLSTQRTQGPAESARNLEYVSVSPTNNTVYASVTHSNRETEIWTPRENDTITIYSTINHSKESKPTFSRATALDNV

SEQ ID NO: 628 - protein, 275 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 629 - protein, 253 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 630 - protein, 308 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 631 - protein, 286 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 632 - protein, 367 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQLWIGLFLMVCLLCVGIFSWCIWKRKGRCSVPAFCSSQAEAPADTPEPTAGHTLYSVLSQGYEKLDTPLRPARQQPTPTSDSSSDSNLTTEEDEDRPEVHKPISGRYEVFDQVTQEGAGHDPAPEGQADYDPVTPYVTEVESVVGENTMYAQVFNLQGKTPVSQKEESSATIYCSIRKPQVVPPPQQNDLEIPESPTYENFT

SEQ ID NO: 633 - protein, 345 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQLWIGLFLMVCLLCVGIFSWCIWKRKGRCSVPAFCSSQAEAPADTPEPTAGHTLYSVLSQGYEKLDTPLRPARQQPTPTSDSSSDSNLTTEEDEDRPEVHKPISGRYEVFDQVTQEGAGHDPAPEGQADYDPVTPYVTEVESVVGENTMYAQVFNLQGKTPVSQKEESSATIYCSIRKPQVVPPPQQNDLEIPESPTYENFT

SEQ ID NO: 634 - protein, 424 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 635 - protein, 402 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 636 - protein, 375 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQIILGLFGLLLLLTCLCGTAWLCCSPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 637 - protein, 353 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQIILGLFGLLLLLTCLCGTAWLCCSPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 638 - protein, 621 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 639 - protein, 599 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 640 - protein, 612 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQIIIYCTGAFLISCMVGSVIVYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 641 - protein, 590 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQIIIYCTGAFLISCMVGSVIVYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 642 - protein, 248 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQGGTVLLLLFVISITTIIVIFLNRRRRRERRDLFTESWDTQKAPNNYRSPISTSQPTNQSMDDTREDIYVNYPTFSRRPKTRV

SEQ ID NO: 643 - protein, 226 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQGGTVLLLLFVISITTIIVIFLNRRRRRERRDLFTESWDTQKAPNNYRSPISTSQPTNQSMDDTREDIYVNYPTFSRRPKTRV

SEQ ID NO: 644 - protein, 225 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWLPIGCAAFVVVCILGCILICWLTKKKYSSSVHDPNGEYMFMRAVNTAKKSRLTDVTL

SEQ ID NO: 645 - protein, 203 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWLPIGCAAFVVVCILGCILICWLTKKKYSSSVHDPNGEYMFMRAVNTAKKSRLTDVTL

SEQ ID NO: 646 - protein, 212 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQMGLAFLVLVALVWFLVEDWLSRKRTRERASRASTWEGRRRLNTQTL

SEQ ID NO: 647 - protein, 190 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQMGLAFLVLVALVWFLVEDWLSRKRTRERASRASTWEGRRRLNTQTL

SEQ ID NO: 648 - protein, 250 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQLVPVFCGLLVAKSLVLSALLVWWGDIWWKTMMELRSLDTQKATCHLQQVTDLPWTSVSSPVEREILYHTVARTKISDDDDEHTL

SEQ ID NO: 649 - protein, 228 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQLVPVFCGLLVAKSLVLSALLVWWGDIWWKTMMELRSLDTQKATCHLQQVTDLPWTSVSSPVEREILYHTVARTKISDDDDEHTL

SEQ ID NO: 650 - protein, 232 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQAGTVLLLRAGFYAVSFLSVAVGSTVYYQGKCLTWKGPRRQLPAVVPAPLPPPCGSSAHLLPPVPGG

SEQ ID NO: 651 - protein, 210 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQAGTVLLLRAGFYAVSFLSVAVGSTVYYQGKCLTWKGPRRQLPAVVPAPLPPPCGSSAHLLPPVPGG

SEQ ID NO: 652 - protein, 662 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSNKPITPSTYVRCLNVGLIRKLSDFIDPQEGWKKLAVAIKKPSGDDRYNQFHIRRFEALLQTGKSPTSELLFDWGTTNCTVGDLVDLLIQNEFFAPASLLLPDAVPKTANTLPSKEAITVQQKQMPFCDKDRTLMTPVQNLEQSYMPPDSSSPENKSLEVSDTRFHSFSFYELKNVTNNFDERPISVGGNKMGEGGFGVVYKGYVNNTTVAVKKLAAMVDITTEELKQQFDQEIKVMAKCQHENLVELLGFSSDGDDLCLVYVYMPNGSLLDRLSCLDGTPPLSWHMRCKIAQGAANGINFLHENHHIHRDIKSANILLDEAFTAKISDFGLARASEKFAQTVMTSRIVGTTAYMAPEALRGEITPKSDIYSFGVVLLEIITGLPAVDEHREPQLLLDIKEEIEDEEKTIEDYIDKKMNDADSTSVEAMYSVASQCLHEKKNKRPDIKKVQQLLQEMTAS

SEQ ID NO: 653 - protein, 640 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSNKPITPSTYVRCLNVGLIRKLSDFIDPQEGWKKLAVAIKKPSGDDRYNQFHIRRFEALLQTGKSPTSELLFDWGTTNCTVGDLVDLLIQNEFFAPASLLLPDAVPKTANTLPSKEAITVQQKQMPFCDKDRTLMTPVQNLEQSYMPPDSSSPENKSLEVSDTRFHSFSFYELKNVTNNFDERPISVGGNKMGEGGFGVVYKGYVNNTTVAVKKLAAMVDITTEELKQQFDQEIKVMAKCQHENLVELLGFSSDGDDLCLVYVYMPNGSLLDRLSCLDGTPPLSWHMRCKIAQGAANGINFLHENHHIHRDIKSANILLDEAFTAKISDFGLARASEKFAQTVMTSRIVGTTAYMAPEALRGEITPKSDIYSFGVVLLEIITGLPAVDEHREPQLLLDIKEEIEDEEKTIEDYIDKKMNDADSTSVEAMYSVASQCLHEKKNKRPDIKKVQQLLQEMTAS

SEQ ID NO: 654 - protein, 374 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 655 - protein, 352 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 656 - protein, 384 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 657 - protein, 362 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 658 - protein, 837 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSASSGMADSANHLPFFFGNITREEAEDYLVQGGMSDGLYLLRQSRNYLGGFALSVAHGRKAHHYTIERELNGTYAIAGGRTHASPADLCHYHSQESDGLVCLLKKPFNRPQGVQPKTGPFEDLKENLIREYVKQTWNLQGQALEQAIISQKPQLEKLIATTAHEKMPWFHGKISREESEQIVLIGSKTNGKFLIRARDNNGSYALCLLHEGKVLHYRIDKDKTGKLSIPEGKKFDTLWQLVEHYSYKADGLLRVLTVPCQKIGTQGNVNFGGRPQLPGSHPATWSAGGIISRIKSYSFPKPGHRKSSPAQGNRQESTVSFNPYEPELAPWAADKGPQREALPMDTEVYESPYADPEEIRPKEVYLDRKLLTLEDKELGSGNFGTVKKGYYQMKKVVKTVAVKILKNEANDPALKDELLAEANVMQQLDNPYIVRMIGICEAESWMLVMEMAELGPLNKYLQQNRHVKDKNIIELVHQVSMGMKYLEESNFVHRDLAARNVLLVTQHYAKISDFGLSKALRADENYYKAQTHGKWPVKWYAPECINYYKFSSKSDVWSFGVLMWEAFSYGQKPYRGMKGSEVTAMLEKGERMGCPAGCPREMYDLMNLCWTYDVENRPGFAAVELRLRNYYYDVVN

SEQ ID NO: 659 - protein, 815 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSASSGMADSANHLPFFFGNITREEAEDYLVQGGMSDGLYLLRQSRNYLGGFALSVAHGRKAHHYTIERELNGTYAIAGGRTHASPADLCHYHSQESDGLVCLLKKPFNRPQGVQPKTGPFEDLKENLIREYVKQTWNLQGQALEQAIISQKPQLEKLIATTAHEKMPWFHGKISREESEQIVLIGSKTNGKFLIRARDNNGSYALCLLHEGKVLHYRIDKDKTGKLSIPEGKKFDTLWQLVEHYSYKADGLLRVLTVPCQKIGTQGNVNFGGRPQLPGSHPATWSAGGIISRIKSYSFPKPGHRKSSPAQGNRQESTVSFNPYEPELAPWAADKGPQREALPMDTEVYESPYADPEEIRPKEVYLDRKLLTLEDKELGSGNFGTVKKGYYQMKKVVKTVAVKILKNEANDPALKDELLAEANVMQQLDNPYIVRMIGICEAESWMLVMEMAELGPLNKYLQQNRHVKDKNIIELVHQVSMGMKYLEESNFVHRDLAARNVLLVTQHYAKISDFGLSKALRADENYYKAQTHGKWPVKWYAPECINYYKFSSKSDVWSFGVLMWEAFSYGQKPYRGMKGSEVTAMLEKGERMGCPAGCPREMYDLMNLCWTYDVENRPGFAAVELRLRNYYYDVVN

SEQ ID NO: 660 - protein, 714 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSGCIKSKGKDSLSDDGVDLKTQPVRNTERTIYVRDPTSNKQQRPVPESQLLPGQRFQTKDPEEQGDIVVALYPYDGIHPDDLSFKKGEKMKVLEEHGEWWKAKSLLTKKEGFIPSNYVAKLNTLETEEWFFKDITRKDAERQLLAPGNSAGAFLIRESETLKGSFSLSVRDFDPVHGDVIKHYKIRSLDNGGYYISPRITFPCISDMIKHYQKQADGLCRRLEKACISPKPQKPWDKDAWEIPRESIKLVKRLGAGQFGEVWMGYYNNSTKVAVKTLKPGTMSVQAFLEEANLMKTLQHDKLVRLYAVVTREEPIYIITEYMAKGSLLDFLKSDEGGKVLLPKLIDFSAQIAEGMAYIERKNYIHRDLRAANVLVSESLMCKIADFGLARVIEDNEYTAREGAKFPIKWTAPEAINFGCFTIKSDVWSFGILLYEIVTYGKIPYPGRTNADVMTALSQGYRMPRVENCPDELYDIMKMCWKEKAEERPTFDYLQSVLDDFYTATEGQYQQQP

SEQ ID NO: 661 - protein, 692 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSGCIKSKGKDSLSDDGVDLKTQPVRNTERTIYVRDPTSNKQQRPVPESQLLPGQRFQTKDPEEQGDIVVALYPYDGIHPDDLSFKKGEKMKVLEEHGEWWKAKSLLTKKEGFIPSNYVAKLNTLETEEWFFKDITRKDAERQLLAPGNSAGAFLIRESETLKGSFSLSVRDFDPVHGDVIKHYKIRSLDNGGYYISPRITFPCISDMIKHYQKQADGLCRRLEKACISPKPQKPWDKDAWEIPRESIKLVKRLGAGQFGEVWMGYYNNSTKVAVKTLKPGTMSVQAFLEEANLMKTLQHDKLVRLYAVVTREEPIYIITEYMAKGSLLDFLKSDEGGKVLLPKLIDFSAQIAEGMAYIERKNYIHRDLRAANVLVSESLMCKIADFGLARVIEDNEYTAREGAKFPIKWTAPEAINFGCFTIKSDVWSFGILLYEIVTYGKIPYPGRTNADVMTALSQGYRMPRVENCPDELYDIMKMCWKEKAEERPTFDYLQSVLDDFYTATEGQYQQQP

SEQ ID NO: 662 - protein, 745 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSGCIKSKENKSPAIKYRPENTPEPVSTSVSHYGAEPTTVSPCPSSSAKGTAVNFSSLSMTPFGGSSGVTPFGGASSSFSVVPSSYPAGLTGGVTIFVALYDYEARTTEDLSFKKGERFQIINNTEGDWWEARSIATGKNGYIPSNYVAPADSIQAEEWYFGKMGRKDAERLLLNPGNQRGIFLVRESETTKGAYSLSIRDWDEIRGDNVKHYKIRKLDNGGYYITTRAQFDTLQKLVKHYTEHADGLCHKLTTVCPTVKPQTQGLAKDAWEIPRESLRLEVKLGQGCFGEVWMGTWNGTTKVAIKTLKPGTMMPEAFLQEAQIMKKLRHDKLVPLYAVVSEEPIYIVTEFMSKGSLLDFLKEGDGKYLKLPQLVDMAAQIADGMAYIERMNYIHRDLRAANILVGENLVCKIADFGLARLIEDNEYTARQGAKFPIKWTAPEAALYGRFTIKSDVWSFGILQTELVTKGRVPYPGMVNREVLEQVERGYRMPCPQGCPESLHELMNLCWKKDPDERPTFEYIQSFLEDYFTATEPQYQPGENL

SEQ ID NO: 663 - protein, 723 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSGCIKSKENKSPAIKYRPENTPEPVSTSVSHYGAEPTTVSPCPSSSAKGTAVNFSSLSMTPFGGSSGVTPFGGASSSFSVVPSSYPAGLTGGVTIFVALYDYEARTTEDLSFKKGERFQIINNTEGDWWEARSIATGKNGYIPSNYVAPADSIQAEEWYFGKMGRKDAERLLLNPGNQRGIFLVRESETTKGAYSLSIRDWDEIRGDNVKHYKIRKLDNGGYYITTRAQFDTLQKLVKHYTEHADGLCHKLTTVCPTVKPQTQGLAKDAWEIPRESLRLEVKLGQGCFGEVWMGTWNGTTKVAIKTLKPGTMMPEAFLQEAQIMKKLRHDKLVPLYAVVSEEPIYIVTEFMSKGSLLDFLKEGDGKYLKLPQLVDMAAQIADGMAYIERMNYIHRDLRAANILVGENLVCKIADFGLARLIEDNEYTARQGAKFPIKWTAPEAALYGRFTIKSDVWSFGILQTELVTKGRVPYPGMVNREVLEQVERGYRMPCPQGCPESLHELMNLCWKKDPDERPTFEYIQSFLEDYFTATEPQYQPGENL

SEQ ID NO: 664 - protein, 731 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSGCVFCKKLEPVATAKEDAGLEGDFRSYGAADHYGPDPTKARPASSFAHIPNYSNFSSQAINPGFLDSGTIRGVSGIGVTLFIALYDYEARTEDDLTFTKGEKFHILNNTEGDWWEARSLSSGKTGCIPSNYVAPVDSIQAEEWYFGKIGRKDAERQLLSPGNPQGAFLIRESETTKGAYSLSIRDWDQTRGDHVKHYKIRKLDMGGYYITTRVQFNSVQELVQHYMEVNDGLCNLLIAPCTIMKPQTLGLAKDAWEISRSSITLERRLGTGCFGDVWLGTWNGSTKVAVKTLKPGTMSPKAFLEEAQVMKLLRHDKLVQLYAVVSEEPIYIVTEFMCHGSLLDFLKNPEGQDLRLPQLVDMAAQVAEGMAYMERMNYIHRDLRAANILVGERLACKIADFGLARLIKDDEYNPCQGSKFPIKWTAPEAALFGRFTIKSDVWSFGILLTELITKGRIPYPGMNKREVLEQVEQGYHMPCPPGCPASLYEAMEQTWRLDPEERPTFEYLQSFLEDYFTSAEPQYQPGDQT

SEQ ID NO: 665 - protein, 709 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSGCVFCKKLEPVATAKEDAGLEGDFRSYGAADHYGPDPTKARPASSFAHIPNYSNFSSQAINPGFLDSGTIRGVSGIGVTLFIALYDYEARTEDDLTFTKGEKFHILNNTEGDWWEARSLSSGKTGCIPSNYVAPVDSIQAEEWYFGKIGRKDAERQLLSPGNPQGAFLIRESETTKGAYSLSIRDWDQTRGDHVKHYKIRKLDMGGYYITTRVQFNSVQELVQHYMEVNDGLCNLLIAPCTIMKPQTLGLAKDAWEISRSSITLERRLGTGCFGDVWLGTWNGSTKVAVKTLKPGTMSPKAFLEEAQVMKLLRHDKLVQLYAVVSEEPIYIVTEFMCHGSLLDFLKNPEGQDLRLPQLVDMAAQVAEGMAYMERMNYIHRDLRAANILVGERLACKIADFGLARLIKDDEYNPCQGSKFPIKWTAPEAALFGRFTIKSDVWSFGILLTELITKGRIPYPGMNKREVLEQVEQGYHMPCPPGCPASLYEAMEQTWRLDPEERPTFEYLQSFLEDYFTSAEPQYQPGDQT

SEQ ID NO: 666 - protein, 236 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRKRGRKKLLYIFKQPFMRPVQTTQEEDGCSCRFPEEEEGGCEL

SEQ ID NO: 667 - protein, 214 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRKRGRKKLLYIFKQPFMRPVQTTQEEDGCSCRFPEEEEGGCEL

SEQ ID NO: 668 - protein, 217 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVLCARPRRSPAQEDGKVYINMPGRG

SEQ ID NO: 669 - protein, 195 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVLCARPRRSPAQEDGKVYINMPGRG

SEQ ID NO: 670 - protein, 234 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSDYMNMTPRRPGPTRKHYQPYAPPRDFAAYRS

SEQ ID NO: 671 - protein, 212 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRSKRSRLLHSDYMNMTPRRPGPTRKHYQPYAPPRDFAAYRS

SEQ ID NO: 672 - protein, 305 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 673 - protein, 283 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 674 - protein, 199 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 675 - protein, 177 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 676 - protein, 21 aa, synthetic construct
MALPVTALLLPLALLLHAARP

SEQ ID NO: 677 - protein, 231 aa, synthetic construct
MSKQRGTFSEVSLAQDPKRQQRKPKGNKSSISGTEQEIFQVELNLQNPSLNHQGIDKIYDCQGLLPPPEKLTAEVLGIICIVLMATVLKTIVLIPFLEQNNSSPNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 678 - protein, 391 aa, synthetic construct
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 679 - protein, 366 aa, synthetic construct
QVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 680 - protein, 472 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 681 - protein, 450 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 682 - protein, 272 aa, synthetic construct
MALPVTALLLPLALLLHAARPTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 683 - protein, 251 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 684 - protein, 221 aa, synthetic construct
MALPVTALLLPLALLLHAARPLQCFCHLCTKDNFTCVTDGLCFVSVTETTDKVIHNSMCIAEIDLIPRDRPFVCAPSSKTGSVTTTYCCNQDHCNKIELPTTVKSSPGLGPVELVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 685 - protein, 200 aa, synthetic construct
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVELVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 686 - protein, 432 aa, synthetic construct
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYISLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 687 - protein, 399 aa, synthetic construct
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYISLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS